(19) United States
(12) Patent Application Publication
(10) Pub. No.: US 2017/0107577 A1
(43) Pub. Date: Apr. 20, 2017

(54) Determining Cancer Aggressiveness, Prognosis and Responsiveness to Treatment

(71) Applicant: THE COUNCIL OF THE QUEENSLAND INSTITUTE OF MEDICAL RESEARCH, Herston, Queensland (AT)

(72) Inventor: Fares Al-Ejeh, Herston, Queensland (AU)

(21) Appl. No.: 15/125,515

(22) PCT Filed: Mar. 11, 2015
(86) PCT No.: PCT/AU2015/050096

§ 371 Date: Sep. 12, 2016

(30) Foreign Application Priority Data
Mar. 11, 2014 (AU) 2014900813
Apr. 3, 2014 (AU) 2014901212
Nov. 21, 2014 (AU) 2014904716

(51) Int. Cl. C12Q 1/68; C07K 16/28; G01N 33/574

(52) U.S. Cl. CPC C12Q 1/6886; G01N 33/57415; C07K 16/2887; C07K 16/2818; C12Q 2600/158; C12Q 2600/118; C07K 2317/24

(57) ABSTRACT

The invention provides methods of determining the aggressiveness, prognosis and response to therapy for particular cancers, which include comparing the expression levels of one or a plurality of differentially expressed genes from one or more 5 functional metagenes, including a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune system metagene, a Metabolic Disease metagene, a Nucleic Acid Metabolism metagene, a Post-Translational Modification metagene, a Protein 10 Synthesis/Modification metagene and a Multiple Networks metagene. The method disclosed herein may be particularly suitable as a companion diagnostic for cancer therapies.

### FIELD

THIS INVENTION relates to cancer. More particularly, this invention relates to methods of determining the aggressiveness of cancers, prognosis of cancers and/or predicting responsiveness to anti-cancer therapy.

### BACKGROUND

Hormone receptors (ER and PR) and HER2 are standard biomarkers used in clinical practice to aid the histopathological classification of breast cancer and management decisions. Hormone receptor (HR)− and HER2− positive tumors benefit from tamoxifen and anti-HER2 therapies, respectively. On the other hand, there are currently no targeted drug therapies for management of triple negative breast cancer (TNBC), which lacks expression of HR/HER2. TNBCs are more sensitive to chemotherapy than HR-positive tumors because they are generally more proliferative, and pathological complete responses (pCR) after chemotherapy are more likely in TNBC than in non-TNBC^{1,2}. Paradoxically, TNBC is associated with poorer survival than non-TNBC, due to more frequent relapse in TNBC patients with residual disease^{1,2}. Only 31% of TNBC patients experience pCR after chemotherapy³, emphasizing the need for targeted therapies.

Transcriptome profiling has been used to dissect the heterogeneity of breast cancer into five intrinsic ‘PAM50’ subtypes; Luminal A, Luminal B, Basal-like, HER-2 and normal-like subtypes that relate to clinical outcomes⁴⁻⁸. Several gene signatures have been developed to predict outcome or response to treatment including: MammaPrint⁹, OncotypeDx^{10,11}, Theros¹²⁻¹⁵. These commercial signatures rely on models that select genes based on clinical phenotypes such as tumor response or survival time. Notwithstanding their clinical utilities, these models fail to identify core biological mechanisms for the phenotypes of interest. Recently, an approach based on biological function-driven gene coexpression signatures, “attractor metagenes”, has been applied to the prediction of survival in certain cancers. However such approaches are at an early stage and much work needs to be done to develop this attractor metagene analysis in relation to cancers in general and also for specific cancers.

### SUMMARY

The present invention relates to the comparison of expression levels of a plurality of differentially expressed genes from one or a plurality of functional metagenes, including a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune system metagene, a Metabolic Disease metagene, a Nucleic Acid Metabolism metagene, a Post-Translational Modification metagene, a Protein Synthesis/Modification metagene and a Multiple Networks metagene; wherein the comparison of expression level of a plurality of genes in these metagenes is used to facilitate determining the aggressiveness of certain cancers. This comparison may also, or alternatively, assist in providing a cancer prognosis for a patient. The invention also relates to predicting the responsiveness of a cancer to an anti-cancer treatment by determining an expression level of one or a plurality of genes associated with one or a plurality of the aforementioned twelve functional metagenes.

The invention further relates to the comparison of expression levels of a specific signature of differentially expressed proteins to facilitate or assist in determining the aggressiveness of a particular cancer, a prognosis for a cancer patient and/or predicting responsiveness to an anti-cancer treatment. One or both of these comparisons may also be integrated with the aforementioned comparison of the expression levels of the plurality genes from one or a plurality of the aforementioned functional metagenes in determining cancer aggressiveness, prognosis and/or treatment.

In a first aspect, the invention relates to a method of determining the aggressiveness of a cancer in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level.

In a second aspect, the invention relates to a method of determining a cancer prognosis for a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis.

In one embodiment of the above aspects, the one or plurality of overexpressed genes and/or the one or plurality of underexpressed genes are selected from one of the aforesaid metagenes. In an alternative embodiment, the one or plurality of overexpressed genes and/or one or the plurality of underexpressed genes are selected from a plurality of the aforesaid metagenes.

Suitably, for the method of the above aspects the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or a plurality of genes listed in Table 21.

In a third aspect, the invention relates to a method of determining the aggressiveness of a cancer in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level

In a fourth aspect, the invention relates to a method of determining a cancer prognosis for a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis.

In one embodiment of the third and fourth aspects, the one or plurality of overexpressed genes and/or the one or plurality of underexpressed genes are selected from one of the aforesaid metagenes. In an alternative embodiment, the one or plurality of overexpressed genes and/or the one or plurality of underexpressed genes are selected from a plurality of the aforesaid metagenes.

Suitably, the Metabolism metagene, the Signalling metagene, the Development and Growth metagene, the Chromosome Segregation/Replication metagene, the Immune Response metagene and/or the Protein Synthesis/Modification metagene comprise one or a plurality of genes listed in Table 22.

In particular embodiments of the method of the third and fourth aspects, the one or plurality of overexpressed genes and/or the one or plurality of underexpressed genes are from one or a plurality of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene.

In a fifth aspect, the invention relates to a method of determining the aggressiveness of a cancer in a mammal, said method including the step of comparing an expression level of one or a plurality of overexpressed genes associated with chromosomal instability and/or an expression level of one or a plurality of underexpressed genes associated with estrogen receptor signalling in one or a plurality of cancer cells, tissues or organs of the mammal, wherein: a higher relative expression level of the one or plurality of overexpressed genes associated with chromosomal instability compared to the one or plurality of underexpressed genes associated with estrogen receptor signalling indicates or correlates with higher aggressiveness of the cancer; and/or a lower relative expression level expression level of the one or plurality of overexpressed genes associated with chromosomal instability compared to the one or plurality of underexpressed genes associated with estrogen receptor signalling indicates or correlates with lower aggressiveness of the cancer compared to a mammal having a higher expression level.

In a sixth aspect, the invention relates to a method of determining a cancer prognosis for a mammal, said method including the step of comparing an expression level of one or a plurality of overexpressed genes associated with chromosomal instability and/or an expression level of one or a plurality of underexpressed genes associated with estrogen receptor signalling in one or a plurality of cancer cells, tissues or organs of the mammal, wherein: a higher relative expression level of the one or plurality of overexpressed genes associated with chromosomal instability compared to the one or plurality of underexpressed genes associated with estrogen receptor signalling indicates or correlates with a less favourable cancer prognosis; and/or a lower relative expression level of the one or plurality of overexpressed genes associated with chromosomal instability compared to the one or plurality of underexpressed genes associated with estrogen receptor signalling indicates or correlates with a more favourable cancer prognosis.

In certain embodiments, the genes associated with chromosomal instability are of a CIN metagene. Non-limiting examples include genes selected from the group consisting of ATP6V1C1, RAP2A, CALM1, COG8, HELLS, KDM5A, PGK1, PLCH1, CEP55, RFC4, TAF2, SF3B3, GP1, PIR, MCM10, MELK, FOXM1, KIF2C, NUP155, TPX2, TTK, CENPA, CENPN, EXO1, MAPRE1, ACOT7, NAE1, SHMT2, TCP1, TXNRD1, ADM, CHAF1A and SYNCRIP. Preferably, the genes are selected from the group consisting of: MELK, MCM10, CENPA, EXO1, TTK and KIF2C.

In certain embodiments, the genes associated with estrogen receptor signalling are of an ER metagene. Non-limiting examples include genes selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3. Preferably, the genes are selected from the group consisting of: MAPT and MYB.

In certain embodiments, the method of the fifth and sixth aspects further including the step of comparing an expression level of one or a plurality of other overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of other underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein: a higher relative expression level of the other overexpressed genes compared to the other underexpressed genes indicates or correlates with higher aggressiveness of the cancer and/or a less favourable cancer prognosis; and/or a lower relative expression level of the other overexpressed genes compared to the other underexpressed genes indicates or correlates with lower aggressiveness of the cancer and/or a more favourable cancer prognosis compared to a mammal having a higher expression level.

In one embodiment, the one or plurality of other overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or plurality of other underexpressed genes are selected from the group consisting of BTN2A2, ERC2, IGH, ME1, MTMR7, SMPDL3B and ZNRD1-AS1.

Suitably, the comparison of the expression level of the overexpressed genes associated with chromosomal instability and/or the expression level of the underexpressed genes associated with estrogen receptor signalling is integrated with the comparison of the expression level of the one or plurality of other overexpressed genes and/or the expression level of the one or plurality of other underexpressed genes to derive a first integrated score.

In a seventh aspect, the invention provides a method of determining the aggressiveness of a cancer in a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level.

In an eighth aspect, the invention provides a method of determining a cancer prognosis for a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis compared to a mammal having a higher expression level.

In one embodiment of the seventh and eighth aspects, the one or plurality of overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment of the seventh and eighth aspects, the one or plurality of underexpressed genes are selected from the group consisting of BTN2A2, ERC2, IGH, ME1, MTMR7, SMPDL3B and ZNRD1-AS1.

In particular embodiments, the method of the first, second, third, fourth, fifth, sixth, seventh and eighth aspects further includes the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein: a higher relative expression level of the overexpressed proteins compared to the underexpressed proteins indicates or correlates with higher aggressiveness of the cancer and/or a less favourable cancer prognosis; and/or a lower relative expression level of the overexpressed proteins compared to the underexpressed proteins indicates or correlates with lower aggressiveness of the cancer and/or a more favourable cancer prognosis compared to a mammal having a higher expression level.

Suitably, the comparison of the expression level of the one or plurality of overexpressed proteins and/or the expression level of the one or plurality of underexpressed proteins is to thereby derive an integrated score. In one particular embodiment, the comparison of the expression level of the one or plurality of overexpressed proteins and/or the expression level of the one or plurality of underexpressed proteins is integrated with:
the comparison of the expression level of the overexpressed genes associated with chromosomal instability and/or the expression level of the underexpressed genes associated with estrogen receptor signalling to derive a second integrated score; or
(ii) the first integrated score to derive a third integrated score; or
(iii) the comparison of the expression level of the overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1 and/or the expression level of the underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 to derive a fourth integrated score; or
(iv) the comparison of the expression level of the overexpressed genes and/or an expression level of the underexpressed genes, wherein the genes are from one or a plurality of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene, to derive a fifth integrated score; or
(v) the comparison of the expression level of the overexpressed genes and/or the expression level of the underexpressed genes, wherein the genes are from one or a plurality of the Metabolism metagene, the Signalling metagene, the Development and Growth metagene, the Chromosome Segregation/Replication metagene, the Immune Response metagene and/or the Protein Synthesis/Modification metagene, to derive a sixth integrated score.
wherein the second, third, fourth, fifth and/or sixth integrated score is indicative of, or correlates with, the aggressiveness and/or prognosis of the cancer in the mammal.

In particular embodiments, the second, third, fourth, fifth and/or sixth integrated score are derived, at least in part, by addition, subtraction, multiplication, division and/or exponentiation.

In a preferred embodiment, the first, second and/or third integrated scores are derived, at least in part, by exponentiation wherein the comparison of the expression level of the other overexpressed genes and the expression level of the other underexpressed genes is raised to the power of
(i) the comparison of the expression level of the overexpressed genes associated with chromosomal instability and/or the expression level of the underexpressed genes associated with estrogen receptor signalling; and/or
(ii) the comparison of the expression level of the overexpressed proteins and/or the expression level of the underexpressed proteins.

In a ninth aspect, the invention provides a method of determining the aggressiveness of a cancer in a mammal, said method including the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein: a higher relative expression level of the one or plurality of overexpressed proteins compared to the one or plurality of underexpressed proteins indicates or correlates with higher aggressiveness of the cancer; and/or a lower relative expression level of the one or plurality of overexpressed proteins compared to the one or plurality of underexpressed proteins indicates or correlates with lower aggressiveness of the cancer compared to a mammal having a higher expression level.

In a tenth aspect, the invention provides a method of determining a cancer prognosis for a mammal, said method including the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein: a higher relative expression level of the one or plurality of overexpressed proteins compared to the one or plurality of underexpressed proteins indicates or correlates with a less favourable cancer prognosis; and/or a lower relative expression level of the one or plurality of overexpressed proteins compared to the one or plurality of underexpressed proteins indicates or correlates with a more favourable cancer prognosis compared to a mammal having a higher expression level.

In an eleventh aspect, the invention provides method of predicting the responsiveness of a cancer to an anti-cancer treatment in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein an altered or modulated relative expression level of the overexpressed genes compared to the underexpressed genes indicates or correlates with relatively increased or decreased responsiveness of the cancer to the anti-cancer treatment.

Suitably, for the present aspect the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or a plurality of genes listed in Table 21.

In a twelfth aspect, the invention provides a method of predicting the responsiveness of a cancer to an anti-cancer treatment in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein an altered or modulated relative expression level of the overexpressed genes compared to the underexpressed genes indicates or correlates with relatively increased or decreased responsiveness of the cancer to the anti-cancer treatment.

In one embodiment of the eleventh and twelfth aspects, the one or plurality of overexpressed genes and/or the one or plurality of underexpressed genes are selected from one of the metagenes. In an alternative embodiment, the one or plurality of overexpressed genes and/or the one or plurality of underexpressed genes are selected from a plurality of the metagenes.

Suitably, the Metabolism metagene, the Signalling metagene, the Development and Growth metagene, the Chromosome Segregation/Replication metagene, the Immune Response metagene and/or the Protein Synthesis/Modification metagene comprise one or a plurality of genes listed in Table 22.

In particular embodiments, the one or plurality of overexpressed genes and the one or plurality of underexpressed genes are from one or a plurality of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene.

According to the method of the eleventh and twelfth aspects, the step of comparing an expression level of one or a plurality of overexpressed genes and/or an expression level of one or a plurality of underexpressed genes includes comparing an average expression level of the one or plurality of overexpressed genes and/or an average expression level of the one or plurality of underexpressed genes. This may include calculating a ratio of the average expression level of the one or plurality of overexpressed genes and the average expression level of the one or plurality of underexpressed genes. Suitably, the ratio provides an aggressiveness score which is indicative of, or correlates with, cancer aggressiveness and a less favourable prognosis. Alternatively, the step of comparing an expression level of one or a plurality of overexpressed genes and/or an expression level of one or a plurality of underexpressed genes includes comparing the sum of expression levels of the one or plurality of overexpressed genes and/or the sum of expression levels of the one or plurality of underexpressed genes. This may include calculating a ratio of the sum of expression levels of the one or plurality of overexpressed genes and/or the sum of expression levels of the one or plurality of underexpressed genes.

In a thirteenth aspect, the invention provides a method of predicting the responsiveness of a cancer to an anti-cancer treatment in a mammal, said method including the step of determining an expression level of one or a plurality of genes associated with chromosomal instability in one or a plurality of non-mitotic cancer cells of the mammal, wherein a higher expression level indicates or correlates with relatively increased responsiveness of the cancer to the anti-cancer treatment

Suitably, the one or plurality of genes associated with chromosomal instability are selected from the group consisting of: TTK, CEP55, FOXM1 and SKIP2 and/or any CIN genes listed in Table 4.

In a fourteenth aspect, the invention provides a method of predicting the responsiveness of a cancer to an anti-cancer treatment in a mammal, said method including the step of comparing an expression level of one or a plurality of overexpressed genes associated with chromosomal instability and/or an expression level of one or a plurality of underexpressed genes associated with estrogen receptor signalling in one or a plurality of cancer cells, tissues or organs of the mammal, wherein an altered or modulated relative expression level of the one or plurality of overexpressed genes associated with chromosomal instability compared to the one or plurality of underexpressed genes associated with estrogen receptor signalling indicates or correlates with relatively increased or decreased responsiveness of the cancer to the anti-cancer treatment.

In certain embodiments, the genes associated with chromosomal instability are of a CIN metagene. Non-limiting examples include genes selected from the group consisting of: ATP6V1C1, RAP2A, CALM1, COG8, HELLS, KDM5A, PGK1, PLCH1, CEP55, RFC4, TAF2, SF3B3, GP1, PIR, MCM10, MELK, FOXM1, KIF2C, NUP155, TPX2, 11K, CENPA, CENPN, EXO1, MAPRE1, ACOT7, NAE1, SHMT2, TCP1, TXNRD1, ADM, CHAF1A and SYNCRIP. Preferably, the genes are selected from the group consisting of: MELK, MCM10, CENPA, EXO1, TTK and KIF2C.

In certain embodiments, the genes associated with estrogen receptor signalling are of an ER metagene. Non-limiting examples include genes selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3. Preferably, the genes are selected from the group consisting of: MAPT and MYB.

Suitably, the method of this aspect further includes the step of comparing an expression level of one or a plurality of other overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of other underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 in one or a plurality of cancer cells, tissues or organs of the mammal, wherein an altered or modulated relative expression level of the one or plurality of other overexpressed genes compared to the one or plurality of other underexpressed genes indicates or correlates with relatively increased or decreased responsiveness of the cancer to the anti-cancer treatment.

In one embodiment, the one or plurality of other overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or plurality of other underexpressed genes are selected from the group consisting of BTN2A2, ERC2, IGH, ME1, MTMR7, SMPDL3B and ZNRD1-AS1.

In certain embodiments, the comparison of the expression level of the one or plurality of other overexpressed genes and/or the expression level of the one or plurality of other underexpressed genes is integrated with the comparison of the expression level of the one or plurality of overexpressed genes associated with chromosomal instability and/or the expression level of the one or plurality of underexpressed genes associated with estrogen receptor signalling to derive a first integrated score, which is indicative of, or correlates with, responsiveness of the cancer to the anti-cancer treatment. By way of example, the first integrated score may be derived, at least in part, by addition, subtraction, multiplication, division and/or exponentiation. Preferably, the integrated score is derived by exponentiation, wherein the comparison of the expression level of the one or plurality of other overexpressed genes and the expression level of the one or plurality of other underexpressed genes is raised to the power of the comparison of the expression level of the one or plurality of overexpressed genes associated with chromosomal instability and the expression level of the one or plurality of underexpressed genes associated with estrogen receptor signalling.

In a fifteenth aspect, the invention provides a method of predicting the responsiveness of a cancer to an anti-cancer treatment in a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the anti-cancer treatment.

In one embodiment, the one or plurality of overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or plurality of underexpressed genes are selected from the group consisting of BTN2A2, ERC2, IGH, ME1, MTMR7, SMPDL3B and ZNRD1-AS1.

Suitably, the method of the eleventh, twelfth, thirteenth, fourteenth and fifteenth aspects further includes the step of comparing an expression level of a one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein an altered or modulated relative expression level of the one or plurality of overexpressed proteins compared to the one or plurality of underexpressed proteins indicates or correlates with relatively increased or decreased responsiveness of the cancer to the anti-cancer treatment.

Suitably, the comparison of the expression level of the one or plurality of overexpressed proteins and/or the expression level of the one or plurality of underexpressed proteins is to thereby derive an integrated score. In one particular embodiment, the comparison of the expression level of the one or plurality of overexpressed proteins and/or the expression level of the one or plurality of underexpressed proteins is integrated with:
(i) the comparison of the expression level of the overexpressed genes associated with chromosomal instability and/or the expression level of the underexpressed genes associated with estrogen receptor signalling to derive a second integrated score; or
(ii) the first integrated score to derive a third integrated score; or
(iii) the comparison of the expression level of the overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1 and/or the expression level of the underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 to derive a fourth integrated score; or
(iv) the comparison of the expression level of the overexpressed genes and an expression level of the underexpressed genes, wherein the genes are from one or a plurality of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene, to derive a fifth integrated score; or
(v) the comparison of the expression level of the overexpressed genes and an expression level of the underexpressed genes, wherein the genes are from one or a plurality of the Metabolism metagene, the Signalling metagene, the Development and Growth metagene, the Chromosome Segregation/Replication metagene, the Immune Response metagene and/or the Protein Synthesis/Modification metagene, to derive a sixth integrated score.
   wherein the second, third, fourth, fifth and/or sixth integrated score is indicative of, or correlates with, responsiveness of the cancer to the anti-cancer treatment.

In particular embodiments the first, second, third, fourth, fifth and/or sixth integrated score are derived, at least in part, by addition, subtraction, multiplication, division and/or exponentiation.

In a preferred embodiment, the first, second and/or third integrated scores are derived, at least in part, by exponentiation wherein the comparison of the expression level of the other overexpressed genes and/or the expression level of the other underexpressed genes is raised to the power of
(i) the comparison of the expression level of the overexpressed genes associated with chromosomal instability and/or the expression level of the underexpressed genes associated with estrogen receptor signalling; and/or
(ii) the comparison of the expression level of the overexpressed proteins and/or the expression level of the underexpressed proteins.

In a sixteenth aspect, the invention provides method of predicting the responsiveness of a cancer to an anti-cancer treatment in a mammal, said method including the step of comparing an expression level of one or a plurality of overexpressed proteins selected from the group consisting of DVL3, PM-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein an altered or modulated relative expression level of the one or plurality of overexpressed proteins compared to the one or plurality of underexpressed proteins indicates or correlates with relatively increased or decreased responsiveness of the cancer to the anti-cancer treatment.

Suitably, the anticancer treatment of the eleventh, twelfth, thirteenth, fourteenth, fifteenth and sixteenth aspects is selected from the group consisting of endocrine therapy, chemotherapy, immunotherapy and a molecularly targeted therapy. In certain embodiments, the anticancer treatment comprises an anaplastic lymphoma kinase (ALK) inhibitor, a BCR-ABL inhibitor, a heat shock protein 90 (HSP90) inhibitor, an epidermal growth factor receptor (EGFR) inhibitor, a poly (ADP-ribose) polymerase (PARP) inhibitor, retinoic acid, a B-cell lymphoma 2 (Bcl2) inhibitor, a gluconeogenesis inhibitor, a p38 mitogen-activated protein kinase (MAPK) inhibitor, a mitogen-activated protein kinase kinase 1/2 (MEK1/2) inhibitor, a mammalian target of rapamycin (mTOR) inhibitor, a phosphatidylinositol-4,5-bisphosphate 3-kinase (PI3K) inhibitor, an insulin-like growth factor 1 receptor (IGF1R) inhibitor, a phospholipase C-γ (PLCγ) inhibitor, a c-Jun N-terminal kinase (JNK) inhibitor, a p21-activated kinase-1 (PAK1) inhibitor, a spleen tyrosine kinase (SYK) inhibitor, a histone deacetylase (HDAC) inhibitor, a fibroblast growth factor receptor (FGFR) inhibitor, an X-linked inhibitor of apoptosis (XIAP) inhibitor, a polo-like kinase 1 (PLK1) inhibitor, an extracellular-signal-regulated kinase 5 (ERK5) inhibitor and combinations thereof.

Suitably, the method of the eleventh, twelfth, thirteenth, fourteenth, fifteenth and sixteenth aspects further includes the step of administering to the mammal a therapeutically effective amount of the anticancer treatment. Preferably, the anticancer treatment is administered when the altered or modulated relative expression level indicates or correlates with relatively increased responsiveness of the cancer to the anti-cancer treatment.

In a seventeenth aspect, the invention provides a method of predicting the responsiveness of a cancer to an immunotherapeutic agent in a mammal, said method including the step of comparing an expression level of one or a plurality of overexpressed genes selected from the group consisting of ADORA2B, CD36, CETN3, CFDP1, KCNG1, LAMA3, NAE1, MAP2K5, PGK1, SF3B3, STAU1 and TXN and/or an expression level of one or a plurality of underexpressed genes selected from the group consisting of APOBEC3A, BTN2A2, BCL2, CAMK4, FBXW4, CAMSAP1, CARHSP1, GSK3B, HCFC1R1, PSEN2, MYB and ZNF593, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the immunotherapeutic agent.

Suitably, the immunotherapeutic agent is an immune checkpoint inhibitor. Preferably, the immune checkpoint inhibitor is or comprises an anti-PD1 antibody or an anti-PDL1 antibody.

In an eighteenth aspect is provided a method of predicting the responsiveness of a cancer to an epidermal; growth factor receptor (EGFR) inhibitor in a mammal, said method including the step of comparing an expression level of one or a plurality of overexpressed genes selected from the group consisting of NAE1, GSK3B, TAF2, MAPRE1, BRD4, STAU1, TAF2, PDCD4, KCNG1, ZNRD1-AS1, EIF4B, HELLS, RPL22, ABAT, BTN2A2, CD1B, ITM2A, BCL2, CXCR4, and ARNT2 and/or an expression level of one or a plurality of underexpressed genes selected from the group consisting of CD1C, CD1E, CD1B, KDM5A, BATF, EVL, PRKCB, HCFC1R1, CARHSP1, CHAD, KIR2DL4, ABHD5, ABHD14A, ACAA1, SRPK3, CFB, ARNT2, NDUFC1, BCL2, EVL, ULBP2, BIN3, SF3B3, CETN3, SYNCRIP, TAF2, CENPN, ATP6V1C1, CD55 and ADORA2B in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the EGFR inhibitor.

In a nineteenth aspect is provided a method of predicting the responsiveness of a cancer to a multikinase inhibitor in a mammal, said method including the step of comparing an expression level of one or a plurality of overexpressed genes selected from the group consisting of SCUBE, CHPT1, CDC1, BTG2, ADORA2B and BCL2, and/or an expression level of one or a plurality of underexpressed genes selected from the group consisting of NOP2, CALR, MAPRE1, KCNG1, PGK1, SRPK3, RERE, ADM, LAMA3, KIR2DL4, ULBP2, LAMA4, CA9, and BCAP31, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the multikinase inhibitor.

Suitably, for the method of the seventeenth, eighteenth and nineteenth aspects, a higher relative expression level of the one or plurality of overexpressed genes compared to the one or plurality of underexpressed genes indicates or correlates with a relatively increased responsiveness of the cancer to the immunotherapeutic agent, EGFR inhibitor or multikinase inhibitor; and/or a lower relative expression level of the one or aplurality of overexpressed genes compared to the one or plurality of underexpressed genes indicates or correlates with a relatively decreased responsiveness of the cancer to the immunotherapeutic agent, EGFR inhibitor and/or multikinase inhibitor.

In some embodiments, the method of the seventeenth, eighteenth and nineteenth aspects further includes the step of administering to the mammal a therapeutically effective amount of the immunotherapeutic agent, the EGFR inhibitor or the multikinase inhibitor respectively. Preferably, the immunotherapeutic agent, the EGFR inhibitor or the multikinase inhibitor is administered when the altered or modulated relative expression level indicates or correlates with relatively increased responsiveness of the cancer to the immunotherapeutic agent, the EGFR inhibitor or the multikinase inhibitor respectively.

Suitably, for the methods of the aforementioned aspects, the step of comparing an expression level of one or a plurality ofoverexpressed genes or proteins and an expression level of one or a plurality of underexpressed genes or proteins, includes comparing an average expression level of the one or plurality of overexpressed genes or proteins and an average expression level of the one or plurality of underexpressed genes or proteins. This may include calculating a ratio of the average expression level of the one or plurality of overexpressed genes or proteins and the average expression level of the one or plurality of underexpressed genes or proteins. Suitably, the ratio provides an aggressiveness score which is indicative of, or correlates with, cancer aggressiveness and a less favourable prognosis. Alternatively, the step of comparing an expression level of one or a plurality of overexpressed genes and an expression level of one or a plurality of underexpressed genes or proteins, includes comparing the sum of expression levels of the one or plurality of overexpressed genes or proteins and the sum of expression levels of the one or plurality of underexpressed genes or proteins. This may include calculating a ratio of the sum of expression levels of the one or plurality of overexpressed genes or protein and the sum of expression levels of the one or plurality of underexpressed genes or proteins.

In certain embodiments of the aforementioned methods, the mammal is subsequently treated for cancer.

In a twentieth aspect, the invention provides a method for identifying an agent for use in the treatment of cancer including the steps of:

(i) contacting a protein product of GRHPR, NDUFC1, CAMSAP1, CETN3, EIF3K, STAU1, EXOSC7, COG8, CFDP1 and/or KCNG1 with a test agent; and

(ii) determining whether the test agent, at least partly, reduces, eliminates, suppresses or inhibits the expression and/or an activity of the protein product.

Suitably, the agent possesses or displays little or no significant off-target and/or nonspecific effects.

Preferably, the agent is an antibody or a small organic molecule.

In a twenty first aspect, the invention provides an agent for use in the treatment of cancer identified by the method of the eighteenth aspect.

In a twenty second aspect, the invention provides a method of treating a cancer in a mammal, including the step of administering to the mammal a therapeutically effective amount of an agent identified by the method of the eighteenth aspect.

Preferably, for the invention of the twentieth, twenty first and twenty second aspects, the cancer has an overexpressed gene selected from the group consisting of GRHPR, NDUFC1, CAMSAP1, CETN3, EIF3K, STAU1, EXOSC7, COG8, CFDP1, KCNG1 and any combination thereof.

Suitably, the method of the aformentioned aspects further includes the step of determining, assessing or measuring the expression level of one or plurality of the overexpressed genes, the underexpressed genes, the overexpressed proteins and/or the underexpressed proteins described herein.

Suitably, the mammal referred to in the aforementioned aspects and embodiments is a human.

In certain embodiments of the invention of the aforementioned aspects, the cancer includes breast cancer, lung cancer inclusive of lung adenocarcinoma and lung squamous cell carcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer inclusive of hepatocellular carcinoma, kidney cancer inclusive of renal clear cell carcinoma and renal papillary cell carcinoma, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto. By way of example, breast cancer includes aggressive breast cancers and cancer subtypes such as triple negative breast cancer, grade 2 breast cancer, grade 3 breast cancer, lymph node positive (LN⁺) breast cancer, HER2 positive (HER2⁺) breast cancer and ER positive (ER⁺) breast cancer, although without limitation thereto.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

The indefinite articles ‘a’ and ‘an’ are used here to refer to or encompass singular or plural elements or features and should not be taken as meaning or defining “one” or a “single” element or feature.

### BRIEF DESCRIPTION OF THE FIGURES

FIG. 1: Correlation of breast cancer subtypes and the aggressiveness gene list. The METABRIC dataset was visualized according to the expression of the 206 genes (Table 4) in the aggressiveness gene list. The aggressiveness score for each tumor was calculated as the ratio of the CIN metagene (average value for CIN genes expression) to the ER metagene (average value for ER genes expression). (A) The expression of the aggressiveness gene list according to the GENIUS histological classification. Box plot shows the aggressiveness score of the histological subtypes. (B) The overall survival of patients in the METABRIC dataset was analyzed according to the aggressiveness score (upper row: by quartiles; lower row: by median) in all patients, non-TNBC patients and in patients with ER+ Grade 2 tumors. The hazard ratio (HR) and confidence interval (CI) and p-value for comparisons of upper quartile vs. lower quartiles (upper row) and at the dichotomy across the median (high vs. low) are shown (Log-rank Test, GraphPad® Prism). The number of patients (n) in each group is shown in brackets.

FIG. 2: Network analysis of the aggressiveness gene list. (A) Ingenuity pathway analysis was performed using direct interactions on the 206 genes in the aggressiveness gene list (red is overexpressed and green is underexpressed). One network of high direct interactions was identified. (B) The genes in the network in A were investigated for their correlation with the aggressiveness score and overall survival (Table 5) and eight genes (MAPT, MYB, MELK, MCM10, CENPA, EXO1, TTK and KIF2C) with the highest correlation were still connected in a direct interaction network. (C) The overall survival of patients in the METABRIC dataset was analyzed according to score from the 8 genes in C (upper row: by quartiles; lower row: by median) in all patients, non-TNBC patients and in patients with ER+ Grade 2 tumors.

FIG. 3: Survival of patients stratified by the 8-genes score in the METABRIC dataset. The overall survival of patients in the METABRIC dataset was analyzed according to the 8-genes score in selected settings in all patients (A) or in ER-positive patients only (B). (A) TP53 mutation was compared in high vs. low 8-genes score (split by the median). The expression of the proliferation marker Ki67 was divided by dichotomy across the median and patients in each of these groups were then stratified according to their 8-genes score (split by quartiles). Disease stages (Stage I-Stage III) were stratified by the median 8-genes score. (B) ER⁺ Grade 3, ER+ lymph node negative (LN−) and ER+ LN+ tumors were stratified by the quartiles.

FIG. 4: The 8-genes score associates with survival of breast cancer patients. Four published datasets were used to validate the 8-genes score as a predictor of survival. The 8-genes score was calculated for tumors in each of the datasets and the survival of patients was stratified according to the median 8-genes score; (A) GSE2990¹⁵, (B) GSE3494⁶⁵, (C) GSE2034⁶⁶ and (D) GSE25066⁵³. The hazard ratio (HR) and confidence interval (CI) and p-value for comparisons high vs. low 8-genes score are shown in the Kaplan-Meier survival curves (Log-rank Test, GraphPad® Prism). The number of patients (n) is shown in brackets. The table in each panel show multivariate survival analysis in the using Cox-proportional hazard model including all available conventional indicators.

FIG. 5: Therapeutic targets in the aggressiveness gene list. (A) The TNBC cell lines, MDA-MB-231, SUM159PT and Hs578T were treated with control siRNA (Scrambled, Sc CTRL) or siRNA targeting the specified genes and the survival of these cells was compared on day 6. Data shown is the average from the three cell lines where each cell line was treated in triplicate. * p<0.05, ** p<0.01 and *** <0.001 from One-Way ANOVA analysis performed using GraphPad® Prism. Data for individual cell lines is shown in Table 5. (B) A panel of breast cancer cell lines was used to prepare lysates for immunoblotting of TTK. Tubulin was used as the loading control. (C) Dose response curves for the treatment of breast cancer cell lines in the absence or presence of escalating doses of the TTK inhibitor (TTKi) AZ3146. The survival of cells was measured using the CellTitre® MTS/MTA assay carried out 6 days after treatment. Percentage survival (n=3 per dose) was calculated as the percentage of the signal from treated cells to that from control cells. (D) The concentration of TTK required to affect the survival of 50% of the cells (IC50) was measured by GraphPad® Prism from the dose response curves in C for each cell line.

FIG. 6: TTK protein expression associates with breast cancer survival. The overall survival of patients in a large cohort of breast cancer patients (n=409) was stratified according to TTK staining by IHC (scores 0-3). Kaplan-Meier survival curves are shown for all patients (A) with four TTK staining (categories 0-3) and (B) two categories (0-2 vs. 3). Log-rank Test and p-value were used for survival curves. (C) The distribution of high TTK staining (category 3) across histological subgroups and mitotic indices. Data shown is the mitotic index (median+range) measured as the number of mitotic cells in 10 high power fields (hpf). The number of tumors with high TTK staining to the total number of tumors in the cohort is shown on the right. High TTK expression distributed across subtypes and did not associate with mitotic index.

FIG. 7: TTK associates with aggressive subtypes and is a therapeutic target. (A) Kaplan-Meier survival curves are shown for Grade 3 tumors, lymph node positive patients (LN⁺) and LN⁺ patients with grade 3 tumors. Log-rank Test and p-value were used for these survival curves. For patients with TNBC, and HER2, survival was statistically significant using the Gehan-Breslow-Wilcoxon test (p-values marked by asterisks) which gives more weight to deaths at early time points. The poorer survival of patients with high Ki67 tumors and high TTK staining was a trend but did not reach significance. Survival curves and statistical analyses were performed using GraphPad® Prism. (B) TNBC and non-TNBC cell lines were treated for 6 days with the specified concentrations of docetaxel (doc) alone, TTK inhibitor (TTKi) alone of the combinations. The survival of cells was measured using the MTS/MTA assay as described in Methods. *** p<0.001 comparing the combination to single agents and to non-TNBC cell lines from Two-Way Anova in GraphPad® Prism. (C) MDA-MB-231 cells were treated with docetaxel or TTKi alone or in combination and collected at 96 hours to perform apoptosis assays by flow cytometry. Early apoptotic cells were defined as annexin V+/7-AAD-.

FIG. 8: Global gene expression meta-analysis of genes deregulated in TNBC, metastatic events and death at 5 years in Oncomine™. (A) TNBC in 8 datasets were compared to non-TNBC, (B) tumors with metastatic events at 5 years were compared to those with no metastatic events at 5 years in 7 datasets and (C) tumors leading to death at 5 years were compared to those that did not lead to death at 5 years were compared in 7 datasets. The datasets used in the comparisons are stated in the legends and the key for the heatmap coloring is also included. The heatmap key denotes the top or bottom x % placement of a gene according to gene rank which is based on the p-value.

FIG. 9: The derivation of the 206 aggressiveness gene list. (A and B) are Venn diagrams for the top overexpressed genes and bottom underexpressed genes shared between TNBC and/or metastasis and death at 5 years analyses in Oncomine™. (C and D) The Venn diagrams from A and B were crossed with genes which were deregulated in TNBC in comparison to adjacent normal breast tissue from the METABRIC dataset. The genes marked in bold in panels C and D are the 206 genes which constitute the unfiltered aggressiveness gene list.

FIG. 10: Common genes between the 206 aggressiveness gene list and metagene attractors. Venn diagrams show common genes (in bold) between the 206 aggressiveness gene list and the chromosomal instability (CIN), lymphocyte-specific and ER attractors (Cheng et al 2013a, Cheng et al 2013b). The table below lists the shared genes. The 6 overexpressed genes (marked in red) and 2 underexpressed genes (marked in green) which constitute the 8-genes signature in this study are shown. Gene set enrichment analysis of the remaining 140 genes which were only present in the 206 gene signature reveal that these genes function in cell cycle.

FIG. 11: Correlation of breast cancer subtypes and the aggressiveness gene list. The METABRIC dataset was visualized according to the expression of the 206 genes in the aggressiveness gene list. The aggressiveness score for each tumor was calculated as the sum of normalized z-score expression values of overexpressed genes divided by that of underexpressed genes. (A and B) The expression of the aggressiveness gene list was visualized according to PAM50 intrinsic subtypes and the integrative clusters classification. Box plots show the aggressiveness score of these subtypes. The shaded lines in box plots mark the median value for the aggressiveness score. *** p<0.001 One-Way ANOVA using GraphPad® Prism. Kaplan-Meier curves are of overall survival of patients in the METABRIC dataset stratified according to the quartiles (left plot) or the median (middle plot) of the aggressiveness score in ER+ patients with Grade 3 tumors. Tumors of the five PAM50 intrinsic subtypes which show high aggressiveness score (higher than the median) did not show statistical difference in overall survival (right plot). The hazard ratio (HR) and the 95% confidence interval (CI) and the p-value are reported using the Log-rank Test.

FIG. 12: Survival of the PAM50 breast cancer subtypes in the METABRIC dataset according to the aggressiveness score. The survival of patients in the METABRIC dataset annotated based on the PAM50 subtypes was analyzed by dichotomy across the median aggressiveness score from the 206 gene list (A) and the reduced 8 gene list (B). The p-value are reported using the Log-rank Test in GraphPad® Prism and show that all tumors with the different PAM50 subtypes but high aggressiveness score did not show a difference in patient survival (left graphs), whereas the PAM50 subtypes showed significantly different survival only in low aggressiveness score setting.

FIG. 13: TTK staining association with patient survival. The overall survival of patients in a large cohort of breast cancer patients (n=409) was stratified according to TTK staining by IHC (scores 0-3). Kaplan-Meier survival curves are shown for all patients (with four TTK staining categories 0-3 and two categories (0-2 vs. 3) with 10 and 20 years follow up. Log-rank Test and p-value were used for survival curves of all patients. There were no statistical differences in the survival of patients with Grade 1, Grade 2 or hormone positive tumors when stratified by TTK expression. Survival curves and statistical analyses were performed using GraphPad® Prism.

FIG. 14: Criteria used for assigning ‘prognostic subgroups’ in this study.

FIG. 15: Panel 1: Overall survival curves of lung cancer patients split by ten (10) CIN and two (2) ER genes as a signature; patients are low or high according to the median of the signature; Panel 2: Survival curves for lung adenocarconima split by ten (10) CIN genes and two (2) ER genes as a signature; patients are low or high according to the median of the signature; Panel 3: Survival curves for lung adenocarconima (10 years) split by ten (10) CIN genes and two (2) ER genes as a signature; patients are low or high according to the median of the signature; Panel 4: Survival curves for lung adenocarconima split by six (6) CIN genes and two (2) ER genes as a signature; patients are low or high according to the median of the signature; and Panel 5: Survival curves for lung adenocarconima (10 years) split by six (6) CIN genes and two (2) ER genes as a signature; patients are low or high according to the median of the signature.

FIG. 16: (A) RNA-Seq data from the breast cancer cohort of The Cancer Genome Atlas (TCGA) data. (B) Recurrence-free survival of breast cancer patients in the TCGA stratified by the Aggressiveness score compared to the OncotypeDx recurrence score. (C) Comparison of copy number variations (CNVs) of breast tumours with high aggressiveness score to those with low aggressiveness score.

FIG. 17: (A) RNA-Seq data from all cancers of The Cancer Genome Atlas (TCGA) data. (B) Recurrence-free survival of all cancer patients in the TCGA stratified by the Aggressiveness score compared to the OncotypeDx recurrence score.

FIG. 18: Recurrence-free survival or overall survival of cancer patients with different cancer types in the TCGA data patients stratified by the 8-genes aggressiveness score.

FIG. 19: Outline of Example 2. Meta-analysis was performed in Oncomine™ using breast cancer datasets irrespective of subtypes or gene expression array platforms used. The global gene expression profiles of breast tumors that led to metastatic or death event within 5 years were compared to those that did not and the top overexpressed (OE) and underexpressed genes (UE) in these comparisons were selected. The commonly deregulated genes in the primary tumors that led to metastatic and death events (depending on the annotation of each dataset) were then interrogated using the online tool KIVI-Plotter™ (n>4000 patients with some overlap with the datasets in Oncomine™). Only genes which associated with relapse-free survival (RFS), distant metastasis-free survival (DMFS) or overall survival (OS) of basal-like breast cancer (BLBC) or ER-negative (ER⁻) breast cancer were selected. The 96 genes from this training were then shortlisted to 28 genes by selecting the most significant and persistent across the different outcomes (RFS, DMFS and OS). The 28-gene signature was then validated in large cohorts of breast cancer gene expression studies including The Cancer Genome Atlas (TCGA) dataset the Research Online Cancer Knowledgebase (ROCK) dataset and the homogenous TNBC dataset for prognostication of ER−, TNBC and BLBC subtypes. Finally, the TN signature was then investigated for association with pathological complete response (pCR) after neoadjuvant chemotherapy in studies which performed gene expression profiling prior to therapy.

FIG. 20: The 28-gene TN signature associates with RFS, DMFS and OS of BLBC and ER− breast cancer. The 21 overexpressed and 7 underexpressed genes were used as a signature in the online tool KM-Plotter. The signature (the average expression of the 21 overexpressed genes and the inverted expression of the 7 underexpressed genes) stratified the RFS, DMFS and OS; low: under the median of the expression of the signature and high: over the median of the expression of the signature. The hazard ratio (HR) and log-rank p-value (p) for the univariate survival analyses were generated by KM-Plotter. n=number of patients.

FIG. 21: The prognostication by the TN score outperforms standard clinicothapological indicators in TNCBC, BLBC and ER− breast cancer subtypes. Two datasets, (A) the TNBC dataset and (B&C) the ROCK dataset, were analyzed for the TN signature and the TN score was calculated as the ratio of the average expression of the 21 overexpressed genes to that of the 7 underexpressed genes. This score was calculated for each tumor and the median TN score over the entire dataset was used to classify tumors as high (above the median) or low (below the median) for the TN score. (A) RFR of TNBC patients in the TNBC cohort stratified by dichotomy across the median TN score in the cohort. Table under the survival curve shows univariate and multivariate survival analysis for the TN score and other available clinical indicators recorded in the dataset. The TN score outperformed all the clinical indicators in the multivariate analysis. (B) RFS and DMFS of BLBC in the ROCK dataset stratified by dichotomy across the median TN score in the dataset. The table under the survival curves shows multivariate survival analysis for the TN score against other available clinical indicators recorded in the dataset. The TN score outperformed all the clinical indicators in the multivariate analysis of BLBC cases. (C) The RFS and DMFS of ER− negative breast cancer were stratified by the TN score (data not shown) and the table shows the multivariate survival analysis that the TN score outperforms clinical indicators in ER⁻ breast cancer cases.

FIG. 22: The TN score stratifies the overall survival of ER− breast cancer patients in the TCGA dataset. The gene expression data using the Illumina HiSeq RNA-seq arrays from the TCGA breast cancer data (n=1106) were used to calculate the TN score for all tumors. Tumors were classified as high or low for the TN score by dichotomy across the median TN score. The overall survival (OS) of ER− breast cancer cases with high TN score were compared to those with low TN score. The table below the survival curve shows that the TN score is more significant than other clinical indicators in univariate survival analysis and it is the only significant prognostic indicator in multivariate survival analysis.

FIG. 23: The TN score associates with pCR after chemotherapy in ER⁻HER2⁻ breast cancer. Gene expression datasets which profiled tumors prior to neoadjuvant chemotherapy and recorded pathological complete responses (pCR) vs. no pCR or residual disease (RD) were analyzed for the TN signature and the TN score was calculated for each tumor. Tumors were classified as high or low TN score by dichotomy across the median TN score in each dataset. Only ER-HER2− cases were used in the data shown in the Figure. (A) Graphs showing the percentage of cases achieving (red bars) or not achieving (black bars) pCR in low and high TN score subgroups. Fisher's exact test was used to analyze the 2×2 contingency tables and the p-value from this test was reported when statistical significance was observed. The dotted line marks the 31% pCR rate reported in literature for TNBC. Each dataset is labeled with the accession number and the chemotherapy regimen used, namely: GSE18728, GSE50948, GSE20271, GSE20194, GSE22226, GSE42822 and GSE23988. Chemotherapy abbreviation: 5-FU, Adriamycin, Cyclophosphamide, Taxane, X: Xeloda, Methotrexate, Epirubicin. (B) The dataset GSE22226 from the ISPY-1 trial was used to compare the TN score and pCR in the prediction of ER⁻ patient survival after neoadjuvant chemotherapy as this dataset also recorded RFS. pCR strongly associated with RFS (first panel) as previously reported. the TN score (next three panel) was not only predictive of survival in the these patients but could also stratified the survival of patients achieving or not achieving pCR, indicated the TN score as an independent prognostic factor for pCR after neoadjuvant chemotherapy.

FIG. 24: Drug sensitivity of cancer cell lines according to the TN score. The large published study by Garnett et al. was investigated where the TN score was calculated for each cell line in the study as described in Methods. The cell lines were classified as high or low TN score according to the median TN score to compare the sensitivity of low TN score cell lines (white boxes) and high TN score cell lines (red boxes). Graphs were prepared using GraphPad® Prism showing sensitivity as −log 10[IC50] in boxes (with median marked by a line) and whiskers (marking the 1st and 3rd quartiles and outliers as dots according to Tukey method for plotting the whiskers and outliers). Unpaired two-tailed t test was used for statistical analysis.

FIG. 25: The iBCR score stratifies the survival of all breast cancer patients irrespective of ER status in the ROCK dataset. The TN and Agro scores were calculated for each tumor in the ROCK dataset (n=1570, Affymetrix) and then the iBCR score was calculated as the TN score to the power of the Agro score. The RFS of all patients and the RFS of ER− or ER+ patients only was compared between high score and low score by dichotomy across the median score for each of the scores. The iBCR score was prognostic in all patients as well as ER− and ER+ subsets with better separation between low score and high score tumors (increased hazard ratio [HR] and limits of the 95% confidence intervals and decreased log rank p-value). Graphs and the univariate survival analysis using the log rank test were performed using GraphPad® Prism.

FIG. 26: The iBCR score stratifies the survival of all breast cancer patients irrespective of ER status in the TCGA dataset. The TN. Agro and the iBCR scores were calculated for each tumor in the TCGA dataset (n=1106, Illumina RNA-Seq). The RFS of all patients and the RFS of ER− or ER+ patients only was compared between high score and low score. As in the results in the ROCK dataset in FIG. 7, The iBCR score was prognostic in all patients as well as ER− and ER+ subsets with better separation between low score and high score tumors.

FIG. 27: The iBCR score associates with RFS and pCR after chemotherapy in the ISPY-1 trial. The dataset GSE22226 from the ISPY-1 trial was used to compare the Agro, TN and the integrated iBCR score in the prognosis and association with pCR after chemotherapy (Adriamycin, Cyclophosphamide and Taxane) in ER⁻HER2⁻ and ER⁺ breast cancer subtypes. Tumors were classified as high or low score by dichotomy across the median of each score in the entire dataset. High iBCR score ER⁻HER2⁻ tumors were less likely to achieve pCR and these patients had poor survival. High iBCR ER⁺ patients were more likely to achieve pCR but since a small number of ER⁺ patients achieved (10/62 [16%]), the survival of high iBCR ER+ patients remained poor. Note that the Agro score identifies all but two ER−HER2− tumors as high score, thus the data from this group should not be interpreted. Also note that the Agro score is highly prognostic of survival and association with pCR in ER⁺ whereas the TN score is not in these patients. The integration of these two scores in the iBCR score has overcame the limitation of each of these subtype-specific scores.

FIG. 28: The iBCR score associates with pCR after chemotherapy in breast cancer. Gene expression datasets with pCR annotation after chemotherapy were used as described in FIG. 5 to calculate the Agro and TN scores and the integrated iBCR score. Tumors were classified as high or low score by dichotomy across the median of each score in each dataset. (A) ER⁻HER2⁻ cases with graphs showing the percentage of cases achieving (red bars) or not achieving (black bars) pCR in low and high score subgroups. (B) ER⁺ cases were analyzed as in A. Fisher's exact test was used to analyze the 2×2 contingency tables and the p-value from this test was reported when statistical significance was observed. Each dataset is labeled with the accession number and the chemotherapy regimen used, namely: GSE18728, GSE50948, GSE20271, GSE20194, GSE22226, GSE42822 and GSE23988. Chemotherapy abbreviation: 5-FU, Adriamycin, Cyclophosphamide, Taxane, X: Xeloda, Methotrexate, Epirubicin.

FIG. 29: The iBCR score stratifies the survival of tamoxifen-treated ER+ patients. The Agro and TN scores and the iBCR score were calculated in two datasets of gene expression profiling prior to tamoxifen therapy: A&B. GSE6532 with 327 patients. 137 untreated and 190 tamoxifen-treated; C: GSE17705 with 298 patients treated with tamoxifen for 5 years. (A) ER++N0 patients with high iBCR score have poor RFS compared low iBCR score counterparts. (B) RFS of all ER+ patients and N0 and N1 subsets stratified by the Agro and iBCR scores. (C) DMFS survival of all ER+ and N0 and N1 subsets stratified by the Agro and iBCR scores. The hazard ratios and log-rank p-values are more significant for the iBCR score than the Agro score although the Agro score was significantly prognostic.

FIG. 30: Drug sensitivity of cancer cell lines according to the iBCR score. The large published study by Garnett et al. was investigated where the iBCR score was calculated for each cell line from the Agro and TN scores. The cell lines were classified as high or low iBCR score according to the median iBCR score to compare the sensitivity of low iBCR score cell lines (white boxes) and high TN score cell lines (red boxes). Results according to low and high Agro score were also included. Graphs were prepared using GraphPad® Prism and unpaired two-tailed t test was used for statistical analysis (n.s. not significant).

FIG. 31: Global gene expression meta-analysis of genes deregulated in primary breast tumors with metastatic events or death at 5 years in Oncomine™. (A) tumors with metastatic events at 5 years were compared to those with no metastatic events at 5 years in 7 datasets and (B) tumors leading to death at 5 years were compared to those that did not lead to death at 5 years were compared in 7 datasets. The datasets used in the comparisons are stated in the legends and the key for the heatmap coloring is also included. The heatmap key denotes the top or bottom x % placement of a gene according to gene rank which is based on the p-value.

FIG. 32: The TN signature outperforms all published signatures for TNBC/BLBC. Relapse-free survival of basal-like breast cancer patients (BLBC) was investigated in the online database KM-Plotter (Affymetrix platform) according to the TN signature in comparison to published TNBC signatures. Hazard ratios (HR) and logrank p-values were generated by KM-Plotter. (A) the TN score vs. signatures (B) from Karn et al. (PLoS One, 2011); from Rody et al. (Breast Cancer Res, 2011) (C) IL8, (D) VEGF, and (E) B-cell metagenes; (F) from Yau et al. (Breast Cancer Res, 2010); (G) from Yu et al. (Clin Cancer Res, 2013); (H) from Lee et al. (PLoS One, 2013 and (I) from Hallet et al. (Sci Rep, 2012).

FIG. 33: The TN score stratified the survival of ER⁻ patients in the Agilent TCGA data. The original TCGA dataset using the Agilent microarrays (n=597) were analyzed for the TN score where patients were assigned as low, intermediate or high for the TN score according to tertiles. The RFS of ER− patients only were then compared according to these tertiles. The stratification was significant according to a log-rank survival test (P<0.0001). High TN score group vs. low TN score group had a hazard ratio (95% confidence interval) of 3.484 (1.035 to 11.23) with a log rank p-value of 0.0179.

FIG. 34: The prognostication by the TN score in ER− and BLBC is not affected by systemic treatment. The online KM-Plotter tool was used to investigate the stratification of RFS, DMFS and OS of ER− breast cancer (top two rows) and BLBC (bottom two rows) in systemically untreated patients (untreated) or in patients who were treated systemically (treated). The HR, the 95% confidence intervals and the log-rank p values were provided by KM-Plotter as well as the number of patients at risk.

FIG. 35: Sensitivity of cancer cell lines to anticancer drugs according to the TN score in the Cancer Cell Line Encyclopedia (CCLE) study. The gene expression data of the cancer cell lines in the study were analyzed to calculate the TN score for each cell line and were assigned to low or high TN score by dichotomy across the median. The IC₅₀ for each of the 24 drugs used in the CCLE study was compared between high and low TN score cell lines and the data shown are those with statistical differences based on unpaired two-tailed t-test performed using GraphPad® Prism.

FIG. 36: Integration of the TN and Agro scores by addition or subtraction. The ROCK dataset was used to study the integration of the TN and Agro score with the aim to develop a test that is breast cancer subtype independent. (A) The raw Agro and TN scores for ER+ (black dots) and ER− (red dots) in the ROCK dataset (each dot represent one patient, n=1570 in total). The two scores are scattered and a method of integration that can retain the information from each score in the relevant breast cancer subtype is necessary. Such methods are tested in this Figure and FIG. 38. (B) Addition method. First column shows the TN score in ER+ tumors with low (white boxes) and high (red boxes) Agro score subgroups (top panel). In the bottom panel, the Agro score in ER− tumors with low (white boxes) and high (red boxes) TN score subgroups. This data shows that the TN score is similar for ER+ tumors with low and high Agro scores and that the Agro score is similar for ER− tumors with low and high TN scores. The lack of statistical differences (independence) suggested that integration is possible. The second column shows the linear correlation between the TN score and Agro score when they were added in each patient for ER+ (top panel) and ER− (bottom panel) patients. In the third column, the TN and Agro scores were plotted against the produced summed score showing that the information from each score is retained in the final summed score for both ER+ (top panel) and ER− (bottom panel) patients. The last column shows the overlap of data from ER+ and ER− patients shown separately in the second and third columns. (C) Identical analysis as that done in B but the integration was tested by subtraction of the TN and Agro score. The linearity of the relationship between the summed score and each of the single scores (TN and Agro score) indicated that information from each score is represented in the final score. The performance of these two methods (addition or subtraction) was tested for association with survival as shown in FIG. 37.

FIG. 37: Comparison of different integration methods of the TN and Agro scores for prognostication in ER− and ER+ RFS in the ROCK dataset. The methods of integration by addition or subtraction (from FIG. 36) or multiplication or division (FIG. 38) were tested for the association of the produced integrated score in the ROCK dataset in ER− or ER+ breast cancer. As shown in the figure, only the addition or multiplication methods were prognostic in ER− breast cancer and the multiplication was more significant in ER+ breast cancer compared to the addition. These two methods are reasonable as subtraction or division methods would reduce the value of one of the scores. Two additional methods were tested, raising one score to the power of the second score since the relationships observed when multiplication and division methods showed exponential or power curves. As shown in the last column (shaded and marked in red box), raising the TN score to the power of the Agro score should superior prognostication in both ER− and ER+ breast cancer subtypes. In fact, the prognostication of this integrated score was better than each of the score in their respective subtypes. The method was therefore used to calculate the integrated Breast Cancer Recurrence (iBCR) score.

FIG. 38: Integration of the TN and Agro scores by division or multiplication. The ROCK dataset was used to study the integration of the TN and Agro as these scores were scattered when plotted against each other (panel A in FIG. 36). (A) The box plots in the first column are identical to those in FIG. 36. The shaded boxes in panel A describe integration by division (top row) or multiplication (bottom row) of the TN and Agro scores. The division produced a power curve and the multiplication produced an exponential curve for the relationship between the TN and Agro scores after dividing them or multiplying them by each other in both ER+ (black dots) and ER− (red dots). The overlay in the last column shows that the differences between ER+ and ER− patients for the scores is retained. These two methods were tested for survival association in FIG. 37 and the multiplication method was suitable. (B) As power and exponential curves were observed in the division and multiplication methods in A, it was reasonable to test integration by raising one score to the power of the second score. As shown in the top row in the overlay or individual plots, the integration by raising the TN score to the power of the Agro score produced a linear relationship in both ER− (red dots) and ER+ (black dots) patients. This method of integration outperformed all other methods when tested for survival association as shown in FIG. 37.

FIG. 39: The iBCR score is prognostic in TNBC patients. In addition to the validation of the iBCR score in the ROCK dataset (Affymetrix) and the TCGA dataset (Illumina dataset) of mixed subtypes of breast cancer, the iBCR score was investigated in the homogenous TNBC dataset. As shown in the right panel, the iBCR was as prognostic (with slight improvement) compared to the TN score. This further validates the development of the integrated score to be a prognostic test in breast cancer irrespective of ER status, unlike previous limited signatures.

FIG. 40: Survival of tamoxifen-treated ER+ patients according to the Agro score vs. Oncotype Dx. (A) RFS and DMFS of node negative (top) and node positive (bottom) ER+ patients treated with tamoxifen in the published study (Loi et al., Clin Oncol, 2007) stratified by the Agro Score (high vs. intermediate vs. low by tertiles). (B) DMFS of node negative or positive ER+ patients treated with tamoxifen for 5 years from the published study (Symmans et al., J Clin Oncol, 2010) was stratified by the tertiles of the Agro Score. (C) RFS and DMFS of node negative (top) and node positive (bottom) ER+ patients treated with tamoxifen in the published study (Loi et al., Clin Oncol, 2007) stratified by the risk groups of the OncotypeDx Recurrence Score. (D) DMFS of node negative or positive ER+ patients treated with tamoxifen for 5 years from the published study (Symmans et al., J Clin Oncol, 2010) was stratified by the risk groups of the OncotypeDx Recurrence Score.

FIG. 41: Comparison of the Agro Score and MammaPrint in the KM-Plotter tool. Distant metastasis-free survival according to the Agro Score (high vs. low) or according to MammaPrint (high vs. low) in all breast cancer patients, ER+, ER+ lymph node negative (LN−) or ER+ lymph node positive (LN+) patients. The KM-Plotter online tool (n=4142 patients). The Agro score outperformed the MammaPrint signature in all patient subsets particularly for ER+ node positive patients.

FIG. 42: Sensitivity of cancer cell lines to anticancer drugs according to the iBCR score in the Cancer Cell Line Encyclopedia (CCLE) study. The gene expression data of the cancer cell lines in the study were analyzed to calculate the TN score for each cell line and were assigned to low or high iBCR score by dichotomy across the median. The IC₅₀ for each of the 24 drugs used in the CCLE study was compared between high and low iBCR score cell lines and the data shown are those with statistical differences based on unpaired two-tailed t-test performed using GraphPad® Prism. As this analysis was also done for the TN score (FIG. 35), results from analysis of the Agro score are also shown in the top row.

FIG. 43: High copy number variations (CNVs) in high Agro score tumors compared to low Agro score tumors. The breast cancer tumors in the TCGA dataset were classified as high or low for the Agro score based on the gene expression data (Illumina HiSeq RNA-seq). (A) The TCGA copy number variations (segmented and after deletion of germline CNV) were visualized using the UCSC Genome Browser to compare patients who were classified from gene expression data as high Agro score patients (top panel) to those classified as low Agro score patients (bottom panel). (B) Presentation of the distribution of clinical indicators such as ER, PR and HER2 status and others. (C) The difference in the CNVs profile of high Agro score patients to the low Agro score patients showing gains (red) and losses (green) of whole chromosome arms in the high Agro score patients, suggesting aneuploidy.

FIG. 44: High Agro and iBCR score cell lines are more sensitive to Aurora kinase inhibitors. Two studies which treated breast cancer cell lines with Aurora kinase inhibitors were analyzed based on the Agro, TN and the iBCR score for these cell lines. As shown in Figure, high Agro score and particularly high iBCR score cell lines were more sensitive to Aurora kinase inhibitors (ENMD-2076: IC50 1.4 μM vs. 5.9 μM for high vs. low iBCR Score cell lines, p=0.0125 t-test; AMG 900: IC50 0.3 nM vs. 0.7 nM for high vs. low iBCR score cell lines, p=0.0308 t-test).

FIG. 45: The iBCR is prognostic in the pan-cancer TCGA data for overall and relapse-free survival. The pan-cancer TCGA data were analyzed for the iBCR gene signature using the UCSC Genome Browser and the data for this signature, survival data and cancer types were downloaded from the browser. Tumors, irrespective of cancer types, were classified into quartiles based on the iBCR signature expression and the overall and relapse free survival were compared across these quartiles. As shown in the top row, overall and relapse-free survival was stratified by the iBCR signature in this pan-cancer dataset. In the far right panel in the top row, the distribution of tumors in each cancer type across the iBCR signature quartile is shown. Cervical cancer for example displays high iBCR signature in the majority of cases whereas on the opposite side, thyroid cancer displays low iBCR signature in all the cases. The lower panels show the stratification of overall survival according to the iBCR score from the pan-cancer dataset where the stratification was statistically significant in log-rank univariate survival analysis. In addition to the breast cancer data shown in paper, the iBCR signature was prognostic in adrenocortical cancer, endometrioid cancer, kidney clear cell cancer, bladder cancer, lower grade glioma and melanoma. The iBCR was also prognostic in lung adenocarcinoma as shown in FIG. 46.

FIG. 46: The iBCR signature is prognostic in lung adenocarcinoma (LUAD). The iBCR signature was tested for prognostication in lung cancer in two large datasets. (A&B) KM-Plotter (Affymetrix data) was used to investigate overall survival of lung adenocarcinoma (A) and squamous cell carcinoma (B). The iBCR signature shows a strong prognostic value in lung adenocarcinoma (LUAD). (C) Multivariate survival analysis was performed in KM-Plotter for the iBCR signature in lung cancer in comparison to available clinical indicators; histological type (lung adenocarcinoma vs. small cell lung cancer) and stage of disease. The iBCR signature outperformed these standard clinical indicators. (D&E) The TCGA data for LUAD (Illumina HiSeq RNA-seq data) were stratified by quartiles or tertiles for the iBCR signature expression to test the association of the iBCR signature with overall survival (D) and relapse-free survival (E), respectively. LUAD patients with high iBCR signature had poorest survival and suffered earlier recurrence and death compared to patients with lower iBCR signature expression. It should be noted that the TCGA data for squamous cell lung carcinoma were also investigated and there was no statistical significance for the association of the iBCR signature and survival, in agreement with the very weak association seen from the KM-Plotter data.

FIG. 47: The sensitivity of breast cancer cell lines treated with 24 drugs according to the iBCR score. Breast cancer cell lines (10 cell lines) were cultured in the absence or presence of escalating doses of 24 small molecular anti-cancer drugs. This published study was re-analyzed to compare the sensitivity (calculated as the −log IC50) between high iBCR score cell lines (5 cell lines: BT-549, MDA-MB-231, MDA-MB-436, MDA-MB-468 and BT-20) to low iBCR score cell lines (5 cell lines: Hs.578T, BT-474, MCF-7, T-47D, and ZR-75-1). The iBCR scores were calculated from the Agro and TN scores using the published gene expression dataset for 51 breast cancer cell lines (Neve et al., Cancer Cell, 2006). High iBCR score cell lines (red bars) were more sensitive than low iBCR score cell lines (white bars) to 13 drugs (shaded in grey) targeting 9 different kinases. Statistical comparison was performed in GraphPad® Prism using two tailed unpaired t-test.

FIG. 48: Proteins and phosphoproteins associated with the iBCR mRNA gene signature. The iBCR score based on the mRNA expression of the 43 genes was used to stratify the patients in the TCGA breast cancer dataset as low, intermediate or high iBCR score. The reverse phase protein arrays (RPPA) from the TCGA breast cancer dataset (n=747 patients) were then compared between the three groups of patients according to the iBCR mRNA signature. (A) Overall survival of ER+ patients according to the iBCR mRNA signature. (B) Significantly up- or down-regulated proteins and phosphoproteins in ER+ patients in the low, intermediate and high iBCR score groups. (C) Overall survival of ER− according to the iBCR mRNA signature. (D) Significantly up- or down-regulated proteins and phosphoproteins in ER− patients in the low, intermediate and high iBCR score groups.

FIG. 49: Prognostication of breast cancer patient survival by integrated mRNA and protein iBCR signature. The deregulated proteins and phosphoproteins in the three iBCR mRNA score groups were investigated for association with survival. Eight downregulated proteins and nine upregulated proteins were highly prognostic as a protein signature (iBCR protein signature). (A) Stratification of overall survival based on the iBCR protein signature (top row) and the integrated iBCR mRNA and protein signature (bottom row) in all breast cancer patients, ER+ and ER− cases. (B) Univariate and multivariate survival analysis using the Cox-proportional hazard model showing that the combined iBCR mRNA/Protein signature outperforms all clinicopathological indicators.

FIG. 50: Proteins and phosphoproteins associated with the iBCR mRNA gene signature. (A) Stratification of lung adenocarcinoma overall survival based on the iBCR mRNA gene signature in the TCGA dataset (n=472 patients). (B) Comparison of proteins phosphoprotein levels between the tumors in the four quartiles of the iBCR mRNA gene signature. (C) Stratification of overall survival of lung adenocarcinoma patients based on six proteins deduced from panel (n=212 patients). (D) The combined iBCR mRNA/Protein signature stratifies the overall survival of lung adenocarcinoma patients (n=212 patients). (E) Multivariate Cox-proportional hazard model for survival analysis showing that the combined iBCR mRNA/Protein score outperforms all clinicopathological indicators in lung adenocarcinoma.

FIG. 51: The iBCR test is prognostic in Kidney renal clear cell carcinoma (KIRC) (left vertical panel), Skin cutaneous melanoma (SKCM) (middle vertical panel) and Uterine corpus endometrioid carcinoma (UCEC) (right vertical panel). (A) Stratification of overall survival based on the iBCR mRNA gene signature. (B) Stratification of overall survival based on iBCR protein signature. (C) Stratification of overall survival based on the combined iBCR mRNA/protein signature.

FIG. 52: The iBCR test is prognostic in Ovarian adenocarcinoma (OVAC) (left vertical panel), Head & Neck squamous cell carcinoma (HNSC) (middle vertical panel) and Colon/Rectal Adenocarcinoma (COREAD) (right vertical panel). (A) Stratification of overall survival based on the iBCR mRNA gene signature. (B) Stratification of overall survival based on iBCR protein signature. (C) Stratification of overall survival based on the combined iBCR mRNA/protein signature.

FIG. 53: The iBCR test is prognostic in Lower Grade Glioma (LGG) (left vertical panel), Bladder urothelial carcinoma (BLCA) (middle vertical panel) and Lung squamous cell carcinoma (LUSC) (right vertical panel). (A) Stratification of overall survival based on the iBCR mRNA gene signature. (B) Stratification of overall survival based on iBCR protein signature. (C) Stratification of overall survival based on the combined iBCR mRNA/protein signature.

FIG. 54: The iBCR test is prognostic in (A) Kidney renal papillary cell carcinoma (KIRP). (B) Cervical squamous cell carcinoma and endocervical adenocarcinoma (CESC), (C) Liver hepatocellular carcinoma (LIHC), (D) Pancreatic ductal adenocarcinoma (PDAC). For these cancer types, the TCGA datasets did not include RPPA arrays; only the iBCR mRNA gene expression test was used.

FIG. 55: Protein-protein interaction of the iBCR mRNA/protein signature. The components of the iBCR test were analysed using the STRING database. The iBCR test (65 components) was significantly enriched (P=5.6E-14) for protein-protein interactions (129 interactions). The confidence of interactions is denoted by increasing thickness of the connecting blue lines. It is noteworthy that the components on the top right which do not show interactions contain several novel genes that are not well characterised. The iBCR test is enriched for several biological functions related to the hallmarks of cancer (refer to Table 20).

FIG. 56: The iBCR test as a companion diagnostic for immunotherapy. (A) Twelve genes from the iBCR test, particularly from the TN component, associated significantly with progression free survival of follicular lymphoma patients treated with pidilizumab+rituximab immunotherapy. The expression profile of the 12 genes in the tumours prior to treatment is shown (red indicates overexpression and green indicates underexpression). White and black boxes denote progression free survival or not, respectively. (B) A score was calculated based on the iBCR signature as the ratio of expression of the overexpressed genes to that of underexpressed genes. The survival of patients based on dichotomy across the median score was compared. The hazard ratio (HR) and the log-rank p-value for the survival comparison between low and high score tumors is shown in panel. (C) Eight patients were profiled pre- and post-treatment and the expression profiles of the 12 genes from the iBCR test were visualised in these patients. A trend for inversion of expression was observed and this was most evident for patient no. 9 who remained free of disease progression. (D) One gene was statistically significant in all patients post-treatment compared to that before treatment. This gene showed a marked different post-treatment vs. pre-treatment for patient no. 9. (E) Survival curve for the same patient group calculated from the gene signature labelled “Follicular Lymphoma” in Table 23. All conventions as per (B) above. Relapse-free survival of patients based on dichotomy across the median score is shown.

FIG. 57: Network analysis of the genes from the meta-analysis of gene expression datasets.

FIG. 58: Functional metagenes associate with breast cancer patient survival.

FIG. 59: The iBCR test as a companion diagnostic for EGFR inhibition and multikinase inhibition. (A) Seventeen genes (see Table 23) from the iBCR test associated significantly with survival of colorectal cancer patients treated with the EGFR inhibitor cetuximab. (B) Sixteen genes (see Table 23) from the iBCR test associated significantly with overall survival of triple negative breast cancer patients treated with the EGFR inhibitor cetuximab combined with cisplatin. (C) Nineteen genes (see Table 23) from the iBCR test associated significantly with progression-free survival of lung cancer patients treated with the EGFR inhibitor erlotinib. (D) Twenty genes (see Table 23) from the iBCR test associated significantly with progression-free survival of lung cancer patients treated with the multikinase inhibitor sorafenib.

### DETAILED DESCRIPTION

The present invention is at least partly predicated on the discovery that there are genes that are associated with tumor aggressiveness and poor clinical outcome based on meta-analysis of published gene expression profiling. More particularly, the overexpression and/or underexpression of these genes (see Table 21) was found to be associated with poor survival in breast cancer. Network analysis using the Ingenuity Pathway Analysis (IPA®) software identified a number of networks or metagenes within these survival-associated genes that possess distinct biological functions as outlined in Table 21. A smaller subset of genes from each network or metagene which consistently associated with patient survival were then selected. The list of these genes and their corresponding functions are shown in Table 22. These genes were divided into six functional metagenes or networks.

The present invention is also at least partly predicated on the discovery that there are genes that are commonly de-regulated in particular subgroups that exemplify aggressive clinical behavior in triple-negative breast cancer (TNBC). More particularly, this is evident in TNBC compared to non-TNBC and normal breast, tumors associated with distant metastasis and/or death compared to their respective counterparts. Initially, a list of 206 recurrently deregulated genes was found to be particularly enriched for chromosomal instability (CIN) and estrogen receptor signaling (ER) metagenes. An aggressiveness score based on the ratio of the expression level of a CIN metagene relative to an ER metagene has been shown to identify aggressive tumors regardless of molecular subtype and clinico-pathologic indicators. Furthermore, depletion of proteins involved in kinetochore binding or chromosome segregation could be therapeutic and significantly reduced the survival of TNBC cell lines in vitro, particularly with regard to TTK. TTK inhibition with small molecule inhibitor affected the survival of TNBC cell lines. Also, TTK mRNA and protein levels were associated with aggressive tumor phenotypes. Mitosis-independent expression of TTK protein was prognostic in TNBC and other aggressive breast cancer subgroups, suggesting that protection of CIN/aneuploidy drives aggressiveness and treatment-resistance. The combination of TTK inhibition with chemotherapy was effective in vitro in the treatment of cells that overexpress TTK, thus providing a therapeutic treatment for the protected CIN phenotype.

Additionally, the present invention is at least partly predicated on the discovery of a second signature of altered gene expression, including 21 overexpressed genes and 7 underexpressed genes, that is highly prognostic in patients with ER⁻ breast cancer, TNBC and basal-like breast cancer (BLBC). Indeed, integration of this 28 gene signature with the aforementioned aggressiveness score or gene signature produces an integrated score which is prognostic in breast cancer independent of ER status. Furthermore, the integrated score was prognostic in cancer broadly irrespective of the cancer type, as well as in specific types of cancer in addition to breast cancer, such as lung adenocarcinoma. Moreover, the 28 gene signature and the integrated score were both shown to be predictive of response to chemotherapy in breast cancer patients, as well as identify those ER⁺ lymph node positive breast cancer patients who would benefit from endocrine therapy. Altered expression of the signatures described herein was also predictive of sensitivity in cancer cell lines and clinically to a range of anticancer therapeutics, and in particular, molecularly targeted inhibitors.

The inventors of the present invention have also identified a protein signature that is highly prognostic in a range of cancers, including breast cancer and lung adenocarcinoma. Furthermore, this protein signature may be integrated with the aforementioned 28 gene signature and aggressive gene signature to provide a robust prognostic indicator in cancer that was shown to outperform known clinicopathological indicators.

In one aspect, the invention relates to a method of determining the aggressiveness of a cancer in a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the plurality of the overexpressed genes compared to the plurality of the underexpressed genes indicates or correlates with higher aggressiveness of the cancer; and/or a lower relative expression level of the plurality of the overexpressed genes compared to the plurality of the underexpressed genes indicates or correlates with lower aggressiveness of the cancer compared to a mammal having a higher expression level.

In a further aspect, the invention relates to a method of determining a cancer prognosis for a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the plurality of overexpressed genes compared to the plurality of underexpressed genes indicates or correlates with a less favourable cancer prognosis; and/or a lower relative expression level of the plurality of overexpressed genes compared to the plurality of underexpressed genes indicates or correlates with a more favourable cancer prognosis.

In one embodiment of the above aspects, the plurality of overexpressed genes and/or the plurality of underexpressed genes are selected from one of the metagenes. In an alternative embodiment, the plurality of overexpressed genes and/or the plurality of underexpressed genes are selected from a plurality of the metagenes.

Suitably, for the method of the above aspects the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or more genes listed in Table 21.

In another aspect, the invention relates to a method of determining the aggressiveness of a cancer in a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the plurality of the overexpressed genes compared to the plurality of the underexpressed genes indicates or correlates with higher aggressiveness of the cancer; and/or a lower relative expression level of the plurality of the overexpressed genes compared to the plurality of the underexpressed genes indicates or correlates with lower aggressiveness of the cancer compared to a mammal having a higher expression level

In yet another aspect, the invention relates to a method of determining a cancer prognosis for a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the plurality of overexpressed genes compared to the plurality of underexpressed genes indicates or correlates with a less favourable cancer prognosis; and/or a lower relative expression level of the plurality of overexpressed genes compared to the plurality of underexpressed genes indicates or correlates with a more favourable cancer prognosis.

Suitably, the Metabolism metagene, the Signalling metagene, the Development and Growth metagene, the Chromosome Segregation/Replication metagene, the Immune Response metagene and/or the Protein Synthesis/Modification metagene comprise one or more genes listed in Table 21.

In particular embodiments of the method of the two aforementioned aspects, the plurality of overexpressed genes and the plurality of underexpressed genes are from one or more of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene. According to the method of the above aspects, the step of comparing an expression level of a plurality of overexpressed genes and an expression level of a plurality of underexpressed genes includes comparing an average expression level of the plurality of overexpressed genes and an average expression level of the plurality of underexpressed genes. This may include calculating a ratio of the average expression level of the plurality of overexpressed genes and the average expression level of the plurality of underexpressed genes. Suitably, the ratio provides an aggressiveness score which is indicative of, or correlates with, cancer aggressiveness and a less favourable prognosis. Alternatively, the step of comparing an expression level of a plurality of overexpressed genes and an expression level of a plurality of underexpressed genes includes comparing the sum of expression levels of the plurality of overexpressed genes and the sum of expression levels of the plurality of underexpressed genes. This may include calculating a ratio of the sum of expression levels of the plurality of overexpressed genes and the sum of expression levels of the plurality of underexpressed genes.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein a “gene” is a nucleic acid which is a structural, genetic unit of a genome that may include one or more amino acid-encoding nucleotide sequences and one or more non-coding nucleotide sequences inclusive of promoters and other 5′ untranslated sequences, introns, polyadenylation sequences and other 3′ untranslated sequences, although without limitation thereto. In most cellular organisms a gene is a nucleic acid that comprises double-stranded DNA.

Non-limiting examples of genes are set forth herein, particularly in Tables 4, 21 and 22, which include Accession Numbers referencing the nucloetide sequence of the gene, or its encoded protein, as are well understood in the art.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Also included are, “variant” nucleic acids that include nucleic acids that comprise nucleotide sequences of naturally occurring (e.g., allelic) variants and orthologs (e.g., from a different species). Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. A non-limilting example is an amplification product or a primer or probe. In particular embodiments, a nucleic acid fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475 and 500 contiguous nucleotides of said nucleic acid.

As used herein, a “polynucleotide” is a nucleic acid having eighty (80) or more contiguous nucleotides, while an “oligonucleotide” has less than eighty (80) contiguous nucleotides. A “probe” may be a single or double-stranded oligonucleotide or polynucleotide, suitably labeled for the purpose of detecting complementary sequences in Northern or Southern blotting, for example. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “template” nucleic acid is a nucleic acid subjected to nucleic acid amplification.

It will be appreciated that the “overexpressed” genes or proteins referred to herein are genes or proteins that are expressed at a higher level in a cancer cell or tissue compared to a corresponding normal or otherwise non-cancerous cell or tissue or reference/control level or sample.

It will be appreciated that the “underexpressed” genes or proteins referred to herein are genes or proteins that are expressed at a lower level in a cancer cell or tissue compared to a corresponding normal or otherwise non-cancerous cell or tissue or reference/control level or sample.

In certain embodiments, the “overexpressed” and “underexpressed” genes referred to herein may form, or be components of, a metagene.

As used herein, a “metagene” is a grouping, cohort or network of a plurality of different genes that display a common, shared or aggregate expression profile, expression level or other expression characteristics that associate with, or are indicative of, a particular function or phenotype. Non-limiting examples include a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene. Table 21 provides non-limiting examples of genes that are components of the aforementioned twelve metagenes. Further non-limiting examples include a Metabolism metagene, a Signalling metagene, a Development and Growth metagene, a Chromosome Segregation/Replication metagene, an Immune Response metagene and a Protein Synthesis/Modification metagene. Table 22 provides non-limiting examples of genes that are components of the aforementioned six metagenes.

In particular embodiments, the plurality of overexpressed genes and/or the plurality of underexpressed genes are selected from one of the metagenes. In this regard, the plurality of overexpressed genes and/or the plurality of underexpressed genes are selected from the same metagene. By way of example, the plurality of overexpressed genes or the plurality of underexpressed genes may be only from one of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and the Multiple Networks metagene. In a further example, both the plurality of overexpressed genes and the plurality of underexpressed genes may be only from one of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and the Multiple Networks metagene.

Alternatively, the plurality of overexpressed genes and/or the plurality of underexpressed genes are selected from a plurality of the metagenes described herein.

By “aggressiveness” and “aggressive” is meant a property or propensity for a cancer to have a relatively poor prognosis due to one or more of a combination of features or factors including: at least partial resistance to therapies available for cancer treatment; invasiveness; metastatic potential; recurrence after treatment; and a low probability of patient survival, although without limitation thereto.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

In certain embodiments, cancers include breast cancer, bladder cancer, colorectral cancer, glioblastoma, lower grade glioma, head & neck cancer, kidney cancer, liver cancer, lung adenocarcinoma, acute myeloid leukaemia, pancreatic cancer, adrenocortical cancer, melanoma and lung squamous cell carcinoma.

Breast cancers include all aggressive breast cancers and cancer subtypes such as triple negative breast cancer, grade 2 breast cancer, grade 3 breast cancer, lymph node positive (LN⁺) breast cancer, HER2 positive (HER2⁺) breast cancer and ER positive (ER⁺) breast cancer, although without limitation thereto.

As used herein, “triple negative breast cancer” (TNBC) is an often aggressive breast cancer subtype lacking or having significantly reduced expression of estrogen receptor (ER) protein, progesterone receptor (PR) protein and HER2 protein. TNBC and other aggressive breast cancers are typically insensitive to some of the most effective therapies available for breast cancer treatment including HER2-directed therapy such as trastuzumab and endocrine therapies such as tamoxifen and aromatase inhibitors.

As used herein, a gene expression level may be an absolute or relative amount of an expressed gene or gene product inclusive of nucleic acids such as RNA, mRNA and cDNA and protein.

As would be appreciated by the skilled artisan, the present invention need not be limited to comparing the expression level of the overexpressed genes and/or proteins with the expression level of the underexpressed genes and/or proteins provided herein. Accordingly, in particular embodiments, the expression level of the overexpressed and/or underexpressed genes and/or proteins is compared to a control level of expression, such as the level of gene and/or protein expression of a “housekeeping” gene in one or more cancer cells, tissues or organs of the mammal.

In further embodiments, the expression level of the overexpressed and/or underexpressed genes and/or proteins is compared to a threshold level of expression, such as a level of gene and/or protein expression in non-aggressive cancerous tissue. A threshold level of expression is generally a quantified level of expression of a particular gene or set of genes, including gene products thereof. Typically, an expression level of a gene or set of genes in a sample that exceeds or falls below the threshold level of expression is predictive of a particular disease state or outcome. The nature and numerical value (if any) of the threshold level of expression will vary based on the method chosen to determine the expression the one or more genes or proteins used in determining, for example, a prognosis, the aggressiveness and/or response to anticancer therapy, in the mammal. In light of this disclosure, any person of skill in the art would be capable of determining the threshold level of gene/protein expression in a mammal sample that may be used in determining, for example, a prognosis, the aggressiveness and/or response to anticancer therapy, using any method of measuring gene or protein expression known in the art, such as those described herein. In one embodiment, the threshold level is a mean and/or median to expression level (median or absolute) of the overexpressed and/or underexpressed genes and/or proteins in a reference population, that, for example, have the same cancer type, subgroup, stage and/or grade as said mammal for which the expression level is determined. Additionally, the concept of a threshold level of expression should not be limited to a single value or result. In this regard, a threshold level of expression may encompass multiple threshold expression levels that could signify, for example, a high, medium, or low probability of, for example, progression free survival.

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., phosphoproteins).

Also provided are protein “variants” such as naturally occurring (eg allelic variants) and orthologs. Preferably, protein variants share at least 70% or 75%, preferably at least 80% or 85% or more preferably at least 90%, 91%, 92%, 93%, 94%, 95%, 96%, 97%, 98% or 99% sequence identity with an amino acid sequence disclosed herein.

Also provided are protein fragments, inclusive of peptide fragments thqat comprise less than 100% of an entire amino acid sequence. In particular embodiments, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375 and 400 contiguous amino acids of said protein.

A “peptide” is a protein having no more than fifty (50) amino acids.

A “polypeptide” is a protein having more than fifty (50) amino acids.

It would be appreciated that in addition to comparing the expression levels of one or more genes or proteins, the methods of the present invention may further include the step of determining, assessing, evaluating, assaying or measuring the expression level of one or more of the overexpressed genes, the underexpressed genes, the overexpressed proteins and/or the underexpressed proteins described herein. The terms “determining”, “measuring”, “evaluating”, “assessing” and “assaying” are used interchangeably herein and may include any form of measurement known in the art, such as those described hereinafter.

Determining, assessing, evaluating, assaying or measuring nucleic acids such as RNA, mRNA and cDNA may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-β replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR and other variations and modifications to “basic” PCR amplification.

Nucleic acid amplification techniques may be performed using DNA or RNA extracted, isolated or otherwise obtained from a cell or tissue source. In other embodiments, nucleic acid amplification may be performed directly on appropriately treated cell or tissue samples.

Nucleic acid hybridization typically includes hybridizing a nucleotide sequence (typically in the form of a probe) to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

Determining, assessing, evaluating, assaying or measuring protein levels may be performed by any technique known in the art that is capable of detecting cell- or tissue-expressed proteins whether on the cell surface or intracellularly expressed, or proteins that are isolated, extracted or otherwise obtained from the cell of tissue source. These techniques include antibody-based detection that uses one or more antibodies which bind the protein, electrophoresis, isoelectric focussing, protein sequencing, chromatographic techniques and mass spectroscopy and combinations of these, although without limitation thereto. Antibody-based detection may include flow cytometry using fluorescently-labelled antibodies that bind the protein, ELISA, immunoblotting, immunoprecipitation, in situ hybridization, immunohistochemistry and immuncytochemistry, although without limitation thereto. Suitable techniques may be adapted for high throughput and/or rapid analysis such as using protein arrays such as a TissueMicroArray™ (TMA), MSD MultiArrays™ and multiwell ELISA, although without limitation thereto.

In certain embodiments, a gene expression level may be assessed indirectly by the measurement of a non-coding RNA, such as miRNA, that regulate gene expression. MicroRNAs (miRNAs or miRs) are post-transcriptional regulators that bind to complementary sequences in the 3′ untranslated regions (3′ UTRs) of target mRNA transcripts, usually resulting in gene silencing. miRNAs are short RNA molecules, on average only 22 nucleotides long. The human genome may encode over 1000 miRNAs, which may target about 60% of mammalian genes and are abundant in many human cell types. Each miRNA may alter the expression of hundreds of individual mRNAs. In particular, miRNAs may have multiple roles in negative regulation (e.g., transcript degradation and sequestering, translational suppression) and/or positive regulation (e.g., transcriptional and translational activation). Additionally, aberrant miRNA expression has been implicated in various types of cancer.

In this regard, an average expression level, or alternatively a sum of the expression levels, may be calculated for the plurality of overexpressed genes and for the plurality of underexpressed genes, to thereby produce or calculate a ratio.

Accordingly, determining cancer aggressiveness and/or a prognosis for a cancer patient in certain embodiments of the present invention further includes determining the ratio of the expression level (e.g. an average or sum of the expression level) of the plurality of overexpressed genes to the expression level (e.g. an average or sum of the expression level) of the plurality of underexpressed genes.

In another aspect of the invention relates to a method of determining the aggressiveness of a cancer in a mammal, said method including the step of comparing an expression level of a plurality of overexpressed genes associated with chromosomal instability and an expression level of a plurality of underexpressed genes associated with estrogen receptor signalling in one or more cancer cells, tissues or organs of the mammal, wherein: a higher relative expression level of the plurality of overexpressed genes associated with chromosomal instability compared to the plurality of underexpressed genes associated with estrogen receptor signalling indicates or correlates with higher aggressiveness of the cancer; and/or a lower relative expression level expression level of the plurality of overexpressed genes associated with chromosomal instability compared to the plurality of underexpressed genes associated with estrogen receptor signalling indicates or correlates with lower aggressiveness of the cancer compared to a mammal having a higher expression level.

In yet another aspect of the invention relates to a method of determining a cancer prognosis for a mammal, said method including the step of comparing an expression level of a plurality of overexpressed genes associated with chromosomal instability and an expression level of a plurality of underexpressed genes associated with estrogen receptor signalling in the mammal, wherein: a higher relative expression level of the plurality of overexpressed genes associated with chromosomal instability compared to the plurality of underexpressed genes associated with estrogen receptor signalling indicates or correlates with a less favourable cancer prognosis; and/or a lower relative expression level of the plurality of overexpressed genes associated with chromosomal instability compared to the plurality of underexpressed genes associated with estrogen receptor signalling indicates or correlates with a more favourable cancer prognosis.

Non-limiting examples of genes in a chromosomal instability (CIN) metagene include ATP6V1C1, RAP2A, CALM1, COG8, HELLS, KDM5A, PGK1, PLCH1, CEP55, RFC4, TAF2, SF3B3, GP1, PIR, MCM10, MELK, FOXM1, KIF2C, NUP155, TPX2, TTK, CENPA, CENPN, EXO1, MAPRE1, ACOT7, NAE1, SHMT2, TCP1, TXNRD1, ADM, CHAF1A and SYNCRIP genes, although without limitation thereto; and an estrogen receptor signalling (ER) metagene may comprise BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3 genes, although without limitation thereto. Table 4 provides further examples of genes that are components of a CIN metagene or that are components of an ER metagene.

An average expression level may be calculated for the CIN metagene and for the ER metagene, to thereby produce or calculate a ratio.

Alternatively, a sum of expression levels may be calculated for the CIN metagene and for the ER metagene, to thereby produce or calculate a ratio.

In certain embodiments, a higher or increased ratio of the average or sum of expression levels of a CIN metagene relative to an ER metagene is associated with, correlates with or is indicative of, higher or increased cancer aggressiveness.

Thus, some embodiments of the invention provide an “aggressiveness score” which is the ratio of CIN metagene expression level (e.g. average or sum of expression of CIN genes) to an ER metagene expression level (e.g average or sum of expression of ER genes).

Accordingly, embodiments of the aforementioned aspects of the invention include determining, assessing or measuring an expression level of a plurality of overexpressed genes associated with chromosomal instability and determining, assessing or measuring an expression level of a plurality of underexpressed genes associated with estrogen receptor signalling. In this regard, reference is made to Table 4 which provides a listing of 206 genes that include genes associated with chromosomal instability and genes associated with estrogen receptor signalling. Preferably, the chromosomal instability genes are of a CIN metagene, comprising genes such as ATP6V1C1, RAP2A, CALM1, COG8, HELLS, KDM5A, PGK1, PLCH1, CEP55, RFC4, TAF2, SF3B3, GP1, PIR, MCM10, MELK, FOXM1, KIF2C, NUP155, TPX2, 11K, CENPA, CENPN, EXO1, MAPRE1, ACOT7, NAE1, SHMT2, TCP1, TXNRD1, ADM, CHAF1A and SYNCRIP, although without limitation thereto. In one preferred embodiment, the chromosomal instability genes are selected from the group consisting of MELK, MCM10, CENPA, EXO1, TTK and KIF2C. Preferably, the estrogen receptor signalling genes are of an ER metagene comprising genes such as BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3, although without limitation thereto. In one preferred embodiment, the estrogen receptor signalling genes are selected from the group consisting of MAPT and MYB.

In certain embodiments, the method of the aforementioned two aspects further includes the step of comparing an expression level of one or more other overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and an expression level of one or more other underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or more cancer cells, tissues or organs of the mammal, wherein: a higher relative expression level of the one or more other overexpressed genes compared to the one or more other underexpressed genes indicates or correlates with higher aggressiveness of the cancer and/or a less favourable cancer prognosis; and/or a lower relative expression level of the one or more other overexpressed genes compared to the one or more other underexpressed genes indicates or correlates with lower aggressiveness of the cancer and/or a more favourable cancer prognosis compared to a mammal having a higher expression level.

In one embodiment, the one or more other overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or more other underexpressed genes are selected from the group consisting of BTN2A2, ERC2, IGH, ME1, MTMR7, SMPDL3B and ZNRD1-AS1.

In this regard, an average expression level, or alternatively a sum of the expression levels, may be calculated for the one or more other overexpressed genes and for the one or more other underexpressed genes, to thereby produce or calculate a ratio.

Accordingly, determining cancer aggressiveness and/or a prognosis for a cancer patient in certain embodiments of the present invention further includes determining the ratio of the expression level (e.g. an average or sum of the expression level) of the one or more other overexpressed genes to the expression level (e.g. an average or sum of the expression level) of the one or more other underexpressed genes.

Detection and/or measurement of expression of the one or more other overexpressed genes and the one or more other underexpressed genes may be performed by any of those methods or combinations thereof described herein (e.g measuring mRNA levels or an amplified cDNA copy thereof and/or by measuring a protein product thereof), albeit without limitation thereto.

Suitably, the comparison of the expression level of the plurality of overexpressed genes associated with chromosomal instability and the expression level of the plurality of underexpressed genes associated with estrogen receptor signalling is integrated with the comparison of the expression level of the one or more other overexpressed genes and the expression level of the one or more other underexpressed genes to derive a first integrated score. In particular embodiments, this may include deriving the first integrated score, at least in part, by addition, subtraction, multiplication, division and/or exponentiation.

By way of example, the comparison of the expression level of the plurality of overexpressed genes associated with chromosomal instability and the expression level of the plurality of underexpressed genes associated with estrogen receptor signalling may be added to, subtracted from, multiplied by, divided by and/or raised to the power of the comparison of the expression level of the one or more other overexpressed genes and the expression level of the one or more other underexpressed genes to derive the first integrated score. Alternatively, the comparison of the expression level of the one or more other overexpressed genes and the expression level of the one or more other underexpressed genes may be added to, subtracted from, multiplied by, divided by and/or raised to the power of the comparison of the expression level of the plurality of overexpressed genes associated with chromosomal instability and the expression level of the plurality of underexpressed genes associated with estrogen receptor signalling to derive the first integrated score.

In a particular preferred embodiment, the first integrated score is derived by exponentiation, wherein the comparison of the expression level of the one or more other overexpressed genes and the expression level of the one or more other underexpressed genes is raised to the power of the comparison of the expression level of the plurality of overexpressed genes associated with chromosomal instability and the expression level of the plurality of underexpressed genes associated with estrogen receptor signalling.

As would be appreciated by the skilled person, the other overexpressed and underexpressed genes described herein may not necessarily be associated with chromosomal instability and estrogen receptor signalling respectively.

In a further aspect, the invention provides a method of determining the aggressiveness of a cancer in a mammal, said method including the step of comparing an expression level of one or more overexpressed genes, wherein the one or more overexpressed genes are selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and an expression level of one or more underexpressed genes, wherein the one or more underexpressed genes are selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or more cancer cells, tissues or organs of the mammal, wherein: a higher relative expression level of the one or more overexpressed genes compared to the one or more underexpressed genes indicates or correlates with higher aggressiveness of the cancer; and/or a lower relative expression level of the one or more overexpressed genes compared to the one or more underexpressed genes indicates or correlates with lower aggressiveness of the cancer compared to a mammal having a higher expression level.

In one embodiment, the one or more overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or more underexpressed genes are selected from the group consisting of BTN2A2, ERC2, IGH, ME1, MTMR7, SMPDL3B and ZNRD1-AS1.

In yet another aspect, the invention provides a method of determining a cancer prognosis for a mammal, said method including the step of comparing an expression level of one or more overexpressed genes, wherein the one or more overexpressed genes are selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and an expression level of one or more underexpressed genes, wherein the one or more underexpressed genes are selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or more cancer cells, tissues or organs of the mammal, wherein: a higher relative expression level of the one or more overexpressed genes compared to the one or more underexpressed genes indicates or correlates with a less favourable cancer prognosis; and/or a lower relative expression level of the one or more overexpressed genes compared to the one or more underexpressed genes indicates or correlates with a more favourable cancer prognosis compared to a mammal having a higher expression level.

In one embodiment, the one or more overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or more underexpressed genes are selected from the group consisting of BTN2A2, ERC2, IGH, ME1, MTMR7, SMPDL3B and ZNRD1-AS1.

In particular embodiments, the method of the aforementioned aspects further includes the step of comparing an expression level of one or more overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and an expression level of one or more underexpressed proteins selected from the group consisting of VEGFR2, HER3, ASNS, MAPK9, ESR1, YWHAE, RAD50, PGR, COL6A1, PEA15 and RPS6, in one or more cancer cells, tissues or organs of the mammal, wherein: a higher relative expression level of the one or more overexpressed proteins compared to the one or more underexpressed proteins indicates or correlates with higher aggressiveness of the cancer and/or a less favourable cancer prognosis; and/or a lower relative expression level of the one or more overexpressed proteins compared to the one or more underexpressed proteins indicates or correlates with lower aggressiveness of the cancer and/or a more favourable cancer prognosis compared to a mammal having a higher expression level.

As would be appreciated by the skilled artisan, the expression level of one or more of the overexpressed proteins and/or one or more of the underexpressed proteins described herein may include one or more phosphorylated forms of said proteins (i.e., a phosphoprotein). In one embodiment, EIF4EBP1 is or comprises one or more phosphoproteins selected from the group consisting of pEIF4EBP1^{S65}, pEIF4EBP1^{T37}, pEIF4EBP1^{T46} and pEIF4EBP1^{T70}. In one embodiment, EGFR is or comprises one or more phosphoproteins selected from the group consisting of pEGFR^{Y1068} and pEGFR^{Y1173}. In one embodiment, HER3 is or comprises pHER3^{Y1289}. In one embodiment, AKT1 is or comprises one or more phosphoproteins selected from the group consisting of pAKT1^{S473} and pAKT1^{T308}. In one embodiment, NFKB1 is or comprises pNFKB1^{S536} In one embodiment, HER2 is or comprises pHER2^{Y1248}. In one embodiment, ESR1 is or comprises pESR1^{S118}. In one embodiment, PEA15 is or comprises pPEA15^{S116}. In one embodiment, RPS6 is or comprises one or more phosphoproteins selected from the group consisting of pRPS6^{S235}, pRPS6^{S236}, pRPS6^{S240} and pRPS6^{S244}.

An average or sum of the expression levels may be calculated for the overexpressed genes, the underexpressed genes, the overexpressed proteins and/or the underexpressed proteins, to thereby produce or calculate a ratio.

Thus, in certain embodiments of the present invention determining cancer aggressiveness and/or a prognosis for a cancer patient includes determining (i) the ratio of the expression level (e.g. an average or sum of the expression level) of the one or more overexpressed genes to the expression level (e.g. an average or sum of the expression level) of the one or more underexpressed genes; and/or (ii) the ratio of the expression level (e.g. an average or sum of the expression level) of the one or more overexpressed proteins to the expression level (e.g. an average or sum of the expression level) of the one or more underexpressed proteins.

Detection and/or measurement of expression of the overexpressed proteins and the underexpressed proteins may be performed by any of those methods or combinations thereof hereinbefore described, albeit without limitation thereto.

Suitably, the comparison of the expression level of the one or more overexpressed proteins and the expression level of the one or more underexpressed proteins is to thereby derive an integrated score. In one particular embodiment, the comparison of the expression level of the one or more overexpressed proteins and the expression level of the one or more underexpressed proteins is integrated with:
(i) the comparison of the expression level of the overexpressed genes associated with chromosomal instability and the expression level of the underexpressed genes associated with estrogen receptor signalling to derive a second integrated score; or
(ii) the first integrated score to derive a third integrated score; or
(iii) the comparison of the expression level of the overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1 and the expression level of the underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 to derive a fourth integrated score; or
(iv) the comparison of the expression level of the overexpressed genes and an expression level of the underexpressed genes, wherein the genes are from one or more of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene, to derive a fifth integrated score; or
(v) the comparison of the expression level of the overexpressed genes and an expression level of the underexpressed genes, wherein the genes are from one or more of the Metabolism metagene, the Signalling metagene, the Development and Growth metagene, the Chromosome Segregation/Replication metagene, the Immune Response metagene and/or the Protein Synthesis/Modification metagene, to derive a sixth integrated score.

In particular embodiments, the second, third, fourth, fifth and/or sixth integrated scores are derived, at least in part, by addition, subtraction, multiplication, division and/or exponentiation. By way of example, the comparison of the expression level of the one or more overexpressed proteins and the expression level of the one or more underexpressed proteins may be added to, subtracted from, multiplied by, divided by and/or raised to the power of (i) the comparison of the expression level of the plurality of overexpressed genes associated with chromosomal instability and the expression level of the plurality of underexpressed genes associated with estrogen receptor signalling; or (ii) the first integrated score. Alternatively, the comparison of the expression level of the plurality of overexpressed genes associated with chromosomal instability and the expression level of the plurality of underexpressed genes associated with estrogen receptor signalling or the first integrated score may be added to, subtracted from, multiplied by, divided by and/or raised to the power of the comparison of the expression level of the one or more overexpressed proteins and the expression level of the one or more underexpressed proteins.

In a further aspect, the invention provides a method of determining the aggressiveness of a cancer in a mammal, said method including the step of comparing an expression level of one or more overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and an expression level of one or more underexpressed proteins selected from the group consisting of VEGFR2, HER3, ASNS, MAPK9, ESR1, YWHAE, RAD50, PGR, COL6A1, PEA15 and RPS6, in one or more cancer cells, tissues or organs of the mammal, wherein: a higher relative expression level of the one or more overexpressed proteins compared to the one or more underexpressed proteins indicates or correlates with higher aggressiveness of the cancer; and/or a lower relative expression level of the one or more overexpressed proteins compared to the one or more underexpressed proteins indicates or correlates with lower aggressiveness of the cancer compared to a mammal having a higher expression level.

In a related aspect, the invention provides a method of determining a cancer prognosis for a mammal, said method including the step of comparing an expression level of one or more overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and an expression level of one or more underexpressed proteins selected from the group consisting of VEGFR2, HER3, ASNS, MAPK9, ESR1, YWHAE, RAD50, PGR, COL6A1, PEA15 and RPS6, in one or more cancer cells, tissues or organs of the mammal, wherein: a higher relative expression level of the one or more overexpressed proteins compared to the one or more underexpressed proteins indicates or correlates with a less favourable cancer prognosis; and/or a lower relative expression level of the one or more overexpressed proteins compared to the one or more underexpressed proteins indicates or correlates with a more favourable cancer prognosis compared to a mammal having a higher expression level.

In particular embodiments of the two aforementioned aspects, one or more of the overexpressed proteins and/or one or more of the underexpressed proteins are or comprise a phosphoprotein hereinbefore described.

An average or sum of the expression levels may be calculated for the one or more overexpressed proteins and the one or more underexpressed proteins, to thereby produce or calculate a ratio as hereinbefore described.

This information with respect to the aggressiveness and/or prognosis of a patient's cancer may prove useful to a physician and/or clinician in determining the most effective course of treatment. A determination of the likelihood for a cancer relapse or of the likelihood of metastasis can assist the physician and/or clinician in determining whether a more conservative or a more radical approach to therapy should be taken. As such, a prognosis may provide for the selection and classification of patients who are predicted to benefit from a given therapeutic regimen.

Accordingly, another aspect of the invention provides a method of predicting the responsiveness of a cancer to an anti-cancer treatment in a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein an altered or modulated relative expression level of the overexpressed genes compared to the underexpressed genes indicates or correlates with relatively increased or decreased responsiveness of the cancer to the anti-cancer treatment.

As would be understood by the skilled person, the relative expression level of a gene or protein may be deemed to be “altered” or “modulated” when the expression level is higher/increased or lower/decreased when compared to a control or reference sample or expression level, such as a threshold level. In one embodiment, a relative expression level may be classified as high if it is greater than a mean and/or median relative expression level of a reference population and a relative expression level may be classified as low if it is less than the mean and/or median relative expression level of the reference population. In this regard, a reference population may be a group of subjects who have the same cancer type, subgroup, stage and/or grade as said mammal for which the relative expression level is determined.

Suitably, for the present aspect the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or more genes listed in Table 21.

In a related aspect, the invention provides a method of predicting the responsiveness of a cancer to an anti-cancer treatment in a mammal, said method including the step of comparing an expression level of a plurality of overexpressed genes and an expression level of a plurality of underexpressed genes in one or more cancer cells, tissues or organs of the mammal, wherein the overexpressed genes and the underexpressed genes are from one or more metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein an altered or modulated relative expression level of the overexpressed genes compared to the underexpressed genes indicates or correlates with relatively increased or decreased responsiveness of the cancer to the anti-cancer treatment.

In one embodiment of the two aforementioned aspects, the plurality of overexpressed genes and/or the plurality of underexpressed genes are selected from one of the metagenes. In an alternative embodiment, the plurality of overexpressed genes and/or the plurality of underexpressed genes are selected from a plurality of the metagenes.

Suitably, the Metabolism metagene, the Signalling metagene, the Development and Growth metagene, the Chromosome Segregation/Replication metagene, the Immune Response metagene and/or the Protein Synthesis/Modification metagene comprise one or more genes listed in Table 22.

In particular embodiments, the plurality of overexpressed genes and the plurality of underexpressed genes are from one or more of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene.

In a related aspect, the invention provides a method of predicting the responsiveness of a cancer to an anti-cancer treatment in a mammal, said method including the step of determining an expression level of one or more genes associated to with chromosomal instability (CIN) in one or more cancer cells of the mammal, wherein a higher expression level indicates or correlates with relatively increased responsiveness of the cancer to the anti-cancer treatment.

As will be described in more detail, overexpression of some CIN genes may be predictive of the responsiveness of a cancer to an anti-cancer treatment, particularly although not exclusively when overexpressed by non-mitotic cancer cells. In this context, by “non-mitotic” means that the cancer cell is not in the mitotic or “M phase” of the cell cycle. Preferably, the non-mitotic cancer cells are in interphase. Broadly, any overexpressed CIN gene set forth Table 4 may be predictive of the responsiveness of a cancer to an anti-cancer treatment. In particular embodiments, the CIN gene is selected from the group consisting of: TTK, CEP55, FOXM1 and SKIP2. In a particularly preferred embodiment, the CIN gene is selected from the group consisting of: TTK, CEP55, FOXM1 and SKIP2 and the cancer is breast cancer. In this regard, the inventors have shown that “bulk” measurements of extracted CIN gene mRNA or encoded protein do not provide a useful indication of whether overexpression of the CIN gene may be predictive of the responsiveness of a cancer to an anti-cancer treatment. More particularly, detection of CIN gene expression by individual cancer cells, particularly non-mitotic or interphase cancer cells, provides a more powerful indication of the responsiveness of a cancer to an anti-cancer treatment.

As previously described, detection and/or measurement of expression of the CIN gene may be performed by measuring RNA (e.g mRNA or an amplified cDNA copy thereof) or by measuring a protein product of a CIN gene. In a particularly preferred embodiment, a protein product of a CIN gene is detected or measured by immunohistochemistry. Typically, although not exclusively, a preferred immunohistochemistry method includes binding an antibody to the protein product of a CIN gene expressed by a cell or tissue and subsequent detection of the bound antibody. By way of example only, the antibody may be unlabelled, directly labelled with an enzyme such as horseradish peroxidase, alkaline phosphatase or glucose oxidase or directly labelled with biotin or digoxigenin. In embodiments where the antibody is unlabelled, a secondary antibody (labelled such as described above) may be used to detect the bound antibody. Biotinylated antibodies may be detected using avidin complexed with an enzyme such as horseradish peroxidase, alkaline phosphatase or glucose oxidase. Suitable enzyme substrates include diaminobanzidine (DAB), permanent red, 3-ethylbenzthiazoline sulfonic acid (ABTS), 5-bromo-4-chloro-3-indolyl phosphate (BCIP), nitro blue tetrazolium (NBT), 3,3′,5,5′-tetramethyl benzidine (TNB) and 4-chloro-1-naphthol (4-CN), although without limitation thereto.

In a further aspect, the invention provides a method of predicting the responsiveness of a cancer to an anti-cancer treatment in a mammal, said method including the step of comparing an expression level of a plurality of overexpressed genes associated with chromosomal instability and an expression level of a plurality of underexpressed genes associated with estrogen receptor signalling in one or more cancer cells, tissues or organs of the mammal, wherein an altered or modulated relative expression level of the overexpressed genes associated with chromosomal instability compared to the underexpressed genes associated with estrogen receptor signalling indicates or correlates with relatively increased or decreased responsiveness of the cancer to the anti-cancer treatment.

In certain embodiments, the genes associated with chromosomal instability are of a CIN metagene. Non-limiting examples include genes selected from the group consisting of: ATP6V1C1, RAP2A, CALM1, COG8, HELLS, KDM5A, PGK1, PLCH1, CEP55, RFC4, TAF2, SF3B3, GP1, PIR, MCM10, MELK, FOXM1, KIF2C, NUP155, TPX2, 11K, CENPA, CENPN, EXO1, MAPRE1, ACOT7, NAE1, SHMT2, TCP1, TXNRD1, ADM, CHAF1A and SYNCRIP. In one preferred embodiment, the chromosomal instability genes are selected from the group consisting of MELK, MCM10, CENPA, EXO1, TTK and KIF2C.

In certain embodiments, the genes associated with estrogen receptor signalling are of an ER metagene. Non-limiting examples include genes selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3. In one preferred embodiment, the estrogen receptor signalling genes are selected from the group consisting of MAPT and MYB.

Suitably, the method of this aspect further includes the step of comparing an expression level of one or more other overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and an expression level of one or more other underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 in one or more cancer cells, tissues or organs of the mammal, wherein an altered or modulated relative expression level of the one or more other overexpressed genes compared to the one or more other underexpressed genes indicates or correlates with relatively increased or decreased responsiveness of the cancer to the anti-cancer treatment.

In one embodiment, the one or more other overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or more other underexpressed genes are selected from the group consisting of BTN2A2, ERC2, IGH, ME1, MTMR7, SMPDL3B and ZNRD1-AS1.

In certain embodiments, the comparison of the expression level of the one or more other overexpressed genes and the expression level of the one or more other underexpressed genes is integrated with the comparison of the expression level of the plurality of overexpressed genes associated with chromosomal instability and the expression level of the plurality of underexpressed genes associated with estrogen receptor signalling to derive a first integrated score as described herein, which is indicative of, or correlates with, responsiveness of the cancer to the anti-cancer treatment.

In another related aspect, the invention provides a method of predicting the responsiveness of a cancer to an anti-cancer treatment in a mammal, said method including the step of comparing an expression level of one or more overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and an expression level of one or more underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or more cancer cells, tissues or organs of the mammal, wherein an altered or modulated relative expression level of the one or more overexpressed genes compared to the one or more underexpressed genes indicates or correlates with relatively increased or decreased responsiveness of the cancer to the anti-cancer treatment.

In one embodiment, the one or more overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593.

In one embodiment, the one or more underexpressed genes are selected from the group consisting of BTN2A2, ERC2, IGH, ME1, MTMR7, SMPDL3B and ZNRD1-AS1.

In particular embodiments, the method of the five aforementioned aspects further includes the step of comparing an expression level of one or more overexpressed proteins selected from the group consisting of DVL3, PM-1, VEGFR2, INPP4B, EIF4EBP1, EGFR, Ku80, HER3, SMAD1, GATA3, ITGA2, AKT1, NFKB1, HER2, ASNS and COL6A1, and an expression level of one or more underexpressed proteins selected from the group consisting of VEGFR2, HER3, ASNS, MAPK9, ESR1, YWHAE, RAD50, PGR, COL6A1, PEA15 and RPS6, in one or more cancer cells, tissues or organs of the mammal, wherein: a higher relative expression level of the one or more overexpressed proteins compared to the one or more underexpressed proteins indicates or correlates with higher aggressiveness of the cancer and/or a less favourable cancer prognosis; and/or a lower relative expression level of the one or more overexpressed proteins compared to the one or more underexpressed proteins indicates or correlates with lower aggressiveness of the cancer and/or a more favourable cancer prognosis compared to a mammal having a higher expression level.

In particular embodiments, one or more of the overexpressed proteins and/or one or more of the underexpressed proteins are or comprise a phosphoprotein hereinbefore described.

An average or sum of the expression levels may be calculated for the overexpressed genes, the underexpressed genes, the overexpressed proteins and/or the underexpressed proteins, to thereby produce or calculate a ratio, as hereinbefore described.

Detection and/or measurement of expression of the overexpressed proteins and the underexpressed proteins may be performed by any of those methods or combinations thereof hereinbefore described, albeit without limitation thereto.

Suitably, the comparison of the expression level of the one or more overexpressed proteins and the expression level of the one or more underexpressed proteins is to thereby derive an integrated score. In one particular embodiment, the comparison of the expression level of the one or more overexpressed proteins and the expression level of the one or more underexpressed proteins is integrated with:
(i) the comparison of the expression level of the overexpressed genes associated with chromosomal instability and the expression level of the underexpressed genes associated with estrogen receptor signalling to derive a second integrated score; or
(ii) the first integrated score to derive a third integrated score; or
(iii) the comparison of the expression level of the overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1 and the expression level of the underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3 to derive a fourth integrated score; or
(iv) the comparison of the expression level of the overexpressed genes and an expression level of the underexpressed genes, wherein the genes are from one or more of the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene, to derive a fifth integrated score; or
(v) the comparison of the expression level of the overexpressed genes and an expression level of the underexpressed genes, wherein the genes are from one or more of the Metabolism metagene, the Signalling metagene, the Development and Growth metagene, the Chromosome Segregation/Replication metagene, the Immune Response metagene and/or the Protein Synthesis/Modification metagene, to derive a sixth integrated score,
   wherein the second, third, fourth, fifth and/or sixth integrated score is indicative of, or correlates with, responsiveness of the cancer to the anti-cancer treatment.

In particular embodiments, the second, third, fourth, fifth and/or sixth integrated scores are derived, at least in part, by addition, subtraction, multiplication, division and/or exponentiation, as hereinbefore described.

In a further related aspect, the invention provides a method of predicting the responsiveness of a cancer to an anti-cancer treatment in a mammal, said method including the step of comparing an expression level of one or more overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and an expression level of one or more underexpressed proteins selected from the group consisting of VEGFR2, HER3, ASNS, MAPK9, ESR1, YWHAE, RAD50, PGR, COL6A1, PEA15 and RPS6, in one or more cancer cells, tissues or organs of the mammal, wherein an altered or modulated relative expression level of the one or more overexpressed proteins compared to the one or more underexpressed proteins indicates or correlates with relatively increased or decreased responsiveness of the cancer to the anti-cancer treatment.

In particular embodiments, one or more of the overexpressed proteins and/or one or more of the underexpressed proteins are or comprise a phosphoprotein hereinbefore described.

It will be appreciated from the foregoing that the invention provides methods that determine the aggressiveness of a cancer, facilitate providing a cancer prognosis for a patient and/or predict the responsiveness of a cancer to an anti-cancer treatment. Particular, broad embodiments of the invention include the step of treating the patient following determining the aggressiveness of the cancer, providing a cancer prognosis and/or predicting the responsiveness of the cancer to anti-cancer treatment. Accordingly, these embodiments relate to using information obtained about the aggressiveness of the cancer, the cancer prognosis and/or the predicted responsiveness of the cancer to anti-cancer treatment to thereby construct and implement an anti-cancer treatment regime for the patient. In a preferred embodiment, this is personalized to a particular patient so that the treatment regime is optimized for that particular patient.

Cancer treatments may include drug therapy, chemotherapy, antibody, nucleic acid and other biomolecular therapies, radiation therapy, surgery, nutritional therapy, relaxation or meditational therapy and other natural or holistic therapies, although without limitation thereto. In particular embodiments, the cancer therapy may target aneuploidy or aneuploid tumours and/or chromosomal instability.

Generally, drugs, biomolecules (e.g antibodies, inhibitory nucleic acids such as siRNA) or chemotherapeutic agents are referred to herein as “anti-cancer therapeutic agents”. In some embodiments relating to breast cancer, the anti-cancer treatment may include HER2-directed therapy such as trastuzumab and endocrine therapies such as tamoxifen and aromatase inhibitors. In other or alternative embodiments, the therapy may include administration of inhibitors of CIN genes or CIN gene products, such as one or more of those listed in Table 4. It will be appreciated that inhibition of the CIN gene product TTK using the specific inhibitor AZ3146 was effective against TNBC cell lines. Furthermore, siRNA-mediated knockdown of the CIN genes 11K, TPX2, NDC80 and PBK was effective against TNBC cell lines.

In certain embodiments, the cancer treatment may be directed at genes or gene products other than those listed in Tables 4, 10, 21 and/or 22. By way of example, the cancer treatment may target genes or gene products such as PLK1^{71,72} or others⁷³⁻⁷⁶ to thereby target aneuploid tumours or tumour cells.

Suitably, when considering (i) the relative expression of one or more of the overexpressed genes of the 29 gene signature (i.e., CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1) when compared to one or more of the underexpressed genes of the 30 gene signature (i.e., BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3); (ii) the relative expression of one or more of the overexpressed proteins (i.e., DVL3, PAI-1, VEGFR2, INPP4B, EIF4EBP1, EGFR, Ku80, HER3, SMAD1, GATA3, ITGA2, AKT1, NFKB1, HER2, ASNS and COL6A1) when compared to one or more of the underexpressed proteins (i.e., VEGFR2, HER3, ASNS, MAPK9, ESR1, YWHAE, RAD50, PGR, COL6A1, PEA15 and RPS6); and/or (iii) the first, second, third and/or fourth integrated score, the anticancer therapeutic agent is selected from the group consisting of a chemotherapy, an endocrine therapy, immunotherapy and a molecularly targeted therapy. In certain embodiments, the anticancer treatment comprises an ALK inhibitor (e.g., TAE684), an Aurora kinase inhibitor (e.g., Alisertib, AMG-900, BI-847325, GSK-1070916A, ilorasertib, MK-8745, danusertib), a BCR-ABL inhibitor (e.g., Nilotinib, Dasatinib, Ponatinib), a HSP90 inhibitor (e.g., Tanespimycin (17-AAG), PF0429113, AUY922, Luminespib, ganetespib, Debio-0932), an EGFR inhibitor (e.g., Afatinib, Erlotinib, Lapatinib, cetuximab), a PARP inhibitor (e.g., ABT-888, AZD-2281), retinoic acid (e.g., all-trans retinoic acid or ATRA), a Bcl2 inhibitor (e.g., ABT-263), a gluconeogenesis inhibitor (e.g., metformin), a p38 MAPK inhibitor (e.g., BIRB0796, LY2228820), a MEK1/2 inhibitor (e.g., trametinib, cobimetinib, binimetinib, selumetinib, pimasertib, refametinib, TAK-733), a mTOR inhibitor (e.g., BEZ235, JW-7-25-1), a PI3K inhibitor (e.g., Idelalisib, buparlisib/apelisib, copanlisib, GSK-2636771, pictilisib, AMG-319, AZD-8186), an IGF1R inhibitor (e.g., BMS-754807, dalotuzumab, ganitumab, linsitinib), a PLCγ inhibitor (e.g., U73122), a JNK inhibitor (e.g., SP600125), a PAK1 inhibitor (e.g., IPA3), a SYK inhibitor (e.g., BAY613606), a HDAC inhibitor (e.g., Vorinostat), an FGFR inhibitor (e.g., Dovitinib), a XIAP inhibitor (e.g., Embelin), a PLK1 inhibitor (e.g., Volasertib, P-937), an ERK5 inhibitor (e.g., XMD8-92), a MPS1/TTK inhibitor (e.g., BAY-1161909) and any combination thereof.

By way of example, patients with a high relative expression level of one or more overexpressed genes, such as those of the 21 gene signature, when compared to one or more underexpressed genes, such as those of the 7 gene signature, a high relative expression level of one or more overexpressed proteins when compared to one or more underexpressed proteins and/or a high integrated score described herein are more likely to respond favourably, such as a pathological complete response, when treated with chemotherapy. In this regard, non-limiting examples of chemotherapy include a pyrimidine analogue (e.g., 5-fluorouracil, capecitabine), a taxane (e.g., paclitaxel), an anthracycline (e.g., doxorubicin, epirubicin), an anti-folate drug (e.g., the dihydrofolate reductase inhibitor methotrexate), an alkylating agent (e.g., cyclophosphamide) or any combination thereof. It would be appreciated that the chemotherapy may be administered as adjuvant, neoadjuvant and/or as standard therapy, alone or in combination with other anticancer therapeutics.

Additionally, in certain embodiments, patients with a high relative expression level of one or more overexpressed genes, such as those of the 29 gene signature, when compared to one or more underexpressed genes, such as those of the 30 gene signature, a high relative expression level of one or more overexpressed proteins when compared to one or more underexpressed proteins and/or a high integrated score described herein may be more likely to respond favourably to (i.e., be more sensitive to) inhibition of HSP90, EGFR, IGF1R, mTOR, PI3K, p38 MAPK, PLCγ, JNK, PAK1, ERK5, XIAP, PLK1 and/or MEK1/2 and may be less likely to respond favourably to (i.e., be less sensitive to) anticancer treatment with an ALK inhibitor, a BCR-ABL inhibitor, a PARP inhibitor, retinoic acid, a Bcl2 inhibitor, a gluconeogenesis inhibitor, a p38 MAPK inhibitor, an FGFR inhibitor, a SYK inhibitor, a HDAC inhibitor and/or an IGF1R inhibitor.

It will also be understood that the gene and protein signatures described herein may be used to identify those poorer prognosis patients, such as those with larger and/or higher grade tumours, who may benefit from one or more additional anticancer therapeutic agents to the typical or standard anti-cancer treatment regime for that particular patient group. By way of example, ER⁺ breast cancer patients with or without lymph node involvement with a high integrated score, and hence a relatively poor prognosis, are more likely to respond favourably to or benefit from chemotherapy and/or endocrine therapy. This may include an improved survival and/or reduced likelihood of tumour recurrence and/or metastasis for these patients.

In certain embodiments, for patients with a high relative expression level of the overexpressed genes of the 21 gene signature when compared to the underexpressed genes of the 7 gene signature and/or a high integrated score, the cancer treatment may be directed at those genes or gene products listed in Tables 13, 15, 16 and 17.

Additionally, for patients with a high relative expression level of the overexpressed proteins when compared to the underexpressed proteins and/or a high integrated score the cancer treatment may be directed at one or more of those proteins listed in Table 19.

It would be appreciated that those methods described herein for predicting the responsiveness of a cancer to an anti-cancer treatment, such as an immunotherapeutic agent, may further include the step of administering to the mammal a therapeutically effective amount of the anticancer treatment. In a preferred embodiment, the anticancer treatment is administered when the altered or modulated relative expression level indicates or correlates with relatively increased responsiveness of the cancer to the anti-cancer treatment.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

The term “therapeutically effective amount” describes a quantity of a specified agent sufficient to achieve a desired effect in a subject being treated with that agent. For example, this can be the amount of a composition comprising one or more agents that binds one or more of the overexpressed and/or underexpressed genes or gene products thereof described herein, necessary to reduce, alleviate and/or prevent a cancer or cancer associated disease, disorder or condition. In some embodiments, a “therapeutically effective amount” is sufficient to reduce or eliminate a symptom of a cancer. In other embodiments, a “therapeutically effective amount” is an amount sufficient to achieve a desired biological effect, for example an amount that is effective to decrease or prevent cancer growth and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

Suitably, the anti-cancer therapeutic agent is administered to a mammal as a pharmaceutical composition comprising a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In particular embodiments of the hereinbefore described methods, the cancer is breast cancer and the one or more overexpressed proteins are selected from the group consisting of DVL3, VEGFR2, INPP4B, EIF4EBP1, EGFR, HER3, SMAD1, NFKB1 and HER2 and the one or more underexpressed proteins are selected from the group consisting of ASNS, MAPK9, YWHAE, RAD50, PGR, COL6A1, PEA15 and RPS6.

In particular embodiments of the hereinbefore described methods, the cancer is lung cancer, such as lung adenocarcinoma, wherein:

(i) the one or more overexpressed genes are selected from the group consisting of GNB2L1, TXN, KCNG1, BCAP31, GSK3B, FOXM1, ZNF593, EXO1, KIF2C, TTK, MELK, CENPA, TPX2, CA9, GRHPR, HCFC1R1,CEP55, MCM10, CENPN and CARHSP1, and the one or more underexpressed genes are selected from the group consisting of BTN2A2, MTMR7, ZNRD1-AS1, MAPT and BTG2; and/or

(ii) the one or more overexpressed proteins are selected from the group consisting of DVL3, PAI-1, Ku80, GATA3, ITGA2 and AKT1, and the one or more underexpressed proteins are selected from the group consisting of ESR1.

In particular embodiments of the hereinbefore described methods, the cancer is kidney cancer, such as renal clear cell carcinoma, wherein:

(i) the one or more overexpressed genes are selected from the group consisting of EIF3K, ADORA2B, KCNG1, BCAP31, EXOSC7, FOXM1, CD55, ZNF593, KIF2C, TTK, MELK, CENPA, TPX2, CEP55, PML, CENPN and CARHSP1, and the one or more underexpressed genes are selected from the group consisting of BCL2 and MAPT; and/or

(ii) the one or more overexpressed proteins are selected from the group consisting of DVL3, PAI-1 and EIF4EBP1, and the one or more underexpressed proteins are selected from the group consisting of HER3, MAPK9, ESR1 and RAD50.

In particular embodiments of the hereinbefore described methods, the cancer is melanoma, such as skin cutaneous melanoma, and wherein:

(i) the one or more overexpressed genes are selected from the group consisting of EIF3K, ADORA2B, GSK3B, EXOSC7, FOXM1, EXO1, KIF2C, CENPA, TPX2, CAMSAP1, MCM10 and ABHD5 and the one or more underexpressed genes are selected from the group consisting of BCAP31, BTN2A2, SMPDL3B, MTMR7, ME1 and BTG2; and/or

(ii) the one or more overexpressed proteins are selected from the group consisting of PAI-1, EIF4EBP1, EGFR, HER3 and Ku80 and the one or more underexpressed proteins are selected from the group consisting of ASNS, MAPK9 and ESR1.

In particular embodiments of the hereinbefore described methods, the cancer is endometrial cancer, such as uterine corpus endometrioid carcinoma, and wherein:

(i) the one or more overexpressed genes are selected from the group consisting of GNB2L1, EIF3K, KCNG1, BCAP31, GSK3B, EXOSC7, FOXM1, ZNF593, EXO1, KIF2C, MAP2K5, TTK, MELK, GRHPR, and PML, and the one or more underexpressed genes is MYB; and/or

(ii) the one or more overexpressed proteins are selected from the group consisting of DVL3, INPP4B, EIF4EBP1 and ASNS and the one or more underexpressed proteins are selected from the group consisting of MAPK9, ESR1 and YWHAE.

In particular embodiments of the hereinbefore described methods, the cancer is ovarian adenocarcinoma and wherein:

(i) the one or more overexpressed genes are selected from the group consisting of GNB2L1, EIF3K, TXN, ADORA2B, KCNG1, GSK3B, STAU1, MAP2K5, and HCFC1R1, and the one or more underexpressed genes are selected from the group consisting of BTN2A2, and ZNRD1-AS1; and/or

(ii) the one or more overexpressed proteins are selected from the group consisting of PAI-1 and VEGFR2 and the one or more underexpressed proteins are selected from the group consisting of ASNS, MAPK9, ESR1, YWHAE and PGR.

In particular embodiments of the hereinbefore described methods, the cancer is head and neck cancer, such as head and neck squamous cell carcinoma, and wherein:

(i) the one or more overexpressed genes are selected from the group consisting of GNB2L1, TXN, ADORA2B, KCNG1, CD55, ZNF593, NDUFC1, and HCFC1R1, and the one or more underexpressed genes are selected from the group consisting of BTN2A2, and MTMR7; and/or

(ii) the one or more overexpressed proteins are selected from the group consisting of PAI-1, INPP4B, EGFR, HER3, SMAD1, GATA3, ITGA2 and COL6A1 and the one or more underexpressed proteins are selected from the group consisting of VEGFR2 and ASNS.

In particular embodiments of the hereinbefore described methods, the cancer is colorectal cancer, such as colorectal adenocarcinoma, and wherein:

(i) the one or more overexpressed genes are selected from the group consisting of EIF3K, TXN, CD55, NDUFC1, HCFC1R1, and PML, and the one or more underexpressed genes are selected from the group consisting of BTN2A2, SMPDL3B, and ME1; and/or

(ii) the one or more overexpressed proteins are selected from the group consisting of DVL3, PAI-1, INPP4B, EIF4EBP1, EGFR and HER3 and the one or more underexpressed proteins are selected from the group consisting of ASNS, MAPK9, YWHAE, RAD50 and PEA15.

In particular embodiments of the hereinbefore described methods, the cancer is glioma, such as lower grade glioma, and wherein:

(i) the one or more overexpressed genes are selected from the group consisting of TXN, BCAP31, STAU1, PML, CARHSP1, and BTN2A2; and/or

(ii) the one or more overexpressed proteins are selected from the group consisting of DVL3, PAI-1, VEGFR2, Ku80, SMAD1 and NFKB1 and the one or more underexpressed proteins are selected from the group consisting of ESR1, YWHAE and PGR.

In particular embodiments of the hereinbefore described methods, the cancer is bladder cancer, such as urothelial carcinoma, and wherein:

(i) the one or more overexpressed genes are selected from the group consisting of ADORA2B, KCNG1, STAU1, MAP2K5, and CAMSAP1, and the one or more underexpressed genes are selected from the group consisting of GNB2L1, EIF3K, TXN, BCAP31, EXOSC7, CD55, NDUFC1, GRHPR, CETN3, BTN2A2, SMPDL3B, and ERC2; and/or

(ii) the one or more overexpressed proteins are selected from the group consisting of DVL3, VEGFR2, Ku80, SMAD1 and AKT1 and the one or more underexpressed proteins is ASNS.

In particular embodiments of the hereinbefore described methods, the cancer is lung cancer, such as lung squamous cell carcinoma, and wherein:

(i) the one or more overexpressed genes are selected from the group consisting of GNB2L1, ZNF593, and SMPDL3B, and the one or more underexpressed genes are selected from the group consisting of GSK3B, MAP2K5, NDUFC1, CAMSAP1, ABHD5, and ME1; and/or

(ii) the one or more overexpressed proteins are selected from the group consisting of DVL3, PAI-1, VEGFR2, INPP4B, EGFR and GATA3 and the one or more underexpressed proteins is ASNS.

In particular embodiments of the hereinbefore described methods, the cancer is adrenocortical carcinoma, and wherein:

the one or more overexpressed genes are selected from the group consisting of GNB2L1, EIF3K, TXN, ADORA2B, KCNG1, BCAP31, FOXM1, ZNF593, EXO1, KIF2C, MAP2K5, TTK, MELK, CENPA, TPX2, GRHPR, CEP55, MCM10, and CENPN, and the one or more underexpressed genes are selected from the group consisting of MTMR7, BCL2, MAPT, MYB, and STC2.

In particular embodiments of the hereinbefore described methods, the cancer is kidney renal papillary cell carcinoma and wherein:

the one or more overexpressed genes are selected from the group consisting of GNB2L1, ADORA2B, KCNG1, GSK3B, FOXM1, CD55, EXO1, KIF2C, STAU1, TTK, MELK, CENPA, TPX2, CA9, CEP55, and MCM10, and the one or more underexpressed genes are selected from the group consisting of SMPDL3B, and BCL2.

In particular embodiments of the hereinbefore described methods, the cancer is pancreatic ductal adenocarcinoma and wherein:

the one or more overexpressed genes are selected from the group consisting of EIF3K, ADORA2B, GSK3B, EXOSC7, FOXM1, CD55, EXO1, STAU1, CAMSAP1, and CETN3 and the one or more underexpressed genes are selected from the group consisting of BTN2A2, SMPDL3B, MTMR7, ME1, BCL2, and ERC2.

In particular embodiments of the hereinbefore described methods, the cancer is liver hepatocellular carcinoma and wherein:

the one or more overexpressed genes are selected from the group consisting of GNB2L1, TXN, EXOSC7, and CA9, and the one or more underexpressed genes is MTMR7.

In particular embodiments of the hereinbefore described methods, the cancer is cervical squamous cell carcinoma and/or endocervical adenocarcinoma and wherein:

the one or more overexpressed genes are selected from the group consisting of STAU1, CA9, and ME1 and the one or more underexpressed genes are selected from the group consisting of EIF3K, TXN, BCAP31, EXOSC7, and ZNRD1-AS1.

Furthermore, in certain embodiments, patients with a high relative expression level of one or more overexpressed genes, such as those of the 29 gene signature, when compared to one or more underexpressed genes, such as those of the 30 gene signature, a high relative expression level of one or more overexpressed proteins when compared to one or more underexpressed proteins and/or a high integrated score as described herein may be more likely to respond favourably to immunotherapy.

Accordingly, one aspect provides a method of predicting the responsiveness of a cancer to an immunotherapeutic agent in a mammal, said method including the step of comparing an expression level of one or more overexpressed genes selected from the group consisting of ADORA2B, CD36, CETN3, KCNG1, LAMA3, MAP2K5, NAE1, PGK1, STAU1, CFDP1, SF3B3 and TXN, and an expression level of one or more underexpressed genes selected from the group consisting of APOBEC3A, BCL2, BTN2A2, CAMSAP1, CAMK4, CARHSP1, FBXW4, GSK3B, HCFC1R1, MYB, PSEN2 and ZNF593, in one or more cancer cells, tissues or organs of the mammal, wherein an altered or modulated relative expression level of the one or more overexpressed genes compared to the one or more underexpressed genes indicates or correlates with relatively increased or decreased responsiveness of the cancer to the immunotherapeutic agent.

In one embodiment the one or more overexpressed genes are selected from the group consisting of ADORA2B, CETN3, KCNG1, MAP2K5, STAU1 and TXN, and/or an expression level of one or more underexpressed genes are selected from the group consisting of BTN2A2, CAMSAP1, CARHSP1, GSK3B, HCFC1R1, and ZNF593.

In one embodiment, the one or more overexpressed genes are selected from the group consisting of ADORA2B, CD36, KCNG1, LAMA3, MAP2K5, NAE1, PGK1, STAU1, CFDP1, and SF3B3 and/or an expression level of one or more underexpressed genes are selected from the group consisting of APOBEC3A, BCL2, BTN2A2, CAMK4, FBXW4, PSEN2 and, MYB.

It would be understood for particular embodiments of the present aspect that one or more other overexpressed genes and/or one or more other underexpressed genes from one or more of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene. such as those listed in Table 21, may be included in the step of comparing an expression level of one or more overexpressed genes and an expression level of one or more underexpressed genes.

Insofar as they relate to cancer, immunotherapy or immunotherapeutic agents use or modify the immune mechanisms of a subject so as to promote or facilitate treatment of a cancer. In this regard, immunotherapy or immunotherapeutic agents used to treat cancer include cell-based therapies, antibody therapies (e.g., anti-PD1 or anti-PDL1 antibodies) and cytokine therapies. These therapies all exploit the phenomenon that cancer cells often have subtly different molecules termed cancer antigens on their surface that can be detected by the immune system of the cancer subject. Accordingly, immunotherapy is used to provoke the immune system of a cancer patient into attacking the cancer's cells by using these cancer antigens as targets.

Non-limiting examples of immunotherapy or immunotherapeutic agents include adalimumab, alemtuzumab, basiliximab, belimumab, bevacizumab, BMS-936559, brentuximab, certolizumab, cituximab, daclizumab, eculizumab, ibritumomab, infliximab, ipilimumab, lambrolkizumab, mepolizumab, MPDL3280A muromonab, natalizumab, nivolumab, ofatumumab, omalizumab, pembrolizumab, pexelizumab, pidilizumab, rituximab, tocilizumab, tositumomab, trastuzumab, ustekinumab, abatacept, alefacept and denileukin diftitox. In particular preferred embodiments, the immunotherapeutic agent is an immune checkpoint inhibitor, such as an anti-PD1 antibody (e.g., pidilizumab, nivolumab, lambrolkizumab, pembrolizumab), an anti-PDL1 antibody (e.g., BMS-936559, MPDL3280A) and/or an anti-CTLA4 antibody (e.g., ipilimumab).

As would be appreciated by the skilled artisan, immune checkpoints refer to a variety of inhibitory pathways of the immune system that are crucial for maintaining self-tolerance and for modulating the duration and/or amplitude of an immune response in a subject. Cancers can use particular immune checkpoint pathways as a major mechanism of immune resistance, particularly against T cells that are specific for tumour antigens. Accordingly, immune checkpoint inhibitors include any agent that blocks or inhibits the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. By way of example, immune checkpoint receptors or receptor ligands that may be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GALS, LAG3, TIM3, B7H3, B7H4, VISTA, KIR, 2B4, CD160 and CGEN-15049. Illustrative immune checkpoint inhibitors include tremelimumab (CTLA-4 blocking antibody), anti-OX40, PD-L1 monoclonal Antibody (Anti-B7-H1; MEDI4736), MK-3475 (PD-1 blocker), nivolumab (anti-PD1 antibody), pidilizamab (CT-011; anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and yervoy/ipilimumab (anti-CTLA-4 checkpoint inhibitor), albeit without limitation thereto.

In one embodiment, the method of predicting the responsiveness of a cancer to an immunotherapeutic agent, may further include the step of administering to the mammal a therapeutically effective amount of the immunotherapeutic agent.

In a related aspect is provided a method of predicting the responsiveness of a cancer to an EGFR inhibitor in a mammal, said method including the step of comparing an expression level of one or more overexpressed genes selected from the group consisting of NAE1, GSK3B, TAF2, MAPRE1, BRD4, STAU1, TAF2, PDCD4, KCNG1, ZNRD1-AS1, EIF4B, HELLS, RPL22, ABAT, BTN2A2, CD1B, ITM2A, BCL2, CXCR4, and ARNT2 and an expression level of one or more underexpressed genes selected from the group consisting of CD1C, CD1E, CD1B, KDM5A, BATF, EVL, PRKCB, HCFC1R1, CARHSP1, CHAD, KIR2DL4, ABHD5, ABHD14A, ACAA1, SRPK3, CFB, ARNT2, NDUFC1, BCL2, EVL, ULBP2, BIN3, SF3B3, CETN3, SYNCRIP, TAF2, CENPN, ATP6V1C1, CD55 and ADORA2B in one or more cancer cells, tissues or organs of the mammal, wherein an altered or modulated relative expression level of the one or more overexpressed genes compared to the one or more underexpressed genes indicates or correlates with relatively increased or decreased responsiveness of the cancer to the EGFR inhibitor.

It would be appreciated that the EGFR inhibitor may be any known in the art, including monoclonal antibody and small molecule inhibitors thereof, such as those hereinbefore described. In particular embodiments, the EGFR inhibitor is or comprises erlotinib and/or cetuximab.

In certain embodiments, the cancer is or comprises lung cancer, colorectal cancer or breast cancer.

In one embodiment, the one or more overexpressed genes are selected from the group consisting of NAE1, GSK3B, and TAF2 and/or the one or more underexpressed genes are selected from the group consisting of CD1C, CD1E, CD1B, KDM5A, BATF, EVL, PRKCB, HCFC1R1, CARHSP1, CHAD, KIR2DL4, ABHD5, ABHD14A, ACAA1, SRPK3, and CFB.

In one embodiment, the one or more overexpressed genes are selected from the group consisting of MAPRE1, BRD4, STAU1, TAF2, GSK3B, PDCD4, KCNG1, ZNRD1-AS1, EIF4B and HELLS and/or the one or more underexpressed genes are selected from the group consisting of ARNT2, NDUFC1, BCL2, ABHD14A, EVL, ULBP2, and BINS.

In one embodiment, the one or more overexpressed genes are selected from the group consisting of RPL22, ABAT, BTN2A2, CD1B, ITM2A, BCL2, CXCR4, and ARNT2 and/or the one or more underexpressed genes are selected from the group consisting of SF3B3, CETN3, SYNCRIP, TAF2, CENPN, ATP6V1C1, CD55 and ADORA2B.

In a related aspect is provided a method of predicting the responsiveness of a cancer to a multikinase inhibitor in a mammal, said method including the step of comparing an expression level of one or more overexpressed genes selected from the group consisting of SCUBE, CHPT1, CDC1, BTG2, ADORA2B and BCL2, and an expression level of one or more underexpressed genes selected from the group consisting of NOP2, CALR, MAPRE1, KCNG1, PGK1, SRPK3, RERE, ADM, LAMA3, KIR2DL4, ULBP2, LAMA4, CA9, and BCAP31, in one or more cancer cells, tissues or organs of the mammal, wherein an altered or modulated relative expression level of the one or more overexpressed genes compared to the one or more underexpressed genes indicates or correlates with relatively increased or decreased responsiveness of the cancer to the EGFR inhibitor.

Multikinase inhibitors typically work by inhibiting multiple intracellular and/or cell surface kinases, some of which may be implicated in tumor growth and metastatic progression of a cancer, thus decreasing tumor growth and replication. It would be appreciated that the multikinase inhibitor may be any known in the art, including small molecule inhibitors, such as those hereinbefore described. Non-limiting examples of multikinase inhibitors include sorafenib, trametinib, dabrafenib, vemurafenib, crizotinib, sunitinib, axitinib, ponatinib, ruxolitinib, vandetanib, cabozantinib, afatinib, ibrutinib and regorafenib. In a particular embodiment, the multikinase inhibitor is or comprises sorafenib.

In one embodiment, the cancer is or comprises lung cancer.

Suitably, with regard to predicting the responsiveness of a cancer to an immunotherapeutic agent, an EGFR inhibitor or a multikinase inhibitor, a higher relative expression level of the one or more overexpressed genes compared to the one or more underexpressed genes indicates or correlates with a relatively increased responsiveness of the cancer to the agent or inhibitor; and/or a lower relative expression level of the one or more overexpressed genes compared to the one or more underexpressed genes indicates or correlates with a relatively decreased responsiveness of the cancer to the agent or inhibitor.

In a further aspect, the invention provides a method for identifying an agent for use in the treatment of cancer including the steps of:

(i) contacting a protein product of GRHPR, NDUFC1, CAMSAP1, CETN3, EIF3K, STAU1, EXOSC7, COG8, CFDP1 and/or KCNG1 with a test agent; and

(ii) determining whether the test agent, at least partly, reduces, eliminates, suppresses or inhibits the expression and/or an activity of the protein product.

Suitably, the cancer is of a type hereinbefore described, albeit without limitation thereto. Preferably, the cancer has an overexpressed gene selected from the group consisting of GRHPR, NDUFC1, CAMSAP1, CETN3, EIF3K, STAU1, EXOSC7, COG8, CFDP1 and KCNG1 and any combination thereof, Suitably, the agent possesses or displays little or no significant off-target and/or nonspecific effects.

Preferably, the agent is an antibody or a small organic molecule.

In embodiments relating to antibody inhibitors, the antibody may be polyclonal or monoclonal, native or recombinant. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative of the protein product of one or more of GRHPR, NDUFC1, CAMSAP1, CETN3, EIF3K, STAU1, EXOSC7, COG8, CFDP1 and KCNG1. For example, the antibodies may be polyclonal antibodies. Such antibodies may be prepared for example by injecting an isolated protein, fragment, variant or derivative of the protein product into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated protein products and/or fragments, variants and/or derivatives thereof.

Typically, the inhibitory activity of candidate inhibitor antibodies may be assessed by in vitro and/or in vivo assays that detect or measure the expression levels and/or activity of the protein products of one or more of GRHPR, NDUFC1, CAMSAP1, CETN3, EIF3K, STAU1, EXOSC7, COG8, CFDP1 and KCNG1 in the presence of the antibody.

In embodiments relating to small organic molecule inhibitors, this may involve screening of large compound libraries, numbering hundreds of thousands to millions of candidate inhibitors (chemical compounds including synthetic, small organic molecules or natural products, for example) which may be screened or tested for biological activity at any one of hundreds of molecular targets in order to find potential new drugs, or lead compounds. Screening methods may include, but are not limited to, computer-based (“in silico”) screening and high throughput screening based on in vitro assays.

Typically, the active compounds, or “hits”, from this initial screening process are then tested sequentially through a series of other in vitro and/or in vivo tests to further characterize the active compounds. A progressively smaller number of the “successful” compounds at each stage are selected for subsequent testing, eventually leading to one or more drug candidates being selected to proceed to being tested in human clinical trials.

At the clinical level, screening a test agent may include obtaining samples from test subjects before and after the subjects have been exposed to a test compound. The levels in the samples of the protein product of the overexpressed genes may then be measured and analysed to determine whether the levels and/or activity of the protein products change after exposure to a test agent. By way of example, protein product levels in the samples may be determined by mass spectrometry, western blot, ELISA and/or by any other appropriate means known to one of skill in the art. Additionally, the activity of the protein products, such as their enzymatic activity, may be determined by any method known in the art. This may include, for example, enzymatic assays, such as spectrophotometric, fluorometric, calorimetric, chemiluminescent, light scattering, microscale thermophoresis, radiometric and chromatographic assays.

It would be appreciated that subjects who have been treated with test agents may be routinely examined for any physiological effects which may result from the treatment. In particular, the test agents will be evaluated for their ability to decrease cancer likelihood or occurrence in a subject. Alternatively, if the test agents are administered to subjects who have previously been diagnosed with cancer, they will be screened for their ability to slow or stop the progression of the cancer as well as induce disease remission.

In a particular embodiment, the invention may provide a “companion diagnostic” whereby the one or more genes that are detected as having elevated expression are the same genes that are targeted by the anti-cancer treatment.

In a related aspect, the invention provides an agent for use in the treatment of cancer identified by the method hereinbefore described.

Suitably, the cancer is of a type hereinbefore described, albeit without limitation thereto. Preferably, the cancer has an overexpressed gene selected from the group consisting of GRHPR, NDUFC1, CAMSAP1, CETN3, EIF3K, STAU1, EXOSC7, COG8, CFDP1, KCNG1 and any combination thereof.

In another related aspect, the invention provides a method of treating a cancer in a mammal, including the step of administering to the mammal a therapeutically effective amount of an agent hereinbefore described.

In this regard, test agents that are identified of being capable of reducing, eliminating, suppressing or inhibiting the expression level and/or activity of a protein product of GRHPR, NDUFC1, CAMSAP1, CETN3, EIF3K, STAU1, EXOSC7, COG8, CFDP1 and/or KCNG1 may then be administered to patients who are suffering from or are at risk of developing cancer. For example, the administration of a test agent which inhibits or decreases the activity and/or expression of the protein product of one or more of the aforementioned genes may treat the cancer and/or decrease the risk cancer, if the increased activity of the biomarker is responsible, at least in part, for the progression and/or onset of the cancer.

Suitably, the cancer is of a type hereinbefore described, albeit without limitation thereto. Preferably, the cancer has an overexpressed gene selected from the group consisting of GRHPR, NDUFC1, CAMSAP1, CETN3, EIF3K, STAU1, EXOSC7, COG8, CFDP1, KCNG1 and any combination thereof.

All computer programs, algorithms, patent and scientific literature referred to herein is incorporated herein by reference.

For the present invention, the database accession number or unique identifier provided herein for a gene or a protein, such as those presented in Tables 4, 5, 10, 15, 16, 17 and 18, as well as the gene and/or protein sequence or sequences associated therewith, are incorporated by reference herein.

So that preferred embodiments of the invention may be fully understood and put into practical effect, reference is made to the following non-limiting examples.

### Example 1

### Materials and Methods

### Meta-Analysis of Global Gene Expression in TNBC

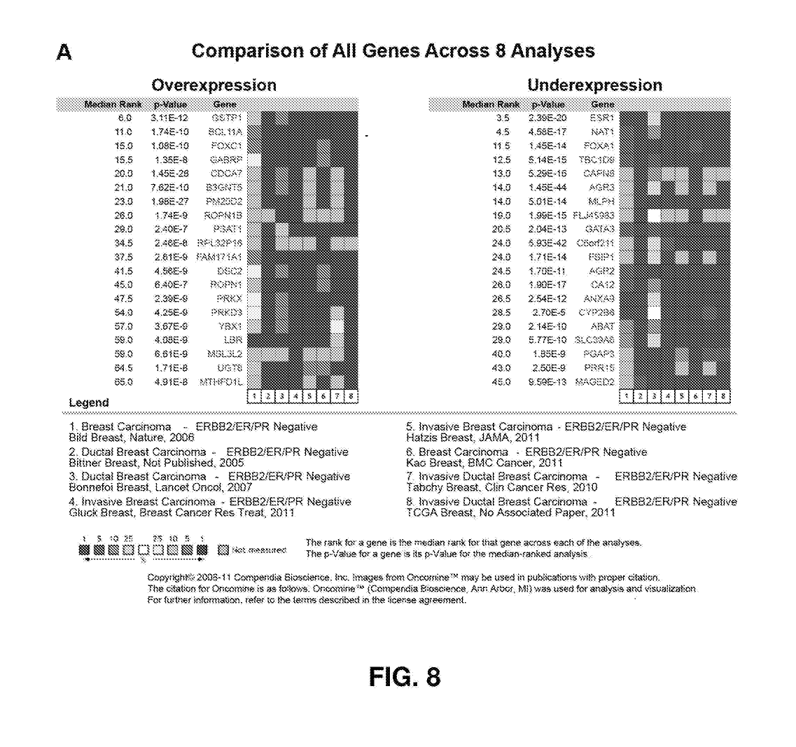

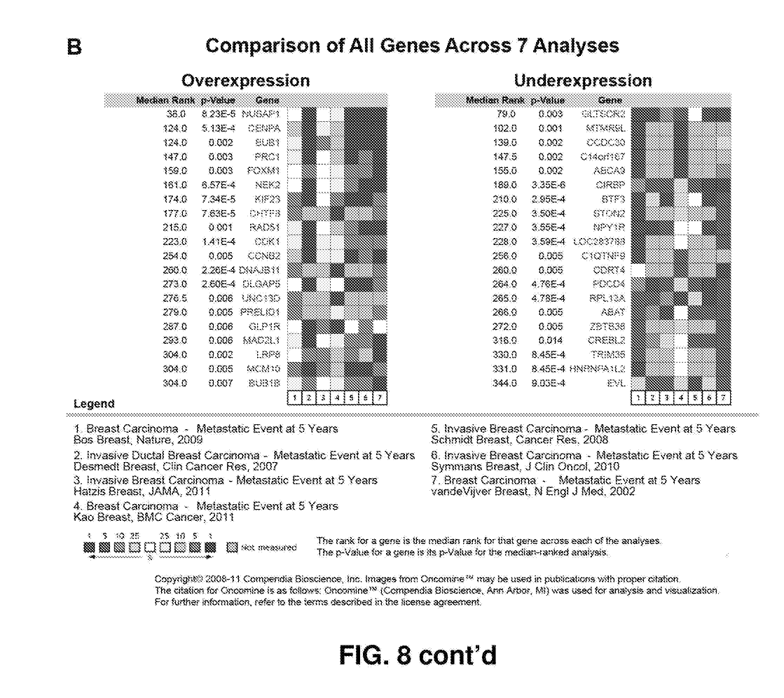

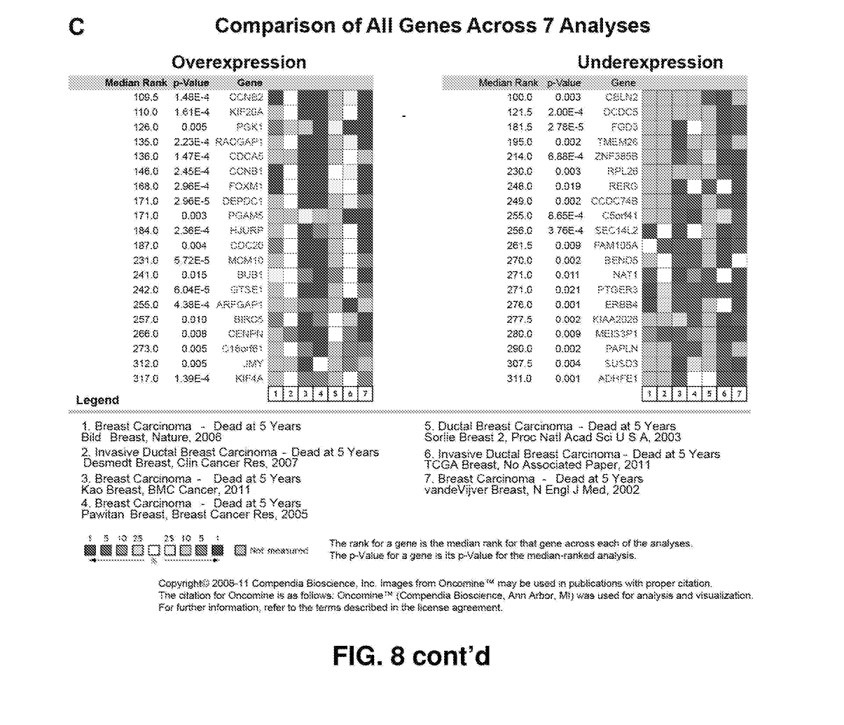

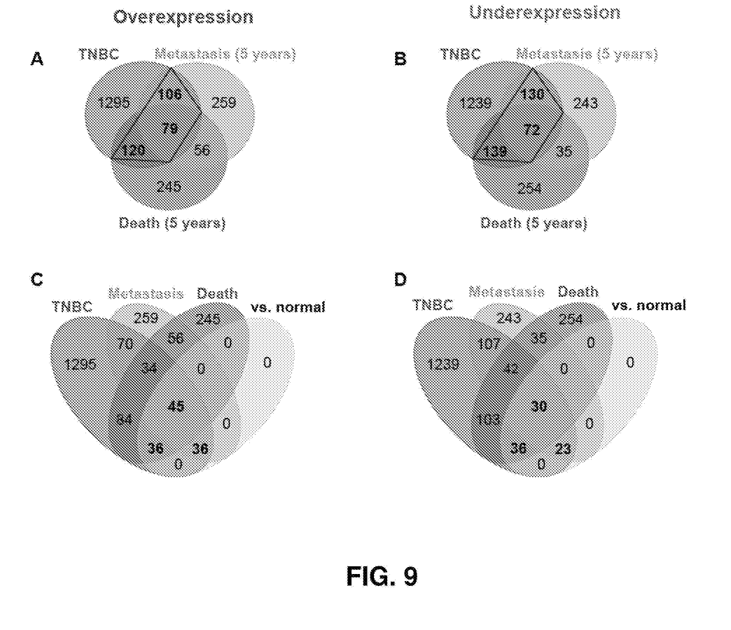

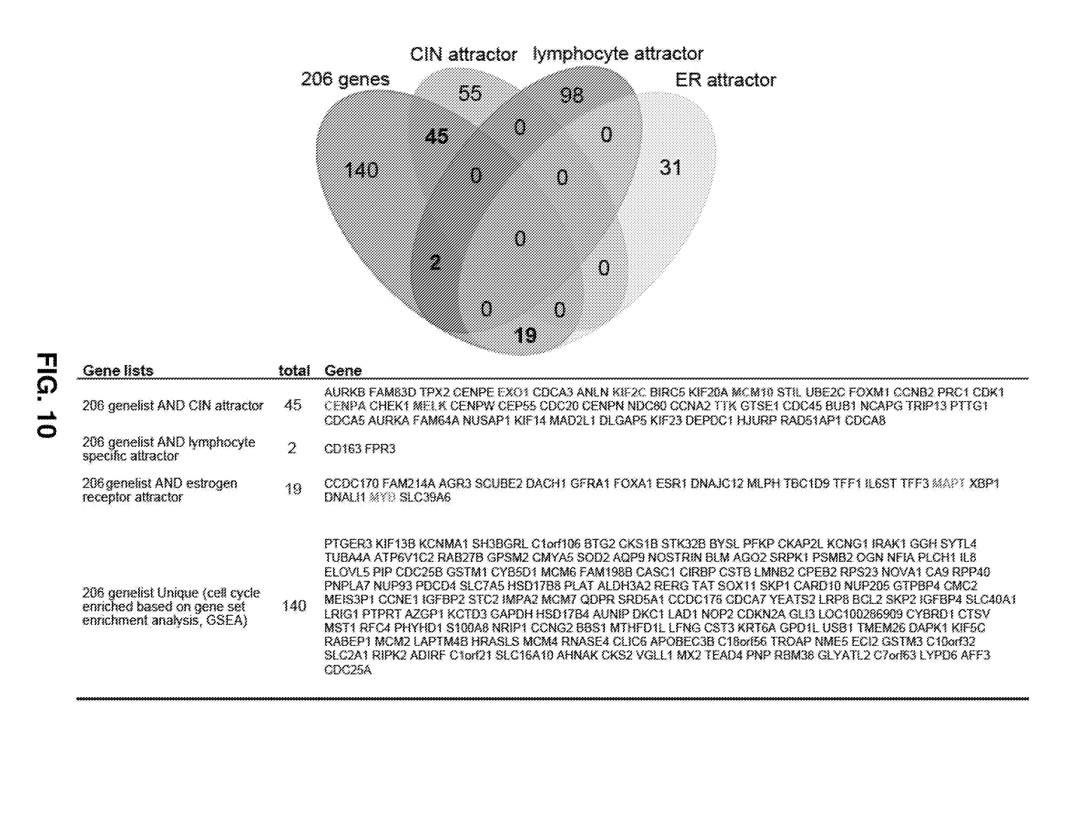

We performed a meta-analysis of global gene expression data in the Oncomine™ database¹⁹ (Compendia Bioscience, MI) using a primary filter for breast cancer (130 datasets), sample filter to use clinical specimens and dataset filters to use mRNA datasets with more than 151 patients (22 datasets). Patients of all ages, gender, disease stages or treatments were included. Three additional filters were applied to perform three independent differential analyses: (1) triple negative (TNBC cases vs. non-TNBC cases, 8 datasets⁴⁹⁻⁵⁶; (2) metastatic event analysis at 5 years (metastatic events vs. no metastatic events, 7 datasets^{53,54,57-61}) and (3) survival at 5 years (patients who died vs. patients who survived, 7 datasets^{49,54,56,58,61-63}). Deregulated genes were selected based on the median p-value of the median gene rank in overexpression or underexpression patterns across the datasets (FIG. 8). The union of these three deregulated gene lists resulted in a gene list of deregulated genes in aggressive breast cancers (FIG. 9). The METBRIC dataset²¹ was used as the validation set for further analysis. The normalized z-score expression data of the METABRIC dataset was extracted from Oncomine™ and imported into BRB-ArrayTools⁶⁴ (V4.2, Biometric Research Branch, NCI, Maryland, USA) with built in R Bioconductor packages. Survival curves for the METABRIC dataset were constructed using GraphPad® Prism v6.0 (GraphPad Software, CA, USA) and the Log-rank (Mantel-Cox) Test was used for statistical comparisons of survival curves.

### Ingenuity Pathway Analysis and Derivation of the Eight Gene List

Pathway analysis was performed using the Ingenuity Pathway Analysis® (Ingenuity Systems®, CA). For pathway analysis in IPA®, we used only direct relationships. After pathway analysis, we set to identify the minimum gene list that recapitulates the aggressiveness 206 gene list. We used the METABRIC dataset to perform statistical filtering in the BRB-ArrayTools software to derive the minimum gene list as follows: (1) the correlation of each gene in the CIN metagene and the ER metagene to the metagene itself was determined by quantitative trait analysis using the Pearson's correlation coefficient (univariate p-value threshold of 0.001); (2) the association of each gene with overall survival using univariate Cox proportional hazards model (univariate test p-value <0.001); and (3) the fold-change of gene expression between high aggressiveness score tumors and low aggressiveness score tumors was calculated for each gene. We selected genes with Pearson's correlation coefficient >0.7 to the metagenes, strongest survival association and more than 2-fold deregulation between high and low agressiveness score tumors. The METABRIC dataset and four publically available datasets were used to validate the 8-genes score. The four datasets (GSE25066⁵³, GSE3494⁶⁵, GSE2990¹⁵, GSE2034⁶⁶) were analyzed as described previously⁶⁷.

### Cell Culture and Drug Treatments

Breast cancer cell lines were obtained from ATCC™ (VA, USA) and cultured as per ATCC™ instructions. All cell lines were regularly tested for mycoplasma and authenticated using STR profiling. For the siRNA screen, siRNA solutions (Shanghai Gene Pharma, China) were used to transfect cells (MDA-MB-231, SUM159PT and Hs578T) with 10 nM of respective siRNA using Lipofectamine® RNAiMAX (Life Technologies, CA, USA). For drug treatments, docetaxel and the TTK inhibitor AZ3146 were purchased from Selleck Chemicals LLC (TX, USA) and diluted in DMSO. Six days after siRNA knockdown or after drug treatments the survival of cells in comparison to control was determined using the CellTiter 96® Assay as per manufacturer instructions (Promega Corporation, WI, USA). For immunoblotting, standard protocols were used and membranes were probed with antibodies against TTK (anti-MPS1 mouse monoclonal antibody [N1] ab11108 (Abcam, Cambridge), and γ-tubulin (Sigma-Aldrich®) then developed using chemiluminescence reagent plus (Milipore, MA, USA). Flow cytometry to quantify apoptosis was performed using Annexin V-Alexa₄₈₈ and 7-AAD (Life Technologies) as per manufacturer instruction using BD FACSCanto II™ flow cytometer (BD Biosciences, CA, USA).

### Breast Cancer Tissue Microarrays, Immunohistochemical and Survival Analysis

The Brisbane Breast Bank collected fresh breast tumor samples from consenting patients; the study was approved by the local ethics committees. Tissue microarrays (TMAs) were constructed from duplicate cores of formalin-fixed, paraffin-embedded (FFPE) breast tumor samples from patients undergoing resection at the Royal Brisbane and Women's Hospital between 1987 and 1994. For biomarker analysis, whole tumor sections or TMAs (depending on the marker) were stained with antibodies against ER, PR, Ki67, HER2, CK5/6, CK14, EGFR and TTK (Table 8), and scored by trained Pathologists. The Vectastain® Universal ABC kit (Vector laboratories, CA) was used for signal detection according to the manufacturer's instructions. Stained sections were scanned at high resolution (ScanScope Aperio, Leica Microsystems, Wetzlar, Germany), and then images were segmented into individual cores for analysis using Spectrum software (Aperio). Survival and other clinical data were collected from the Queensland Cancer Registry and original diagnostic Pathology reports, and in addition we performed an internal histopathological review (SRL) of representative tumor sections from each case, stained with H&E. For analysis of HER2-amplification TMAs were analyzed using HER2 CISH. Criteria for assigning prognostic subgroups in this study are summarized in FIG. 14.

### Other Statistical Analysis

Statistical analyses were prepared using GraphPad® Prism v6.0. The types of tests used are stated in Figure Legends. Univariate and multivariate Cox proportional hazards regression analyses were performed using MedCalc for Windows, version 12.7 (MedCalc Software, Ostend, Belgium).

### Results

### Meta-Analysis of Gene Expression Profiles in TNBC

We performed a meta-analysis of published gene expression data, irrespective of platform, using the Oncomine™ database¹⁹ (version 4.5). We compared the expression profiles of 492 TNBC cases vs. 1382 non-TNBC cases in 8 datasets and found 1600 overexpressed and 1580 underexpressed genes in the TNBC cases (cutoff median p-value across the 8 datasets <1×10⁻⁵ from a Student's t-test, FIG. 8). We also compared the expression profiles of primary breast cancers from 512 patients who developed metastases vs. 732 patients who did not develop metastases at 5 years (7 datasets in total) to identify 500 overexpressed and 480 underexpressed genes in the metastasis cases (cutoff median p-value across the 7 datasets <0.05 from a Student's t-test, FIG. 8). Finally, we compared the expression profiles of 232 primary breast tumors from patients who died within 5 years vs. 879 patients who survived in 7 datasets and found 500 overexpressed and 500 underexpressed genes in the poor survivors (cutoff median p-value across the 7 datasets <0.05 from a Student's t-test, FIG. 8). The union of these analyses—genes deregulated in TNBC and in tumors that metastasized or resulted in death within 5 years—generated a gene list of 305 overexpressed and 341 underexpressed genes (FIGS. 9A&B). The deregulated genes from our analyses did not consider deregulation in comparison to normal breast tissue. To identify cancer-related genes, we used the METABRIC (Molecular Taxonomy of Breast Cancer International Consortium) dataset²¹ as a validation dataset. Of the 305 overexpressed and 341 underexpressed genes identified in the meta-analysis, 117 overexpressed and 89 underexpressed genes (206 genes) were deregulated in TNBC (250 cases) vs. 144 adjacent normal tissue (1.5 fold-change cutoff; FIGS. 9C&D).

### Clinicopathological Features of the Aggressiveness Gene List

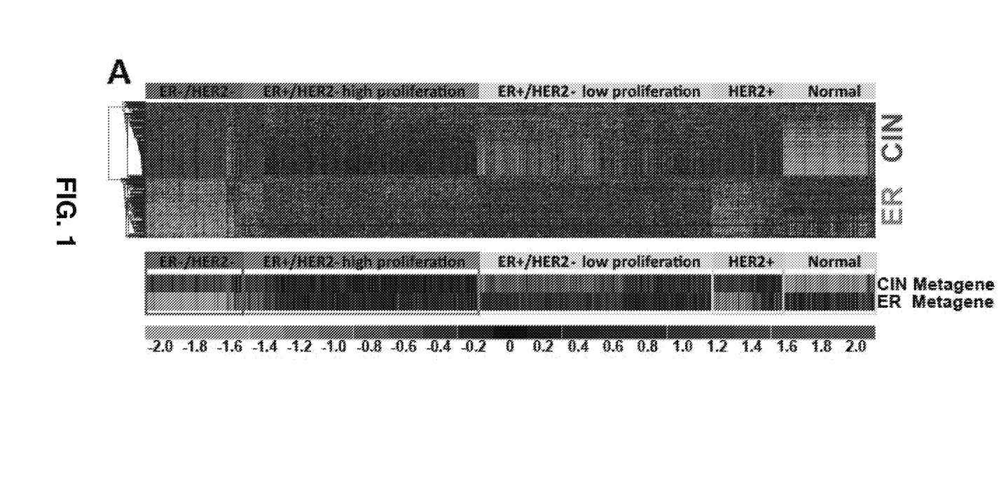

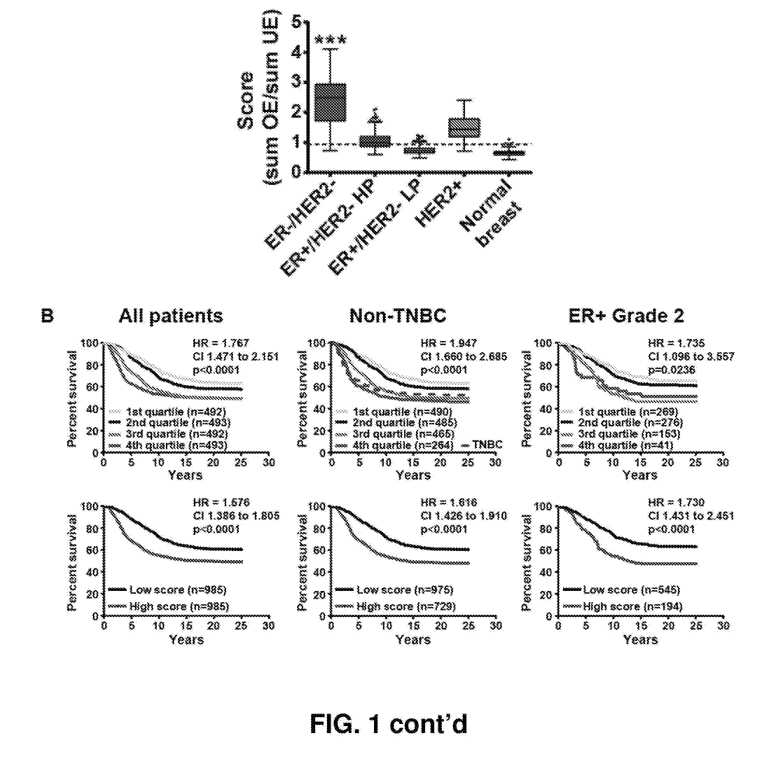

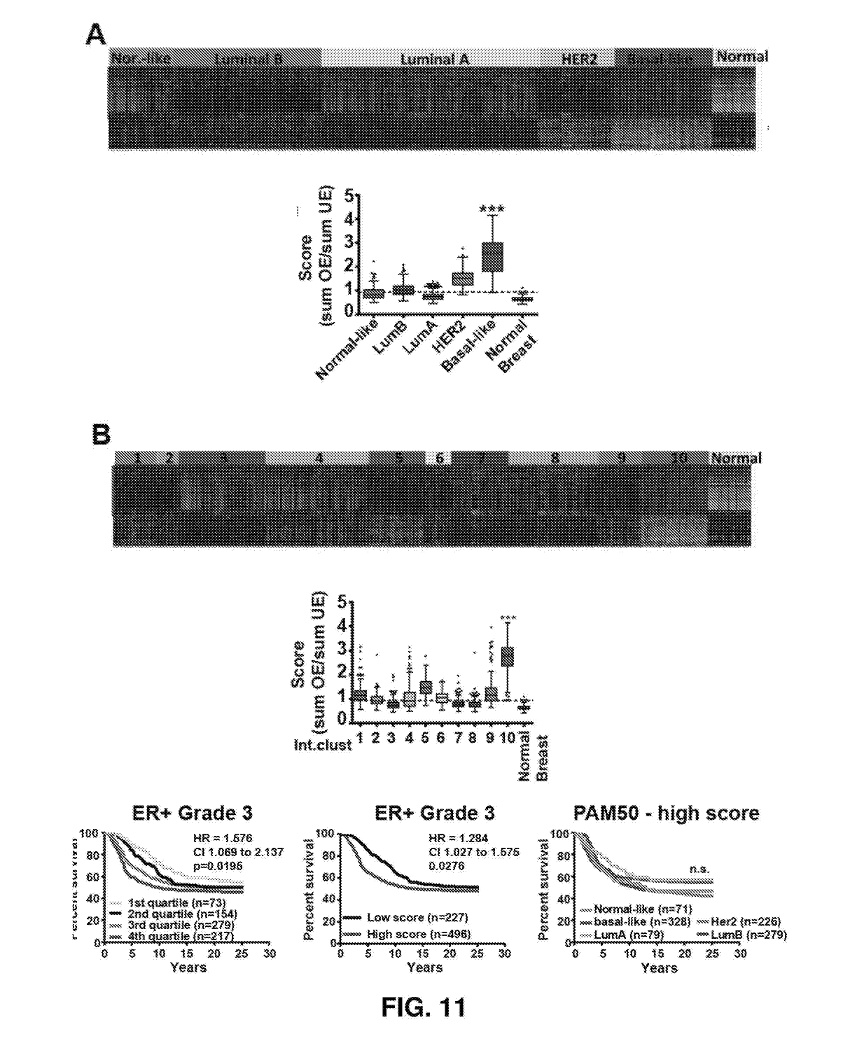

We compared the 206 genes from the above analysis, we called the “aggressiveness gene list” (Table 4), to the recently described metagene attractors^{16,17} and found that 45 of the overexpressed genes were in the CIN metagene, whereas 19 of the underexpressed genes were in the ER metagene (FIG. 10). The expression of the aggressiveness gene list was visualized in the METABRIC dataset, stratified according to the histological subtypes by the GENUIS classification²². As shown in FIG. 1A, ER⁻/HER2⁻ (TNBC), in comparison to adjacent normal breast tissue, showed the highest upregulation of CIN genes (red in the heat map) and downregulation of ER signaling genes (green in the heat map). Tumors of other subtypes showed a range of deregulation of these genes. To quantify these trends, we calculated the “aggressiveness score” as the ratio of the CIN metagene (average of expression of CIN genes) to the ER metagene (average of expression of ER genes). The aggressiveness score was highest for ER⁻/HER⁻ (TNBC), followed by HER2⁺ then ER⁺ tumors (box plot in FIG. 1). We also analyzed the aggressiveness score in the five intrinsic breast cancer subtypes predefined by the PAM50 classification′ and the ten integrative clustering (intClust) subtypes defined by combined clustering of gene expression and copy number data subtypes²¹ (FIG. 11). The aggressiveness score was highest in the basal-like and the intClust 10 subtypes which are enriched for TNBC and have poor prognosis.

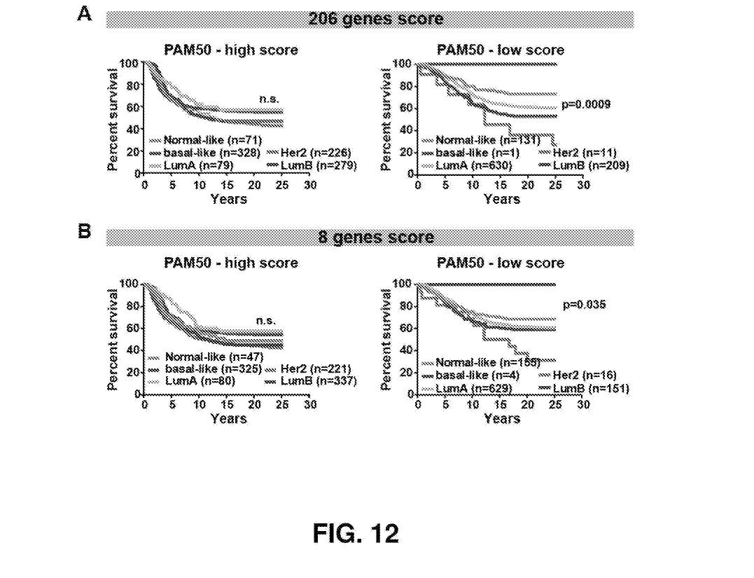

Interestingly, tumors of various subtypes scored higher than the median aggressiveness score (line in box plots in FIG. 1 and FIG. 11). To this end, we examined the overall survival of patients in the METABRIC dataset stratified by quartiles and also dichotomized by the median of the aggressiveness score. Tumors with high aggressiveness score had worse survival than those with low aggressiveness score. The survival of patients with non-TNBC tumors with high aggressiveness score had poor survival that was similar to TNBC patients (FIG. 1B). Among ER⁺ tumors we found that high aggressiveness score predicted poor survival in both Grade 2 (FIG. 1B) and Grade 3 (FIG. 11) tumors. Tumors with high aggressiveness score showed poor survival regardless of the PAM50 intrinsic breast cancer subtypes (FIG. 11). The PAM50 classifier was prognostic only in low aggressiveness score tumors (FIG. 12).

One Network of Direct Interactions in the Aggressiveness Gene List Associates with Patient Survival

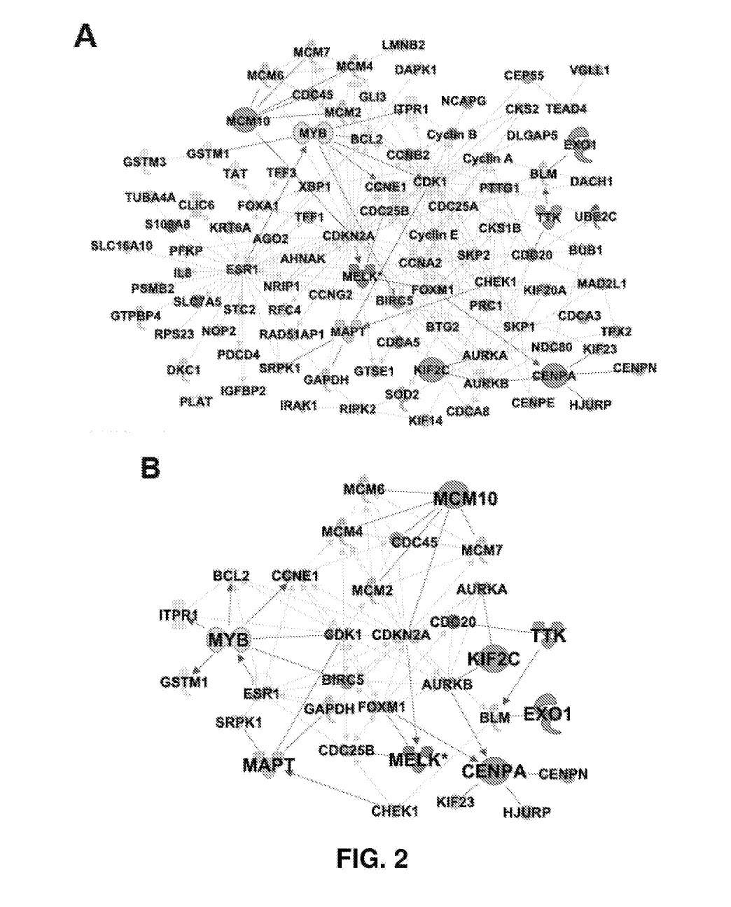

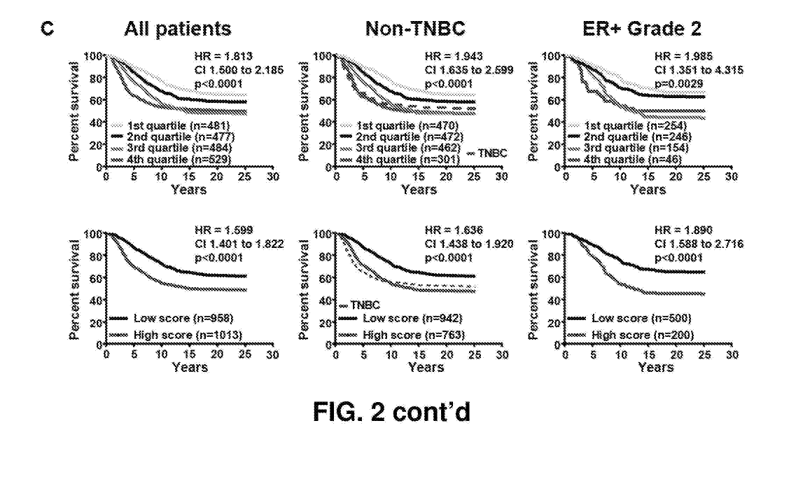

We performed network analysis on the aggressiveness gene list using the Ingenuity Pathway Analysis (IPA®) and found a network with direct interactions between 97 of the 206 deregulated genes (FIG. 2A). To find the minimal genes that represent the aggressiveness genes and this network, the 97 genes in this network were analyzed for their correlation with the CIN or ER metagenes and overall survival in the METABRIC dataset (Table 5). We selected genes according to the following criteria: (1) highest correlation with the metagenes (Pearson's correlation coefficient >0.7); (2) association with overall survival (Cox proportional hazards model, p<0.001), and (3) more than 2-fold deregulation with least standard deviation of expression between high and low aggressiveness score tumors. These analyses identified two genes from the ER metagene (MAPT and MYB) and six genes from the CIN metagene (MELK, MCM10, CENPA, EXO1, TTK and KIF2C). These 8 genes were maintained in a directly connected network (FIG. 2B). The classification of tumors (high vs. low across the median) from these eight genes, again representing the ratio of CIN and ER metagenes, predicted the classification from the 206 genes with 95% sensitivity and 97% specificity by prediction of microarray (PAM) analysis (data not shown). Importantly, a high score from these eight genes identified poor survival in all patients, non-TNBC patients and ER⁺ Grade 2 (FIG. 2C).

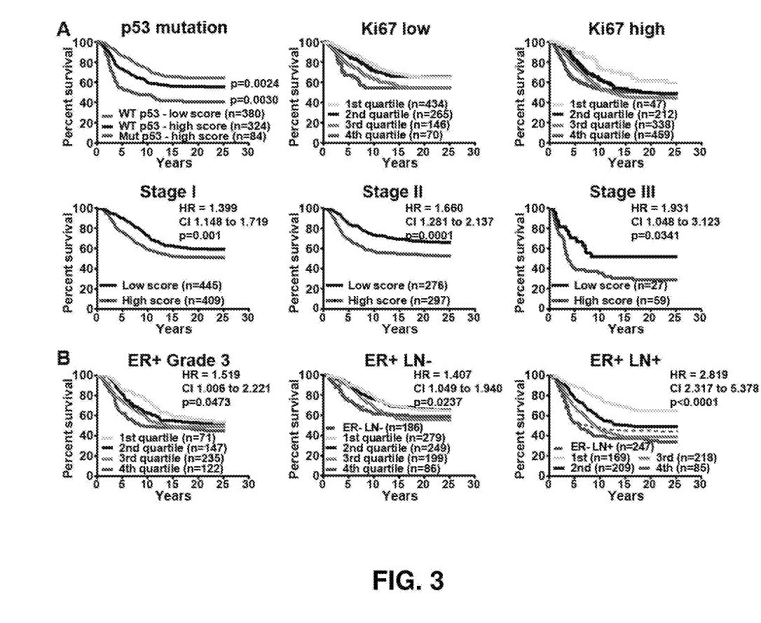

Next, we explored the 8-genes score for prognosis in several molecular and histological settings in the METABRIC dataset. The survival of patients with tumors with wild-type TP53 were stratified by the 8-genes score (FIG. 3A). Patients with mutant TP53, which were mainly of high score, showed worse survival than those with wild-type TP53, suggesting that TP53 mutation is an independent prognostic factor. Patients with tumors with low or high expression of the proliferation marker Ki67 were stratified by the 8-genes score suggesting that the 8-genes score is independent of proliferation (FIG. 3A). We also found that the 8-genes score stratified the survival of patients from all stages of disease (Stage I-Stage III, FIG. 3A). We focused on ER⁺ and found that, as in the case of ER⁺ Grade 2 tumors (FIG. 2C); the 8-genes score stratified the survival of patients with ER⁺ Grade 3 tumors (FIG. 3B). Importantly, the 8-genes score identified ER⁺LN⁻ and ER⁺LN⁺ patients who had poor survival similar to ER⁻LN⁻ and ER⁻LN⁺ patients, respectively (FIG. 3B). High 8-genes score identified poor survival of patients with tumors of all PAM50 subtypes and the prognostication by PAM50 classification was only evident in low 8-genes score tumors (FIG. 12).

### The 8-Genes Aggressiveness Score in Multivariate Survival Analysis

To exclude the possibility that the aggressiveness score—calculated using the 206 genes or the 8 genes—was redundant; we performed multivariate Cox-proportional hazards model analysis in the METABRIC dataset (with Illumina platform) in comparison to conventional clinical variables and current gene signatures. As detailed in Table 1, the aggressiveness scores significantly associated with patient survival when compared with conventional variables and outperformed MammaPrint⁹, OncotypeDx^{10,11}, proliferation/cell cycle^{16,20} and CIN²⁰ signatures. Moreover, our aggressiveness scores outperformed the CIN4 classier²³ which was recently developed from the CIN signature.

We validated the six CIN and two ER genes in univariate survival association using the online tool Kaplan-Meier (KM)-plotter²⁴ (Tables 6 & 7) which has the gene expression and survival data of more than 2000 patients (but are not part of the METABRIC dataset). We found that the collective expression of the six overexpressed genes (MELK, MCM10, CENPA, EXO1, TTK and KIF2C) significantly associated with relapse free survival (RFS) and distant metastasis free survival (DMFS) in all patients, ER⁺ patients, lymph node negative (LN⁻) or positive (LN⁺) patients (Table 6). The two underexpressed genes (MAPT and MYB) also significantly associated with RFS and DMFS in these patient groups (Table 7).

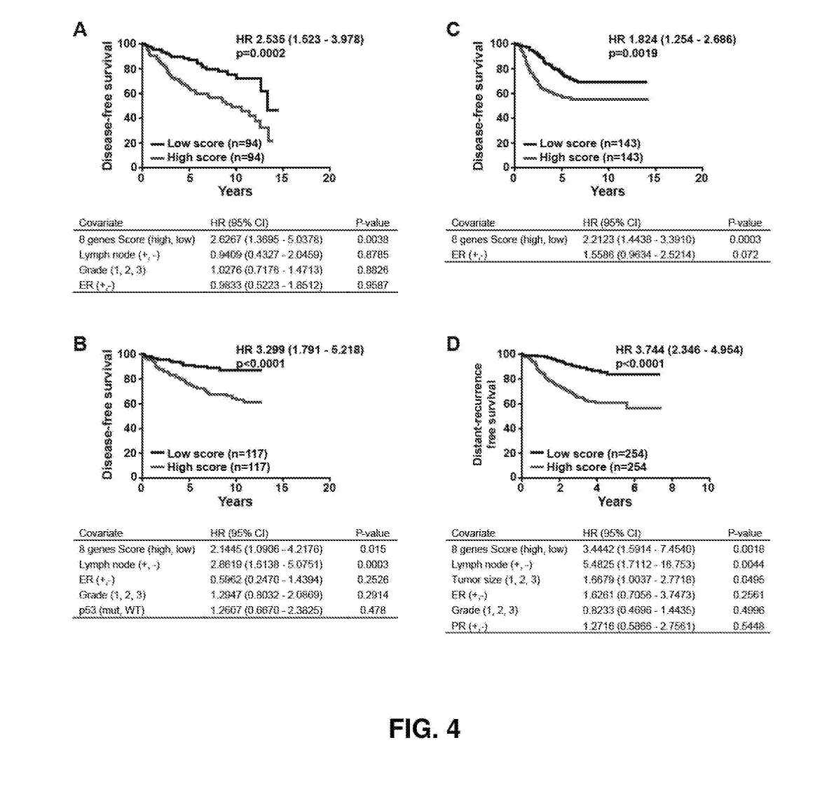

More importantly, we performed multivariate survival analysis of the 8-genes score in four datasets (with Affymetrix platform from the Gene Expression Omnibus [GEO]; GSE2990, GSE3494, GSE2034 and GSE25066). Again, the score was significantly associated with survival in a multivariate Cox-proportional hazards model in every dataset tested (FIG. 4). Altogether, we found that in multiple datasets that used different platforms, the 8-genes score identified patients with poor survival independently of other clinico-pathologic indicators and outperforming current signatures.

### Therapeutic Targets in the Aggressiveness Gene List

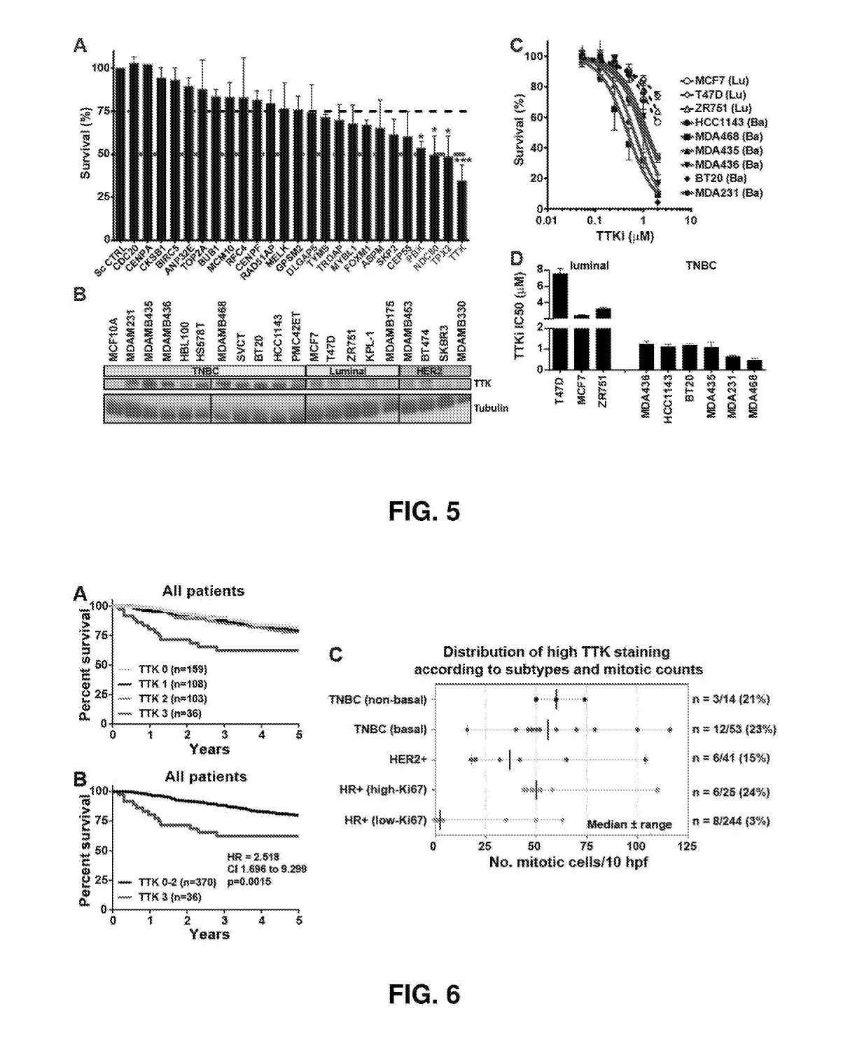

The overexpressed genes in the CIN metagene are involved in or regulate mitosis, spindle assembly and checkpoint, kinetochore attachment, chromosome segregation and mitotic exit. Thus it is not surprising that several of the overexpressed genes are targets for molecular inhibitors, such as CDK1^{25,26} and AURKA/AURKB²⁷ and have been trialed pre-clinically and clinically²⁸. To this end, we performed siRNA depletion against 25 genes of the CIN metagene in three TNBC cell lines, MDA-MB-231, SUM159PT and Hs578T. We found that knockdown of four genes (11K, TPX2, NDC80 and PBK) consistently affected the survival of these cells (FIG. 5A and Table 5). The knockdown of TTK showed the worst survival and since it was in the 8-genes score we selected TTK for further studies. We found that TTK protein was higher in TNBC cell lines compared to the near-normal MCF10A cell line, and luminal/HER2 cell lines (FIG. 5B). Next, we used the specific TTK inhibitor (TTKi), AZ3146, against a panel of breast cancer cell lines and found that TNBC cell lines were more sensitive to the TTKi (FIG. 5C).

### TTK Expression in Aggressive Tumors and Potential for Combination Therapy

To further study the potential of TTK as therapeutic target, we investigated TTK expression at the mRNA and protein levels in breast cancer patients. We analyzed the correlation of TTK mRNA expression, dichotomized at the median, with clinicopathological indicators in the METABRIC dataset of 2000 patients (Table 2). High TTK mRNA expression associated with younger age of tumor diagnosis, larger tumor size, higher tumor grade, higher Ki67 expression, TP53 mutations, an ER/PR negative tumor phenotype, HER2 positivity and TNBC. Based on PAM50 subtyping, high TTK mRNA was associated with luminal B, HER2-enriched and basal-like tumors.

We also analyzed TTK expression in a cohort of breast cancer patients (406 patients) by IHC. TTK and its activity is detected at all stages of the cell cycle, however, it is upregulated during mitosis²⁹. Thus, we observed TTK staining in non-mitotic cells to define high TTK levels (score of 3) in order to exclude the bias of elevated TTK level during mitosis. Similar to TTK mRNA, high TTK protein level (Table 3) associated with high tumor grade, high Ki67 expression and TNBC status (particularly basal TNBC). Moreover, in agreement with the TTK mRNA associations with the PAM50 intrinsic subtypes, high TTK protein was observed in HER2-positive and proliferative ER⁺/HER2⁻ tumors (most related to luminal B) but low TTK protein in non-proliferative ER⁺/HER2⁻ tumors (most related to luminal A). In addition to these associations with aggressive phenotypes, we also found that high TTK protein significantly associated with aggressive histological features including ductal histology, pushing tumor border, lymph node involvement, nuclear pleomorphism, lymphocytic infiltration and higher mitotic scores (Table 3). Altogether, like the high aggressiveness score from the 206 or 8 genes, high level of TTK mRNA and protein span across breast cancer subtypes marking aggressive behavior.

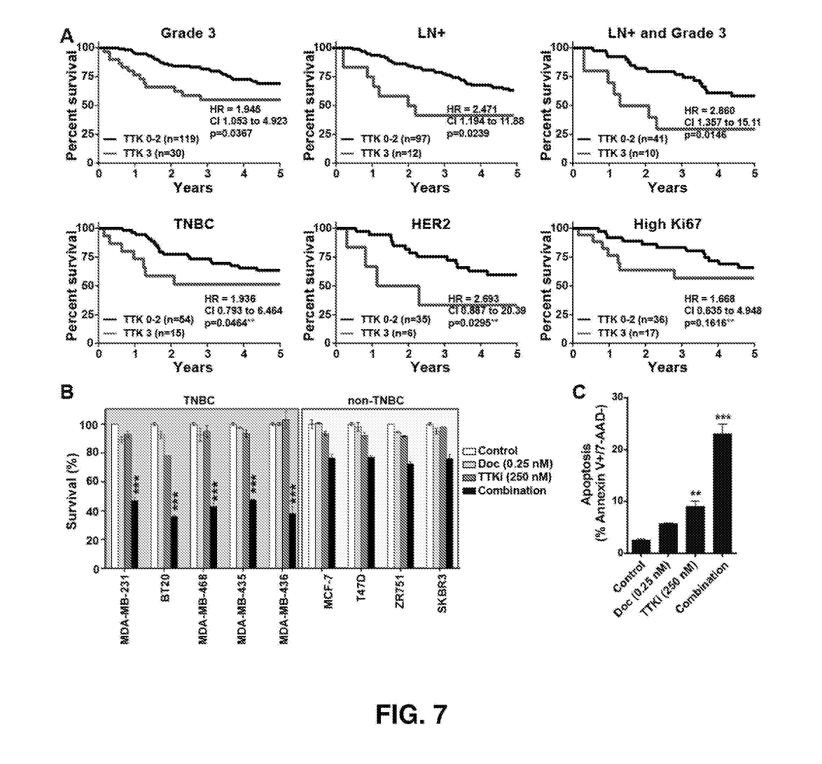

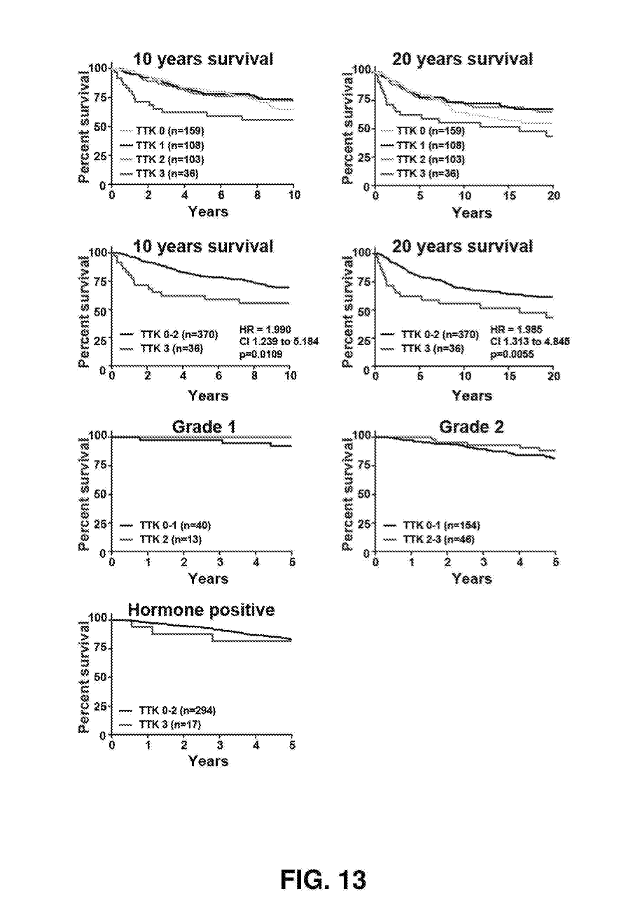

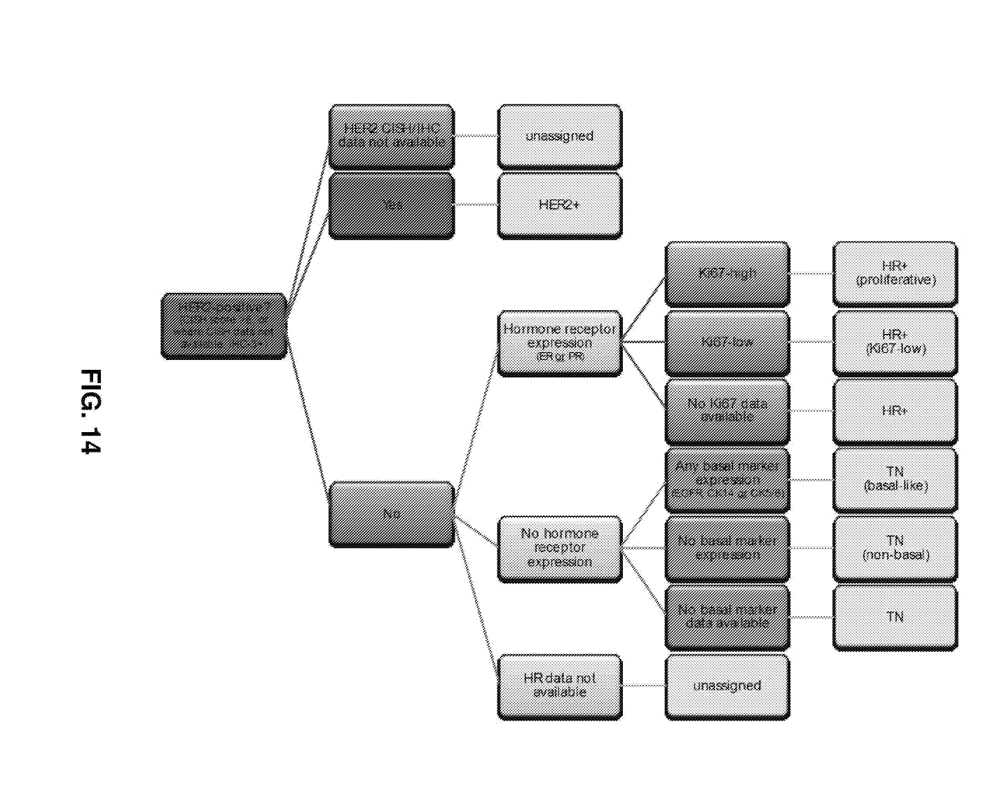

We examined the association of TTK protein level with patient survival and found that breast tumors with high TTK staining (category 3) had worse survival than other staining groups at 5 years (FIGS. 6A&B) and 10 and 20 years (FIG. 13). Importantly, high TTK staining (category 3) was not restricted to a particular histological subgroup or to tumors with high mitotic index (FIG. 6C). Next, we focused on prognostication of aggressive subgroups (Grade 3, lymph node positive, TNBC, HER2 or high Ki67) and found that high TTK protein level identified exceptionally aggressive tumors that lead to poor survival of less than 2 years (FIG. 7A). Finally, to exploit our finding that TTK, as a part of the aggressiveness score, was associated with aggressive breast tumors and that TTK inhibition was effective in TNBC cell lines that overexpress this protein (FIG. 5), we investigated the therapeutic potential of combining TTK inhibition with chemotherapy. We found that TTKi synergized with docetaxel at very low (sub-lethal doses) in the treatment of TNBC cell lines which overexpress TTK in comparison to cell lines which do not (FIG. 7B) and that this combination induced apoptotic cell death (FIG. 7C).

### CIN Metagene and ER Metagenes in Lung Adenocarcinoma

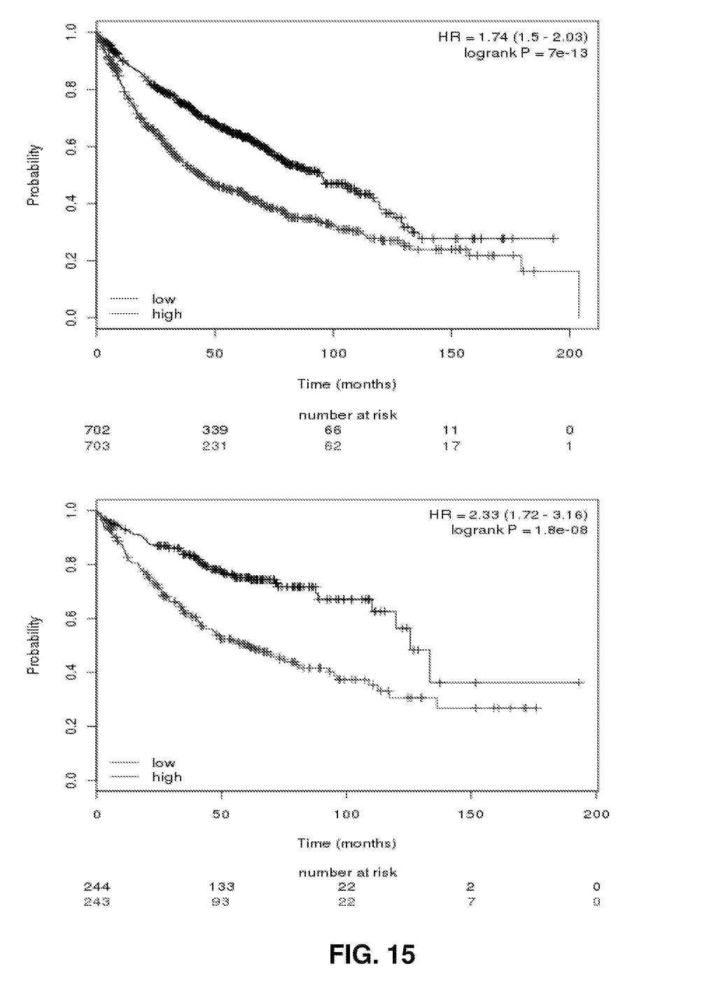

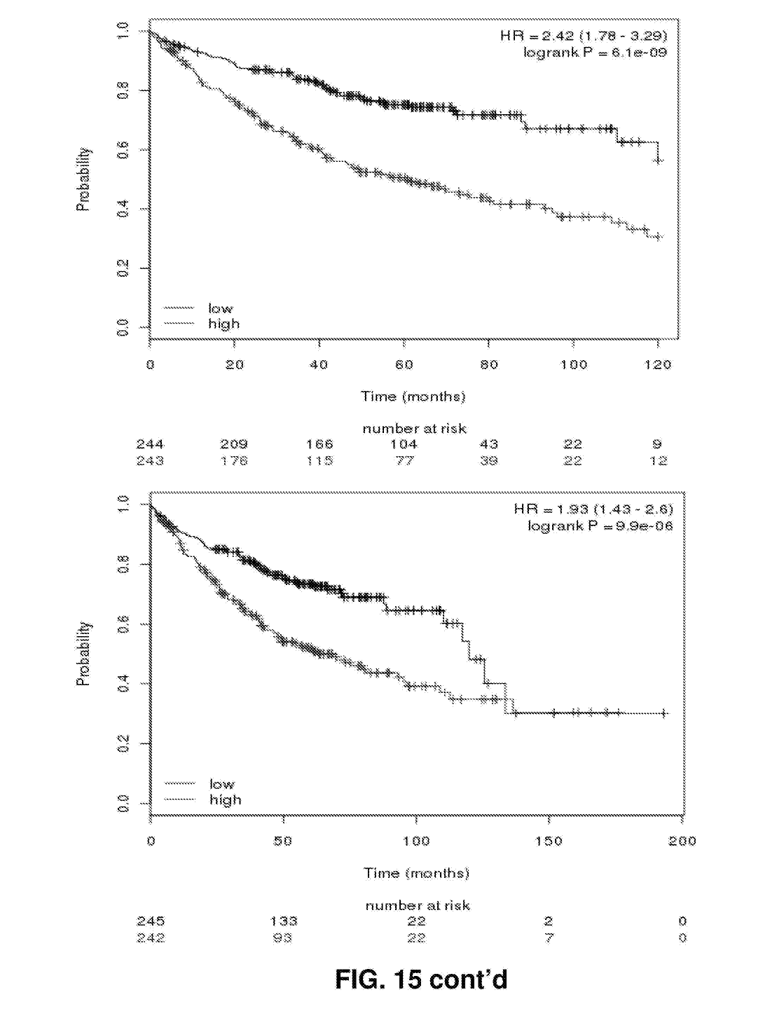

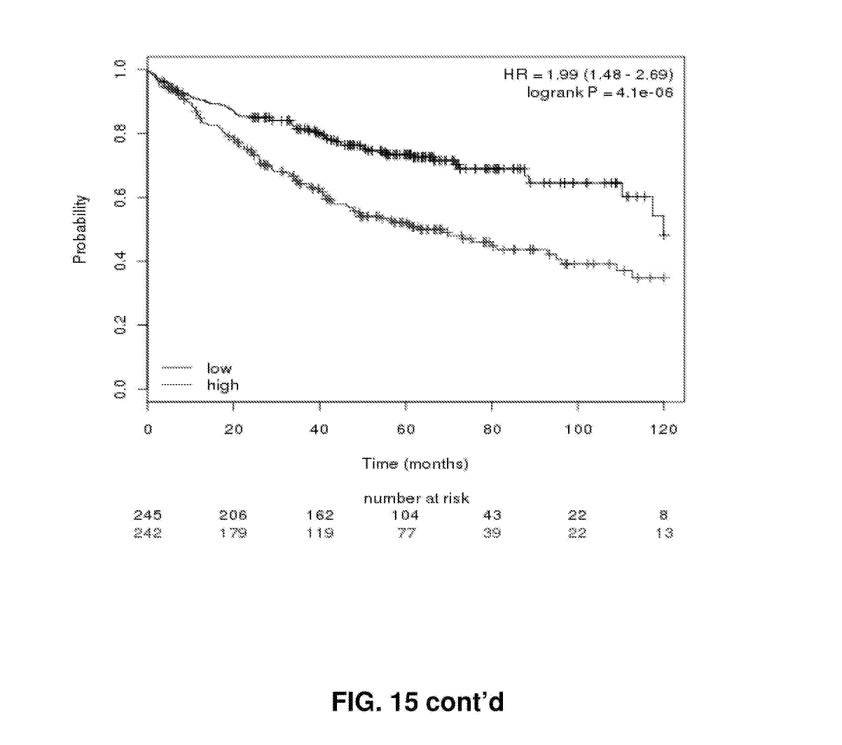

There is also reason to believe that the metagene signature may work for other cancers, such as lung cancer. FIG. 15 provides overall survival curves of lung cancer patients split by ten (10) CIN genes that include the aforementioned six (6) (genes as well as CENPN, CEP55, FOXM1 and TPX2; and the two (2) ER genes MAPT and MYB as a signature; patients are low or high according to the median of the signature. The signature outperformed tumour grade and disease stage and remained significant when adjusted for AJCC T (size) and N (lymph node) stages (tumour size (T stage) and lymph node status (N stage) in multivariate Cox regression analysis in lung cancer patients (Table 9). In particular, the signature was prognostic in lung adenocarcinoma. The prognostication of lung adenocarcinoma was significant even when including a minimal gene set of 6 CIN genes and 2 ER genes.

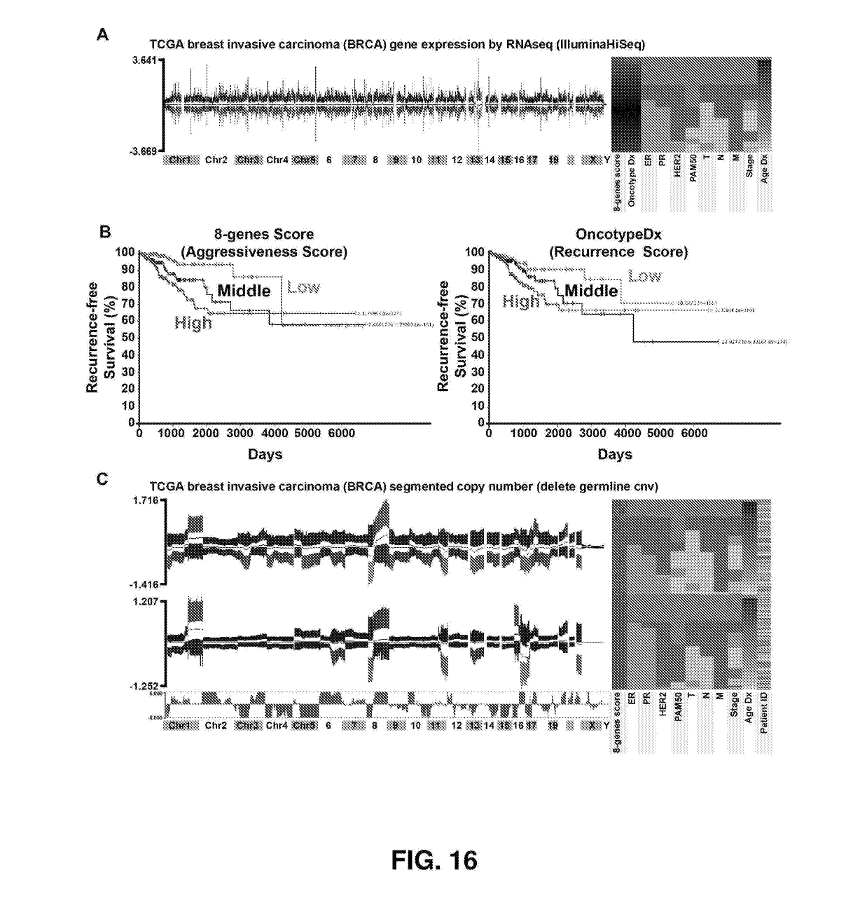

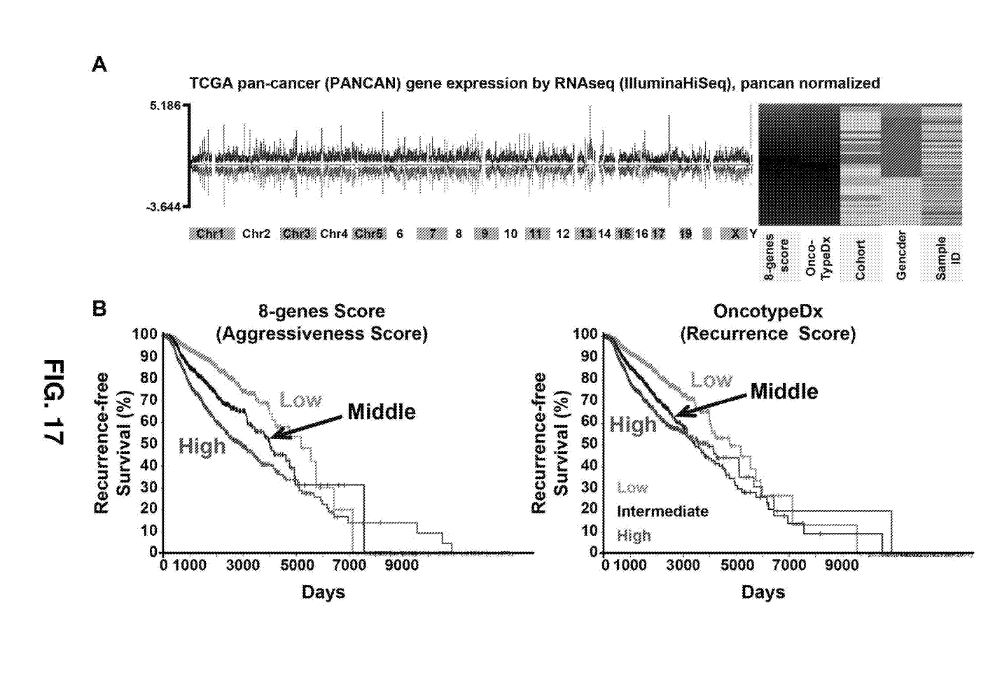

In FIG. 16A we show the global gene expression (by RNAseq) of the breast cancer patients in the TCGA dataset. From these data the 8-genes score (Aggressiveness score) and the OncotypeDx (Recurrence score) were investigated for association with survival. The 8-genes score stratified breast cancer survival better than the OncotypeDx (FIG. 16B). Further, the 8-genes score (Aggressiveness score) identified tumours with high genomic copy number variations involving whole chromosome arms deletions and duplications reflecting aneuploidy (FIG. 16C).

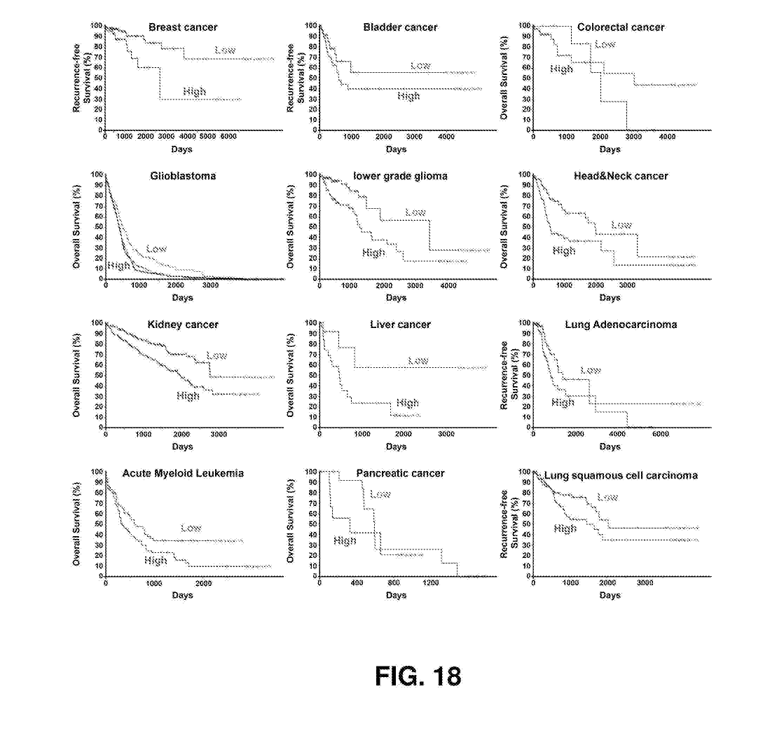

We also find that the 8-genes score (Aggressiveness score) stratifies the survival of all cancers collectively in the TCGA data better than the OncotypeDx (FIG. 17) and that the 8-genes score (Aggressiveness score) was prognostic in each of the tested cancers (FIG. 18). Similarly, as in breast cancer (FIG. 16C), the 8-genes score (Aggressiveness score) identified tumors of all cancer types with high genomic copy number variations involving whole chromosome arms deletions and duplications reflecting aneuploidy (data not shown). These cancer types include breast cancer, bladder cancer, colorectral cancer, glioblastoma, lower grade glioma, head & neck cancer, kidney cancer, liver cancer, lung adenocarcinoma, abute myeloid leukaemia, pancreatic cancer and lung squamous cell carcinoma.

### DISCUSSION

This meta-analysis of gene expression in the Oncomine™ database identified a list of 206 was enriched with two core biological functions/metagenes; chromosomal instability (CIN) and ER signaling. We calculated the aggressiveness score, the ratio of CIN to ER metagenes, which associated with overall survival of breast cancer. A core of eight genes (six CIN genes and two ER signaling genes) was representative and recapitulated the correlations with outcome from the 206 genes. The score from the six CIN genes to the 2 ER signaling genes, 8-genes score, associated with survival in several breast cancer datasets. Our aggressiveness scores outperformed conventional variable and published signatures in multivariate survival analysis. Particularly in ER⁺ tumors, some cases have survival as poor as that of the aggressive HER2⁺ and TNBC subtypes. Our data suggest that the interplay of cancer-related biological functions, namely CIN and ER signaling, are better predictors of phenotypes than single genes or single functions. This notion is in line with recent studies showing that the interaction of biologically-driven predictors provide better prognosis^{16,17,30}. Recently, all ER⁻ tumors were described to have a high level of CIN metagene, however, it was not clear that ER⁺ tumors could be described as low CIN tumors¹⁶. In our study, we clarify that ER⁺ disease contains a considerable fraction of tumors that have high level of CIN genes and that the relationship between CIN and ER genes is a powerful predictor of survival in these patients.

The fidelity of chromosome segregation is ensured by the proper attachment of the microtubules from the mitotic spindle to the kinetochores of chromosomes in a tightly regulated process and CIN refers to the missegregation of whole chromosomes thus producing aneuploidy³¹. Using aneuploidy as a surrogate marker for CIN, Carter et al developed a gene signature and found that this “CIN signature” predicts clinical outcome in multiple cancers²⁰. More recently, a minimal gene set that captures the CIN signature, CIN4 (AURKA, FOXM1, TOP2A and TPX2) was described as the first clinically applicable qPCR derived measure of tumor aneuploidy from FFPE tissue. Since Grade 2 tumors heterogeneous characteristics in terms of clinical outcome, the significance of the CIN4 classier is the stratification of Grade 2 tumors into good and poor prognosis groups²³. Our aggressiveness scores were prognostic in all tumor grades and disease stages (stages I-III and lymph node negative and positive) and outperformed the CIN signature and the CIN4 classier in multivariate survival analysis in the METABRIC dataset. Strikingly, but in agreement with previous studies^{32,33}, the prognostication using the CIN metagene and our aggressiveness scores from gene expression levels were restricted to ER⁺ disease but not in the TNBC or HER2 subtypes. This may be explained that ER⁻ tumors have a high level of CIN metagene as per our results and published previously¹⁶. However, our results with TTK protein level clearly demonstrate that TNBC, HER2, high grade, lymph node positive and proliferative tumors contain subgroups with high TTK levels exclusive of mitotic cells and have poorer survival than those with low TTK expression or TTK expression in mitotic cells. We propose that there are two types of high expression of CIN genes that may not be clearly differentiated by mRNA expression studies. One form of elevated CIN genes relates to high level of mitosis and proliferation whereas the second form that we measured by IHC exclusive of mitotic cells is driven by another aggressive phenotype; protection of aneuploidy and genomic instability. The recent study of the CIN4 classifier lends support to our proposition. In this study, using flow cytometry to measure aneuploidy by DNA content, the authors found that a substantial proportion of tumors with high CIN4 scores have a normal DNA ploidy and that a significant proportion of aneuploid cases had low CIN4 score²³.

Chromosome missegregation and aneuploidy enhance genetic recombination and defective DNA damage repair³⁴ to drive a “mutator phenotype” required for oncogenesis³⁵. Genomic instability caused by deregulated mitotic spindle assembly checkpoint (SAC) and aneuploidy has been termed “non-oncogene addiction”^{36,37}. It is tempting to suggest that CIN and aneuploidy are exploited by breast cancer stem cells which are high in TNBC³⁸ due to the link between cancer stem cells, aneuploidy and therapy resistance^{39,40}. This is supported by studies that implicate several genes involved in the SAC and chromosome segregation in tumor initiation, progression and cancer stem cells, e.g. AURKA in ovarian cancer⁴¹, MELK/FOXM1 in glioblastoma^{42,43}, MELK⁴⁴ and MAD2⁴⁵ in breast cancer and SKP2 in several cancers⁴⁶. The role of CIN genes to protecting aneuploidy could provide an insight to the paradox that TNBC show a better response to chemotherapy due to higher level of proliferation, yet these tumors have poorer outcome. We propose that resistance in TNBC could be attributed to the ability of aneuploid cells to adapt and drive recurrence. At least in vivo, chemotherapy has been shown to induce the proliferation quiescent aneuploid cells as a mechanism for therapy resistance³⁹. We envisage that the high level of the CIN metagene in TNBC, particularly genes involved in chromosome segregation, is protective of this state. Indeed, one study found that a high level of TTK is protective of aneuploidy in breast cancer cells and its silencing reduces the tumorigenicity of breast cancer cell lines in vivo⁴⁷. Our results from the patient cohort demonstrate that high TTK protein expression exclusive of mitosis was indeed prognostic aggressive tumors and support the concept that protection from aneuploidy and genomic instability is an aggressive phenotype that drives poor outcome.

Our results with the TTK molecular inhibitor, in agreement with published studies using siRNA depletion^{47,48}, supports the idea of targeting chromosomal segregation in tumors with a high CIN phenotype as a therapeutic strategy. We also suggest that while TTK is high in TNBC as previously described^{47,48}, a considerable proportion of non-TNBC tumors that display aggressive features also show an elevated level of CIN genes, and would benefit from such targeted therapies. To our knowledge the combination of sub-lethal doses of taxanes with TTK inhibition has not been investigated so far in breast cancer, but in other cancers^{33,50-53}. Our results reveal that TTK inhibition indeed sensitizes breast cancer cells with high TTK to docetaxel.

Referring particularly in FIGS. 16-18, as well as the 8-genes score (Aggressiveness score) being prognostic for the survival of cancer patients after treatment, the aggressiveness score also identifies tumors with high copy number variations involving whole chromosome arms reflecting aneuploid status. Thus, the aggressiveness score may also serve as a companion diagnostic for drugs that target aneuploidy by means of targeting genes listed in Table 4, inclusive of the 8 genes used to produce the aggressiveness score (such as TTK⁶⁷⁻⁷⁰) or by other drugs that target the aneuploidy state (such as PLK1^{71,72} or others⁷³⁻⁷⁶).

In conclusion, our study emphasizes that classification of breast cancer based on biological phenotypes facilitates understanding the drivers of oncogenic phenotypes and therapeutic potentials. Importantly, our studies demonstrate that IHC assessment of CIN genes, exemplified by TTK here; provide better characterization and understanding for the contribution of CIN to tumor aggressiveness and prognosis.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to herein is incorporated herein by reference in their entirety.

### REFERENCES

1. Liedtke C, Mazouni C, Hess K R, Andre F, Tordai A, Mejia J A et al. Response to neoadjuvant therapy and long-term survival in patients with triple-negative breast cancer. J Clin Oncol 2008; 26: 1275-1281.
2. Carey L A, Dees E C, Sawyer L, Gatti L, Moore D T, Collichio F et al. The triple negative paradox: primary tumor chemosensitivity of breast cancer subtypes. Clin Cancer Res 2007; 13: 2329-2334.
3. von Minckwitz G, Untch M, Blohmer J U, Costa S D, Eidtmann H, Fasching P A et al. Definition and impact of pathologic complete response on prognosis after neoadjuvant chemotherapy in various intrinsic breast cancer subtypes. J Clin Oncol 2012; 30: 1796-1804.
4. Sorlie T, Perou C M, Tibshirani R, Aas T, Geisler S, Johnsen H et al. Gene expression patterns of breast carcinomas distinguish tumor subclasses with clinical implications. Proceedings of the National Academy of Sciences of the United States of America 2001; 98: 10869-10874.
5. Perou C M, Sorlie T, Eisen M B, van de Rijn M, Jeffrey S S, Rees C A et al. Molecular portraits of human breast tumours. Nature 2000; 406: 747-752.
6. Weigelt B, Hu Z, He X, Livasy C, Carey L A, Ewend M G et al. Molecular portraits and 70-gene prognosis signature are preserved throughout the metastatic process of breast cancer. Cancer research 2005; 65: 9155-9158.
7. Hu Z, Fan C, Oh D S, Marron J S, He X, Qaqish B F et al. The molecular portraits of breast tumors are conserved across microarray platforms. BMC genomics 2006; 7: 96-107.
8. Parker J S, Mullins M, Cheang M C, Leung S, Voduc D, Vickery T et al. Supervised risk predictor of breast cancer based on intrinsic subtypes. J Clin Oncol 2009; 27: 1160-1167.
9. van't Veer L J, Dai H, van de Vijver M J, He Y D, Hart A A, Mao M et al. Gene expression profiling predicts clinical outcome of breast cancer. Nature 2002; 415: 530-536.
10. Paik S, Shak S, Tang G, Kim C, Baker J, Cronin M et al. A multigene assay to predict recurrence of tamoxifen-treated, node-negative breast cancer. The New England journal of medicine 2004; 351: 2817-2826.
11. Buyse M, Loi S, van't Veer L, Viale G, Delorenzi M, Glas A M et al. Validation and clinical utility of a 70-gene prognostic signature for women with node-negative breast cancer. Journal of the National Cancer Institute 2006; 98: 1183-1192.
12. Loi S, Haibe-Kains B, Desmedt C, Lallemand F, Tutt A M, Gillet C et al. Definition of clinically distinct molecular subtypes in estrogen receptor-positive breast carcinomas through genomic grade. J Clin Oncol 2007; 25: 1239-1246.
13. Ma X J, Salunga R, Dahiya S, Wang W, Carney E, Durbecq V et al. A five-gene molecular grade index and HOXB13 IL17BR are complementary prognostic factors in early stage breast cancer. Clin Cancer Res 2008; 14: 2601-2608.
14. Ma X J, Wang Z, Ryan P D, Isakoff S J, Barmettler A, Fuller A et al. A two-gene expression ratio predicts clinical outcome in breast cancer patients treated with tamoxifen. Cancer cell 2004; 5: 607-616.
15. Sotiriou C, Wirapati P, Loi S, Harris A, Fox S, Smeds J et al. Gene expression profiling in breast cancer: understanding the molecular basis of histologic grade to improve prognosis. Journal of the National Cancer Institute 2006; 98: 262-272.
16. Cheng W Y, Ou Yang T H, Anastassiou D. Biomolecular events in cancer revealed by attractor metagenes. PLoS Comput Biol 2013; 9: e1002920.
17. Cheng W Y, Ou Yang T H, Anastassiou D. Development of a prognostic model for breast cancer survival in an open challenge environment. Sci Transl Med 2013; 5: 181ra150.
18. Dai H, van't Veer L, Lamb J, He Y D, Mao M, Fine B M et al. A cell proliferation signature is a marker of extremely poor outcome in a subpopulation of breast cancer patients. Cancer research 2005; 65: 4059-4066.
19. Rhodes D R, Yu J, Shanker K, Deshpande N, Varambally R, Ghosh D et al. ONCOMINE: a cancer microarray database and integrated data-mining platform. Neoplasia (New York, N.Y. 2004; 6: 1-6.
20. Carter S L, Eklund A C, Kohane I S, Harris L N, Szallasi Z. A signature of chromosomal instability inferred from gene expression profiles predicts clinical outcome in multiple human cancers. Nature genetics 2006; 38: 1043-1048.
21. Curtis C, Shah S P, Chin S F, Turashvili G, Rueda O M, Dunning M J et al. The genomic and transcriptomic architecture of 2,000 breast tumours reveals novel subgroups. Nature 2012; 486: 346-352.
22. Haibe-Kains B, Desmedt C, Rothe F, Piccart M, Sotiriou C, Bontempi G. A fuzzy gene expression-based computational approach improves breast cancer prognostication. Genome Biol 2010; 11: R18.
23. Szasz A M, Li Q, Eklund A C, Sztupinszki Z, Rowan A, Tokes A M et al. The CIN4 chromosomal instability qPCR classifier defines tumor aneuploidy and stratifies outcome in grade 2 breast cancer. PLoS ONE 2013; 8: e56707.
24. Gyorffy B, Lanczky A, Eklund A C, Denkert C, Budczies J, Li Q et al. An online survival analysis tool to rapidly assess the effect of 22,277 genes on breast cancer prognosis using microarray data of 1,809 patients. Breast cancer research and treatment 2010; 123: 725-731.
25. Rizzolio F, Tuccinardi T, Caligiuri I, Lucchetti C, Giordano A. CDK inhibitors: from the bench to clinical trials. Curr Drug Targets 2010; 11: 279-290.
26. Horiuchi D, Kusdra L, Huskey N E, Chandriani S, Lenburg M E, Gonzalez-Angulo A M et al. MYC pathway activation in triple-negative breast cancer is synthetic lethal with CDK inhibition. The Journal of experimental medicine 2012; 209: 679-696.
27. Manchado E, Guillamot M, Malumbres M. Killing cells by targeting mitosis. Cell death and differentiation 2012.
28. Janssen A, Medema R H. Mitosis as an anti-cancer target. Oncogene 2011; 30: 2799-2809.
29. Stucke V M, Sillje H H, Arnaud L, Nigg E A. Human Mps1 kinase is required for the spindle assembly checkpoint but not for centrosome duplication. The EMBO journal 2002; 21: 1723-1732.
30. Nagalla S, Chou J W, Willingham M C, Ruiz J, Vaughn J P, Dubey P et al. Interactions between immunity, proliferation and molecular subtype in breast cancer prognosis. Genome Biol 2013; 14: R34.
31. Bakhoum S F, Compton D A. Chromosomal instability and cancer: a complex relationship with therapeutic potential. The Journal of clinical investigation 2012; 122: 1138-1143.
32. Roylance R, Endesfelder D, Gorman P, Burrell R A, Sander J, Tomlinson I et al. Relationship of extreme chromosomal instability with long-term survival in a retrospective analysis of primary breast cancer. Cancer Epidemiol Biomarkers Prev 2011; 20: 2183-2194.
33. Birkbak N J, Eklund A C, Li Q, McClelland S E, Endesfelder D, Tan P et al. Paradoxical relationship between chromosomal instability and survival outcome in cancer. Cancer research 2011; 71: 3447-3452.
34. Janssen A, van der Burg M, Szuhai K, Kops G J, Medema R H. Chromosome segregation errors as a cause of DNA damage and structural chromosome aberrations. Science (New York, N.Y. 2011; 333: 1895-1898.
35. Kolodner R D, Cleveland D W, Putnam C D. Cancer. Aneuploidy drives a mutator phenotype in cancer. Science (New York, N.Y. 2011; 333: 942-943.
36. Luo J, Solimini N L, Elledge S J. Principles of cancer therapy: oncogene and non-oncogene addiction. Cell 2009; 136: 823-837.
37. Hanahan D, Weinberg R A. Hallmarks of cancer: the next generation. Cell 2011; 144: 646-674.
38. Al-Ejeh F, Smart C E, Morrison B J, Chenevix-Trench G, Lopez J A, Lakhani S R et al. Breast cancer stem cells: treatment resistance and therapeutic opportunities. Carcinogenesis 2011; 32: 650-658.
39. Kusumbe A P, Bapat S A. Cancer stem cells and aneuploid populations within developing tumors are the major determinants of tumor dormancy. Cancer research 2009; 69: 9245-9253.
40. Liang Y, Zhong Z, Huang Y, Deng W, Cao J, Tsao G et al. Stem-like cancer cells are inducible by increasing genomic instability in cancer cells. The Journal of biological chemistry 2010; 285: 4931-4940.
41. Do T V, Xiao F, Bickel L E, Klein-Szanto A J, Pathak H B, Hua X et al. Aurora kinase A mediates epithelial ovarian cancer cell migration and adhesion. Oncogene 2014; 33: 539-549.
42. Joshi K, Banasavadi-Siddegowda Y, Mo X, Kim S H, Mao P, Kig C et al. MELK-dependent FOXM1 phosphorylation is essential for proliferation of glioma stem cells. Stem cells (Dayton, Ohio) 2013; 31: 1051-1063.
43. Gu C, Banasavadi-Siddegowda Y K, Joshi K, Nakamura Y, Kurt H, Gupta S et al. Tumor-specific activation of the C-JUN/MELK pathway regulates glioma stem cell growth in a p53-dependent manner. Stem cells (Dayton, Ohio) 2013; 31: 870-881.
44. Hebbard L W, Maurer J, Miller A, Lesperance J, Hassell J, Oshima R G et al. Maternal embryonic leucine zipper kinase is upregulated and required in mammary tumor-initiating cells in vivo. Cancer research 2010; 70: 8863-8873.
45. Schvartzman J M, Duijf P H, Sotillo R, Coker C, Benezra R. Mad2 is a critical mediator of the chromosome instability observed upon Rb and p53 pathway inhibition. Cancer cell 2011; 19: 701-714.
46. Chan C H, Morrow J K, Li C F, Gao Y, Jin G, Moten A et al. Pharmacological inactivation of Skp2 SCF ubiquitin ligase restricts cancer stem cell traits and cancer progression. Cell 2013; 154: 556-568.
47. Daniel J, Coulter J, Woo J H, Wilsbach K, Gabrielson E. High levels of the Mps1 checkpoint protein are protective of aneuploidy in breast cancer cells. Proceedings of the National Academy of Sciences of the United States of America 2011; 108: 5384-5389.
48. Maire V, Baldeyron C, Richardson M, Tesson B, Vincent-Salomon A, Gravier E et al. TTK/hMPS1 is an attractive therapeutic target for triple-negative breast cancer. PLoS ONE 2013; 8: e63712.
49. Bild A H, Yao G, Chang J T, Wang Q, Potti A, Chasse D et al. Oncogenic pathway signatures in human cancers as a guide to targeted therapies. Nature 2006; 439: 353-357.
50. Bittner M. Expression Project for Oncology—Breast Samples. International Genomics Consortium, Phoeniz, Ariz. 85004 Oncomine. Not Published 2005/01/15
51. Bonnefoi H, Potti A, Delorenzi M, Mauriac L, Campone M, Tubiana-Hulin M et al. Validation of gene signatures that predict the response of breast cancer to neoadjuvant chemotherapy: a substudy of the EORTC 10994/BIG 00-01 clinical trial. The lancet oncology 2007; 8: 1071-1078.
52. Gluck S, Ross J S, Royce M, McKenna E F, Jr., Perou C M, Avisar E et al. TP53 genomics predict higher clinical and pathologic tumor response in operable early-stage breast cancer treated with docetaxel-capecitabine+/−trastuzumab. Breast cancer research and treatment 2012; 132: 781-791.
53. Hatzis C, Pusztai L, Valero V, Booser D J, Esserman L, Lluch A et al. A genomic predictor of response and survival following taxane-anthracycline chemotherapy for invasive breast cancer. JAMA 2011; 305: 1873-1881.
54. Kao K J, Chang K M, Hsu H C, Huang A T. Correlation of microarray-based breast cancer molecular subtypes and clinical outcomes: implications for treatment optimization. BMC cancer 2011; 11: 143.
55. Tabchy A, Valero V, Vidaurre T, Lluch A, Gomez H, Martin M et al. Evaluation of a 30-gene paclitaxel, fluorouracil, doxorubicin, and cyclophosphamide chemotherapy response predictor in a multicenter randomized trial in breast cancer. Clin Cancer Res 2010; 16: 5351-5361.
56. TCGA. Comprehensive molecular portraits of human breast tumours. Nature 2012; 490: 61-70.
57. Bos P D, Zhang X H, Nadal C, Shu W, Gomis R R, Nguyen D X et al. Genes that mediate breast cancer metastasis to the brain. Nature 2009; 459: 1005-1009.
58. Desmedt C, Piette F, Loi S, Wang Y, Lallemand F, Haibe-Kains B et al. Strong time dependence of the 76-gene prognostic signature for node-negative breast cancer patients in the TRANSBIG multicenter independent validation series. Clin Cancer Res 2007; 13: 3207-3214.
59. Schmidt M, Bohm D, von Tome C, Steiner E, Puhl A, Pilch H et al. The humoral immune system has a key prognostic impact in node-negative breast cancer. Cancer research 2008; 68: 5405-5413.
60. Symmans W F, Hatzis C, Sotiriou C, Andre F, Peintinger F, Regitnig P et al. Genomic index of sensitivity to endocrine therapy for breast cancer. J Clin Oncol 2010; 28: 4111-4119.
61. van de Vijver M J, He YD, van't Veer L J, Dai H, Hart A A, Voskuil D W et al. A gene-expression signature as a predictor of survival in breast cancer. The New England journal of medicine 2002; 347: 1999-2009.
62. Pawitan Y, Bjohle J, Amler L, Borg A L, Egyhazi S, Hall P et al. Gene expression profiling spares early breast cancer patients from adjuvant therapy: derived and validated in two population-based cohorts. Breast Cancer Res 2005; 7: R953-964.
63. Sorlie T, Tibshirani R, Parker J, Hastie T, Marron J S, Nobel A et al. Repeated observation of breast tumor subtypes in independent gene expression data sets. Proceedings of the National Academy of Sciences of the United States of America 2003; 100: 8418-8423.
64. Zhao Y, Simon R. BRB-ArrayTools Data Archive for human cancer gene expression: a unique and efficient data sharing resource. Cancer Inform 2008; 6: 9-15.
65. Miller L D, Smeds J, George J, Vega V B, Vergara L, Ploner A et al. An expression signature for p53 status in human breast cancer predicts mutation status, transcriptional effects, and patient survival. Proceedings of the National Academy of Sciences of the United States of America 2005; 102: 13550-13555.
66. Wang Y, Klijn J G, Zhang Y, Sieuwerts A M, Look M P, Yang F et al. Gene-expression profiles to predict distant metastasis of lymph-node-negative primary breast cancer. Lancet 2005; 365: 671-679.
67. Al-Ejeh F, Shi W, Miranda M, Simpson P T, Vargas A C, Song S et al. Treatment of triple-negative breast cancer using anti-EGFR-directed radioimmunotherapy combined with radiosensitizing chemotherapy and PARP inhibitor. J Nucl Med 2013; 54: 913-921.
67. Colombo R, Caldarelli M, Mennecozzi M, Giorgini M L, Sola F, Cappella P et al. Targeting the mitotic checkpoint for cancer therapy with NMS-P715, an inhibitor of MPS1 kinase. Cancer research 2010; 70: 10255-10264.
68. Janssen A, Kops G J, Medema R H. Elevating the frequency of chromosome mis-segregation as a strategy to kill tumor cells. Proceedings of the National Academy of Sciences of the United States of America 2009; 106: 19108-19113.
69. Janssen A, Medema R H. Mitosis as an anti-cancer target. Oncogene 2011; 30: 2799-2809.
70. Janssen A, van der Burg M, Szuhai K, Kops G J, Medema R H. Chromosome segregation errors as a cause of DNA damage and structural chromosome aberrations. Science (New York, N.Y. 2011; 333: 1895-1898.
71. Degenhardt Y, Lampkin T. Targeting Polo-like kinase in cancer therapy. Clin Cancer Res 2010; 16: 384-389.
72. Strebhardt K, Ullrich A. Targeting polo-like kinase 1 for cancer therapy.

Nature reviews 2006; 6: 321-330.
73. Malumbres M, Barbacid M. Cell cycle kinases in cancer. Curr Opin Genet Dev 2007; 17: 60-65.
74. Manchado E, Guillamot M, Malumbres M. Killing cells by targeting mitosis. Cell death and differentiation 2012.
75. Manchado E, Malumbres M. Targeting aneuploidy for cancer therapy. Cell 2011; 144: 465-466.
76. Colombo R, Moll J. Destabilizing aneuploidy by targeting cell cycle and mitotic checkpoint proteins in cancer cells. Curr Drug Targets 2010; 11: 1325-1335.

TABLE 1
Univariate and multivariate survival analysis of the aggressiveness score in the METABRIC dataset

| | Univariate Cox-proportional hazards | | Multivariate Cox-proportional hazards | |
|---|---|---|---|---|
| | model | | model (stepwise) | |
| | HR (95% CI) | p-value | HR (95% CI) | p-value |
| 206 genes score | 1.6173 (1.4174-1.8454) | <0.0001 | 1.5188 (1.3227-1.7440) | <0.0001 |
| (high, low) | | | | |
| 8 genes score | 1.5853 (1.2883-1.8103) | <0.0001 | 1.4760 (1.2198-1.6344) | 0.0001 |
| (high, low) | | | | |
| Lymph node | 1.8594 (1.6289-2.1224) | <0.0001 | 1.6807 (1.4610-1.9334) | <0.0001 |
| (+, −) | | | | |
| Tumor size | 1.4354 (1.2813-1.6080) | <0.0001 | 1.3666 (1.1642-1.6041) | <0.0001 |
| (T1, T2, T3) | | | | |
| HER2 status | 1.4565 (1.2537-1.6920) | <0.0001 | 1.1983 (1.0183-1.4101) | 0.0302 |
| (+, −) | | | | |
| Tumor grade | 1.3500 (1.2095-1.5067) | <0.0001 | ns | ns |
| (T1, T2, T3) | | | | |
| Ki67 | 1.4184 (1.2399-1.6226) | <0.0001 | ns | ns |
| (+, −) | | | | |
| MammaPrint | 1.3320 (1.1669-1.5204) | <0.0001 | ns | ns |
| (high, low) | | | | |
| CIN4 | 1.5310 (1.3413-1.7476) | <0.0001 | ns | ns |
| (high, low) | | | | |
| CIN75 | 1.5004 (1.3132-1.7143) | <0.0001 | ns | ns |
| (high, low) | | | | |
| Cell Cycle | 1.5018 (1.3145-1.7158) | <0.0001 | ns | ns |
| (high, low) | | | | |
| ER status | 1.3016 (1.1167-1.5170) | 0.0008 | ns | ns |
| (+, −) | | | | |
| OncotypeDx | 1.2672 (1.0909-1.4720) | 0.0021 | ns | ns |
| (L, I, H) | | | | |
| Treatment | 1.1646 (0.9753-1.2639) | 0.0939 | ns | ns |
| (yes, no) | | | | |
| Age | 1.1235 (0.8480-1.4886) | 0.4196 | ns | ns |
| (<40, >40) | | | | |

HR: Hazard Ratio.
CI: confidence interval.
ns: not significant.
OncoTypeDx scores are low (L, <18), intermediate (I, 18-31), high (H > 31).
All variables were included in the multivariate Cox-proportional hazards model analysis and by stepwise model, only significant co-variants were included in the final analysis shown in Table.

TABLE 2
Correlation of TTK mRNA level and clinico-pathological
indicators in the METABRIC dataset

| Comparison | TTK Low | TTK high | X² |
|---|---|---|---|
| Tumor size | | | |
| <2 cm | 346 (18%) | 280 (14%) | p < 1.0E−6 |
| >2 cm <5 cm | 509 (26%) | 685 (35%) | p = 3.2E−5 |
| >5 cm | 60 (3%) | 92 (5%) | p = 1.25E−2 |
| Tumor Grade | | | |
| Grade 1 | 137 (7%) | 33 (2%) | p < 1.0E−6 |
| Grade 2 | 479 (25%) | 296 (16%) | p < 1.0E−6 |
| Grade 3 | 251 (13%) | 706 (37%) | p < 1.0E−6 |
| Ki67 expression | | | |
| Low | 826 (39%) | 242 (11%) | |
| High | 237 (11%) | 831 (39%) | p < 1.0E−6 |
| Immunohistochemical subtypes | | | |
| ER negative | 71 (4%) | 369 (19%) | p < 1.0E−6 |
| ER positive | 827 (42%) | 681 (35%) | |
| PR negative | 306 (15%) | 637 (32%) | p < 1.0E−6 |
| PR positive | 617 (31%) | 432 (22%) | |
| HER2 negative | 802 (40%) | 744 (37%) | |
| HER2 positive | 118 (6%) | 323 (16%) | p < 1.0E−6 |
| non-TNBC | 885 (45%) | 840 (43%) | |
| Triple negative (TNBC) | 29 (1%) | 221 (11%) | p < 1.0E−6 |
| Intrinsic subtypes | | | |
| Luminal A | 552 (28%) | 169 (9%) | p < 1.0E−6 |
| Luminal B | 142 (7%) | 350 (18%) | p < 1.0E−6 |
| HER2-enriched | 40 (2%) | 200 (10%) | p < 1.0E−6 |
| Normal-like | 161 (8%) | 41 (2%) | p < 1.0E−6 |
| Basal-like | 26 (1%) | 305 (15%) | p < 1.0E−6 |
| Age (years) | | | |
| <50 | 167 (8%) | 259 (13%) | p = 8.68E−4 |
| 50-74 | 485 (24%) | 549 (27%) | ns |
| 75-100 | 282 (14%) | 253 (13%) | ns |
| TP53 mutation | | | |
| Wildtype | 390 (48%) | 331 (40%) | |
| Mutant | 14 (2%) | 85 (10%) | p < 1.0E−6 |

X²: Chi square test performed using GraphPad ® Prism.
ns not significant

TABLE 3
Associations between TTK protein expression and clinico-pathological indicators

| Parameter | TTK (0-1) | TTK (2) | TTK (3) | P value^{#} |
|---|---|---|---|---|
| Histological type | | | | |
| Ductal NOS | 147 (60.7%) | 67 (27.7%) | 28 (11.6%) | 0.0265 |
| Lobular | 43 (76.8%) | 10 (17.9%) | 3 (5.4%) | |
| Mixed ducto-lobular | 31 (88.6%) | 4 (11.4%) | 0 (0.0%) | |
| Metaplastic | 9 (56.3%) | 7 (43.8%) | 0 (0.0%) | |
| Tubular/cribifonn | 8 (80.0%) | 2 (20.0%) | 0 (0.0%) | |
| Other special types (incl mixed) | 37 (66.1%) | 14 (25.0%) | 5 (8.9%) | |
| Overall grade | | | | |
| 1 | 43 (76.8%) | 13 (23.2%) | 0 (0.0%) | <0.0001 |
| 2 | 162 (77.5%) | 41 (19.6%) | 6 (2.9%) | |
| 3 | 73 (47.7%) | 50 (32.7%) | 30 (19.6%) | |
| Mitotic score | | | | |
| 1 | 193 (79.8%) | 44 (18.2%) | 5 (2.1%) | <0.0001 |
| 2 | 33 (61.1%) | 18 (33.3%) | 3 (5.6%) | |
| 3 | 52 (43.0%) | 42 (34.7%) | 27 (22.3%) | |
| Nuclear pleomorphism score | | | | |
| 1-2 | 164 (75.2%) | 49 (22.5%) | 5 (2.3%) | <0.0001 |
| 3 | 115 (57.2%) | 55 (27.4%) | 31 (15.4%) | |
| Tubule score | | | | |
| 1 | 10 (76.9%) | 3 (23.1%) | 0 (0.0%) | ns |
| 2 | 52 (69.3%) | 20 (26.7%) | 3 (4.0%) | |
| 3 | 216 (65.5%) | 81 (24.5%) | 33 (10.0%) | |
| Lymph node status | | | | |
| Positive | 77 (62.1%) | 41 (33.1%) | 6 (4.8%) | 0.0056 |
| Negative | 81 (73.0%) | 18 (16.2%) | 12 (10.8%) | |
| Tumor size | | | | |
| <2 cm | 112 (68.3%) | 40 (24.4%) | 12 (7.3%) | ns |
| 2-5 cm | 104 (66.2%) | 38 (24.20) | 15 (9.60) | |
| >5 cm | 19 (61.3%) | 6 (19.40) | 6 (19.40) | |
| Lymphovascular invasion | | | | |
| Absent | 214 (67.3%) | 77 (24.2%) | 27 (8.5%) | ns |
| Present | 63 (63.6%) | 27 (27.3%) | 9 (9.1%) | |
| Lymphocytic infiltrate | | | | |
| Absent | 119 (78.3%) | 28 (18.40o) | 5 (3.3%) | 0.0007 |
| Mild | 115 (63.9%) | 47 (26.1%) | 18 (10.0%) | |
| Moderate | 36 (53.7%) | 23 (34.3%) | 8 (11.9%) | |
| Severe | 7 (41.2%) | 6 (35.3%) | 4 (23.5%) | |
| Central scarring/fibrosis | | | | |
| Absent | 254 (67.7%) | 90 (24.00) | 31 (8.3%) | ns |
| Present | 25 (56.8%) | 14 (31.80) | 5 (11.40) | |
| Tumor border | | | | |
| Infiltrative | 250 (69.1%) | 88 (24.3%) | 24 (6.6%) | 0.0003 |
| Pushing (<50%) | 11 (36.7%) | 11 (36.7%) | 8 (26.7%) | |
| Pushing (>50%) | 16 (64.0%) | 5 (20.0%) | 4 (16.0%) | |
| Ki67 expression (20% threshold) | | | | |
| Low | 240 (71.6%) | 77 (23.0%) | 18 (5.4%) | <0.0001 |
| High | 14 (25.9%) | 23 (42.6%) | 17 (31.5%) | |
| Prognostic subgroups | | | | |
| HER2+ | 21 (51.2%) | 14 (34.1%) | 6 (14.6%) | <0.0001 |
| HR+/HER2-neg (Ki67-high) | 6 (24.0%) | 13 (52.0%) | 6 (24.0%) | |
| HR+/HER2-neg (Ki67-low) | 196 (76.0%) | 53 (20.5%) | 9 (3.5%) | |
| TN (basal-like) | 23 (41.80) | 20 (36.4%) | 12 (21.8%) | |
| TN (non-basal) | 10 (71.40) | 1 (7.10) | 3 (21.40) | |

TMAs were scored by two independent assessors according to the following categories: 0, negative; 1, weak and focal staining (pooled with negative cases for this analysis); 2, moderate-strong focal staining (collectively <50%) of tumour cells); 3 = moderate-strong diffuse staining (>50% of tumour cells). Regarding % cells stained, we disregarded mitotic cells to assess mitosis-independent TTK expression.
^{#}Chi square test (GraphPad ® Prism. ns: not significant)

TABLE 4
The aggressiveness genelist (206 genes)

| Input | Approved Name | HGNC ID | Location |
|---|---|---|---|
| ADIRF | adipogenesis regulatory factor | HGNC: 24043 | 10q23.31 |
| AFF3 | AF4/FMR2 family, member 3 | HGNC: 6473 | 2q11.2-q12 |
| AGO2 | argonaute RISC catalytic component 2 | HGNC: 3263 | 8q24.3 |
| AGR3 | anterior gradient 3 homolog (Xenopus laevis) | HGNC: 24167 | 7p21.1 |
| AHNAK | AHNAK nucleoprotein | HGNC: 347 | 11q12-q13 |
| ALDH3A2 | aldehyde dehydrogenase 3 family, member | HGNC: 403 | 17p11.2 |
| | A2 | | |
| ANLN | anillian, actin binding protein | HGNC: 14082 | 7p15-p14 |
| APOBEC3B | apolipoprotein B mRNA editing enzyme, | HGNC: 17352 | 22q13.1-q13.2 |
| | catalytic polypeptide-like 3B | | |
| AQP9 | aquaporin 9 | HGNC: 643 | 15q |
| ATP6V1C2 | ATPase, H+ transporting, lysosomal 42 kDa, | HGNC: 18264 | 2p25.1 |
| | V1 subunit C2 | | |
| AUNIP | aurora kinase A and ninein interacting | HGNC: 28363 | 1p36.11 |
| | protein | | |
| AURKA | aurora kinase A | HGNC: 11393 | 20q13 |
| AURKB | aurora kinase B | HGNC: 11390 | 17p13.1 |
| AZGP1 | alpha-2-glycoprotein 1, zinc-binding | HGNC: 910 | 7q22.1 |
| BBS1 | Bardet-Biedl syndrome 1 | HGNC: 966 | 11q13 |
| BCL2 | B-cell CLL/lymphoma 2 | HGNC: 990 | 18q21.3 |
| BIRC5 | baculoviral IAP repeat containing 5 | HGNC: 593 | 17q25.3 |
| BLM | Bloom syndrome, RecQ helicase-like | HGNC: 1058 | 15q26.1 |
| BTG2 | BTG family, member 2 | HGNC: 1131 | 1q32 |
| BUB1 | BUB1 mitotic checkpoint serine/threonine | HGNC: 1148 | 2q13 |
| | kinase | | |
| BYSL | bystin-like | HGNC: 1157 | 6p21.1 |
| C10orf32 | chromosome 10 open reading frame 32 | HGNC: 23516 | 10q24.33 |
| C18orf56 | chromosome 18 open reading frame 56 | HGNC: 29553 | 18p11.32 |
| C1orf106 | chromosome 1 open reading frame 106 | HGNC: 25599 | 1q32.1 |
| C1orf21 | chromosome 1 open reading frame 21 | HGNC: 15494 | 1q25 |
| C7orf63 | chromosome 7 open reading frame 63 | HGNC: 26107 | 7q21.13 |
| CA9 | carbonic anhydrase IX | HGNC: 1383 | 9p13.3 |
| CARD10 | caspase recruitment domain family, member | HGNC: 16422 | 22q13.1 |
| | 10 | | |
| CASC1 | cancer susceptibility candidate 1 | HGNC: 29599 | 12p12.1 |
| CCDC170 | coiled-coil domain containing 170 | HGNC: 21177 | 6q25.1 |
| CCDC176 | coiled-coil domain containing 176 | HGNC: 19855 | 14q24.3 |
| CCNA2 | cyclin A2 | HGNC: 1578 | 4q27 |
| CCNB2 | cyclin B2 | HGNC: 1580 | 15q21.3 |
| CCNE1 | cyclin E1 | HGNC: 1589 | 19q12 |
| CCNG2 | cyclin G2 | HGNC: 1593 | 4q21.22 |
| CD163 | CD163 molecule | HGNC: 1631 | 12p13 |
| CDC20 | cell division cycle 20 | HGNC: 1723 | 1p34.1 |
| CDC25A | cell division cycle 25A | HGNC: 1725 | 3p21 |
| CDC25B | cell division cycle 25B | HGNC: 1726 | 20p13 |
| CDC45 | cell division cycle 45 | HGNC: 1739 | 22q11.21 |
| CDCA3 | cell division cycle associated 3 | HGNC: 14624 | 12p13.31 |
| CDCA5 | cell division cycle associated 5 | HGNC: 14626 | 11q13.1 |
| CDCA7 | cell division cycle associated 7 | HGNC: 14628 | 2q31.1 |
| CDCA8 | cell division cycle associated 8 | HGNC: 14629 | 1p34.3 |
| CDK1 | cyclin-dependent kinase 1 | HGNC: 1722 | 10q21.2 |
| CDKN2A | cyclin-dependent kinase inhibitor 2A | HGNC: 1787 | 9p21 |
| CENPA | centromere protein A | HGNC: 1851 | 2p23.3 |
| CENPE | centromere protein E, 312 kDa | HGNC: 1856 | 4q24-q25 |
| CENPN | centromere protein N | HGNC: 30873 | 16q23.2 |
| CENPW | centromere protein W | HGNC: 21488 | 6q22.32 |
| CEP55 | centrosomal protein 55 kDa | HGNC: 1161 | 10q24.1 |
| CHEK1 | checkpoint kinase 1 | HGNC: 1925 | 11q24.2 |
| CIRBP | cold inducible RNA binding protein | HGNC: 1982 | 19p13.3 |
| CKAP2L | cytoskeleton associated protein 2-like | HGNC: 26877 | 2q13 |
| CKS1B | CDC28 protein kinase regulatory subunit 1B | HGNC: 19083 | 1q21.2 |
| CKS2 | CDC28 protein kinase regulatory subunit 2 | HGNC: 2000 | 9q22 |
| CLIC6 | chloride intracellular channel 6 | HGNC: 2065 | 21q22.12 |
| CMC2 | COX assembly mitochondrial protein 2 | HGNC: 24447 | 16q23.2 |
| | homolog (S. cerevisiae) | | |
| CMYA5 | cardiomyopathy associated 5 | HGNC: 14305 | 5q14.1 |
| CPEB2 | cytoplasmic polyadenylation element binding | HGNC: 21745 | 4p15.33 |
| | protein 2 | | |
| CST3 | cystatin C | HGNC: 2475 | 20p11.2 |
| CSTB | cystatin B (stefin B) | HGNC: 2482 | 21q22.3 |
| CTSV | cathepsin V | HGNC: 2538 | 9q22.33 |
| CYB5D1 | cytochrome b5 domain containing 1 | HGNC: 26516 | 17p13.1 |
| CYBRD1 | cytochrome b reductase 1 | HGNC: 20797 | 2q31 |
| DACH1 | dachshund homolog 1 (Drosophila) | HGNC: 2663 | 13q22 |
| DAPK1 | death-associated protein kinase 1 | HGNC: 2674 | 9q34.1 |
| DEPDC1 | DEP domain containing 1 | HGNC: 22949 | 1p31.2 |
| DKC1 | dyskeratosis congenita 1, dyskerin | HGNC: 2890 | Xq28 |
| DLGAP5 | discs, large (Drosophila) homolog-associated | HGNC: 16864 | 14q22.3 |
| | protein 5 | | |
| DNAJC12 | DnaJ (Hsp40) homolog, subfamily C, | HGNC: 28908 | 10q21.3 |
| | member 12 | | |
| DNALI1 | dynein, axonemal, light intermediate chain 1 | HGNC: 14353 | 1p35.1 |
| EC12 | enoyl-CoA delta isomerase 2 | HGNC: 14601 | 6p24.3 |
| ELOVL5 | ELOVL fatty acid elongase 5 | HGNC: 21308 | 6p21.1-p12.1 |
| ESR1 | estrogen receptor 1 | HGNC: 3467 | 6q24-q27 |
| EXO1 | exonuclease 1 | HGNC: 3511 | 1q42-q43 |
| FAM198B | family with sequence similarity 198, member | HGNC: 25312 | 4q32.1 |
| | B | | |
| FAM214A | family with sequence similarity 214, member | HGNC: 25609 | 15q21.2-q21.3 |
| | A | | |
| FAM64A | family with sequence similarity 64, member | HGNC: 25483 | 17p13.2 |
| | A | | |
| FAM83D | family with sequence similarity 83, member | HGNC: 16122 | 20 |
| | D | | |
| FOXA1 | forkhead box A1 | HGNC: 5021 | 14q12-q13 |
| FOXM1 | forkhead box M1 | HGNC: 3818 | 12p13 |
| FPR3 | formyl peptide receptor 3 | HGNC: 3828 | 19q13.3-q13.4 |
| GAPDH | glyceraldehyde-3-phosphate dehydrogenase | HGNC: 4141 | 12p13.31 |
| GFRA1 | GDNF family receptor alpha 1 | HGNC: 4243 | 10q25-q26 |
| GGH | gamma-glutamyl hydrolase (conjugase, | HGNC: 4248 | 8q12.3 |
| | folylpoly gammaglutamyl hydrolase) | | |
| GLI3 | GLI family zinc finger 3 | HGNC: 4319 | 7p13 |
| GLYATL2 | glycine-N-acyltransferase-like 2 | HGNC: 24178 | 11q12.1 |
| GPD1L | glycerol-3-phosphate dehydrogenase 1-like | HGNC: 28956 | 3p22.3 |
| GPSM2 | G-protein signaling modulator 2 | HGNC: 29501 | 1p13.3 |
| GSTM1 | glutathione S-transferase mu 1 | HGNC: 4632 | 1p13.3 |
| GSTM3 | glutathione S-transferase mu 3 (brain) | HGNC: 4635 | 1p13.3 |
| GTPBP4 | GTP binding protein 4 | HGNC: 21535 | 10p15-p14 |
| GTSE1 | G-2 and S-phase expressed 1 | HGNC: 13698 | 22q13.2-q13.3 |
| HJURP | Holliday junction recognition protein | HGNC: 25444 | 2q37.1 |
| HRASLS | HRAS-like suppressor | HGNC: 14922 | 3q29 |
| HSD17B4 | hydroxysteroid (17-beta) dehydrogenase 4 | HGNC: 5213 | 5q2 |
| HSD17B8 | hydroxysteroid (17-beta) dehydrogenase 8 | HGNC: 3554 | 6p21.3 |
| IGFBP2 | insulin-like growth factor binding protein 2, | HGNC: 5471 | 2q33-q34 |
| | 36 kDa | | |
| IGFBP4 | insulin-like growth factor binding protein 4 | HGNC: 5473 | 17q12-q21.1 |
| IL6ST | interleukin 6 signal transducer (gp130, | HGNC: 6021 | 5q11.2 |
| | oncostatin M receptor) | | |
| IL8 | interleukin 8 | HGNC: 6025 | 4q13-q21 |
| IMPA2 | inositol (myo)-1 (or 4)-monopliphatase 2 | HGNC: 6051 | 18p11.2 |
| IRAK1 | interleukin-1 receptor-associated kinase 1 | HGNC: 6112 | Xq28 |
| KCNG1 | potassium voltage-gated channel, subfamily | HGNC: 6248 | 20q13 |
| | G, member 1 | | |
| KCNMA1 | potassium large conductance calcium- | HGNC: 6284 | 10q22 |
| | activated channel, subfamily M, alpha | | |
| | member1 | | |
| KCTD3 | potassium channel tetramerization domain | HGNC: 21305 | 1q41 |
| | containing 3 | | |
| KIF13B | kinesin family member 13B | HGNC: 14405 | 8p21 |
| KIF14 | kinesin family member 14 | HGNC: 19181 | 1q32.1 |
| KIF20A | kinesin family member 20A | HGNC: 9787 | 5q31 |
| KIF23 | kinesin family member 23 | HGNC: 6392 | 15q23 |
| KIF2C | kinesin family member 2C | HGNC: 6393 | 1p34.1 |
| KIF5C | kinesin family member 5C | HGNC: 6325 | 2q23 |
| KRT6A | keratin 6A | HGNC: 6443 | 12q13.13 |
| LAD1 | ladinin 1 | HGNC: 6472 | 1q25.1-q32.3 |
| LAPTM4B | lysosomal protein transmembrane 4 beta | HGNC: 13646 | 8q22.1 |
| LFNG | LFNG O-fucosylpeptide 3-beta-N- | HGNC: 6560 | 7p22.3 |
| | acetylglucosaminyltransferase | | |
| LMNB2 | lamin B2 | HGNC: 6638 | 19p13.3 |
| LOC100286909 | — | — | — |
| LRIG1 | leucine-rich repeats and immunoglobulin- | HGNC: 17360 | 3p14 |
| | like domains 1 | | |
| LRP8 | low density lipoprotein receptor-related | HGNC: 6700 | 1p32.3 |
| | protein 8, apolipoprotein e receptor | | |
| LYPD6 | LY6/PLAUR domain containing 6 | HGNC: 28751 | 2q23.2 |
| MAD2L1 | MAD2 mitotic arrest deficient-like 1 (yeast) | HGNC: 6763 | 4q27 |
| MAPT | microtubule-associated protein tau | HGNC: 6893 | 17q21 |
| MCM10 | inthromosoine maintenance complex | HGNC: 18043 | 10p13 |
| | component 10 | | |
| MCM2 | minichromosome maintenance complex | HGNC: 6944 | 3q21 |
| | component 2 | | |
| MCM4 | minichromosome maintenance complex | HGNC: 6947 | 8q12-q13 |
| | component 4 | | |
| MCM6 | minichromosome maintenance complex | HGNC: 6949 | 2q14-q21 |
| | component 6 | | |
| MCM7 | minichromosome maintenance complex | HGNC: 6950 | 7q21.3-q22.1 |
| | component 7 | | |
| MEIS3P1 | Meis homeobox 3 pseudogene 1 | HGNC: 7002 | 17p12 |
| MELK | maternal embryonic leucine zipper kinase | HGNC: 16870 | 9p13.1 |
| MLPH | melanophilin | HGNC: 29643 | 2q37.2 |
| MST1 | macrophage stimulating 1 (hepatocyte | HGNC: 7380 | 3p21 |
| | growth factor-like) | | |
| MTHFD1L | methylenetetrahydrofolate dehydrogenase | HGNC: 21055 | 6q25.1 |
| | (NADP + dependent) 1-like | | |
| MX2 | myxovirus (influenza virus) resistance 2 | HGNC: 7533 | 21q22.3 |
| | (mouse) | | |
| MYB | v-myb avian myeloblastosis viral oncogene | HGNC: 7545 | 6q22-q23 |
| | homolog | | |
| NCAPG | non-SMC condensin-1 complex, subunit G | HGNC: 24304 | 4p15.32 |
| NDC80 | NDC80 kinetochore complex component | HGNC: 16909 | 18p11.31 |
| NFIA | nuclear factor I/A | HGNC: 7784 | 1p31.3-p31.2 |
| NME5 | NME/NM23 family member 5 | HGNC: 7853 | 5q31.2 |
| NOP2 | NOP2 nucleolar protein | HGNC: 7867 | 12p13 |
| NOSTRIN | nitric oxide synthase trafficker | HGNC: 20203 | 2q31.1 |
| NOVA1 | neuro-oncological ventral antigen 1 | HGNC: 7886 | 14q12 |
| NRIP1 | nuclear receptor interacting protein 1 | HGNC: 8001 | 21q11.2 |
| NUP205 | nucleoporin 205 kDa | HGNC: 18658 | 7q31.32 |
| NUP93 | nucleoporin 93 kDa | HGNC: 28958 | 16q13 |
| NUSAP1 | nucleoporin and spindle associated protein 1 | HGNC: 18538 | 15q14 |
| OGN | osteoglycin | HGNC: 8126 | 9q22 |
| PDCD4 | programmed cell death 4 (neoplastic | HGNC: 8763 | 10q24 |
| | transformation inhibitor) | | |
| PFKP | phosphofructokinase, platelet | HGNC: 8878 | 10p15.3-p15.2 |
| PHYHD1 | phytanoyl-CoA dioxygenase domain | HGNC: 23396 | 9q34.13 |
| | containing 1 | | |
| PIP | prolactin-induced protein | HGNC: 8993 | 7q32-qter |
| PLAT | plasminogen activator, tissue | HGNC: 9051 | 8p11.21 |
| PLCH1 | phospholipase C, eta 1 | HGNC: 29185 | 3q25 |
| PNP | purine nucleoside phosphorylase | HGNC: 7892 | 14q11.2 |
| PNPLA7 | patatin-like phospholipase domain containing | HGNC: 24768 | 9q34.3 |
| | 7 | | |
| PRC1 | protein regulator of cytokinesis 1 | HGNC: 9341 | 15q26.1 |
| PSMB2 | proteasome (prosome, macropain) subunit, | HGNC: 9539 | 1p34.2 |
| | beta type, 2 | | |
| PTGER3 | prostaglandin E receptor 3 (subtype EPS3) | HGNC: 9595 | 1p31.2 |
| PTPRT | protein tyrosine phosphatase, receptor type, | HGNC: 9682 | 20q12-q13 |
| | T | | |
| PTTG1 | pituitary tumor-transforming 1 | HGNC: 9690 | 5q35.1 |
| QDPR | quinoid dihydropteridine reductase | HGNC: 9752 | 4p15.31 |
| RAB27B | RAB27B, member RAS oncogene family | HGNC: 9767 | 18q21.2 |
| RABEP1 | rabaptin, RAB GTPase binding effector | HGNC: 17677 | 17p13.2 |
| | protein 1 | | |
| RAD51AP1 | RAD51 associated protein 1 | HGNC: 16956 | 12p13.2-p13.1 |
| RBM38 | RNA binding motif protein 38 | HGNC: 15818 | 20q13.31 |
| RERG | RAS-like, estrogen-regulated, growth | HGNC: 15980 | 12p13.1 |
| | inhibitor | | |
| RFC4 | replication factor C (activator 1) 4, 37 kDa | HGNC: 9972 | 3q27 |
| RIPK2 | receptor-interacting serine-threonine kinase 2 | HGNC: 10020 | 8q21 |
| RNASE4 | ribonuclease, RNase A family, 4 | HGNC: 10047 | 14q11 |
| RPP40 | ribonuclease P/MRP 40 kDa subunit | HGNC: 20992 | 6p25.1 |
| RPS23 | ribosomal protein S23 | HGNC: 10410 | 5q14.2 |
| S100A8 | S100 calcium binding protein A8 | HGNC: 10498 | 1q12-q22 |
| SCUBE2 | signal peptide, CUB domain, EGF-like 2 | HGNC: 30425 | 11p15.3 |
| SH3BGRL | SH3 domain binding glutamic acid-rich | HGNC: 10823 | Xq13.3 |
| | protein like | | |
| SKP1 | S-phase kinase-associated protein 1 | HGNC: 10899 | 5q31 |
| SKP2 | S-phase kinase-asseciated protein 2, E3 | HGNC: 10901 | 5p13 |
| | ubiquitin protein ligase | | |
| SLC16A10 | solute carrier family 16 (aromatic amino acid | HGNC: 17027 | 6q21-q22 |
| | transporter), member 10 | | |
| SLC2A1 | solute carrier family 2 (facilitated glucose | HGNC: 11005 | 1p34.2 |
| | transporter), member 1 | | |
| SLC39A6 | solute carrier family 39 (zinc transporter), | HGNC: 18607 | 18q12.2 |
| | member 6 | | |
| SLC40A1 | solute carrier family 40 (iron-regulated | HGNC: 10909 | 2q32 |
| | transporter), member 1 | | |
| SLC7A5 | solute carrier family 7 (amino acid | HGNC: 11063 | 16q24.3 |
| | transporter light chain, L system), member 5 | | |
| SOD2 | superoxide dismutase 2, mitochondrial | HGNC: 11180 | 6q25 |
| SOX11 | SRY (sex determining region Y)-box 11 | HGNC: 11191 | 2p25 |
| SRD5A1 | steroid-5-alpha-reductase, alpha polypeptide | HGNC: 11284 | 5p15.31 |
| | 1 (3-oxo-5 alpha-steroid delta 4- | | |
| | dehydrogenase alpha 1) | | |
| SRPK1 | SRSF protein kinase 1 | HGNC: 11305 | 6p21.31 |
| STC2 | stanniocalcin 2 | HGNC: 11374 | 5q35.2 |
| STIL | SCL/TAL1 interrupting locus | HGNC: 10879 | 1p32 |
| STK32B | serine/threonine kinase 32B | HGNC: 14217 | 4p16 |
| SYTL4 | synaptotagmin-like 4 | HGNC: 15588 | Xq21.33 |
| TAT | tyrosine aminotransferase | HGNC: 11573 | 16q22.1 |
| TBC1D9 | TBC1 domain family, member 9 (with | HGNC: 21710 | 4q31.1 |
| | GRAM domain) | | |
| TEAD4 | TEA domain family member 4 | HGNC: 11711 | 12p13.3-p13.2 |
| TFF1 | trefoil factor 1 | HGNC: 11755 | 21q22.3 |
| TFF3 | trefoil factor 3 (intestinal) | HGNC: 11757 | 21q22.3 |
| TMEM26 | transmembrane protein 26 | HGNC: 28550 | 10q21.3 |
| TPX2 | TPX2, microtubule-associated, homolog | HGNC: 1249 | 20q11.2 |
| | (Xenopus laevis) | | |
| TRIP13 | thyroid hormone receptor interactor 13 | HGNC: 12307 | 5p15 |
| TROAP | trophinin associated protein | HGNC: 12327 | 12q13.12 |
| TTK | TTK protein kinase | HGNC: 12401 | 6q13-q21 |
| TUBA4A | tubulin, alpha 4a | HGNC: 12407 | 2q36.1 |
| UBE2C | ubiquitin-conjugating enzyme E2C | HGNC: 15937 | 20q13.12 |
| USB1 | U6 snRNA biogenesis 1 | HGNC: 25792 | 16q13 |
| VGLL1 | vestigial like 1 (Drosophila) | HGNC: 20985 | Xq26.3 |
| XBP1 | X-box binding protein 1 | HGNC: 12801 | 22q12.1 |
| YEATS2 | YEATS domain containing 2 | HGNC: 25489 | 3q27.3 |

TABLE 6
Association of the 6 overexpressed genes in the 8-genes
score with RFS and DMFS at 5 and 10 years

| | | RFS | | | |
|---|---|---|---|---|---|
| | | 5 years | | 10 years | |
| Patient subgroup and cut-off | | HR (95% CI) | p-value | HR (95% CI) | p-value |
| All | Median | 2.52 (2.17-2.93) | <1.00E−16 | 2.16 (1.90-2.47) | <1.00E−16 |
| | Quartile | 3.03 (2.43-3.78) | <1.00E−16 | 2.46 (2.09-2.89) | <1.00E−16 |
| | Tertile | 2.83 (2.35-3.41) | <1.00E−16 | 2.70 (2.24-3.27) | <1.00E−16 |
| ER+ | Median | 2.67 (2.25-3.17) | <1.00E−16 | 2.28 (1.96-2.65) | <1.00E−16 |
| | Quartile | 2.90 (2.29-3.67) | <1.00E−16 | 2.64 (2.16-3.23) | <1.00E−16 |
| | Tertile | 2.87 (2.34-3.53) | <1.00E−16 | 2.51 (2.11-2.99) | <1.00E−16 |
| LN− | Median | 2.76 (2.19-3.48) | <1.00E−16 | 2.25 (1.84-2.74) | 2.20E−16 |
| | Quartile | 2.76 (2.00-3.80) | 1.10E−10 | 2.51 (1.91-3.29) | 5.10E−12 |
| | Tertile | 2.92 (2.21-3.87) | 4.30E−15 | 2.53 (2.00-3.19) | 1.00E−15 |
| LN+ | Median | 2.20 (1.57-3.08) | 2.40E−06 | 1.88 (1.40-2.52) | 1.90E−05 |
| | Quartile | 3.19 (1.87-5.44) | 6.60E−06 | 2.56 (1.67-3.94) | 8.10E−06 |
| | Tertile | 2.45 (1.61-3.37) | 1.70E−05 | 2.05 (1.45-2.91) | 4.00E−05 |
| | | DMFS | | | |
| | | 5 years | | 10 years | |
| Patient subgroup and cut-off | | HR (95% CI) | p-value | HR (95% CI) | p-value |
| All | Median | 2.87 (2.17-3.79) | 8.90E−15 | 2.37 (1.87-3.01) | 1.90E−13 |
| | Quartile | 3.64 (2.41-5.52) | 5.80E−11 | 3.43 (2.41-4.88) | 3.20E−12 |
| | Tertile | 3.53 (2.48-5.04) | 8.70E−14 | 2.92 (2.18-3.90) | 3.70E−14 |
| ER+ | Median | 3.43 (2.49-4.74) | 1.30E−15 | 2.63 (2.00-3.45) | 4.20E−13 |
| | Quartile | 3.41 (2.21-5.27) | 8.80E−09 | 3.27 (2.26-4.71) | 2.20E−11 |
| | Tertile | 3.87 (2.62-5.74) | 8.10E−13 | 3.07 (2.34-4.20) | 2.30E−13 |
| LN− | Median | 4.84 (2.53-9.26) | 1.40E−07 | 2.80 (2.00-3.93) | 4.20E−10 |
| | Quartile | 4.86 (2.82-9.37) | 2.70E−10 | 4.46 (2.61-7.60) | 1.80E−09 |
| | Tertile | 3.98 (2.61-6.07) | 4.20E−12 | 3.76 (2.46-5.74) | 4.50E−11 |
| LN+ | Median | 2.12 (1.19-3.80) | 9.40E−03 | 2.16 (1.28-3.62) | 3.00E−03 |
| | Quartile | 2.97 (1.18-7.44) | 1.50E−02 | 2.87 (1.31-6.29) | 6.00E−03 |
| | Tertile | 3.51 (1.50-8.21) | 2.00E−03 | 2.88 (1.47-5.68) | 1.40E−03 |

p-values are from Log rank test from KM-plotter

TABLE 7
Association of the 2 overexpressed genes in the 8-genes
score with RFS and DMFS at 5 and 10 years

| | | RFS | | | |
|---|---|---|---|---|---|
| | | 5 years | | 10 years | |
| Patient subgroup and cut-off | | HR (95% CI) | p-value | HR (95% CI) | p-value |
| All | Median | 0.53 (0.46-0.61) | <1.00E−16 | 0.59 (0.52-0.67) | 5.60E−16 |
| | Quartile | 0.53 (0.35-0.61) | <1.00E−16 | 0.59 (0.52-0.68) | 8.20E−14 |
| | Tertile | 0.49 (0.43-0.57) | <1.00E−16 | 0.57 (0.50-0.65) | <1.00E−16 |
| ER+ | Median | 0.60 (0.51-0.71) | 2.10E−09 | 0.65 (0.56-0.76) | 2.60E−08 |
| | Quartile | 0.62 (0.48-0.79) | 1.30E−04 | 0.69 (0.54-0.86) | 1.20E−03 |
| | Tertile | 0.54 (0.45-0.66) | 6.00E−10 | 0.63 (0.52-0.76) | 5.60E−07 |
| LN− | Median | 0.61 (0.49-0.76) | 9.00E−06 | 0.71 (0.58-0.86) | 4.20E−04 |
| | Quartile | 0.56 (0.44-0.72) | 3.50E−06 | 0.63 (0.50-0.79) | 6.00E−05 |
| | Tertile | 0.53 (0.42-0.66) | 2.20E−08 | 0.62 (0.50-0.76) | 4.60E−06 |
| LN+ | Median | 0.58 (0.42-0.80) | 9.70E−04 | 0.70 (0.52-0.93) | 1.30E−02 |
| | Quartile | 0.59 (0.42-0.84) | 3.00E−03 | 0.70 (0.50-0.98) | 3.50E−02 |
| | Tertile | 0.57 (0.41-0.78) | 4.00E−04 | 0.68 (0.50-0.91) | 9.70E−03 |
| | | DMFS | | | |
| | | 5 years | | 10 years | |
| Patient subgroup and cut-off | | HR (95% CI) | p-value | HR (95% CI) | p-value |
| All | Median | 0.59 (0.47-0.74) | 2.50E−06 | 0.59 (0.47-0.74) | 2.50E−06 |
| | Quartile | 0.58 (0.46-0.74) | 4.40E−06 | 0.58 (0.46-0.74) | 4.40E−06 |
| | Tertile | 0.57 (0.46-0.71) | 4.90E−07 | 0.57 (0.46-0.71) | 4.90E−07 |
| ER+ | Median | 0.62 (0.48-0.81) | 3.00E−04 | 0.62 (0.48-0.81) | 3.00E−04 |
| | Quartile | 0.56 (0.39-0.82) | 2.40E−03 | 0.56 (0.39-0.82) | 2.40E−03 |
| | Tertile | 0.57 (0.42-0.78) | 3.00E−04 | 0.57 (0.42-0.78) | 3.00E−04 |
| LN− | Median | 0.74 (0.54-1.00) | 4.60E−02 | 0.74 (0.54-1.00) | 4.50E−02 |
| | Quartile | 0.64 (0.46-0.89) | 7.00E−03 | 0.64 (0.46-0.89) | 7.00E−03 |
| | Tertile | 0.60 (0.44-0.81) | 1.00E−03 | 0.60 (0.44-0.81) | 1.00E−03 |
| LN+ | Median | 0.58 (0.35-0.96) | 3.20E−02 | 0.58 (0.35-0.96) | 3.20E−02 |
| | Quartile | 0.49 (0.29-0.83) | 6.90E−03 | 0.49 (0.29-0.83) | 6.90E−03 |
| | Tertile | 0.56 (0.34-0.92) | 2.10E−02 | 0.56 (0.34-0.92) | 2.10E−02 |

p-values are from Log rank test from KM-plotter

TABLE 8
details of antibodies and immunohistochemistry conditions
used for breast cancer TMA analysis in this study

| | | | | | | | Cut-off used for |
|---|---|---|---|---|---|---|---|
| | | | | | Antigen | Cellular | classification as |
| Antibody | Clone | Species | Source | Dilution | Retrieval* | Localization | ‘positive’ |
| ER | 6F11 | Mouse | Novocastra | 1:100 | Citrate | Nucleus | >1% |
| PR | 1A6 | Mouse | Novocastra | 1:200 | Citrate | Nucleus | >1% |
| HER2 | CB11 | Rabbit | Dako | 1:200 | Citrate | Cell | 3+ (>30%) |
| | | | | | | Membrane | |
| CK5/6 | D5/16B4 | Mouse | Chemicon | 1:400 | Citrate | Membrane + | Any positivity |
| | | | | | | Cytoplasm | |
| CK14 | LL002 | Mouse | Novocastra | 1:40 | Citrate | Membrane + | Any positivity |
| | | | | | | Cytoplasm | |
| EGFR | 31G7 | Mouse | Invitrogen | 1:100 | EDTA | Cell | Any positivity |
| | | | | | | Membrane | |
| Ki-67 | MIB-1 | Mouse | Dako | 1:200 | Citrate | Nucleus | Any positivity (20% |
| | | | | | | | cells stained classed |
| | | | | | | | as ‘Ki67-high’) |
| TTK | N1 | Mouse | Abcam | 1:100 | EDTA | Cytoplasm | 0 Negative |
| | | | | | | | 1 weak and focal |
| | | | | | | | staining |
| | | | | | | | 2 moderate-strong |
| | | | | | | | focal staining |
| | | | | | | | (collectively <50% |
| | | | | | | | tumor cells) |
| | | | | | | | 3 moderate-strong |
| | | | | | | | diffuse staining |
| | | | | | | | (>50% tumor cells) |
| | | | | | | | Regarding estimating |
| | | | | | | | % of cells stained, we |
| | | | | | | | disregarded mitotic |
| | | | | | | | cells to assess |
| | | | | | | | mitosis-independent |
| | | | | | | | expression of TTK |

*Antigen retrieval in 0.01M citric acid buffer (pH 6.0) at 125° C. for 5 min in a pressure cooker, or in 0.001M Tris/EDTA; pH 8.8, at 105° C. for 15 min in a pressure cooker.

TABLE 9
Multivariate analyses

| | P | Hazard | | P | Hazard | | P | Hazard |
|---|---|---|---|---|---|---|---|---|
| Covariants | value | Ratio | Covariants | value | Ratio | Covariants | value | Ratio |
| Grade | 0.7045 | 1.04 | Stage | 0 | 1.46 | AJCC | 0.0002 | 1.35 |
| | | (0.86- | | | (1.26- | stage T | | (1.16- |
| | | 1.25) | | | 1.68) | | | 1.58) |
| 10CIN | 0.0002 | 1.59 | 10CIN | 0 | 2.2 | AJCC | 0 | 1.73 |
| 2ER | | (1.25- | 2ER | | (1.73- | stage N | | (1.5- |
| signature | | 2.04) | signature | | 2.79) | | | 1.99) |
| | | | | | | 10CIN | 0.0075 | 1.35 |
| | | | | | | 2ER | | (1.08- |
| | | | | | | signature | | 1.69) |

### Example 2

### Materials and Methods

Meta-analysis of global gene expression in TNBC

We performed a meta-analysis of global gene expression data in the Oncomine™ database [37] (Compendia Bioscience, Ann Arbor, Mich.) using a primary filter for breast cancer (130 datasets), sample filter to use clinical specimens and dataset filters to use mRNA datasets with more 151 patients (22 datasets). Two additional filters were applied to perform two independent differential analyses. The first differential was metastatic event analysis at 5 years (metastatic events vs. no metastatic events, 7 datasets [51, 56-61]) and the second differential analysis was survival at 5 years (patients who died vs. patients who survived. 7 datasets [39, 57, 59, 61-64]). Deregulated genes were selected based on the median p-value of the median gene rank in overexpression or underexpression patterns across the datasets for each of the two differential analyses.

Deriving the 28-Signature (the TN Signature) The online tool KM-Plotter [38] which collates gene expression data from Affymterix platform for more than 40(K) breast cancer patients were used for developing the 28-gene signature. From the deregulated genes in primary tumors which led to metastatic or death events within 5 years discovered in the meta-analysis in Oncomine™, 166 genes were common in both survival events. These genes were then interrogated one by one in KM-Plotter restricting the univariate survival analysis to ER⁻ or BLBC subtypes. Genes which significantly associated with relapse-free survival (RFS). distant metastasis-free survival (DMFS) or overall survival (OS) in either ER⁻ or BLBC subtypes were short selected. The 96 genes that were significant in this filtering where then sorted for their level of significance as well as the prevalence of significance across the different survival outcomes (RFS. DMFS and OS) and across ER⁻ and BLBC subtypes. Based on this sorting, six groups of gene lists were obtained with different levels of survival association (Table 14). Each of these groups were then used as a metagene and the average expression of genes in each group was investigated for association with survival in KM-Plotter in ER and BLBC subtypes. Based on these analysis, four groups were selected and two were excluded. Furthermore, for two groups, the top 4 and 3 genes were found to be more prognostic than the rest of the group and these were selected. In total, the 7 genes (which their downregulation associates with poor survival) from these two groups and 21 genes (which their upregulation associates with poor survival) in the other two groups were selected to test for association with survival in KM-Plotter. These 28 genes showed the highest association with survival as a gene signature compared to any single gene in the original list or any groups from this list. These 28 genes were selected as the triple negative (TN) signature and was subjected to validation as described below.

### Validation of the TN Signature in Breast Cancer Cohorts

Three large breast cancer gene expression datasets were used for validation. The Research Online Cancer Knowledgebase (ROCK) dataset [40] (GSE47561; n=1570 patients) and the homogenous TNBC dataset [32] (GSE31519; n=579 TNBC patients) were obtained from Gene Expression Omnibus (GEO) and the data was imported into BRB-ArrayTools [65] (V4.2, Biometric Research Branch, NCI, Maryland, USA) with built in R Bioconductor packages. The Cancer Genome Atlas (TCGA) dataset [39]; using the Illumina HiSeq RNA-Seq arrays (n=1106 patients) or the Agilent custom arrays (Agilent 04502A-07-3) on 597 patients of the 1106 total patients, were obtained from the UCSC Genome Browser [66, 67]. The TN signature was investigated in each of these datasets where a score was devised to quantify the signature; the TN score=average expression of the 21 genes whose overexpression associated with poor survival÷average expression of the 7 genes whose underexpression associated with poor survival. The TN score for each tumor in each dataset was calculated and tumors were assigned as high or low TN score tumors by dichotomy across the median TN score in each dataset. In some cases, tertiles of the TN score in each dataset were used to classify tumors as high, intermediate or low TN score tumors and in other cases the quartiles of the TN score were used to classify tumors in the 1^{st}, 2^{nd}, 3^{rd} or 4^{th} quartiles. The survival of patients in high (over the median, last tertile of the 4th quartile) vs. low TN score groups was compared. Survival analyses were constructed using GraphPad® Prism v6.0 (GraphPad Software, CA, USA) and the Log-rank (Mantel-Cox) Test was used for statistical comparisons of survival curves.

Association of the TN Score and Signatures with Pathological Complete Responses (pCR) after Neoadjuvant Chemotherapy and Response to Endocrine Therapy

Datasets which performed gene expression profiling prior to neoadjuvant chemotherapy or endocrine therapy alone were obtained from GEO. The datasets used in this study for neoadjuvant chemotherapy and recorded pathological complete response (pCR) include: GSE18728 [42], GSE50948 [43], GSE20271 [44], GSE20194 [45]. GSE22226 [41, 46], GSE42822 [47] and GSE23988 [48]. For datasets which performed gene expression profiling prior to endocrine therapy (tamoxifen) and recorded patient survival include: GSE6532 [25] and GSE17705 [51]. These datasets using the Affymetrix gene expression array platforms were imported into BRB-ArrayTools and normalized as described previously [68]. Each tumor in the datasets were assigned as high or low score for our signatures as described in the previous sections. The rate of pCR after chemotherapy or the survival of patients after endocrine therapy were compared between high score tumors and low score tumors using GraphPad® Prism.

### Global Gene Expression Profiles Comparison by Class Comparison

Global gene expression comparison was carried out to compare tumors with high TN or iBCR scores to those with low TN or iBCR scores to characterize additional differences between these tumors and identify deregulated genes which could be suitable as for drug targeting. These comparisons were carried out in the large cohort of 1570 patients in the ROCK dataset and BRB-ArrayTools was used to perform the Class Comparison test. The two classes were high vs. low score tumors and the parameters selected in this plugin in ArrayTools were as follows: Type of univariate test used=Two-sample T-test; Class variable=TN score (high or low) or iBCR score (high or low); fold-change cutoff=1.5 fold; Permutation p-values for significant genes were computed based on 10000 random permutations and Nominal significance level of each univariate test: 0.05. The results from these analyses are shown in Tables 13 and 15-17.

Integration of the Agro and TN Signatures in the integrated Breast Cancer Recurrence (iBCR) Score

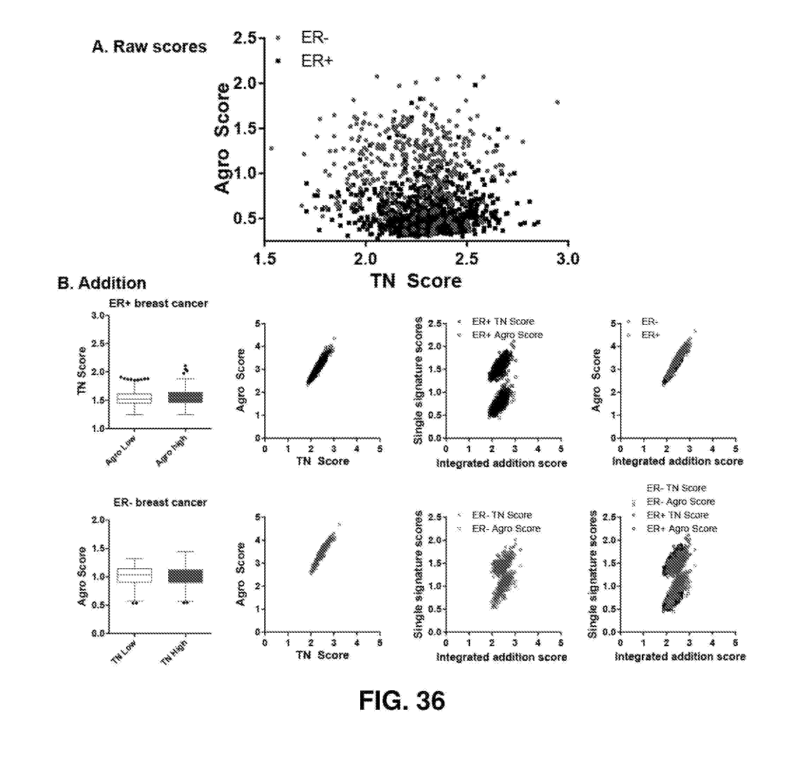

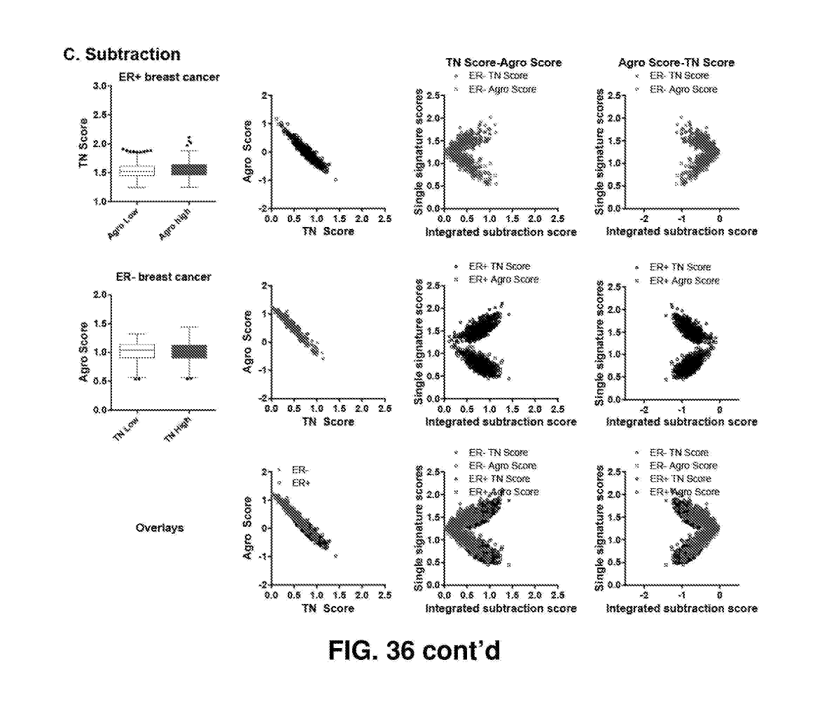

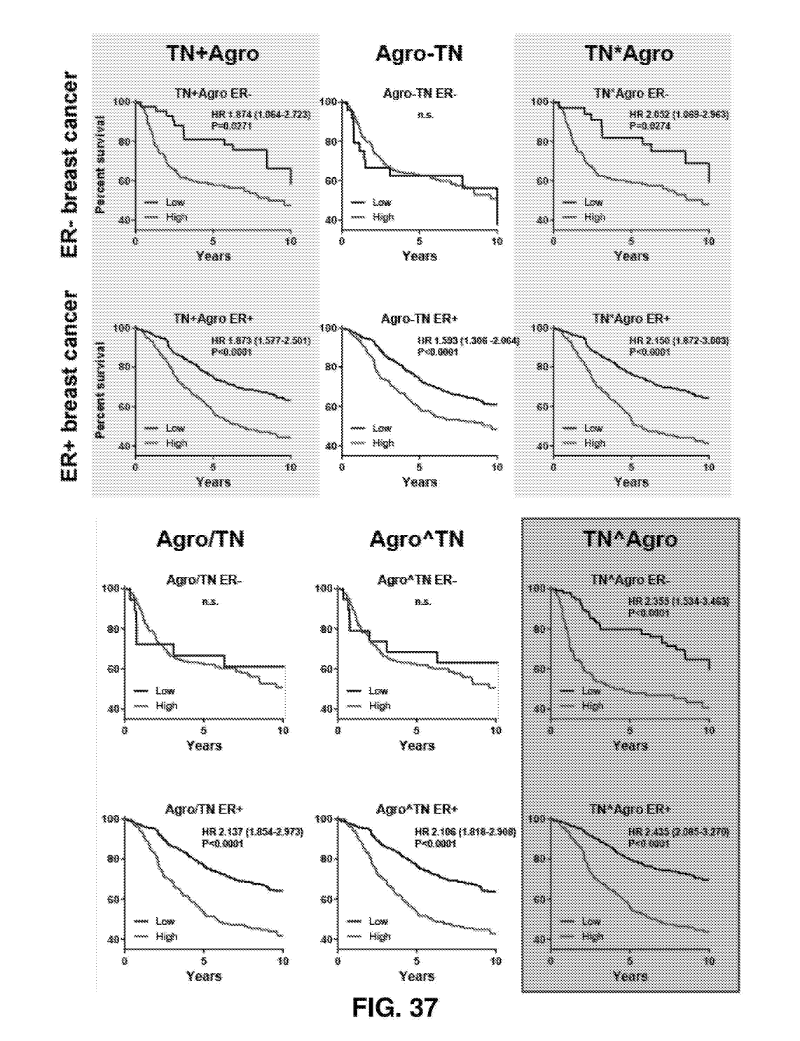

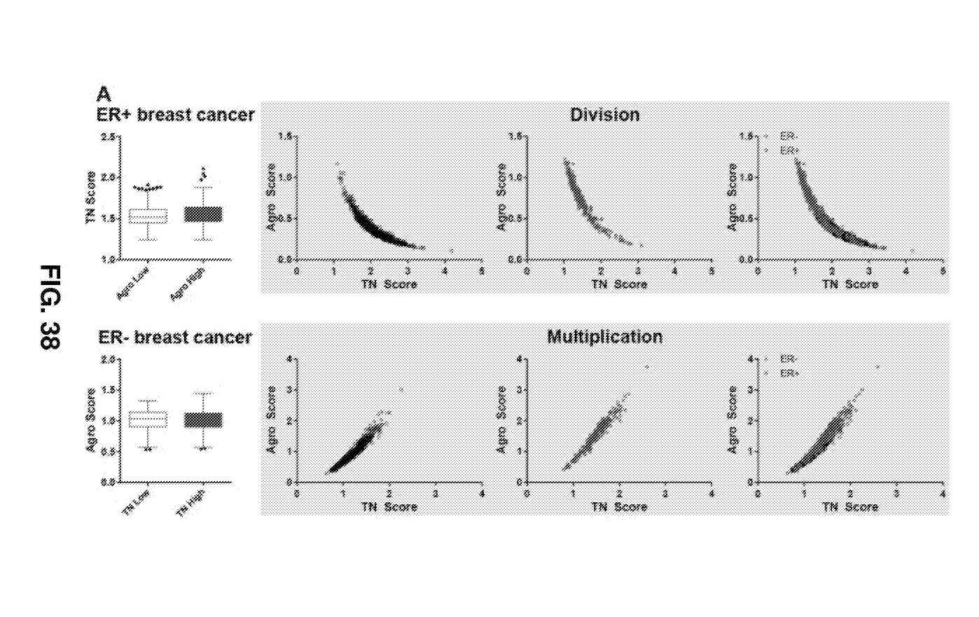

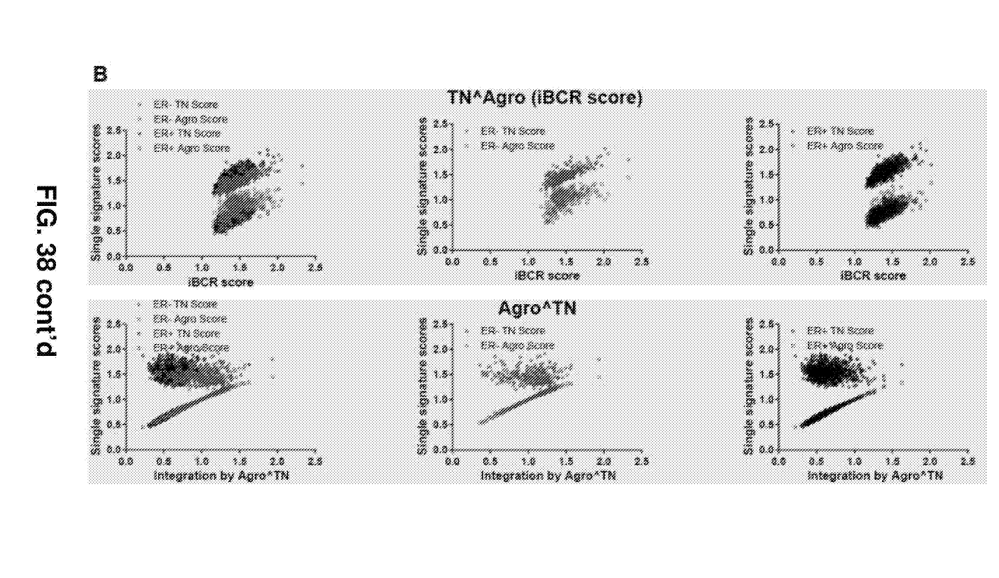

We previously published the Aggressiveness (Agro) signature and score also from meta-analysis and extensive validation and show that this signature is prognostic in ER+ breast cancer [36]. To test whether the Agro signatures could be integrated with the TN signature (prognostic in ER breast cancer) to produce an integrated test that is independent of ER status, several integration methods were investigated. The hypothesis behind the integration methods was to identify a direct relationship that can describe the relationship between the TN and Agro scores in both ER⁻ and ER⁺ breast cancer subtypes that is also in direct relationship with the integrated score. In other words, the integrated score would retain the information from each the Agro and TN scores relevant to their prognostic value in ER⁺ and ER⁻ breast cancers, respectively. The ROCK dataset was used to test the different methods of integration and the performance of these methods in the stratification of survival of ER⁺ and ER⁻ breast cancer. The addition or subtraction of the scores produced a direct relationship between the TN and Agro score and the produced integrated score (FIG. 36). These two methods were then analyzed for prognostication of ER⁺ and ER⁻ subtypes in the ROCK dataset and only the addition method retained prognostication in ER⁻ breast cancer (FIG. 37). Similarly, multiplying and dividing the TN and Agro scores were lit tested and an exponential and power curve relationships described the relation between the two scores and with the integrated score (FIG. 38). Again, these two methods were tested from prognostication in the ROCK dataset and only the multiplication method retained prognostication in ER⁻ breast cancer (FIG. 37), Because the multiplication and division methods produced exponential and power curves for the relationship between the scores, integration by raising one score to the power of the other score appeared reasonable. Exponential and power curves are the result of power equations. Indeed, integration by raising the TN score to the power of the Agro score was highly prognostic in both ER⁺ and ER⁻ breast cancers (FIGS. 37 and 38). This integrated score, the integrated Breast Cancer Recurrence (iBCR) score was in fact more prognostic in ER⁺ and ER⁻ patients in the ROCK dataset than the single Agro and TN scores, respectively. The iBCR score was validated in the ROCK and homogenous TNBC datasets (Affymetrix platform), the TCGA dataset (Illumina RNA-Seq platform) and the ISPY-1 trial dataset (GSE22226 [41, 46], Agilent platform), illustrating the platform-independence of the iBCR score which is driven by the platform independence of the Agro and TN signatures as they were discovered from meta-analysis irrespective of array platforms used from independent studies.

### Mining Drug Screen Studies

Two large studies which treated large panels of cancer cell lines with large panels of anticancer drugs were investigated to determine whether cell lines with high Agro, TN or iBCR scores show different sensitivity to particular anticancer drugs in comparison to cancer cell lines with low Agro, TN or iBCR scores. Briefly, the datasets of gene expression profiling from Genentech (mRNA Cancer Cell Line Profiles GSE10843), Pfizer (Pfizer Molecular Profile Data for Cell Line GSE34211) and Broad Institute/Novartis (Cancer Cell Line Encyclopedia [CCLE] GSE3613) were obtained from GEO and imported into ArrayTools as described earlier. The Agro, TN and iBCR scores for all the cell lines profiled were calculated and cell lines were assigned as high or low for each of the scores based on dichotomy across the median in each dataset. For cell lines which were profiled in more than one dataset, the average scores were used. Using this data, the sensitivity of cancer cell lines with high and low Agro, TN or iBCR scores was compared to those with low scores to anticancer drugs was investigated in two studies [49, 50]. Drugs which had significantly different IC50 in high score cell lines compared to low score cell lines are described herein. Statistical significance was determined from unpaired two-tailed t-test using GraphPad® Prism.

### Other Statistical Analysis

Univariate and multivariate Cox proportional hazards regression analyses were performed using MedCalc for Windows, version 12.7 (MedCalc Software, Ostend, Belgium).

### Results

### Meta-Analysis of Gene Expression Profile in Oncomine™

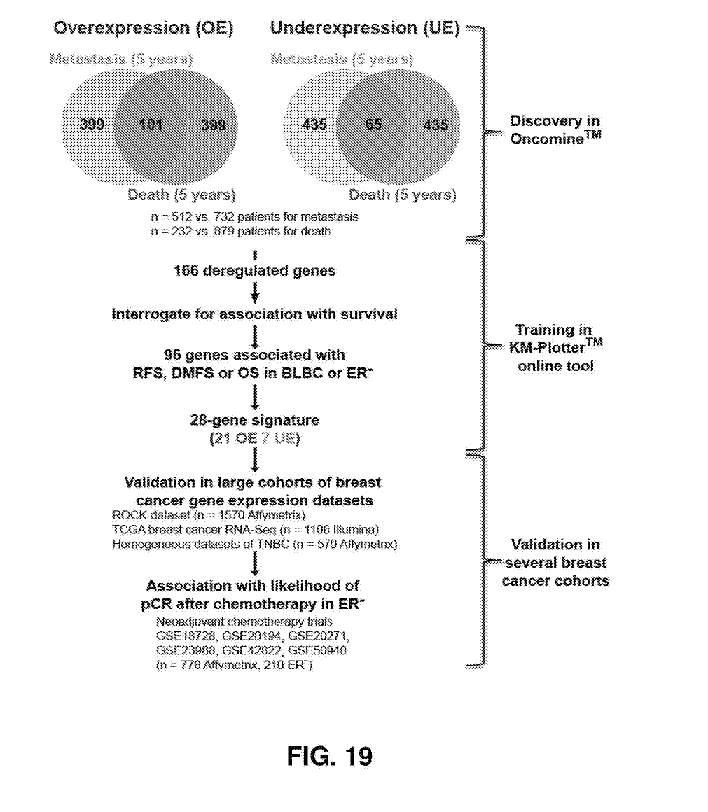

We performed a meta-analysis of published gene expression data, irrespective of platform or breast cancer subtype, using the Oncomine™ database [37] (version 4.5). We were able to compared the expression profiles of primary breast tumors from 512 patients who developed metastases vs. 732 patients who did not develop metastases at 5 years (7 datasets in total) to identify 500 overexpressed genes and 500 underexpressed genes in the metastasis cases (cutoff median p-value across the datasets <0.05 from a Student's t-test, FIG. 31). We also compared the expression profiles of 232 primary breast tumors from patients who died within 5 years vs. 879 patients who survived in 7 datasets and found 500 overexpressed genes and 500 underexpressed genes in the poor survivors (cutoff median p-value across the datasets <0.05 from a Student's t-test, FIG. 31). Since several datasets were annotated for one of these outcomes but not both, we rationalized that the union of these analyses is more appropriate particularly that death is the most likely outcome in metastatic disease. The union of the over- and expressed genes in tumors that associated with metastasis and those that associated with death within 5 years revealed common 101 overexpressed and 65 underexpressed genes (FIG. 19). These 166 deregulated genes were then subjected to training using the online tool KM-plotter 1381 to derive a 28 gene signature as described in methods below followed by validation of this signature, the TN signature, in several large cohorts of breast cancer gene expression datasets (FIG. 19).

### The TN Signature is Prognostic in TNBC, BLBC and ER⁻ Breast Cancer Subtypes

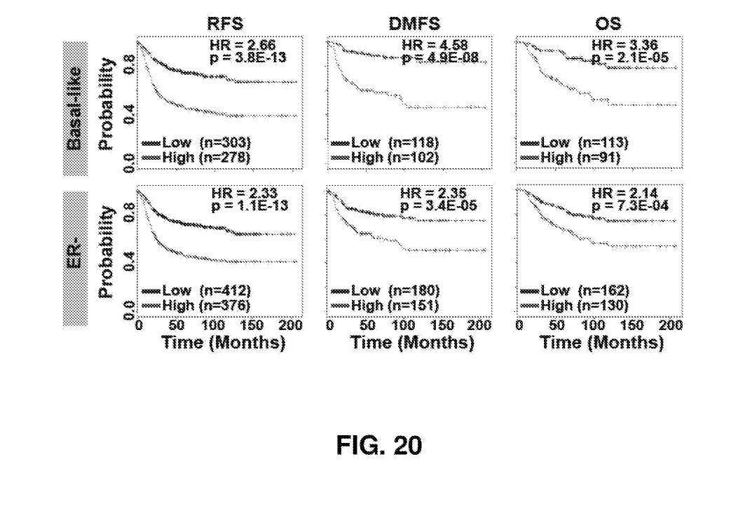

The 166 deregulated genes in primary breast tumors that associated with poor outcome discovered from the Oncomine™ meta-analysis were interrogated using KM-Plotter. The overexpression of 31 genes and the underexpression of 65 genes associated with RFS, DMFS or OS of BLBC or ER− breast cancer (Table 14). Based on the level of significance in univariate survival analysis and the prevalence of this significance across the different disease outcomes (RFS, DMFS and OS), a list of 21 overexpressed and 7 underexpressed genes (Table 1) were shortlisted as a signature with the strongest association with survival in both BLBC and ER breast cancer subtypes (FIG. 20).

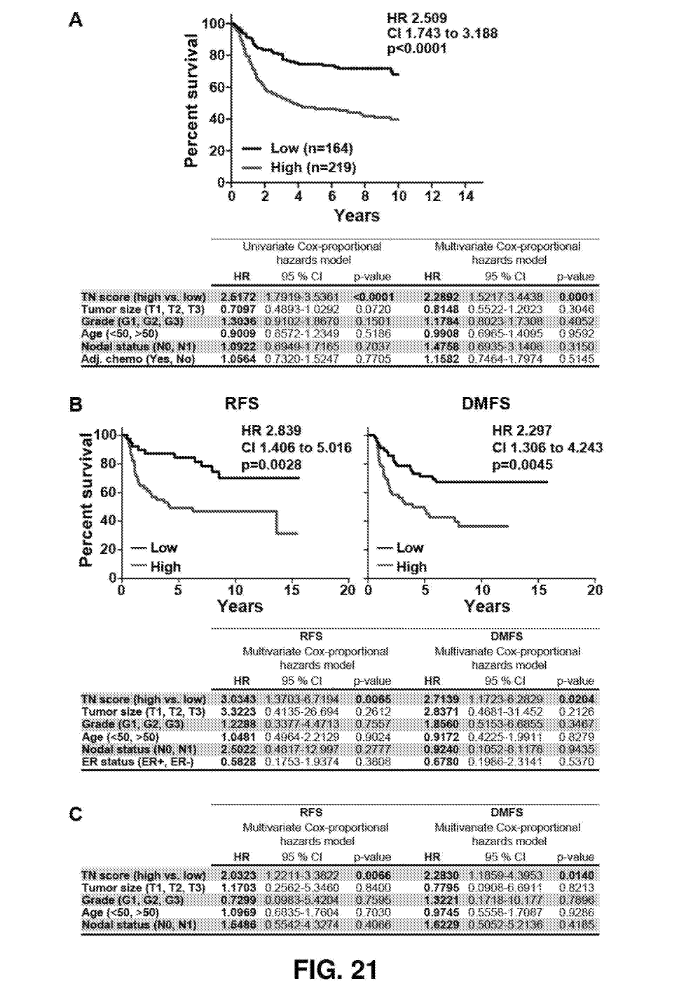

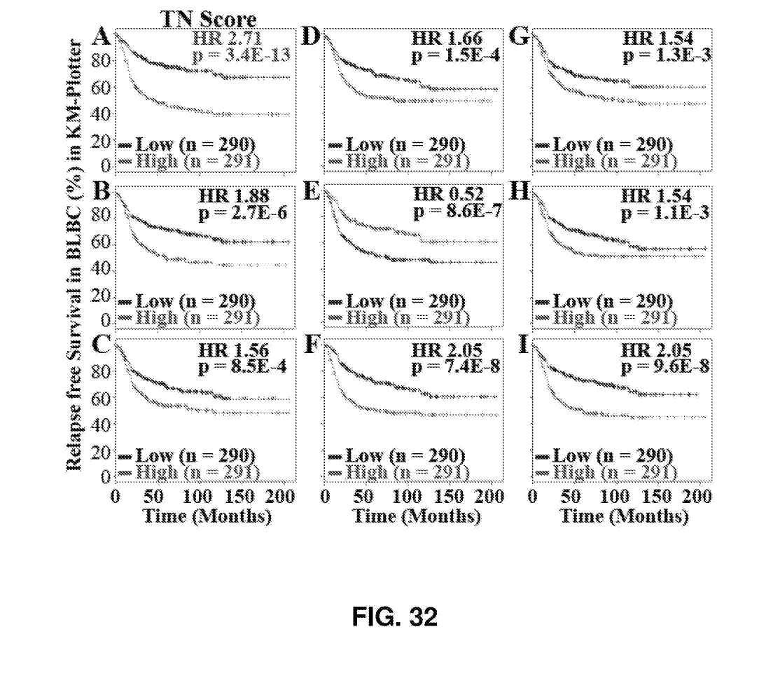

The 28-gene signature, the TN signature, was then validated in multivariate survival analysis in two breast cancer cohorts, the homogenous TNBC dataset [32] and the Research Online Cancer Knowledgebase (ROCK) dataset [40]. We devised a score to quantify trends in the TN signature, the TN score, which is calculated as the ratio of the average expression of the 21 overexpressed genes to that of the 7 underexpressed genes. Dichotomy across the median TN score stratified the survival of TNBC (FIG. 21A). BLBC (FIG. 21B) and ER− (FIG. 21C) patients and outperformed all standard clinicopathological indicators. These analyses indicated that the TN score is an independent prognostic factor that identified TNBC, BLBC or ER⁻ patients with poor survival irrespective to tumor size and grade, patient age, lymph node status or treatment. The TN signature also outperformed all previously published signatures that are prognostic in ER, TNBC or BLBC subtypes [30-35] (FIG. 32).

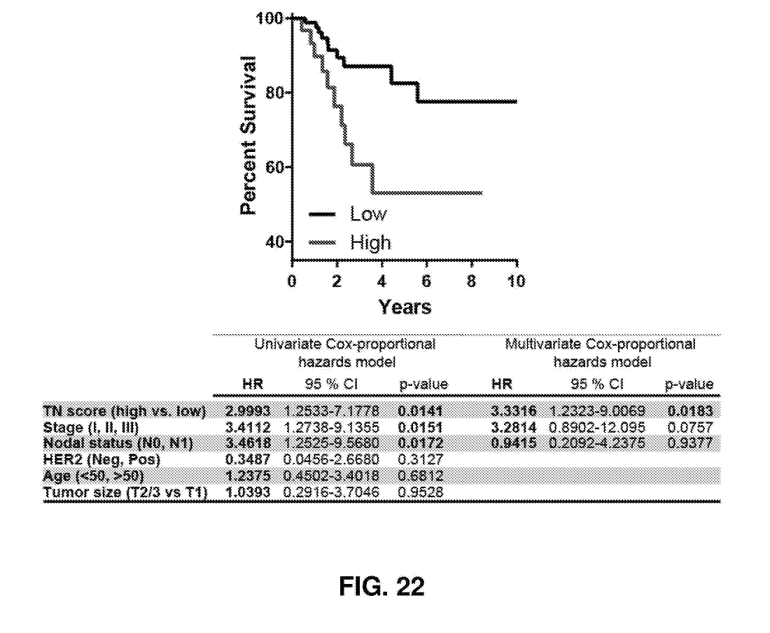

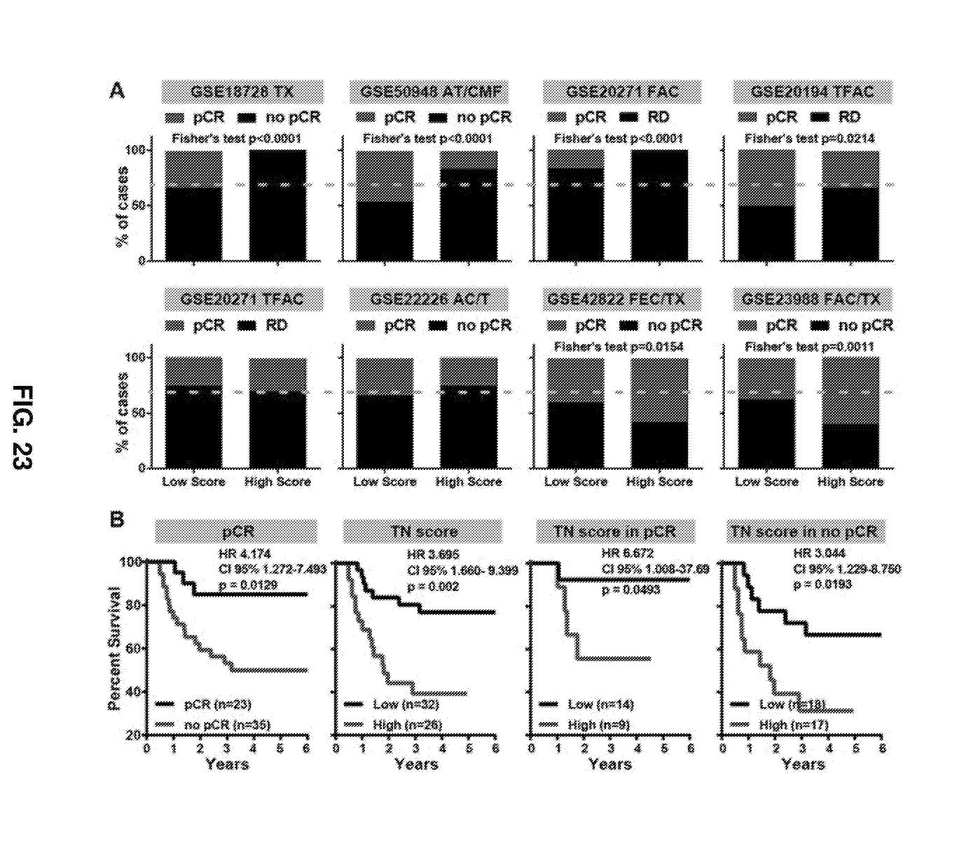

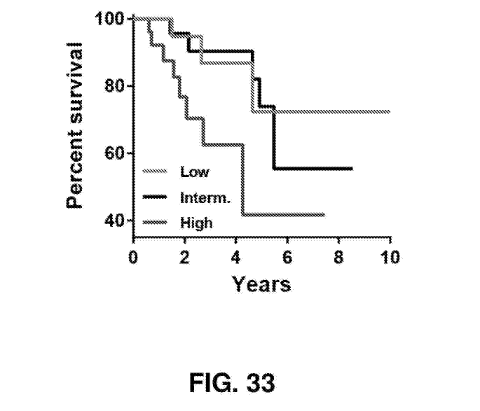

While the discovery of the signature in Oncomine™ included datasets using the Affymterix, Illumina and Agilent platforms, the training and validation above was limited to the Affymterix platform. Thus, we validated the TN score in The Cancer Genome Atlas (TCGA) dataset [39] which used the lumina HiSeq RNA-seq platform. As shown in FIG. 22, the RFS of ER⁻ patients in the TCGA dataset was stratified by TN score and this stratification outperformed that by standard clinicopathological indicators. The original TCGA publication used Agilent custom arrays (Agilent 04502A-07-3) on 597 patients and we analyzed the prognosis of the TN score in this data. The TN score stratified the survival of ER patients in the Agilent TCGA data (FIG. 33). Altogether, the prognostic value of the TN signature/score was validated in large, independent cohorts of breast cancer in TNBC, BLBC and ER⁻ breast cancer subtypes irrespective of the gene expression array platforms used.

The TN Score and the Likelihood of pCR after Chemotherapy

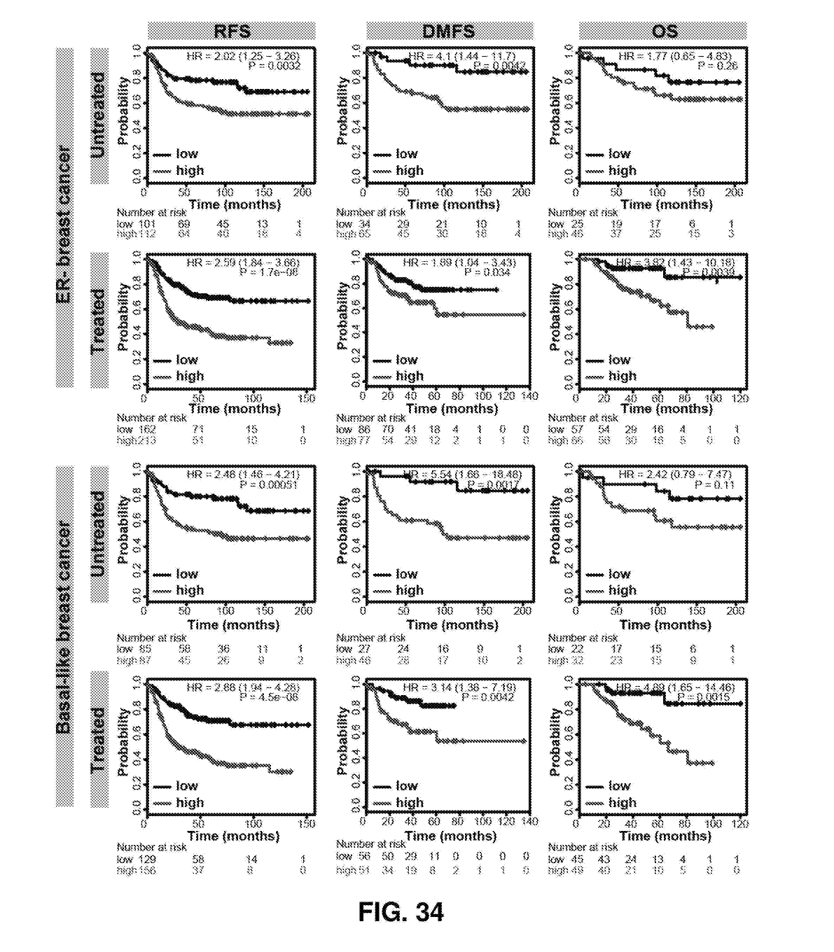

Chemotherapy is a standard therapy for ER⁻ breast cancer and the only mode of therapy for ER⁻HER2⁻ (TNBC) breast cancer. Although, pathological complete response (pCR) differs by receptor status, it remains highly predictive of survival within the different breast cancer subtypes [41]. Given the association of the TN score with outcome in TNBC, BLBC and ER⁻ breast cancer, we questioned whether this score is also associated with pCR after chemotherapy. To this end. we analyzed publically available datasets of neoadjuvant chemotherapy trials which recorded pCR and performed pre-treatment gene expression profiling. As shown in FIG. 23A, pCR after chemotherapy in ER⁻/HER2⁻ patients was less likely after TX (GSE18728), AT/CMF (GSE50948) or FAC (GSE20271) chemotherapy regimens when these patients had a high TN score. TFAC chemotherapy regimen was less likely to produce pCR in high TN score tumors in one study (GSE20194) but without a significant association in a second study (GSE20271), ER⁻HER2⁻ tumors with high TN score had a trend to lower response to AC/T chemotherapy (GSE22226 AC/T). In contrast, pCR was achieved in 57% and 60% of ER⁻HER⁺ tumors with high TN score after treatment with the FEC/TX (GSE42822) and FAC/TX (GSE23988) regimens, respectively. Altogether, the rate of pCR stratified by the TN score was significantly different in either the low or high TN score tumor from the reported general 31% pCR rate in TNBC [9] (dotted line in FIG. 23A). In one dataset, the ISPY-1 trial (GSE22226). the relapse-free survival (RFS) was also recorded. As shown in FIG. 23B, pCR was a strong predictor of RFS in ER⁻HER2⁻ breast cancer as previously published [41]. The TN score was not only a strong predictor of RFS after chemotherapy but also could stratify the survival of patients who achieved pCR further in addition to the stratification of patients who did not achieve pCR to good and poor prognosis groups (FIG. 23B). This data indicates that the TN score is independent and has additional value to monitoring pCR after neoadjuvant chemotherapy in ER⁻HER2⁻ (TNBC) breast cancer patients. To further illustrate the utility of the TN score, we analyzed ER⁻ and BLBC patient outcome in KM-plotter for systemically untreated and treated patients separately. As summarized in Table 11 (FIG. 34 for survival curves), the TN signature was prognostic in either systemically untreated or treated ER− and BLBC subtypes.

### Therapeutic Targets Based on the TN Signature

The overexpressed genes in the TN signature contains novel genes which have limited literature describing their function, particularly in cancer. These genes includes GRHPR, NDUFC1, CAMSAP1, CETN3, EIF3K, STAU1, EXOSC7 and KCNG1. These genes are novel candidates for future studies to investigate the effect of their knockdown on the survival of ER⁻ or TNBC breast cancer cell lines. In addition, we took two approaches to identify possible therapeutic strategies envisioned by the TN signature to benefit the poor survival of patients identified by this signature. First, we compared the global gene expression profile of TNBC/BLBC tumors with high TN score to those with low TN score. Secondly, we analyzed published pre-clinical studies which treated cancer cell lines with panels of molecularly targeted drugs to determine whether cell lines with high TN score display sensitive to particular drugs. In the first approach, a class comparison between the global gene expression profiles of BLBC or ER− tumors with high TN score to those with low TN score was carried out in the ROCK dataset. In comparison to low TN score BLBC tumors, high TN score BLBC tumors overexpressed 171 probes and underexpressed 251 probes (Table 15). In a similar analysis, high TN score ER⁻ tumors overexpressed 307 probes and underexpressed 332 probes (Table 16). Of the overexpressed probes, 87 probes (82 genes) were commonly overexpressed in high TN score BLBC and ER⁻ breast cancer compared to low TN score counterparts. Of the 87 probes, 39 probes were prognostic in BLBC and ER− breast cancer (marked in bold in Table 15). More importantly, the 87 probes include genes which encode several kinases, enzymes and ion channels which could be targets or current for future drug development for the treatment of the high TN score tumors that have poor outcome.

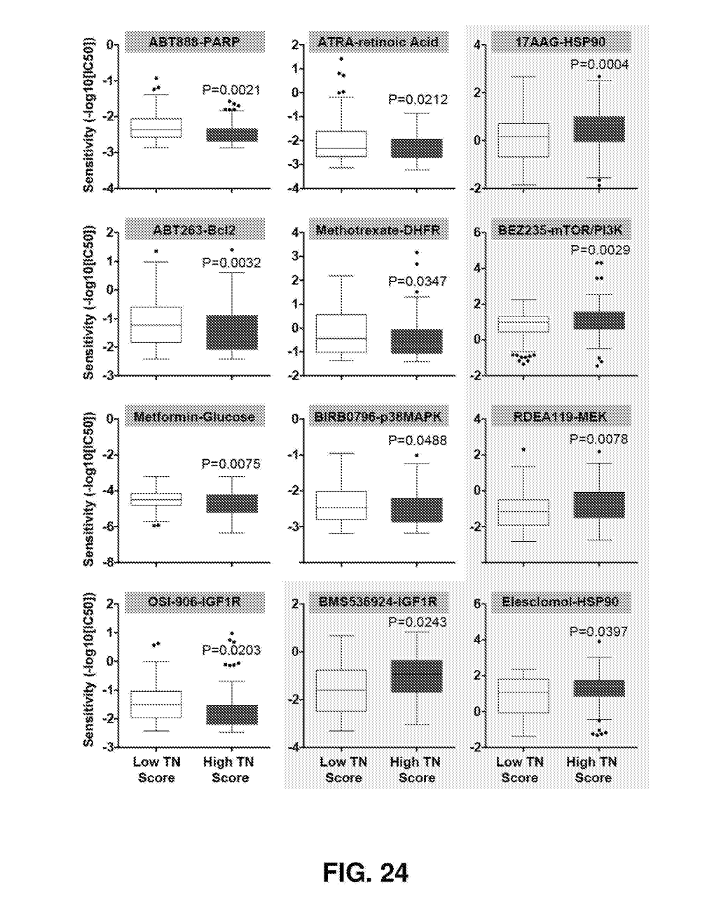

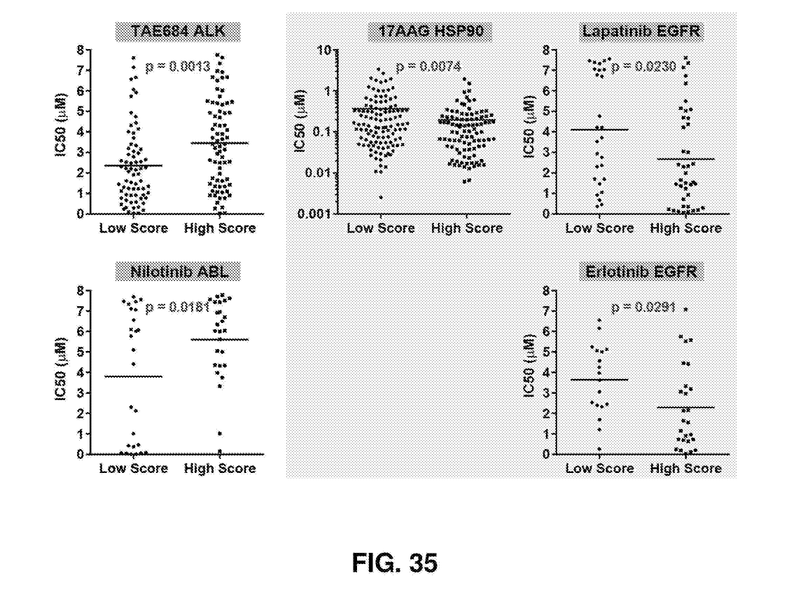

In the second approach, published studies which surveyed panels of molecular drugs against, cancer cell lines were analyzed. The Cancer Cell Line Encyclopedia (CCLE) study [50] investigated the pharmacological profiles for 24 anticancer drugs across 479 cancer cell lines which were also profiled with gene expression arrays. We calculated the TN score for each cell line in this study and compared the sensitivity of these cell lines to the anticancer drugs according to the TN score. Cancer cell lines with high TN score were less sensitive to inhibition of ALK (TAE684) and BCR-ABL (Nilotinib) but more sensitive to the inhibition of HSP90 (Tanespimycin [17-AAG]) and EGFR (Erlotinib or Lapatinib) (FIG. 35). In a similar method, we also analyzed a second large study. Garnett et al. [49], which tested 130 drugs against more than 600 cancer cell lines. As shown in FIG. 24, cell lines with high TN score were less sensitive to inhibition of PARP (ABT-888). retinoic acid (ATRA), Bcl2 (ABT-263), DHFR (methotrexate), glucose (metformin) and p38MAPK (BIRB 0796). Two IGF1R inhibitors showed different results; high TN score cell lines were less sensitive to the OSI-906 inhibitor but more sensitive to the BMS-536924 inhibitor. As shown in FIG. 24, cell lines with high TN score were also sensitive to HSP90 inhibition (17-AAG and Elesclomol) in agreement with the findings from the CCLE study (FIG. 35), High TN score cell lines were also more sensitive to mTOR/PI3K (BEZ235) and MEK (RDEA-119) inhibition.

### Integration of the TN Score and the Aggressiveness Score

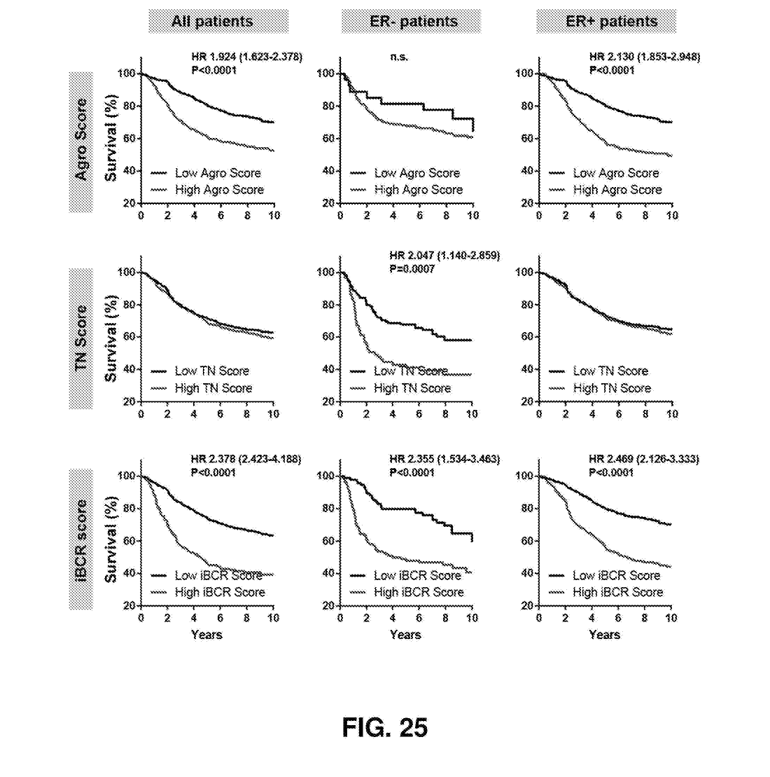

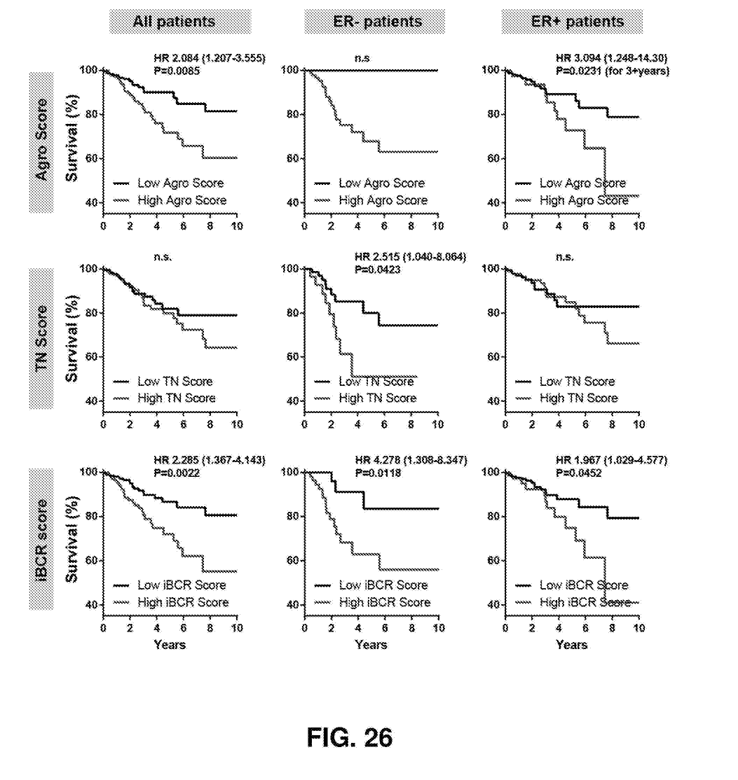

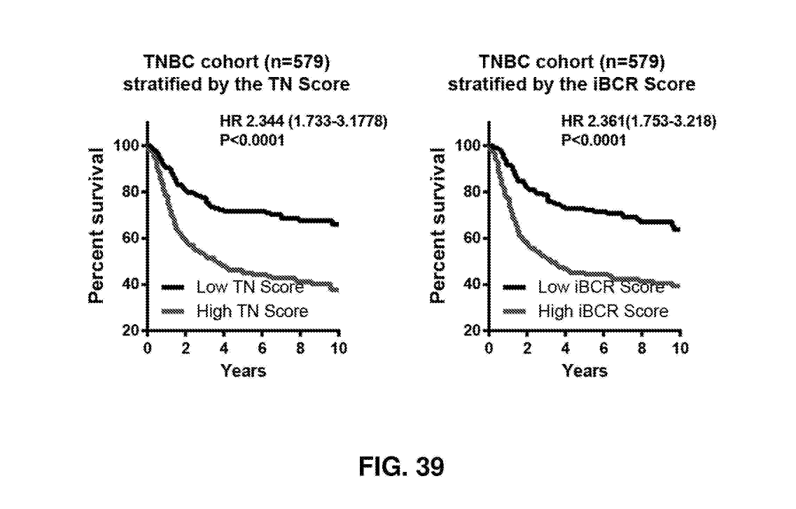

We have recently published the aggressiveness gene signature/score (Agro score) [36] from a meta-analysis in Oncomine™ and validated that this score is prognostic in ER⁺ breast cancer at the gene level. ER⁻ breast cancer, BLBC and TNBC almost consistently express high level of the Agro score thus this signature was not prognostic in these subtypes. We further showed that one of these genes, TTK/MPS1, is upregulated in TNBC cell lines and some ER− negative cell lines, and that TTK is a therapeutic target in these cell lines. Moreover, we showed that the TTK protein level by immunohistochemistry (IHC) is prognostic in very aggressive subgroups of breast cancer including high grade, proliferative tumors, lymph node positive, TNBC and HER2⁺ subtypes [36]. The integration of the TN gene signature (prognostic in ER⁻/BLBC/TNBC) and the Agro gene signature (prognostic in ER⁺) would allow one integrated signature and score which will be prognostic in breast cancer irrespective of subtypes. As detailed in the methods section, the addition, subtraction, multiplication or division of the TN and Agro scores were investigated in the ROCK dataset to identify a direct relationship that would retain the information provided from each of the scores. A linear relationship was observed by the addition or subtraction of the TN and Agro scores (FIG. 36), but only the integration by addition was prognostic in ER− patients (FIG. 37). On the other hand, the multiplication and division of the TN and Agro score produced exponential and power curves relationships, respectively (FIG. 38). Only the multiplication of the scores was prognostic in ER− breast cancer (FIG. 37). Since multiplication and division produced exponential and power curves for the relationship between the TN and Agro score, we also tested integration by one score raised to the power of the second score. Indeed, the TN score raised to the power of Agro score was highly prognostic in ER− and ER+ patients in the ROCK dataset (FIG. 37). This method to integrate the TN and Agro scores, the integrated breast cancer recurrence (iBCR) score, was prognostic in all patients, ER and ER+ patients in the ROCK dataset (FIG. 25) and the TCGA dataset (FIG. 26). Moreover, the iBCR score was as prognostic as the TN score in the homogenous TNBC dataset [32] (FIG. 39), supporting the iBCR score as prognostic test in breast cancer.

The iBCR Score and the Likelihood of pCR after Chemotherapy

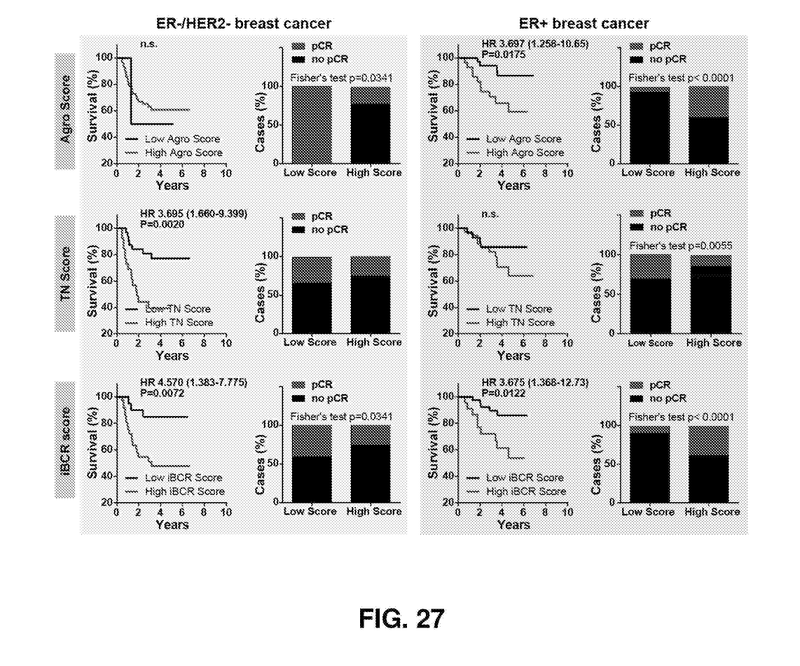

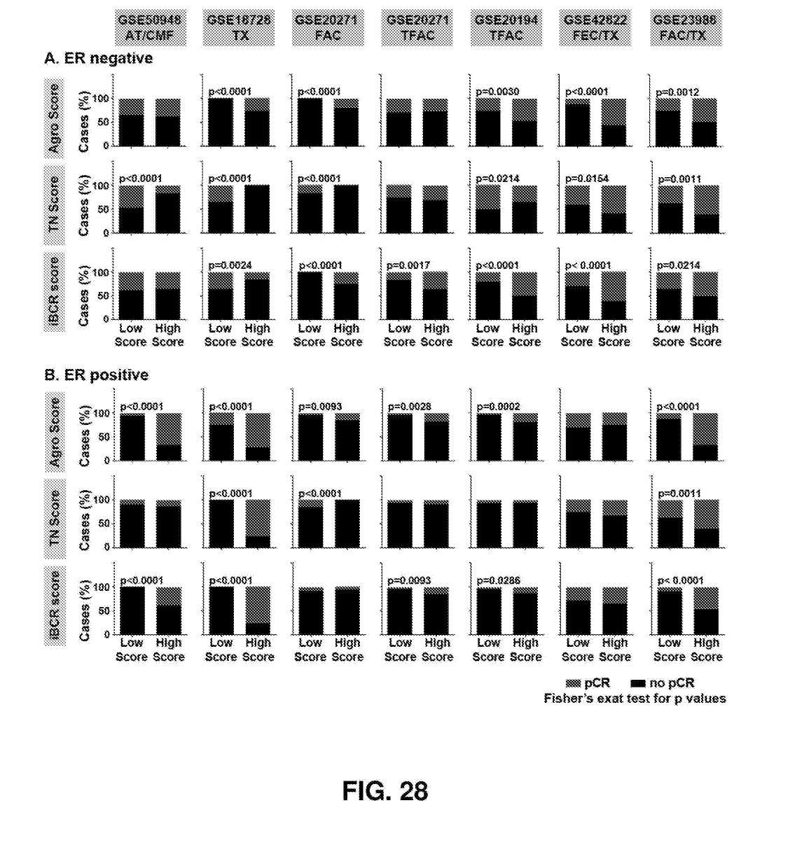

The association of the iBCR score with patient survival and the likelihood of pCR after chemotherapy was investigated in the ISPY-1 trial (GSE22226). The RFS of ER⁻/MER2⁻ patients was stratified by iBCR score better than the TN score alone (FIG. 27). High iBCR score ER⁻/HER2⁻ patients were less likely to achieve pCR (FIG. 27), which could explain the poorer survival of these patients. In ER⁺ breast cancer, the iBCR score stratified the RFS patients similarly to the Agro score. Although higher likelihood pCR was observed in high iBCR score ER+ tumors (FIG. 27), this subgroup had poor RFS. This can be explained by the small number of ER⁺ patients who achieved pCR (10/62 [16%] vs. 10/34 [29%] in ER⁻HER2⁻). These results provide further validation and evidence for the value of the iBCR score as a single test which incorporates the Agro score (prognostic in ER⁺) and the TN score (prognostic in ER⁻) The results in FIG. 25 from the ROCK dataset (Affymetrix platform), FIG. 26 from the TCGA dataset (Illumina platform) and FIG. 27 from the ISPY-1 trial (Agilent platform) also provide evidence for the robustness of the Agro and TN scores and the derived iBCR score across independent studies across the three major gene expression array platforms. Next, the association of the iBCR score with pCR was investigated in other neoadjuvant chemotherapy datasets in both ER-HER2⁻ and ER⁺ patients. pCR was less likely in high iBCR ER/HER patients after TX (GSE18728) chemotherapy regimen and not different to low iBCR ER−/HER2− patients when treated with AT/CMF (GSE50948). In the other datasets, pCR was more likely in high iBCR score ER−/HER2− patients after treatment with FAC (GSE20271), TFAC (GSE20271 and GSE20194), FEC/TX (GSE42822) and FAC/TX (GSE23988) neoadjuvant chemotherapy regimens (FIG. 28A).

As shown in the summary from these four studies in Table 12, of the total 183 ER HER2⁻ patients, 120 patients (65.6%) had high iBCR score and of these 54 patients (29.5%) achieved pCR while 66 patients (36.1%) did not achieve pCR. The larger number of patients with high iBCR score that did not achieving pCR (66/120, 55%) and that recurrence may be observed on high iBCR score patients after pCR (55/120, 45%) could explain the poorer survival of high iBCR score ER⁻HER2⁻ patients (40-50% survival at 10 years in FIG. 25 and FIG. 26). Based on these studies and that chemotherapy is the mainstay in the treatment of ER⁻/HER2⁻ breast cancer, low iBCR score patients may be spared from additional treatments particularly if they achieve pCR after chemotherapy. On the other hand, high iBCR ER-HER2− patients and particularly those who do not achieve pCR should be offered additional therapy which could be based on the upregulated genes in the Agro or TN signatures or based on other overexpressed genes in these tumors (Tables 15 and 16) or from the pre-clinical analysis we performed from drug sensitivity studies (FIGS. 24 and 35). High iBCR score in ER⁺ was associated with higher likelihood of pCR after AT/CMF (GSE50948), TX (GSE18728), TFAC (GSE20271 and GSE20194) and FAC/TX (GSE23988) neoadjuvant chemotherapy regimens (FIG. 38B). Despite this higher pCR likelihood, high iBCR ER+ patients have poorer survival (FIGS. 25 and 26) which could be explained by the small number of ER+ patients who achieve pCR (of the 207 ER⁺ patients in the above five studies, 5 [2.5%] with low iBCR and 20 [9.7%] with high iBCR score achieved pCR). Thus. for ER+ breast cancer where a decision about including chemotherapy with the standard endocrine therapy in the treatment planning may be informed by the iBCR score. The value of the iBCR score in the treatment planning of ER+ patients is the described next section.

The iBCR Score and the Treatment of ER⁺ Breast Cancer

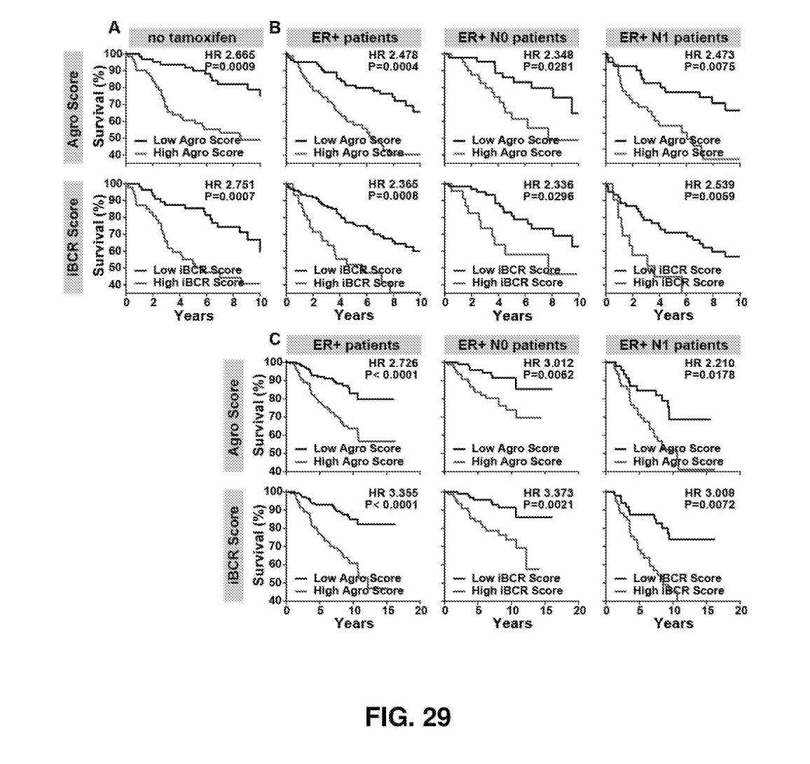

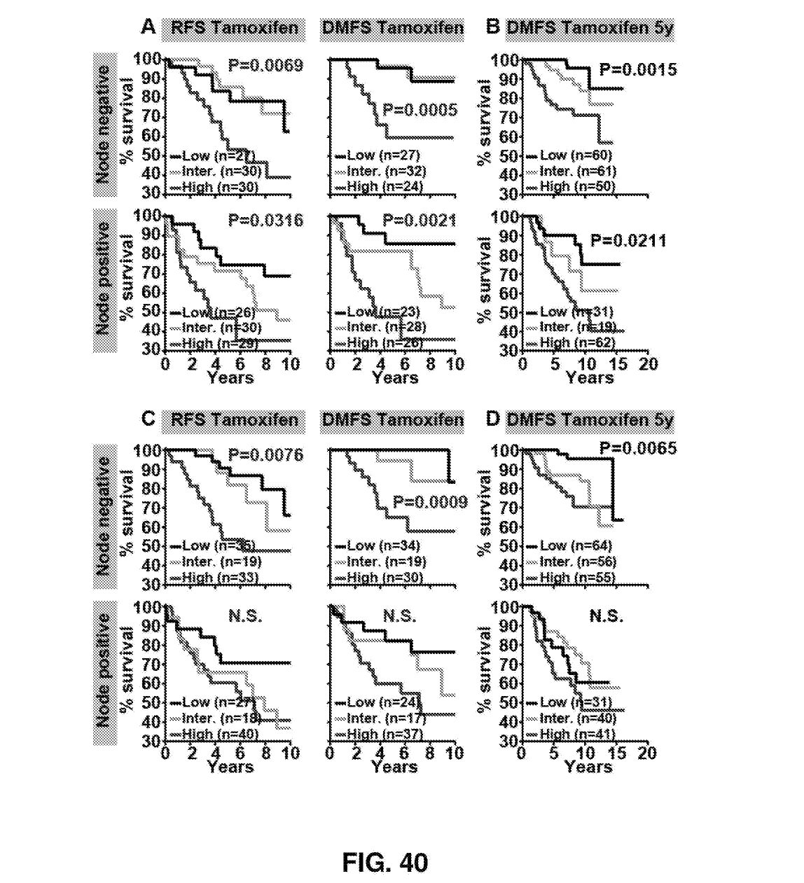

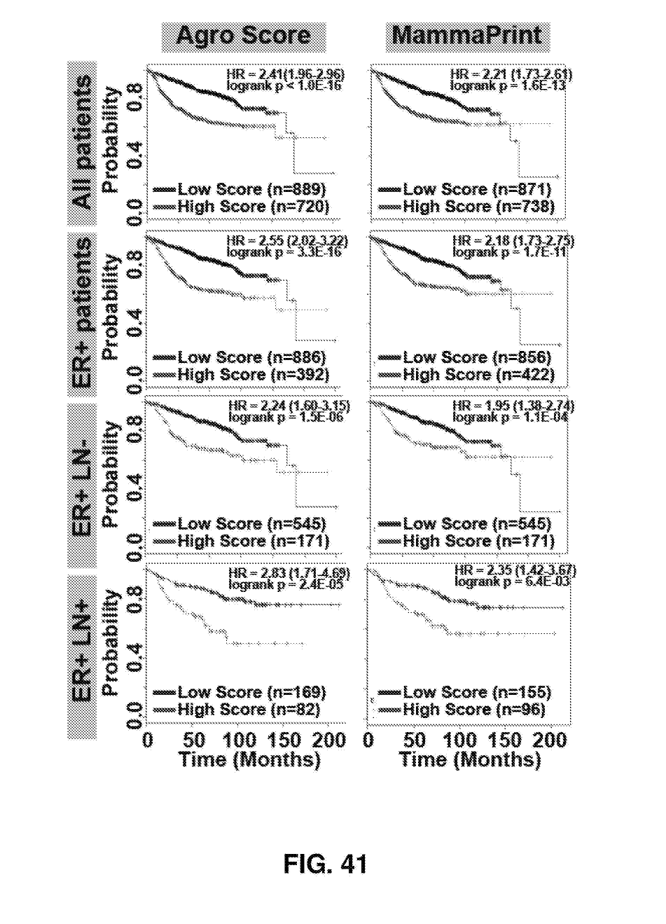

ER⁺ breast cancer patients are treated with endocrine therapy, particularly tamoxifen. When these patients are lymph node positive (N0), adjuvant chemotherapy is also included. For lymph node negative (N0) ER⁺ patients, decision to include chemotherapy is less certain as good prognosis patients (small and lower grade tumors) would be over-treated if chemotherapy is included whereas poorer prognosis patients (larger and higher grade tumors) would be under-treated if chemotherapy is not included. This clinical decision has been the motivation for the development of Oncotype Dx® recurrence score, the MammaPrint and more recently the PAM50 risk of recurrence score. We have previously published that the Agro score outperformed the Oncotype Dx and the MammaPrint tests in multivariate survival analysis in the METABRIC dataset of 2000 patients [36] This finding is further supported by direct comparison of the Agro score to Oncotype Dx (FIG. 40) and MammaPrint (FIG. 41) in all ER⁺ patients and in the N0 and N1 subsets. For the iBCR score, as shown in FIG. 29A, this score was prognostic in ER⁺ N0 patients who were not treated with tamoxifen indicating that high iBCR ER⁺ N0 patients should be treated with tamoxifen. When ER+ N0 or N1 patients are treated with tamoxifen, the iBCR score can still identify patients who have poor RFS (FIG. 29B) and DMFS (FIG. 29C). Thus, ER+ N0 or N1 patients with high iBCR score may benefit from the inclusion of adjuvant chemotherapy in their treatment as these patients may experience better pCR (FIG. 2813). Nonetheless, as pCR. rate in ER⁺ is not high, high iBCR score ER+ patients, particularly N1, should be offered additional targeted therapies. The type of targeted therapies for these patients is suggested in the next section.

The iBCR Score Predicts Therapies for ER⁻/HER2⁻ and ER⁺ and Breast Cancer Subtypes

The overexpressed genes in the Agro and TN signature contain targetable genes which could be useful for therapeutic intervention against the high iBCR tumors which have poor survival after the standard treatments. Similar to the analysis performed for the TN signature above, we took two approached to identify additional possible targets in the high iBCR score breast tumors. In the first approach, a class comparison between the global gene expression profiles of ER⁺ or ER⁻ tumors with high iBCR score to those with low iBCR score was carried out in the ROCK dataset. The produced gene-list (1178 probes, data not shown) was then filtered by comparison to normal breast tissue which was also profiled in this dataset. In comparison to low iBCR score tumors and normal breast tissue, high iBCR score tumors overexpressed 204 probes (181 genes) and underexpressed 124 probes (116 genes) (Table 17). Of the 181 overexpressed genes, 134 genes were specifically upregulated in high iBCR score ER⁺ vs. normal breast and low iBCR ER⁺ and 95 genes were specifically upregulated in high iBCR score ER⁻ vs. normal breast and low iBCR ER⁻. As shown in Table 13, 49 genes were uniquely upregulated in high iBCR score ER− tumors compared to low score iBCR score ER⁻ tumors and normal breast tissue. Similar comparison revealed that high iBCR score ER⁺ tumors have unique upregulation of 86 genes. High iBCR score ER and ER⁺ tumors commonly overexpressed 46 genes in comparison to low score iBCR counterparts and normal breast tissue. These genes encode several kinases, enzymes and ion channels which could be targets for current or future drug development for the treatment of the high iBCR score tumors with poor outcome. Of the downregulated probes, a particularly interesting hit was the micro-RNA (miRNA) hsa-mir-568 (9.3- and 2.2-fold downregulated in high iBCR score ER⁻ vs. normal breast and low iBCR score ER⁻, respectively; 5.6- and 2.9-fold downregulated in high iBGR score ER⁺ vs. normal breast and low iBCR score ER⁺, respectively). This downregulated miRNA in the high iBCR score tumors targets several of the upregulated genes in these tumors, particularly those which are upregulated compared to normal breast tissue (Table 18). This miRNA could be a genomic-based treatment against high iBCR score breast cancers.

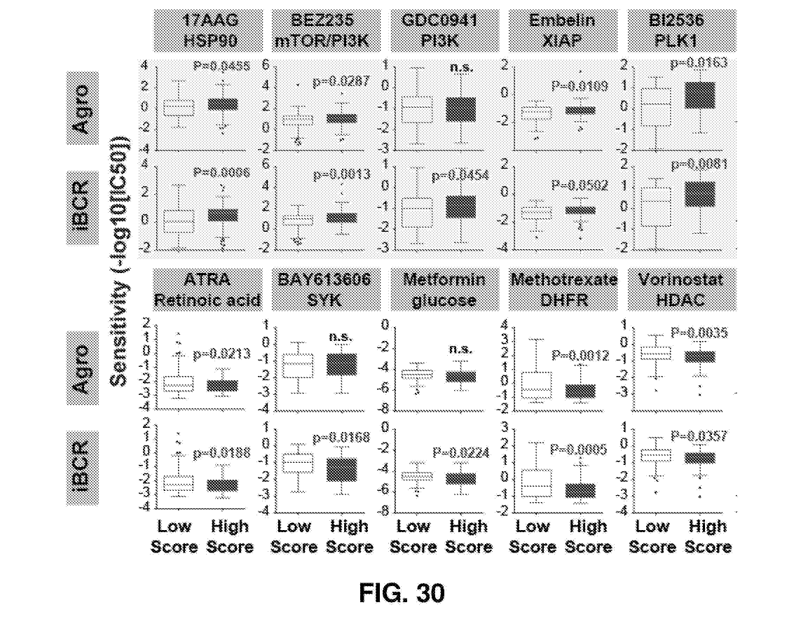

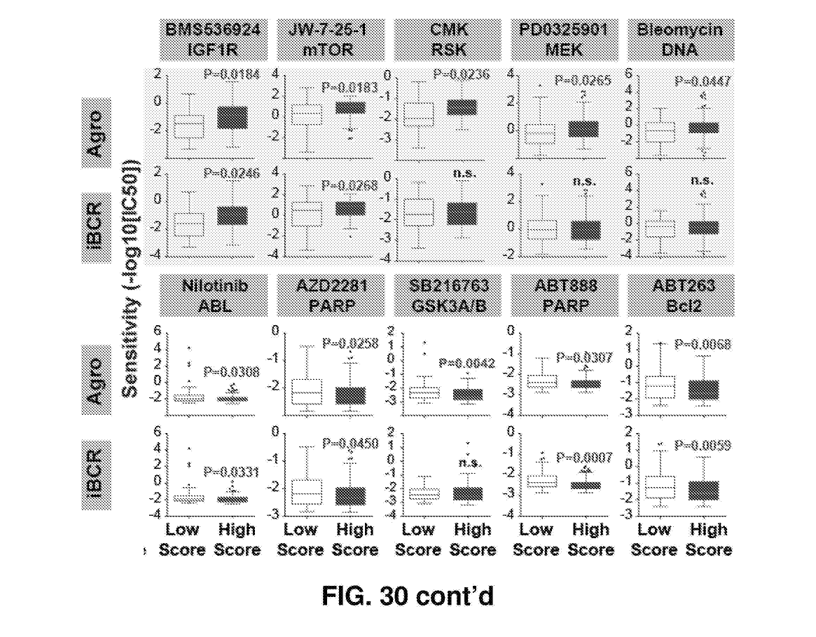

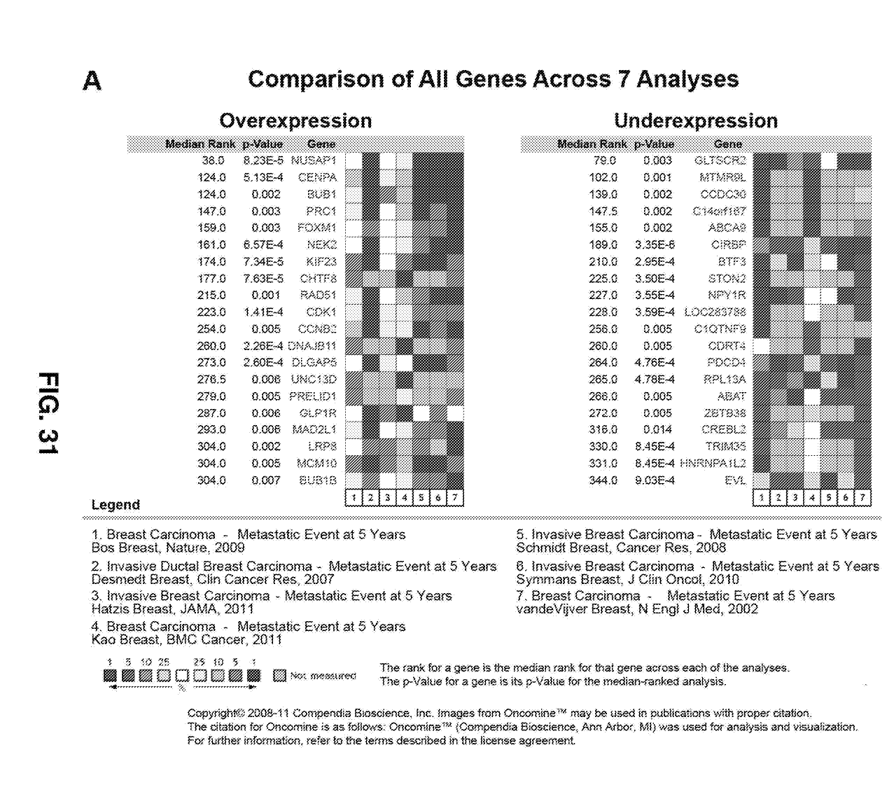

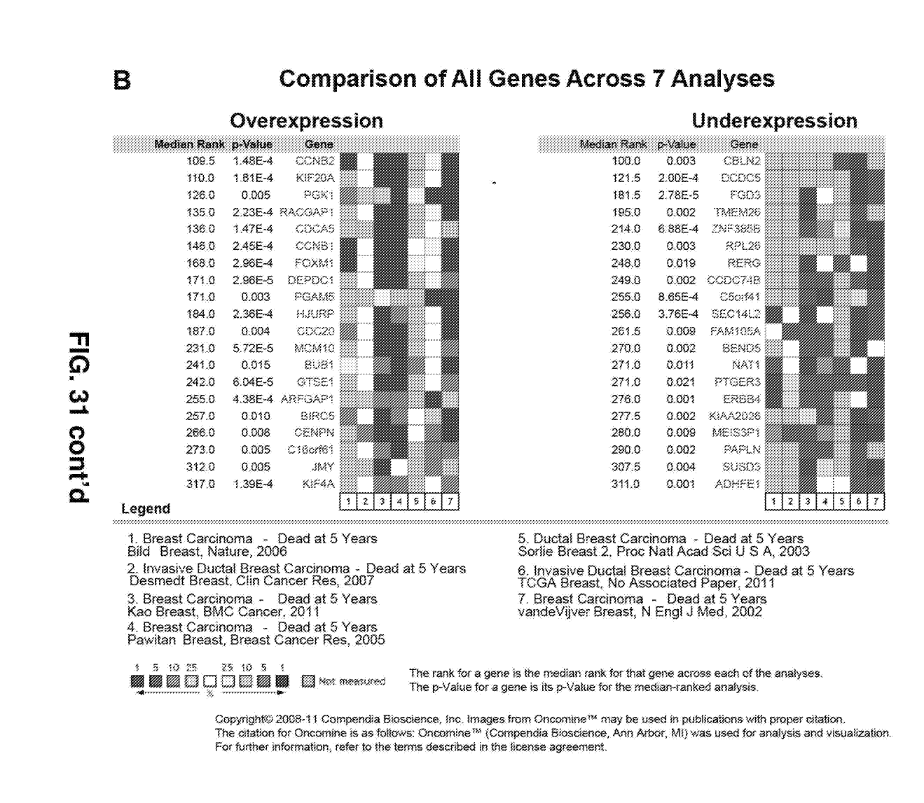

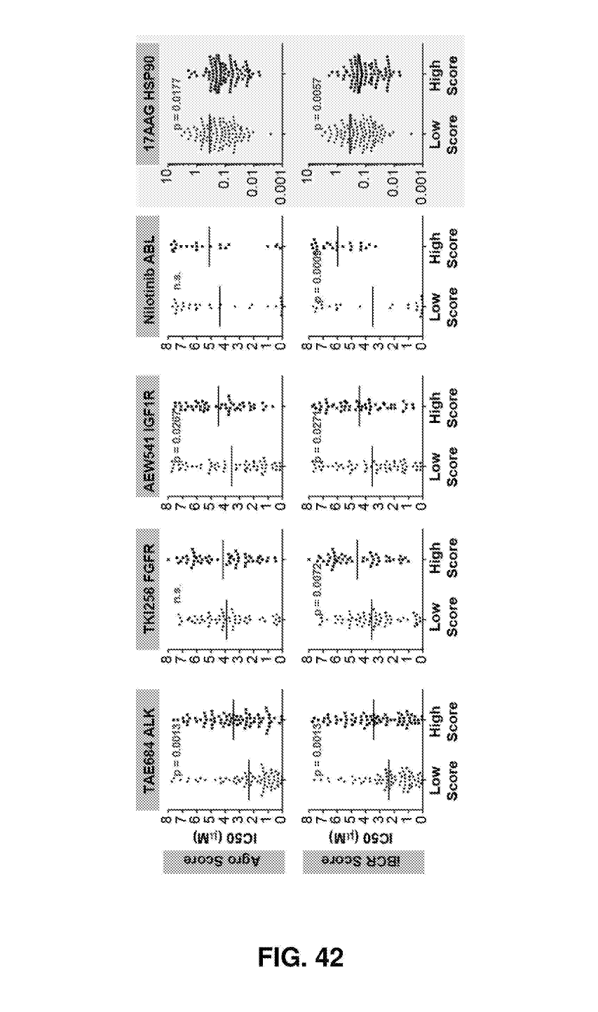

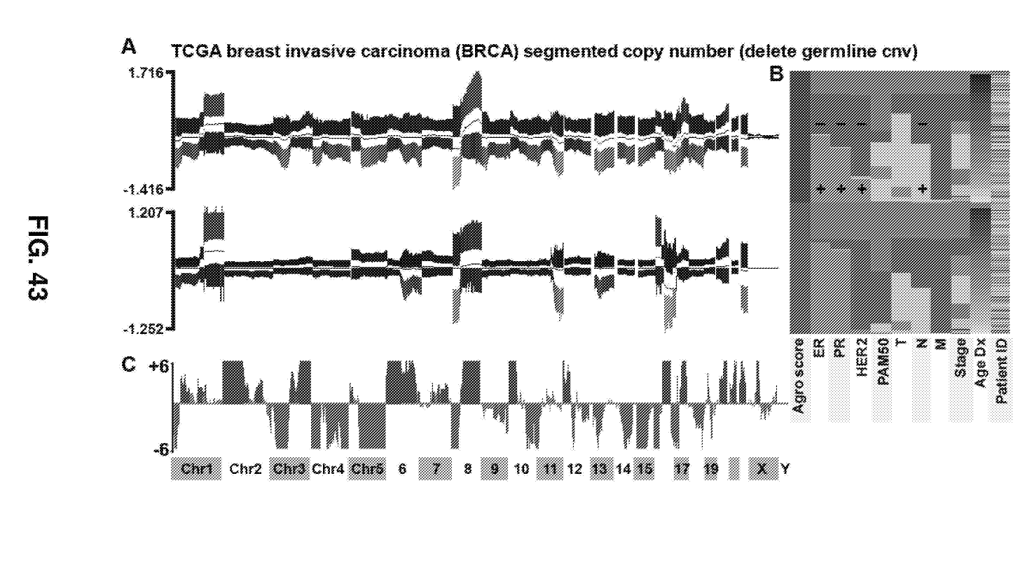

IS In the second approach, again similar to the above analysis for the TN score, published studies of drug screens were analyzed for the association of the iBCR score with sensitivity of cancer cell lines to anti-cancer drugs. In the CCLE study (FIG. 42), cancer cell lines with high iBCR score were less sensitive to inhibition of ALK (TAE684) and BCR-ABL (Nilotinib) similar to results from the TN score. In addition. high iBCR cell lines were less sensitive to inhibition of FGFR (TKI258) and IGF1R (AEW541). High iBCR score cell lines were more sensitive to the inhibition of HSP90 (Tanespimycin [17-AAG]) (FIG. 42). In the second large study by Garnett et al. [49], high iBCR score cell lines were more sensitive to low iBCR score cell lines to 8 anticancer drugs (FIG. 30). These include inhibitors of HSP90 (17AAG), mTOR/PI3K (BEZ235) and IGF1R (BMS-536924) as also observed in the TN score results. Additionally, high iBCR score cell lines were more sensitive to inhibition of PI3K (GDC0941). mTOR (JW-7-25-1), XIAP (Embelin) and PLK1 (B1-2536) which also matched results from Agro score results (FIG. 30). The Agro score also identified sensitivity to inhibition of RSK (CMK). MEK (PD0325901) and DNA damage (Bleomycin). Similar to results from high TN score, high iBCR score cell lines were also less sensitive to the inhibition of PARP (ABT-888 and AZD-2281), retinoic acid (ATRA). Bcl2 (ABT-263), DHFR (methotrexate) and glucose (metformin). Additionally, high iBCR score cell lines were less sensitive to inhibition of SYK (BAY613606), HDAC (Vorinostat) and BCR-ABL (Nilotinib) and p38MAPK (BIRB 0796). High Agro score cell lines were less sensitive to an additional drug against GSK3A/B (SB216763). Altogether, the TN score (FIGS. 24 and 35) and the Agro score and the combined iBCR score (FIGS. 30 and 42) associate with sensitivity to several anticancer drugs and future experimental validation would establish these scores as companion diagnostic for these drugs and benefit breast cancer patients by directing these drugs to the high score patients with poor survival.

Sensitivity of Breast Cancer Cell Lines to Targeted Inhibitors According to the iBCR Score

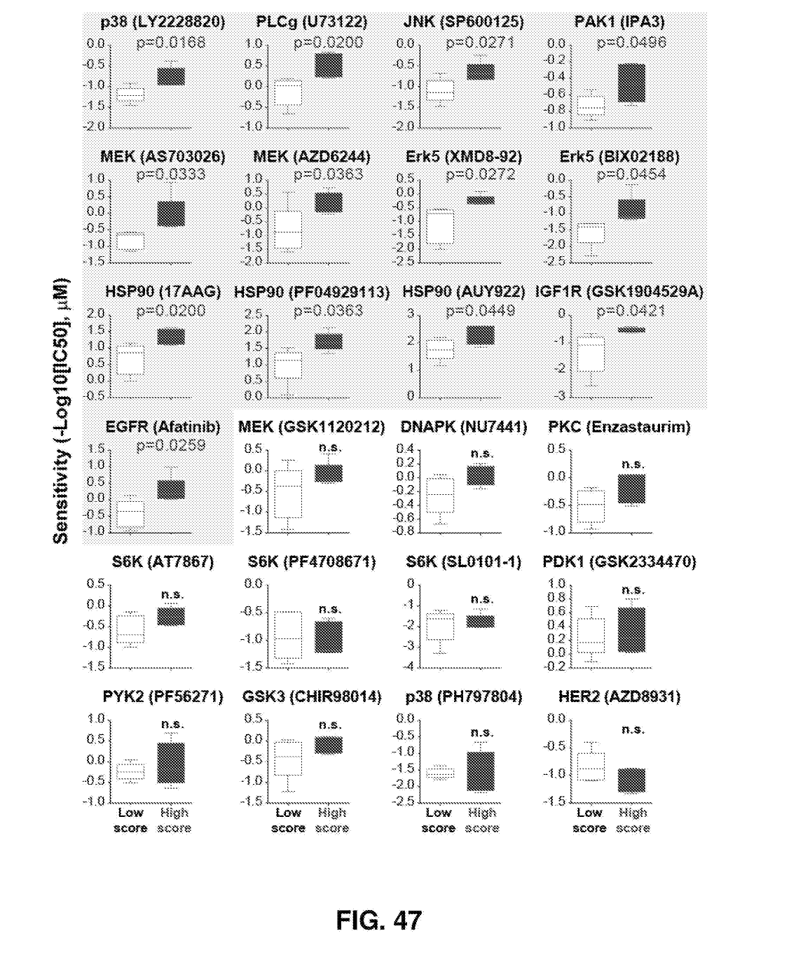

Breast cancer cell lines (10 cell lines); BT-549, MDA-MB-231, MDA-MB-436, MDA-MB-468, BT-20, Hs.578T, BT-474, MCF-7, T-47D, and ZR-75-1, were cultured in the absence or presence of escalating doses of 24 anti-cancer drugs. The survival of cells was determined six days in comparison to untreated cells using the MTS/MTA assay. The response of the cell lines to the drugs was analyzed in GraphPad® Prism using a dose response curve to calculate the log₁₀ of IC50 (IC50 is the dose required to kill 50% of the cells). Sensitivity was presented as the −log₁₀[IC50]. This drug screen which we published previously (Al-Ejeh et al., Oncotarget, 2014) was re-analyzed according to the iBCR score. The gene expression datasets of 51 breast cancer cell lines by Neve et al. (Cancer Cell, 2006), was analyzed to calculate the Agro and TN scores for each cell line to calculate the iBCR score. Each cell line was assigned as low of high iBCR score by dichotomy across the median of all the cell lines in the Neve et al. dataset. Based on the low or high iBCR score classification, the sensitivity of the 10 cell lines used in our screen was compared between high iBCR score cell lines (5 cell lines) to low iBCR score cell lines (5 cell lines). As shown in FIG. 47, high iBCR score cell lines were significantly more sensitive to the inhibition of p38MAPK (LY2228820). PLC□ (U73122), INK (SP600125), PAK1 MEK (AS703026 and AZD6244), ERK5 (XMD 8-92 and BIX02188). HSP90 (17-AAa PF0429113 and AUY922), IGF1R (GSK1904529A) and EGFR (Afatinib). The results from our screen are in agreement with the higher sensitivity of high iBCR score cancer cell lines to HSP90, IGF1R and MEK inhibitors we identified from the two previously published large cell line studies.

### Discussion

Our meta-analysis of gene expression datasets in the Oncomine™ database has previously identified a signature, the Aggressiveness signature (Agro signature), which was prognostic in ER⁺ breast cancer. We validated one of the genes in this signature, TTK/MPS1, by IEC and found that TTK positivity in interphase cells (exclusive of mitotic cells) was prognostic in highly aggressive breast cancers such as high grade, high grade and lymph node positive and highly proliferative (Ki67 positive) cases [36]. In this study, we used our meta-analysis approach to identify a second signature, the triple negative signature (TN signature), which was highly prognostic in ER⁻, TNBC and BLBC subtypes. The TN signature outperformed all standard clincopatholical indicators in multivariate survival analysis and also outperformed published signatures in ER− breast cancer. We were also able to integrate the Agro signature (prognostic in ER⁺ breast cancer) to produce the integrated Breast Cancer Recurrence (iBCR) test. The two signatures and the iBCR were validated in large independent cohorts of breast cancer studies irrespective of the gene expression arrays used indicating the experimenter/technology independence of our signatures. Importantly, both the Agro and TN signatures and the iBCR test associated with response and outcome after endocrine therapy for ER⁺ and neoadjuvant chemotherapy for ER: and ER⁺ breast cancers. Moreover, by comparison of the global gene expression profiles of high iBCR score tumors to low iBCR score tumors, we were able to identify several overexpressed targets which can be used for the targeted therapy of these poor prognosis patients who are not really benefiting from the current treatment standards. In addition, mining of large preclinical studies of drug screens against cancer cell lines showed that the signatures and iBCR score predict higher sensitivity of cell lines to particular drugs. Thus. the signatures and the iBCR test could be used as a companion diagnostic to direct targeted therapies to those patients who would benefit from these treatments to increase their low survival rates. Altogether, our studies have not only extensively illustrated the potential of our signatures in personalized medicine, but may also shed light for future studies to understand the underlying mechanisms for the aggressiveness of tumors that the iBCR test identified that lead to poor survival To date, there is an unmet medical need for the prognostication of ER− breast cancer and the development of effective therapies against these tumors particularly when lacking HER2 expression. Chemotherapy remains to be the only standard therapy in these patients and the response rate after chemotherapy in the neoadjuvant setting is reported as 31% in ER HER2⁻ (TNBC) patients [9]. Identifying patients who would truly benefit from chemotherapy would aid clinicians to determine patients who may require longer or additional treatment regimens including investigational clinical trial enrolment. Our signatures and the iBCR score predict higher pCR after chemotherapy in patients who have high scores compared to those with low score. The low score patients have better survival and may not require additional therapy. On the other hand, despite the higher pCR in high score patients, this patient subgroup still has poor survival and recurrences were present even after achieving pCR in high score patients when we analyzed the data from the 1SPY-1 trial. Our results from comparative analysis and mining pre-clinical drug screens identified several targets and sensitivity to drugs in development. Thus, ER− and particularly TNBC patients with high scores for our signatures/iBCR test may benefit from the inclusion of therapies envisioned by these signatures to increase their survival rates. Such clinical development will depend on future prospective validation of our signatures and the iBCR test in clinical trials and pre-clinical studies.

In ER⁺ breast cancer, three commercial tests exist for clinical decisions to spare or include adjuvant chemotherapy with the standard endocrine therapy; Oncotype Dx®, MammaPrint® and Prosigna®. These have been validated for ER⁺ lymph node negative (N0) breast cancer patients treated with endocrine therapy whether patients with high risk according to these tests are recommended for adjuvant chemotherapy. Our signatures and the iBCR test outperformed these tests in a direct comparison in ER⁺ N0 patient-survival after tamoxifen therapy. Moreover, our tests also predicted the response of ER⁺ patients to chemotherapy and importantly could predict sensitivity to targeted therapies. The current commercial tests do not have this capability. Importantly, our signatures and the iBCR test was also prognostic in the subgroup with unmet need, ER⁺ lymph node positive breast cancer (ER⁺ N1). The survival of these patients was stratified to poor and good prognosis groups by our signatures and iBCR test which also informed whether these patients are benefiting from endocrine therapy. Clinical validation of our signatures and the iBCR test along with validation of drug sensitivity predictions would aid the development of new treatment regimens for ER⁺ patients who are at high risk of relapse or metastatic spread after the current treatment standards.

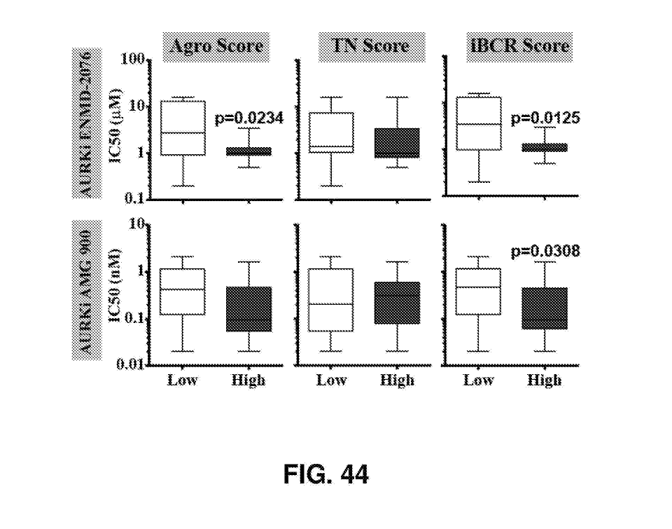

The comparison of aggressive ER⁻ tumors identified by our signatures to their counterparts and to normal breast tissue identified several kinases, enzymes (redox particularly) and potassium channels which could inform new directions in developing targeted treatments against ER⁻ breast cancer. On the other hand, for aggressive ER⁺ tumors identified by our signatures, although targets were not restricted to cell cycle and proliferation, these functions were notably enriched. This high proliferation profile could explain the higher pCR in these tumors after chemotherapy as proliferative tumors would be more responsive to chemotherapeutics. Nonetheless, we have previously clarified that the overexpressed genes in the Agro signature, thus the iBCR test, are genes that are involved in kinetochore binding and chromosome segregations and that the signature is prognostic even in proliferative tumors (high Ki67 expression) [36]. Deregulation of genes involved in chromosome segregation would produce aneuploidy and chromosomal instability (CIN) [52]. At least in viva, chemotherapy has been shown to induce the proliferation quiescent aneuploid cells as a mechanism for therapy resistance [53]. In support of the notion that high Agro Score is related to aneuploidy, analysis of the copy number variations (CNVs) TCGA data showed that high Agro score tumours, compared to low Agro score tumors, have high level of CNVs, particularly those involving whole chromosomes or chromosome arms (FIG. 43). Thus, although proliferation may be a characteristic of high Agro/iBCR score ER⁺ tumors, these tumors appear to be aneuploid. In line with this notion, the sensitivity of high Agro/iBCR score cell lines to PLK1 and HSP90 inhibition (FIG. 30) and aurora kinase inhibitors (FIG. 44) support that high Agro/iBCR scores predict sensitivity to anti-aneuploid therapy. PLK1 and Aurora kinases are classical targets in aneuploidy and HSP90 inhibition has been reported to selectively kill aneuploid cancer cells [54]. HSP90 sensitivity was also found for high TN score tumors and interestingly, we have previously identified HSP90 as a target in TNBC by kinome profiling of breast cancer. We showed that HSP90 inhibition in combination therapy is effective in vitro and in vivo [55]. We propose that anti-aneuploid drugs should be effective against ER⁺ tumors with high Agro/iBCR scores including PLK1, Aurora kinase and HSP90 inhibitors and that HSP90 inhibition should be effective in high TN/iBCR score ER⁻ tumors. While other therapies envisioned by our signatures and the iBCR test should also be investigated, the above targets represent first line targets for initial validation and development.

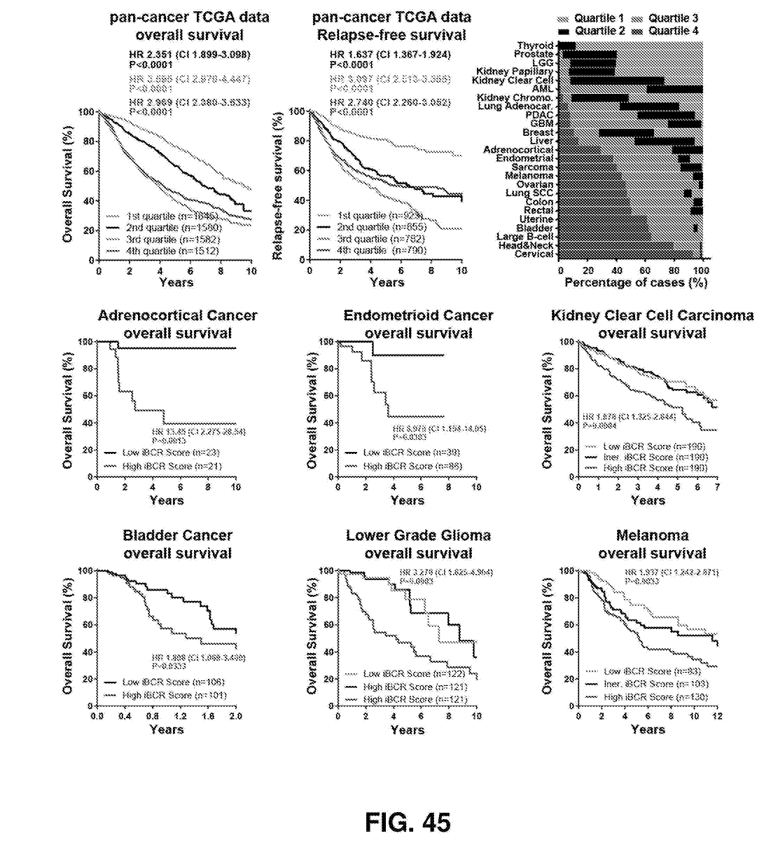

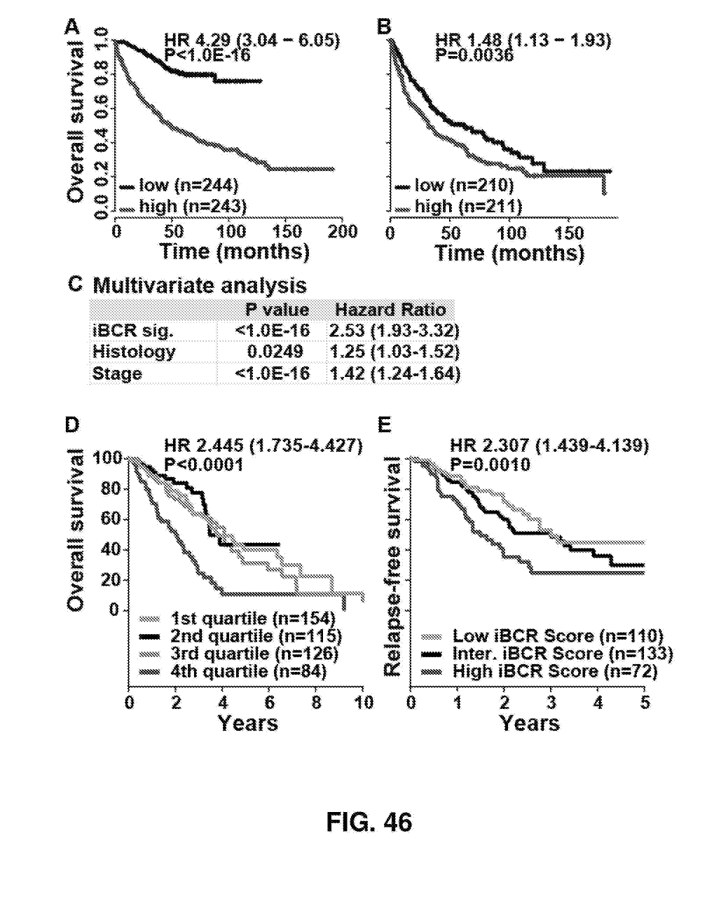

In conclusion, our meta-analysis in Oncomine™ and extensive subsequent validation and analysis have developed novel signatures and an integrated genomic test for the prognosis of breast cancer and prediction of response to standard treatments irrespective of ER status. The novel signatures and their integration also have the potential as companion diagnostic tests for several classes of targeted therapies in breast cancer patients who suffer poor survival. Future validation and clinical development of our signatures and the iBCR test holds a great potential and impact on personalized and precision medicine for breast cancer. Finally, it should be noted that the iBCR test has value in the prognosis of several other cancers (FIG. 45) and particularly in lung adenocarcinoma (FIG. 46), thus our approach and novel signatures may extend benefit to other cancer types.

### REFERENCES

1. Kang, S. P., M. Martel, and L. N. Harris, Triple negative breast cancer: current understanding of biology and treatment options. Curr Opin Obstet Gynecol, 2008. 20(1): p. 40-6.
2. Schneider, B. P., et al., Triple-negative breast cancer: risk factors to potential targets. Clin Cancer Res, 2008. 14(24): p. 8010-8.
3. Rakha, E. A., J. S. Reis-Filho, and I. O. Ellis, Basal-like breast cancer: a critical review. J Clin Oncol, 2008. 26(15): p. 2568-81.
4. Fulford, L. G., et al., Basal-like grade III invasive ductal carcinoma of the breast: patterns of metastasis and long-term survival. Breast Cancer Res, 2007. 9(1): p. R4.
5. Goldstein, L, J., et al., Concurrent doxorubicin plus docetaxel is not more effective than concurrent doxorubicin plus cyclophosphamide in operable breast cancerivith 0 to 3 positive axillary nodes: North American Breast Cancer Intergroup Trial E 2197. J Clin Oncol, 2008. 26(25): p. 4092-9.
6. Kean, B., et al., Prognostic impact of clinicopathologic parameters in stage II/III breast cancer treated with neoadjuvant docetaxel and doxorubicin chemotherapy: paradoxical features of the triple negative breast cancer. BMC Cancer, 2007. 7: p. 203.
7. Liedtke, C., et at, Response to neoadjuvant therapy and long-term survival in patients with triple-negative breast cancer. J Clin Oncol, 2008. 26(8): p. 1275-81.
8. Carey, L. A., et al., The triple negative paradox: primary tumor chemosensitivity of breast cancer subtypes. Clin Cancer Res, 2007. 13(8): p. 2329-34.
9. von Minckwitz, G., et al., Definition and impact of pathologic complete response on prognosis after neoadjuvant chemotherapy in various intrinsic breast cancer subtypes. J Clin Oncol, 2012. 30(15): p. 1796-804.
10. Sorlie, T., et at, Gene expression patterns of breast carcinomas distinguish tumor subclasses with clinical implications. Proc Natl Acad Sci USA, 2001. 98(19): p. 10869-74.
11. Perou, C. M., et al., Molecular portraits of human breast tumours. Nature, 2000. 406(6797): p. 747-52.
12. Hu, Z., et al., The molecular portraits of breast tumors are conserved across microarray platforms. BMC Genomics, 2006. 7: p. 96-107.
13. Parker, J. S., et al., Supervised Risk Predictor of Breast cancer Based on. Intrinsic Subtypes. J Clin Oncol, 2009.
14. Weigelt, B., et al., Breast cancer molecular profiling with single sample predictors: a retrospective analysis. Lancet Oncol. 11(4): p. 339-49.
15. Weigelt, B., et al., Molecular portraits and 70-gene prognosis signature are preserved throughout the metastatic process of breast cancer. Cancer Res, 2005. 65(20): p. 9155-8.
16, Parker, J. S., et al., Supervised risk predictor of breast cancer based on intrinsic subtypes. J Clin Oncol, 2009. 27(8): p. 1160-7.
17, Lehmann, B. D., et al., Identification of human triple-negative breast cancer subtypes and preclinical models for selection of targeted therapies. J Clin Invest, 2011. 121(7); p. 2750-67.
18. Shah. S. P. et al., The clonal and mutational evolution spectrum of primary triple-negative breast cancers. Nature, 2012. 486(7403): p. 395-9.
19. Irshad, S., P. Ellis, and A. Tutt, Molecular heterogeneity of triple-negative breast cancer and its clinical implications. Cuff Opin Oncol, 2011. 23(6): p. 566-77.
20. Criscitiello, C. et al., Understanding the biology of triple-negative breast cancer. Ann Oncol, 2012. 23 Suppl 6: p. vi13-8.
21. Masuda, R, et al., Differential response to neoadjuvant chemotherapy among 7 triple-negative breast cancer molecular subtypes. Clin Cancer Res, 2013. 19(19): p. 5533-40.
22. van't Veer, Li., et al., Gene expression profiling predicts clinical outcome of breast cancer. Nature, 2002. 415(6871): p. 530-6.
23. Paik, S., et al., A multigene assay to predict recurrence of tamoxifen-treated. node-negative breast cancer. N Engl J Med, 2004. 351(27): p. 2817-26,
24, Buyse, M., et al., Validation and clinical utility of a 70-gene prognostic signature for women with node-negative breast cancer. J Natl Cancer Inst, 2006. 98(17): p. 1183-92.
25. Loi, S., et al., Definition of clinically distinct molecular subtypes in estrogen receptor-positive breast carcinomas through genomic grade. J Clin Oncol, 2007. 25(10): p. 1239-46.
26. Ma, X. J., et al., A five-gene molecular grade index and HOXB13:IL17BR are complementary prognostic factors in early stage breast cancer. Clin Cancer Res, 2008. 14(9): p. 2601-8.
27, Ma, X. J., et al., A two-gene expression ratio predicts clinical outcome in breast cancer patients treated with tamoxifen. Cancer Cell, 2004, 5(6): p. 607-16.
28. Sotiriou, C., et al., Gene expression profiling in breast cancer . . . understanding the molecular basis of histologic grade to improve prognosis. Journal of the National Cancer Institute, 2006. 98(4): p. 262-72.
29. Dowsett, M., et al., Comparison of PAM50 risk of recurrence score with oncotype DX and IHC4 for predicting risk of distant recurrence after endocrine therapy. J Clin Oncol, 2013. 31(22): p. 2783-90.
30. Yau, C., et al., A multigene predictor of metastatic outcome in early stage hormone receptor-negative and triple-negative breast cancer. Breast Cancer Res, 2010. 12(5): p. R85.
31. Rody, A., et al., A clinically relevant gene signature in triple negative and basal-like breast cancer. Breast Cancer Res, 2011. 13(5): p. R97.
32. Karn, T., et al., Homogeneous datasets of triple negative breast cancers enable the identification of novel prognostic and predictive signatures. PLoS One, 2011. 6(12): p. e28403.
33. Yu, K. D., et al., Identification of prognosis-relevant subgroups in patients with chemoresistant triple-negative breast cancer. Clin Cancer Res, 2013. 19(10): p. 2723-33.
34. Lee, U., et al., A prognostic gene signature for metastasis-free survival of triple negative breast cancer patients. PLoS One, 2013. 8(12): p. e82125.
35. Hallett, R. M., et al., A gene signature for predicting outcome in patients with basal-like breast cancer. Sci Rep, 2012. 2: p. 227.
36. Al-Ejeh, F., et al., Mew-analysis of the global gene expression profile of triple-negative breast cancer identifies genes for the prognostication and treatment of aggressive breast cancer. Oncogenesis, 2014. 3: p. e
37. Rhodes, D. R., et al., ONCOMINE: a cancer microarray database and integrated data-mining platform. Neoplasia, 2004. 6(1): p. 1-6.
38. Gyorffy, B., et al., An online survival analysis tool to rapidly assess the effect of 22,277 genes on breast cancer prognosis using microarray data of 1,809 patients. Breast Cancer Res Treat, 2010. 123(3): p. 725-31.
39. TCGA, Comprehensive molecular portraits of human breast tumours. Nature, 2012. 490(7418): p. 61-70.
40, Ur-Rehman, S., et al., ROCK: a resource for integrative breast cancer data analysis. Breast Cancer Res Treat, 2013. 139(3): p. 907-21.
41. Esserman, L. J., et al., Pathologic complete response predicts recurrence-free survival more effectively by cancer subset: results from the I-SPY 1 TRIAL-CALGB 150007/150012, ACRIN 6657, J Clin Oncol, 2012. 30(26): p. 3242-9.
42. Korde, L. A., et al., Gene expression pathway analysis to predict response to neoadjuvant docetaxel and capecitabine for breast cancer. Breast Cancer Res Treat, 2010. 119(3); p. 685-99.
43. Prat, A., et al., Research-based PAM50 subtype predictor identifies higher responses and improved survival outcomes in. HER2-positive breast cancer in the NOAH study. Chin Cancer Res, 2014. 20(2): p. 511-21.
44, Tabchy, A., et al., Evaluation of a 30-gene paclitaxel, fluorouracil, doxorubicin, and cyclophosphamide chemotherapy response predictor in a multicenter randomized trial in breast cancer. Clin Cancer Res, 2010. 16(21): p. 5351-61.
45. Popovici, V., et al., Effect of training-sample size and classification difficulty on the accuracy of genomic predictors. Breast Cancer Res, 2010. 12(1): p. R5.
46. Esserman, L. J., et al., Chemotherapy response and recurrence-free survival in neoadjuvant breast cancer depends on biomarker profiles: results from the I-SPY 1 TRIAL (CALGB 150007/150012; ACRIN 6657). Breast Cancer Res Treat, 2012. 132(3): p. 1049-62.
47. Shen, K., et al., Cell line derived multi-gene predictor of pathologic response to neoadjuvant chemotherapy in breast cancer: a validation study on US Oncology 02-103 clinical trial. BMC Med Genomics, 2012. 5: p. 51.
48. Iwamoto, T., et al., Gene pathways associated with prognosis and chemotherapy sensitivity in molecular subtypes of breast cancer. J Natl Cancer Inst, 2011. 103(3): p. 264-72.
49. Garnett, M. J., et al., Systematic identification of genomic markers of drug sensitivity in cancer cells. Nature, 2012. 483(7391): p. 570-5.
50. Barretina, J., et al., The Cancer Cell Line Encyclopedia enables predictive modelling of anticancer drug sensitivity. Nature, 2012. 483(7391): p. 603-7.
51. Symmans, W. F., et al., Genomic index of sensitivity to endocrine therapy for breast cancer. J Clin Oncol, 2010. 28(27): p. 4111-9.
52, Bakhoum, S. F. and D. A. Compton, Chromosomal instability and cancer: a complex relationship with therapeutic potential. J Clin Invest, 2012, 122(4): p. 1138-43.
53. Kusumbe, A. P. and S. A. Bapal, Cancer stem cells and aneuploid populations within developing tumors are the major determinants of tumor dormancy. Cancer Res, 2009. 69(24): p. 9245-53.
54. Tang, Y. C., et al., identification of aneuploidy-selective antiproliferation compounds, Cell, 2011. 144(4): p. 499-512.
55. Al-Ejeh, F., et al., Kinome profiling reveals breast cancer heterogeneity and identifies targeted therapeutic opportunities for triple negative breast cancer. Oncotarget, 2014.
56. Bos, P. D., et al., Genes that mediate breast cancer metastasis to the brain, Nature, 2009. 459(7249): p. 1005-9.
57. Desmedt, C., et al., Strong time dependence of the 76-gene prognostic signature for node-negative breast cancer patients in the TRANSBIG multicenter independent validation series, Clin Cancer Res, 2007. 13(11): p. 3207-14.
58. Hatzis, C., et al., A genomic predictor of response and survival following taxane-anthracycline chemotherapy for invasive breast cancer. JAMA, 2011. 305(18): p. 1873-81.
59. Kao, K J., et al., Correlation of microarray-based breast cancer molecular subtypes and clinical outcomes; implications for treatment optimization. BMC Cancer, 2011. 11: p. 143.
60. Schmidt, M., et al., The humoral immune system has a key prognostic impact in node-negative breast cancer. Cancer Res, 2008. 68(13): p. 5405-13.
61. van de Vijver, M. J., et al., A gene-expression signature as a predictor of survival in breast cancer. N Engl J Med, 2002. 347(25): p, 1999-2009.
62. Bild, A. H., et al., Oncogenic pathway signatures in human cancers as a guide to targeted therapies. Nature, 2006. 439(7074): p. 353-7.
63. Pawitan, Y., et al., Gene expression profiling spares early breast cancer patients from adjuvant therapy; derived and validated in two population-based cohorts. Breast Cancer Res, 2005. 7(6): p. R953-64.
64. Sorlie, To, et al., Repeated observation of breast tumor subtypes in independent gene expression data sets. Proc Natl Acad Sci USA. 2003. 100(14): p. 8418-23,
65, Zhao, Y. and R. Simon, BRB-ArrayTools Data Archive for human cancer gene expression: a unique and efficient data sharing resource. Cancer Inform, 2008. 6: p. 9-15.
66. Cline, M. S., et al., Exploring TCGA Pan-Cancer data at the UCSC Cancer Genomics Browser. Sci Rep, 2013. 3: p. 2652.
67. Goldman, M., et al., The UCSC Cancer Genomics Browser: update 2013. Nucleic Acids Res, 2013, 41(Database issue): p. D949-54.
68. Al-Ejeh, F., et al., Treatment of triple-negative breast cancer using anti-EGFR-directed radioimmunotherapy combined with radiosensitizing chemotherapy and PARR inhibitor. J Nucl Med, 2013, 54(6): p. 913-21.
69. Diamond, J. R., et al., Predictive biomarkers of sensitivity to the aurora and angiogenic kinase inhibitor ENMD-2076 in preclinical breast cancer models. Clin Cancer Res, 2013. 19(1): p. 291-303,
70. Kalous, O., et al., AMG 900, pan-Aurora kinase inhibitor, preferentially inhibits the proliferation of breast cancer cell lines with dysfunctional p53. Breast Cancer Res Treat, 2013. 141(3): p. 397-408,

TABLE 10
The 28-gene signature discovered from a meta-analysis of gene
expression data in breast cancer in Oncomine ™

| Gene | Affymetrix | | |
|---|---|---|---|
| Symbol | probe | Entrez | Gene name |
| ↑ABHD5 | 213935_at | 51099 | abhydrolase domain containing 5; 1- |
| | | | acylglycerol-3-phosphate O-acyltransferase |
| ↑ADORA | 205891_at | 136 | adenosine A2b receptor |
| 2B | | | |
| ↑BCAP31 | 200837_at | 10134 | B-cell receptor-associated protein 31 |
| ↑CA9 | 205199_at | 768 | carbonic anhydrase IX |
| ↑CAMSA | 212711_at | 157922 | calmodulin regulated spectrin-associated |
| P1 | | | protein 1 |
| ↑CARHSP | 218384_at | 23589 | calcium regulated heat stable protein 1, |
| 1 | | | 24 kDa |
| ↑CD55 | 201926_s_at | 1604 | CD55 molecule, decay accelerating factor |
| | | | for complement (Cromer blood group) |
| ↑CETN3 | 209662_at | 1070 | centrin, EF-hand protein, 3 |
| ↑EIF3K | 221494_x_at | 27335 | eukaryotic translation initiation factor 3, |
| | | | subunit K |
| ↑EXOSC7 | 212627_s_at | 23016 | exosome component 7 |
| ↑GNB2L1 | 200651_at | 10399 | guanine nucleotide binding protein (G |
| | | | protein), beta polypeptide 2-like 1 |
| ↑GRHPR | 214864_s_at | 9380 | glyoxylate reductase/hydroxypyruvate |
| | | | reductase |
| ↑GSK3B | 209945_s_at | 2932 | glycogen synthase kinase 3 beta |
| ↑HCFC1R | 218537_at | 54985 | host cell factor C1 regulator 1 (XPO1 |
| 1 | | | dependent) |
| ↑KCNG1 | 214595_at | 3755 | potassium voltage-gated channel, subfamily |
| | | | G, member 1 |
| ↑MAP2K5 | 211370_s_at | 5607 | mitogen-activated protein kinase kinase 5 |
| ↑NDUFC1 | 203478_at | 4717 | NADH dehydrogenase (ubiquinone) 1, |
| | | | subcomplex unknown, 1, 6 kDa |
| ↑PML | 206503_x_at | 5371 | promyelocytic leukemia |
| ↑STAU1 | 208948_s_at | 6780 | staufen, RNA binding protein, homolog 1 |
| | | | (Drosophila) |
| ↑TXN | 216609_at | 7295 | thioredoxin |
| ↑ZNF593 | 204175_at | 51042 | zinc finger protein 593 |
| ↓BTN2A2 | 205298_s_at | 10385 | butpophilin, subfamily 2, member A2 |
| ↓ERC2 | 213938_at | 26059 | ELKS/RAB6-interacting/CAST family |
| | | | member 2 |
| ↓IGH | 211649_x_at | 3492 | immunoglobulin heavy locus |
| ↓ME1 | 211204_at | 4199 | malic enzyme 1, NADP (+)-dependent, |
| | | | cytosolic |
| ↓MTMR7 | 217292_at | 9108 | myotubularin related protein 7 |
| ↓SMPDL3 | 205309_at | 27293 | sphingomyelin phosphodiesterase, acid-like |
| B | | | 3B |
| ↓ZNRD1- | 215985_at | 80862 | ZNRD1 antisense RNA 1 |
| ↓AS1 | | | |

TABLE 11
The TN signature is prognostic in ER−
and BLBC irrespective of systemic therapy.

| | | Untreated | | | Treated | | |
|---|---|---|---|---|---|---|---|
| | | HR | CI 95% | p-value | HR | CI 95% | p-value |
| ER− | RFS | 2.02 | 1.25-3.26 | 3.20E−03 | 2.59 | 1.84-3.60 | 1.70E−08 |
| | DMFS | 4.10 | 1.44-11.7 | 4.20E−03 | 1.89 | 1.04-3.43 | 3.40E−02 |
| | OS | 1.77 | 0.65-4.83 | 0.26 | 3.82 | 1.43-10.18 | 3.90E−03 |
| BLBC | RFS | 2.48 | 1.46-4.21 | 5.10E−04 | 2.88 | 1.94-4.28 | 4.50E−08 |
| | DMFS | 5.54 | 1.66-18.48 | 1.70E−03 | 3.14 | 1.38-7.19 | 4.20E−03 |
| | OS | 2.42 | 0.79-7.47 | 0.11 | 4.89 | 1.65-14.46 | 1.50E−03 |

The 28-gene signature was used as described in FIG. 2 in the online tool KM-plotter but restricting the analysis on ER− or BLBC patients who were untreated systemically or systemically treated. The survival curves for RFS. DMFS and OS are .shown in FIG. 34; only the hazard ratio (HR), the 95% confidence interval (CI 95%) and the log-rank p-value from these curves are reported in the Table.

TABLE 12
The likelihood of pCR in ER-HER2-patients
according to the iBCR score

| | pCR | pCR | Sum |
|---|---|---|---|
| Low Score | 12 (6.6%) | 51 (27.9%) | 63 (34.4%) |
| High Score | 54 (29.5%) | 66 (36.1%) | 120 (65.6%) |
| Total | 66 (36.1%) | 117 (63.1%) | 183 (100%) |

ER-/HER2-patients stratified by low and high iBCR scores from four studies were compared for achieving or not achieving pCR after four chemography regimens: FAC (GSE20271), TFAC (GSE20271 and GSE20194), FEC/TX (GSE42822) and FAC/TX (GSE42822) and FAC/TX (GSE23988)

TABLE 13
Upregulated genes in high iBCR score tumors compared to low iBCR tumors and normal breast tissue

| | | | | | | | Common in high IBCR | | |
|---|---|---|---|---|---|---|---|---|---|
| High iBCR score ER− vs. low iBCR | | | High iBCR score ER+ vs. | | | | score ER−/+ vs. low iBCR | | |
| score ER− and normal breast | | | low iBCR score ER+ and normal breast | | | | score and normal | | |
| ACE2 | HMGB3 | | ACP1 | ENO1 | MCM4 | | ASPM | HN1 | |
| ADM | IL8 | | APOBEC3B | EPRS | MCM6 | RANBP1 | AURKA | KCNK1 | |
| AR | IMPA2 | | ATAD2 | EXOSC4 | MCM7 | RECQL4 | BIRC5 | KIF4A | |
| BNIP3 | KYNU | | AURKB | FADS1 | MRPL13 | RFC2 | BUB1 | MKI67 | |
| C1orf106 | LBP | | BOP1 | FANCI | MRPL15 | RMDN1 | BUB1B | MLF1IP | |
| CALML5 | LRP8 | | CACYBP | GINS2 | MSH6 | RSAD2 | CCNB1 | MMP1 | |
| CBS | MAGEA3 | TDO2 | CALU | GTSE1 | MYBL2 | SHMT2 | CCNB2 | MTFR1 | |
| CCL18 | MAGEA6 | TMEM45A | CCNA2 | H2AFZ | NCAPG | SMC4 | CCNE2 | NDC80 | YKT6 |
| CD24 | ME1 | TMSB15A | CCT2 | HELLS | NDUFS8 | SPAG5 | CDC20 | NEK2 | ZWINT |
| CLIC3 | MMP12 | VEGFA | CDCA3 | HMMR | NUDT21 | SQLE | CDC6 | NUSAP1 | |
| CORO1C | PFKP | VGLL1 | CEBPG | HSPH1 | NUTF2 | STIP1 | CDK1 | PDXK | |
| CP | PHLDA2 | | CKS1B | KIAA0101 | OIP5 | TACC3 | CDKN3 | PHB | |
| CRISP3 | PTPN12 | | CXCL10 | KIF11 | PBK | TBCE | CENPF | PRC1 | |
| DDC | QPRT | | CXCL11 | KIF14 | PCNA | TIMM17A | CKS2 | PTTG1 | |
| ECT2 | S100A7 | | DERL1 | KIF20A | PGK1 | TMPO | CNIH4 | RRM2 | |
| EZH2 | S100A9 | | DHFR | KPNA2 | PLOD2 | TSN | CNTNAP2 | S100A8 | |
| FABP7 | SCD | | DNPH1 | KPNA4 | PRAME | TYMS | DDA1 | S100P | |
| FAR2 | SLC7A5 | | DONSON | LAPTM4B | PSMA7 | UBE2S | DLGAP5 | SPP1 | |
| GABBR2 | SOD2 | | DSCC1 | LMNB1 | PSMC3 | UCK2 | DTL | TK1 | |
| GALNT3 | SOX11 | | EIF4EBP1 | LSM4 | RACGAP1 | WHSC1 | ESRP1 | TOP2A | |
| GMPS | SRD5A1 | | EIF5A | LY6E | RAD21 | ZWILCH | GINS1 | TRIP13 | |
| GPSM2 | ST14 | | EMC8 | MAD2L1 | RAD54B | | HIST1H2BG | UBE2C | |

TABLE 18
Upregulated targets of the downregulated hsa-mir-568 in high iBCR
score ER−/ER+ tumors.

| Fold- | | | | | | |
|---|---|---|---|---|---|---|
| change ↑ | ProbeSet | Symbol | Name | EntrezID | Accession | UGCluster |
| 3.0 | 220085_at | HELLS | helicase, lymphoid- | 3070 | NM_018063 | Hs.655830 |
| | | | specific | | | |
| 2.5 | 201291_s_at | TOP2A | topoisomerase (DNA) II | 7153 | AU159942 | Hs.156346 |
| | | | alpha 170 kDa | | | |
| 2.4 | 203213_at | CDK1 | cyclin-dependent kinase 1 | 983 | AL524035 | Hs.732435 |
| 2.3 | 212009_s_at | STIP1 | stress-induced- | 10963 | AL553320 | Hs.337295 |
| | | | phosphoprotein 1 | | | |
| 1.7 | 203755_at | BUB1B | BUB1 mitotic checkpoint | 701 | NM_001211 | Hs.513645 |
| | | | serine/threonine kinase B | | | |
| 1.6 | 205282_at | LRP8 | low density lipoprotein | 7804 | NM_004631 | Hs.280387 |
| | | | receptor-related protein 8, | | | |
| | | | apolipoprotein e receptor | | | |
| 1.6 | 202697_at | NUDT21 | nudix (nucleoside | 11051 | NM_007006 | Hs.528834 |
| | | | diphosphate linked moiety | | | |
| | | | X)-type motif 21 | | | |
| 1.5 | 209053_s_at | WHSC1 | Wolf-Hirschhorn | 7468 | BE793789 | Hs.113876 |
| | | | syndrome candidate 1 | | | |
| 1.9 | 202134_s_at | WWTR1 | WW domain containing | 25937 | NM_015472 | Hs.594912 |
| | | | transcription regulator 1 | | | |
| 1.9 | 213906_at | MYBL1 | v-myb myeloblastosis | 4603 | AW592266 | Hs.445898 |
| | | | viral oncogene homolog | | | |
| | | | (avian)-like 1 | | | |
| 1.8 | 206348_s_at | PDK3 | pyruvate dehydrogenase | 5165 | NM_005391 | Hs.296031 |
| | | | kinase, isozyme 3 | | | |
| 1.8 | 219927_at | FCF1 | FCF1 small subunit (SSU) | 51077 | NM_015962 | Hs.579828 |
| | | | processome component | | | |
| | | | homolog (S. cerevisiae) | | | |
| 1.8 | 209757_s_at | MYCN | v-myc myelocytomatosis | 4613 | BC002712 | Hs.25960 |
| | | | viral related oncogene, | | | |
| | | | neuroblastoma derived | | | |
| | | | (avian) | | | |
| 1.8 | 217562_at | FAM5C | family with sequence | 339479 | BF589529 | Hs.65765 |
| | | | similarity 5, member C | | | |
| 1.7 | 219875_s_at | DESI2 | desumoylating | 51029 | NM_016076 | Hs.498317 |
| | | | isopeptidase 2 | | | |
| 1.7 | 215305_at | PDGFRA | platelet-derived growth | 5156 | H79306 | Hs.74615 |
| | | | factor receptor, alpha | | | |
| | | | polypeptide | | | |
| 1.7 | 219434_at | TREM1 | triggering receptor | 54210 | NM_018643 | Hs.283022 |
| | | | expressed on myeloid cells 1 | | | |
| 1.7 | 217834_s_at | SYNCRIP | synaptotagmin binding, | 10492 | NM_006372 | Hs.571177 |
| | | | cytoplasmic RNA | | | |
| | | | interacting protein | | | |
| 1.6 | 205646_s_at | PAX6 | paired box 6 | 5080 | NM_000280 | Hs.270303 |
| 1.6 | 205796_at | TCP11L1 | t-complex 11, testis- | 55346 | NM_018393 | Hs.655341 |
| | | | specific-like 1 | | | |
| 1.6 | 222269_at | APOOL | apolipoprotein O-like | 139322 | W87634 | Hs.512181 |
| 1.6 | 219311_at | CEP76 | centrosomal protein | 79959 | NM_024899 | Hs.236940 |
| | | | 76 kDa | | | |
| 1.6 | 214708_at | SNTB1 | syntrophin, beta 1 | 6641 | BG484314 | Rs.46701 |
| | | | (dystrophin-associated | | | |
| | | | protein A1, 59 kDa, basic | | | |
| | | | component 1) | | | |
| 1.6 | 210073_at | ST8SIA1 | ST8 alpha-N-acetyl- | 6489 | L32867 | Hs.408614 |
| | | | neuraminide alpha-2,8- | | | |
| | | | sialyltransferase 1 | | | |
| 1.6 | 205490_x_at | GJB3 | gap junction protein, beta | 2707 | BF060667 | Hs.522561 |
| | | | 3, 31 kDa | | | |
| 1.6 | 219944_at | CLIP4 | CAP-GLY domain | 79745 | NM_024692 | Hs.122927 |
| | | | containing linker protein | | | |
| | | | family, member 4 | | | |
| 1.6 | 206357_at | OPA3 | optic atrophy 3 (autosomal | 80207 | NM_025136 | Hs.466945 |
| | | | recessive, with chorea and | | | |
| | | | spastic paraplegia) | | | |
| 1.6 | 219262_at | SUV39H2 | suppressor of variegation | 79723 | NM_024670 | Hs.554883 |
| | | | 3-9 homolog 2 | | | |
| | | | (Drosophila) | | | |
| 1.5 | 201602_s_at | PPP1R12A | protein phosphatase 1, | 4659 | BE737620 | Hs.49582 |
| | | | regulatory subunit 12A | | | |
| 1.5 | 216008_s_at | ARIH2 | ariadne homolog 2 | 10425 | AV694434 | Hs.633601 |
| | | | (Drosophila) | | | |
| 1.5 | 200671_s_at | SPTBN1 | spectrin, beta, non- | 6711 | N92501 | Hs.503178 |
| | | | erythrocytic 1 | | | |
| 1.5 | 210041_s_at | PGM3 | phosphoglucomutase 3 | 5238 | BC001258 | Hs.661665 |
| 1.5 | 206376_at | SLC6A15 | solute carrier family 6 | 55117 | NM_018057 | Hs.44424 |
| | | | (neutral amino acid | | | |
| | | | transporter), member 15 | | | |

Bolded genes upregulated in high iBCR score ER−/ER+ vs. normal breast.

### Example 3

The iBCR test described herein was developed from a meta-analysis of gene expression profiles of breast cancer. This test is based on the expression of 43 genes which are prognostic as a signature in breast cancer irrespective of subtype. This test was also found to be prognostic in lung adenocarcinoma. Patients with high iBCR score have much poorer overall survival than patients with low iBCR score.

In the current study. The Cancer Genome Atlas (TCGA) datasets for several cancer types were investigated for three purposes. First, to determine the differences in at the protein level between high iBCR score breast cancer cases to low iBCR score breast cancer cases. This comparison was also carried out for lung adenocarcinoma. Secondly. to determine whether deregulated proteins/phosphoproteins between high and low iBCR score tumours are prognostic. Finally, the prognostic value of the iBCR mRNA signature and associated protein signature are prognostic in other cancer types profiled by the TCGA.

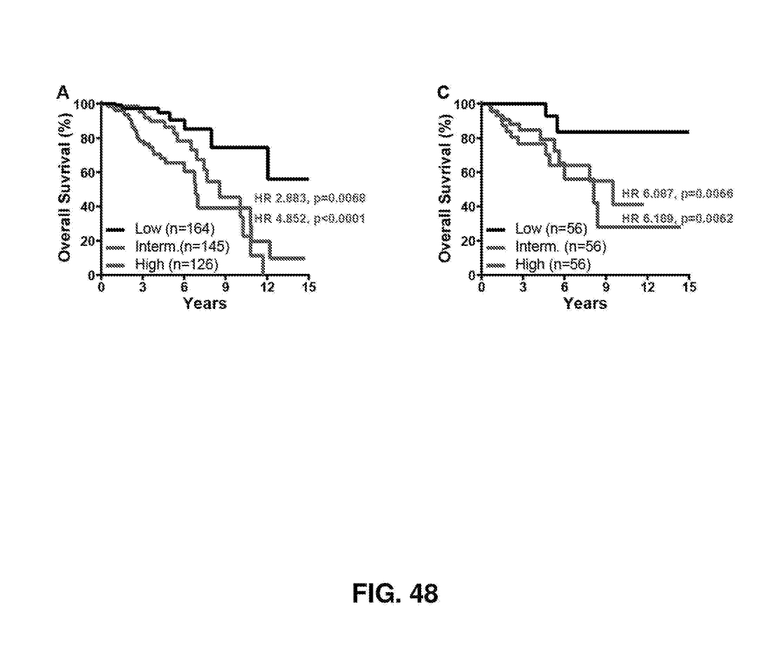

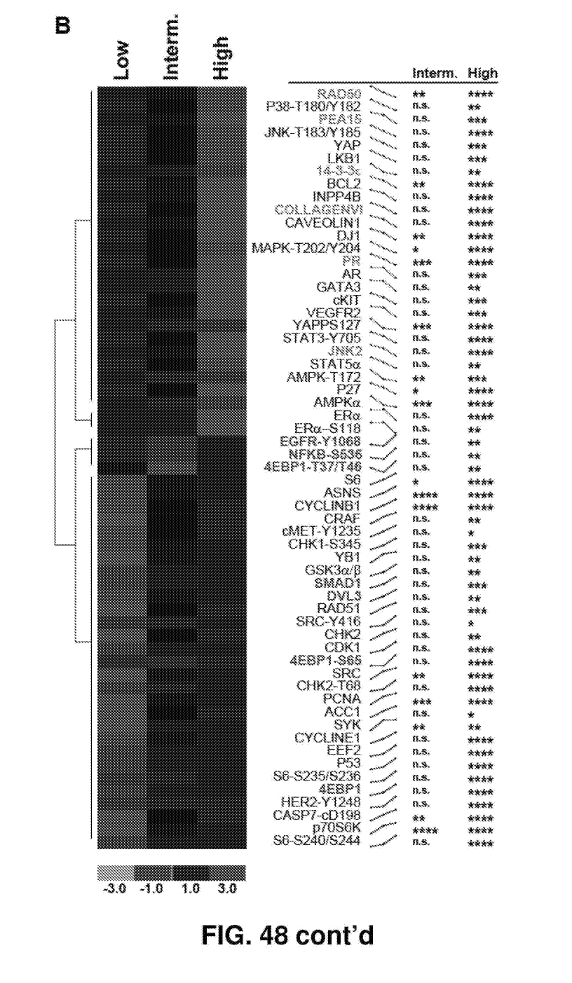

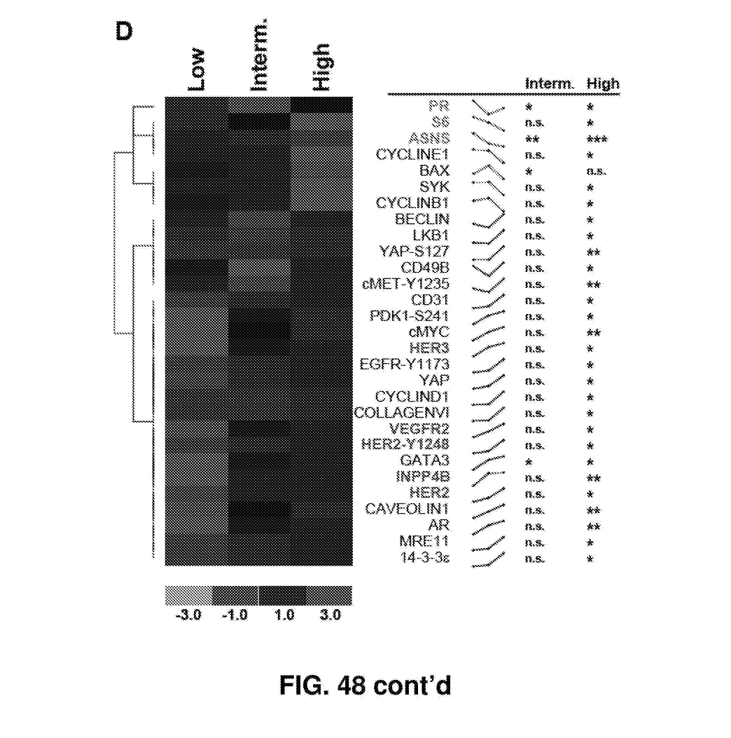

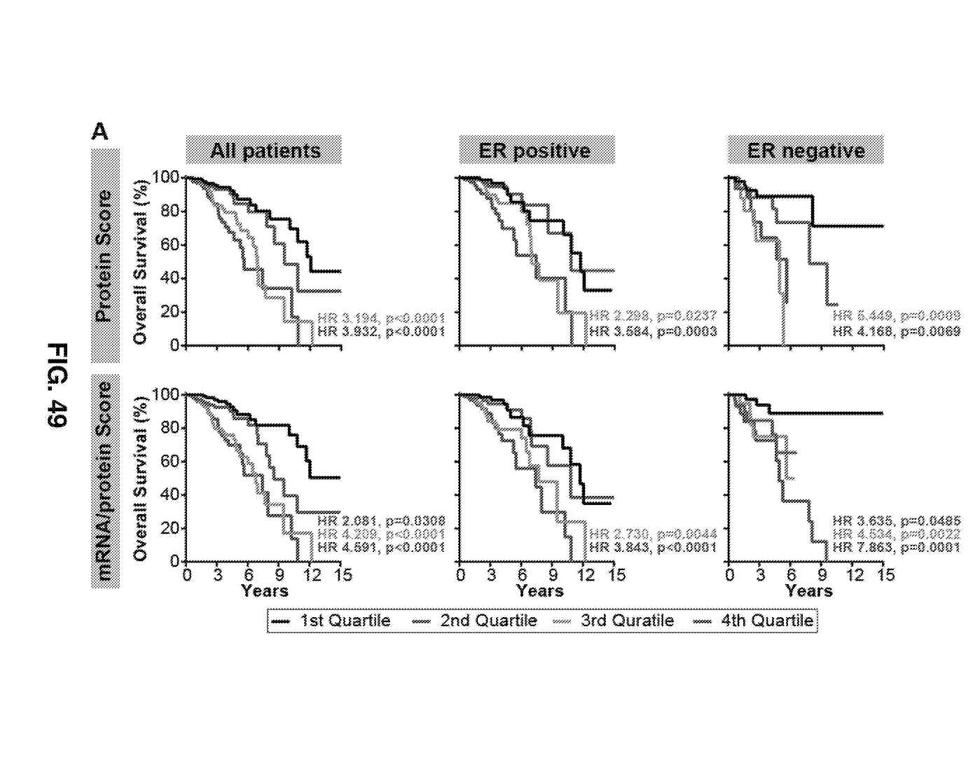

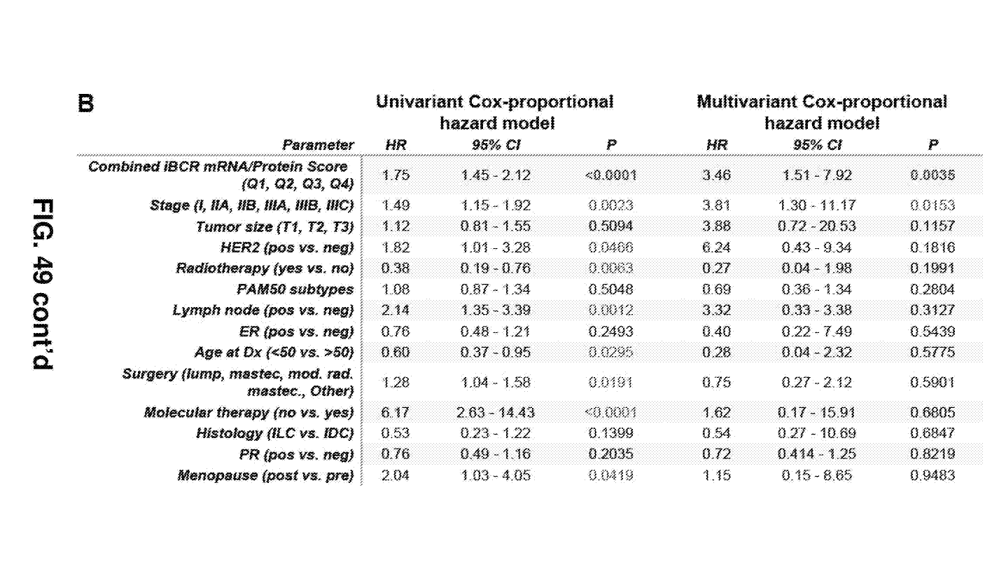

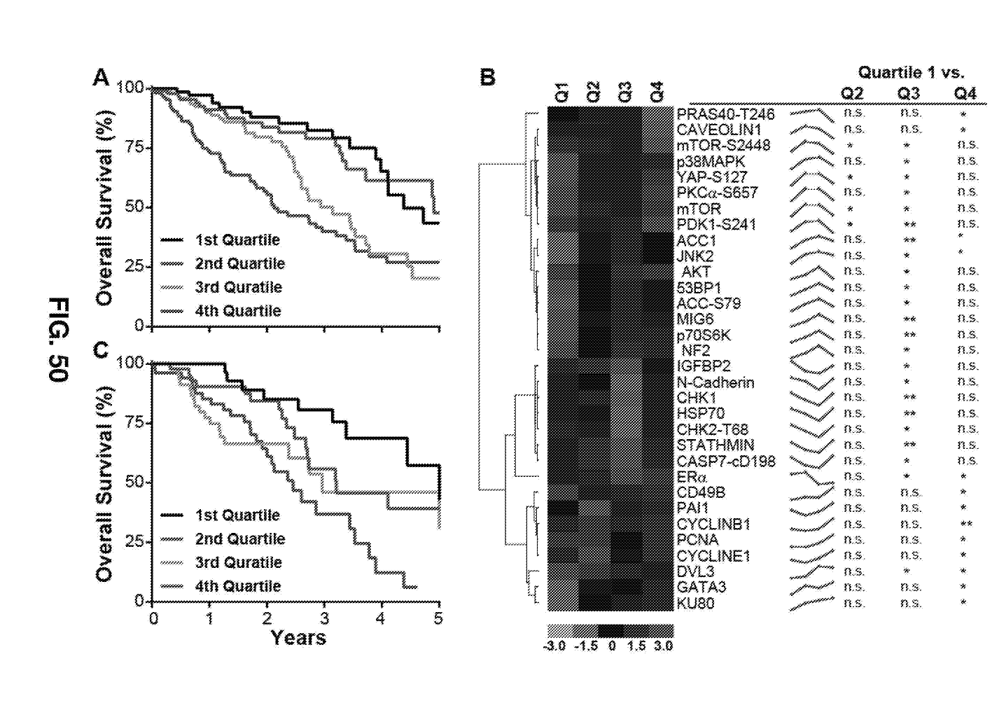

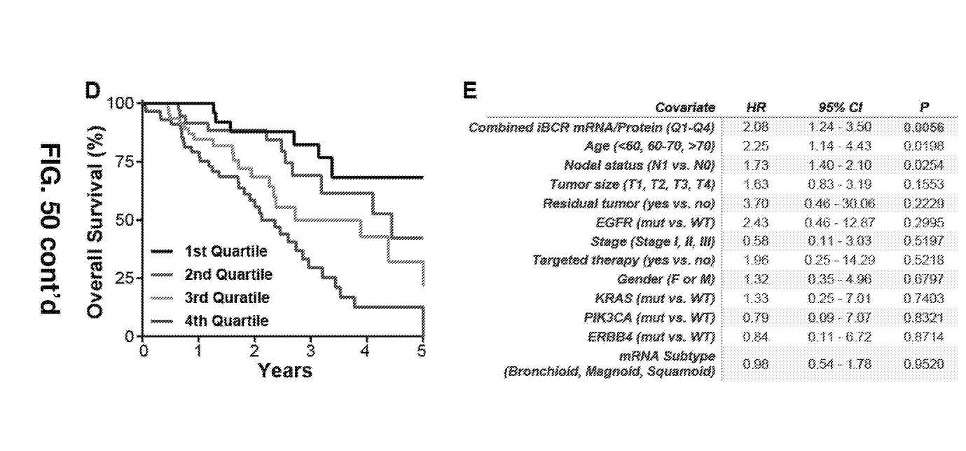

As shown in FIGS. 48A&B, comparison of the reverse phase protein array (RPPA) data between ER+ breast cancer cases with high iBCR score and low iBCR score identified several deregulated proteins and phosphoproteins between these two patient subgroups. Similar analysis in ER− breast cancer cases with high iBCR score compared to those with low iBCR score also identified deregulated proteins and phosphoproteins between these two patient subgroups (FIGS. 48C&D). These significantly deregulated proteins and phosphoproteins were then tested for association with overall survival. The upregulation of 9 and down regulation of proteins/phosphoproteins were highly prognostic in breast cancer Importantly, the integration of the iBCR mRNA and protein signatures is the most significant indicator of overall survival of breast cancer patients irrespective of subtypes and in comparison to all known clinicopathological indicators (FIG. 49B).

Similar analysis in the lung adenocarcinoma TCGA dataset identified proteins/phosphoproteins based on the iBCR mRNA signature which are prognostic as a protein signature (FIG. 50A-C). The integration of the iBCR mRNA/protein signatures were highly prognostic and outperformed the standard clinicopathological indicators in lung adenocarcinoma (FIGS. 50D&E).

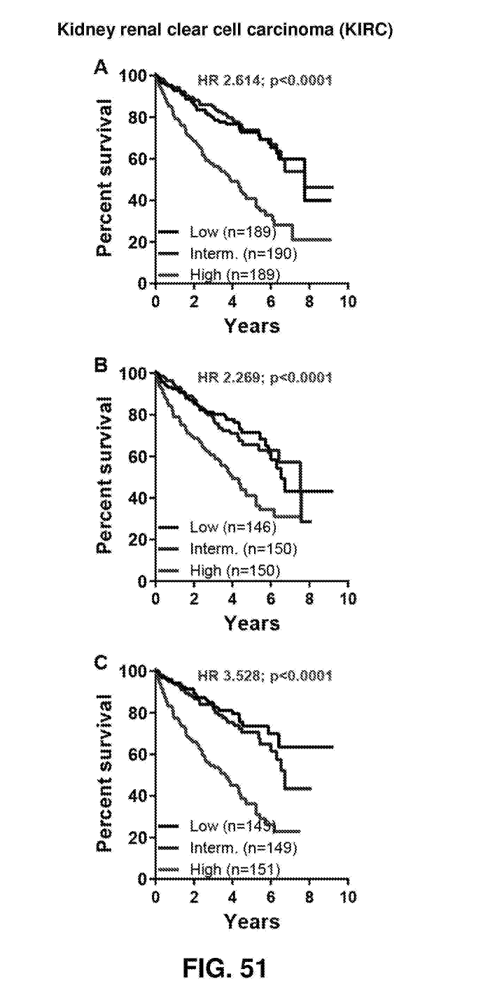

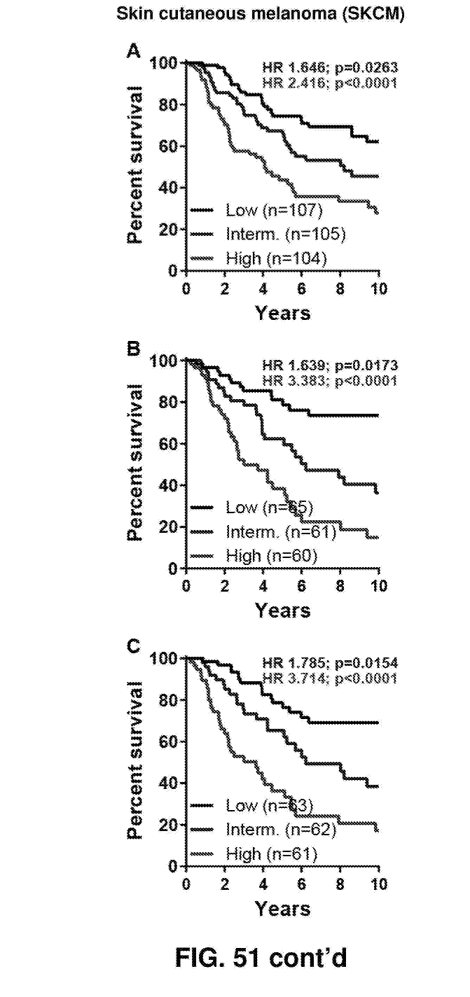

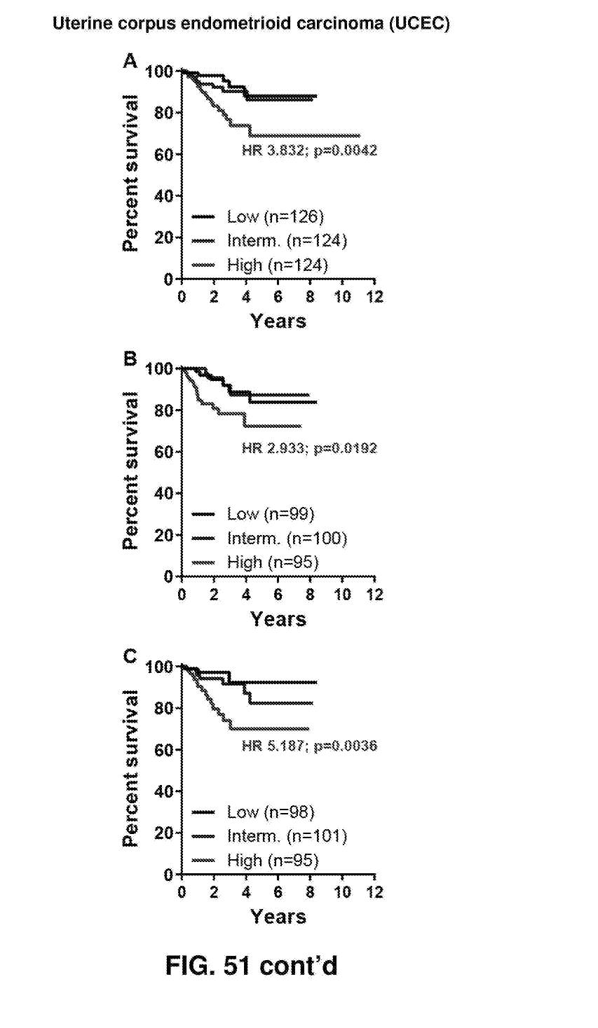

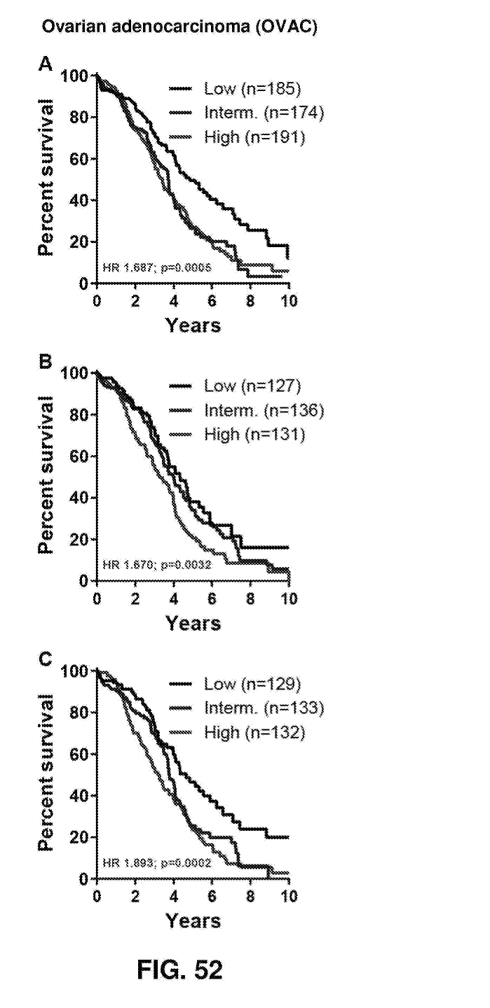

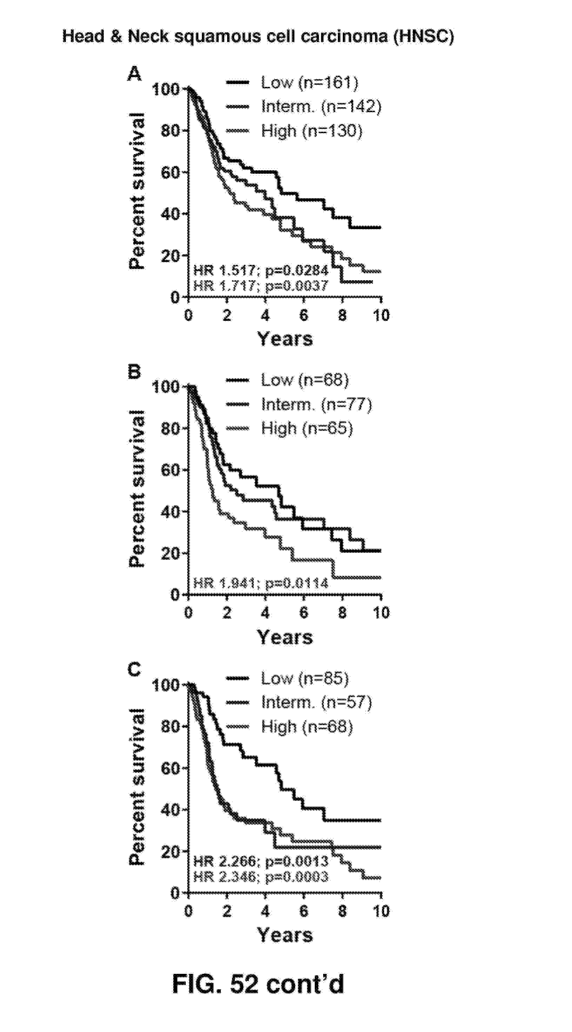

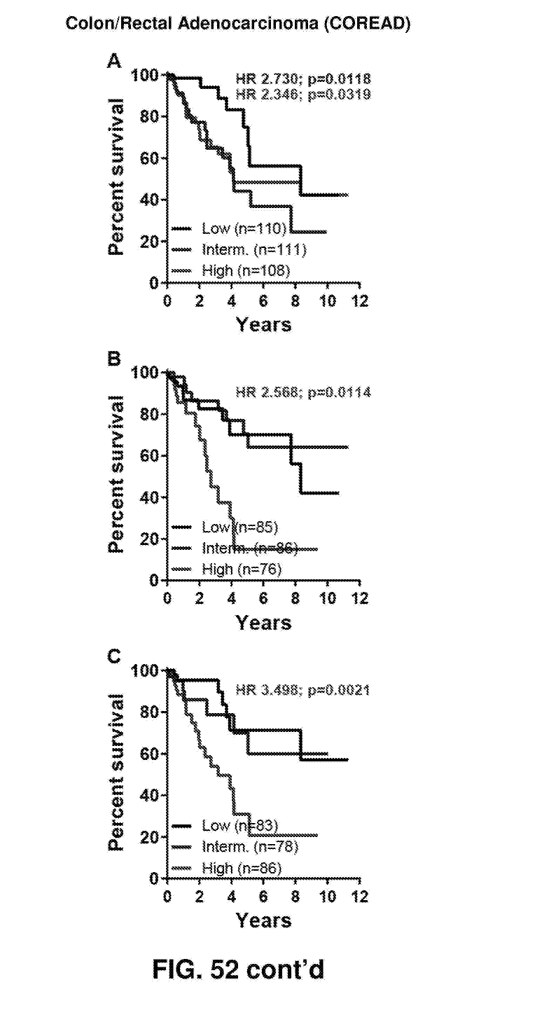

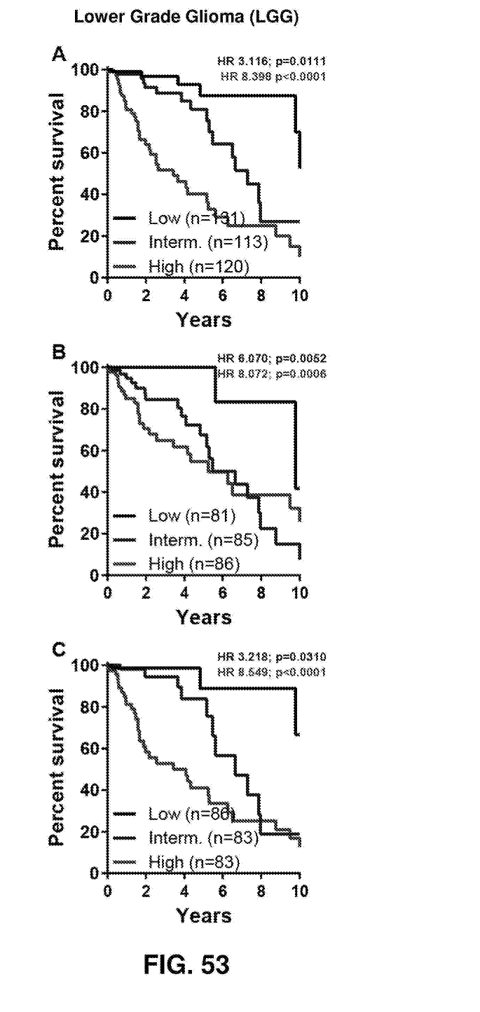

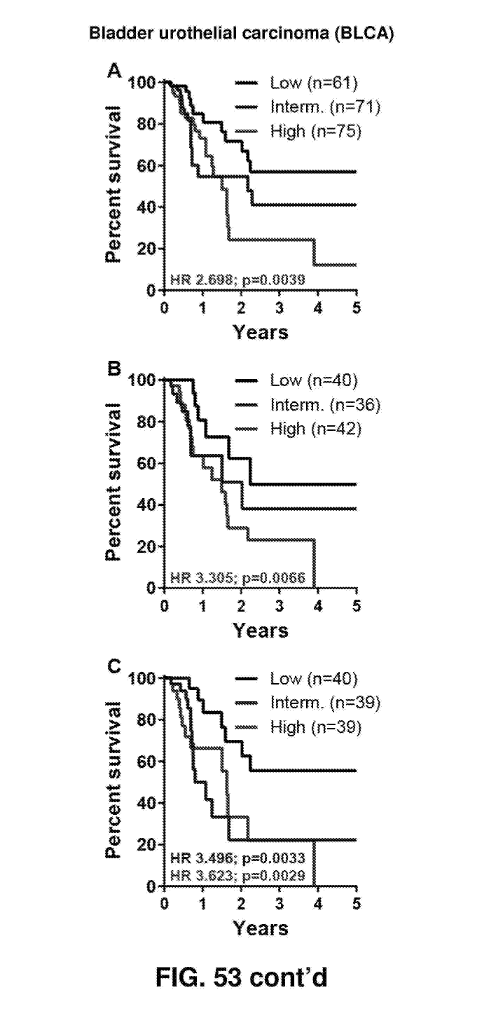

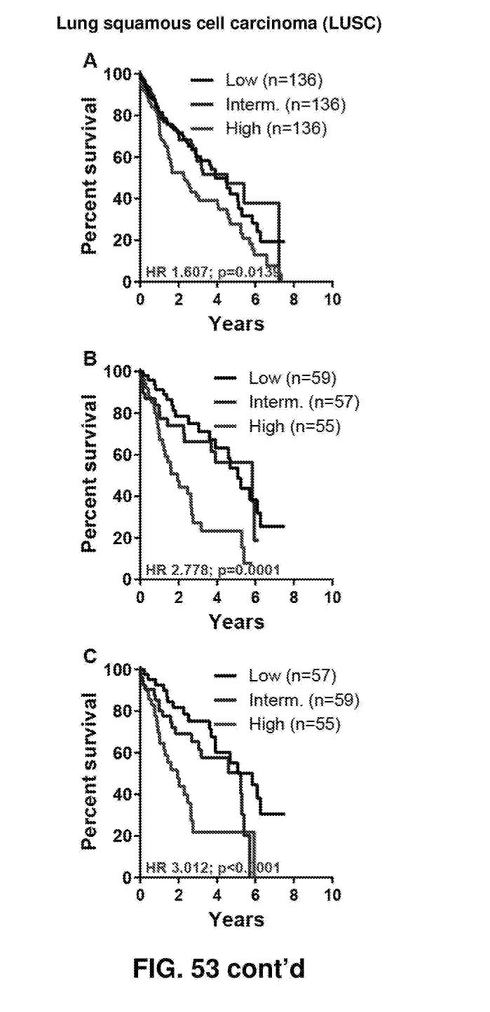

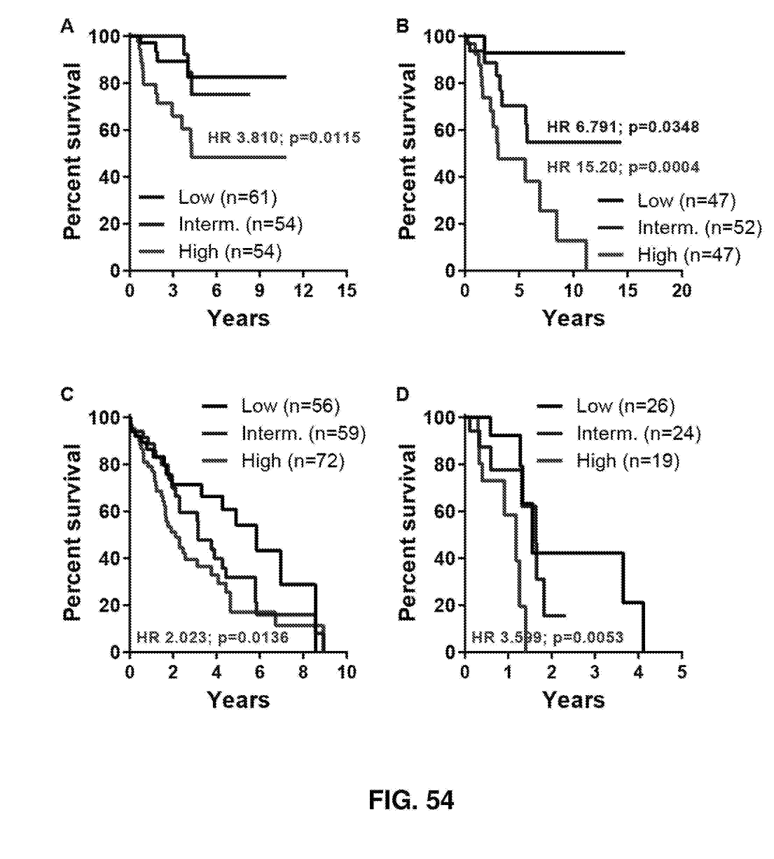

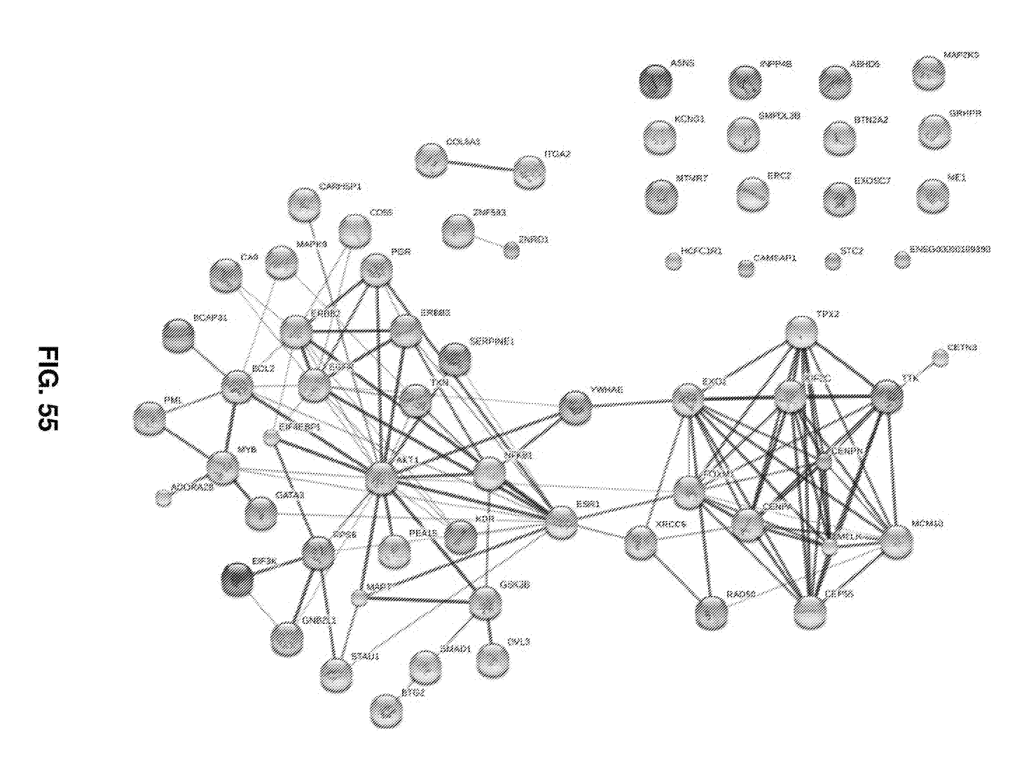

Table 19 summarises the 43 genes at the mRNA level and 2 proteins/phosphoproteins in the iBCR test. The components which were prognostic in breast cancer (FIG. 48 & FIG. 49) and lung adenocarcinoma (FIG. 50) are labelled in Table 19. Next, the association of the mRNA and protein/phosphoprotein levels of the genes in Table 19 with overall survival was tested in other cancer types. The deregulation of mRNA and protein levels of the iBCR test components that associate with overall survival is summarised in Table 19. For each cancer type, the marked components were used as a signature and the stratification of overall survival of kidney renal clear cell carcinoma (KIRC), skin cutaneous melanoma (SKCM), uterine corpus endometrioid carcinoma (UCEC), ovarian adenocarcinoma (OVAC), head and neck squamous cell carcinoma (HNSC), colon/rectal adenocarcinoma (COREAD), lower grade glioma (LGG), bladder urothelial carcinoma (BLCA). lung squamous cell carcinoma (LUSC), kidney renal papillary cell carcinoma (KIRP), cervical squamous cell carcinoma and endocervical adenocarcinoma (CESC), liver hepatocellular carcinoma (LIHC) and pancreatic ductal adenocarcinoma (PDAC). is shown FIGS. 51 to 54.

In conclusion, the iBCR test including the mRNA and protein components (Table 19) is a highly prognostic test in all cancers tests. This test identifies aggressive human cancers and is enriched for protein-protein interactions (FIG. 55) as well as biological functions related to the hallmarks of cancer (Table 20).

TABLE 20
Enrichment of biological functions related to the hallmarks of cancer
in the iBCR test

| | | | | | P-VALUE |
|---|---|---|---|---|---|
| | | # | | P-VALUE | BONFERR |
| GO ID | TERM | GENES | P-VALUE | FDR | ONI |
| GO: 0009 | response to endogenous stimulus | 22 | 9.17E− | 1.13E− | 1.13E−06 |
| 719 | | | 11 | 06 | |
| GO: 1901 | response to oxygen-containing | 18 | 9.10E− | 2.90E− | 1.13E−03 |
| 700 | compound | | 08 | 04 | |
| GO: 0032 | regulation of cellular protein | 20 | 138E− | 2.90E− | 1.96E−03 |
| 268 | metabolic process | | 07 | 04 | |
| GO: 0035 | intracellular signal transduction | 20 | 1.66E− | 2.90E− | 2.05E−03 |
| 556 | | | 07 | 04 | |
| GO: 0010 | response to grganonitrogen | 14 | 1.80E− | 2.90E− | 2.22E−03 |
| 243 | compound | | 07 | 04 | |
| GO: 0010 | response to organic substance | 24 | 1.82E− | 2.90E− | 2.25E−03 |
| 033 | | | 07 | 04 | |
| GO: 0000 | mitotic cell cycle | 14 | 1.83E− | 2.90E− | 2.27E−03 |
| 278 | | | 07 | 04 | |
| GO: 0051 | regulation of transport | 18 | 1.87E− | 2.90E− | 2.32E−03 |
| 049 | | | 07 | 04 | |
| GO: 0031 | positive regulation of protein | 15 | 2.68E− | 3.41E− | 3.32E−03 |
| 401 | modification process | | 07 | 04 | |
| GO: 0022 | cell cycle process | 16 | 2.86E− | 3.41E− | 3.34E−03 |
| 402 | | | 07 | 04 | |
| GO: 0044 | positive regulation of | 18 | 3.47E− | 3.41E− | 4.30E−03 |
| 093 | function | | 07 | 04 | |
| GO: 0051 | negative regulation of transport | 10 | 3.75E− | 3.41E− | 4.64E−03 |
| 051 | | | 07 | 04 | |
| GO: 0042 | response to drug | 11 | 3.76E− | 3.41E− | 4.66E−03 |
| 493 | | | 07 | 04 | |
| GO: 0007 | cell cycle | 18 | 3.85E− | 3.41E− | 4.77E−03 |
| 049 | | | 07 | 04 | |
| GO: 0009 | response to mechanical stimulus | 8 | 4.36E− | 3.60E− | 5.40E−03 |
| 612 | | | 07 | 04 | |
| GO: 0001 | positive regulation of protein | 13 | 5.76E− | 4.13E− | 7.13E−03 |
| 934 | phosphorylation | | 07 | 04 | |
| GO: 0008 | cell proliferation | 13 | 6.10E− | 4.13E− | 7.55E−03 |
| 283 | | | 07 | 04 | |
| GO: 0009 | positive regulation of signal | 16 | 6.12E− | 4.13E− | 7.57E−03 |
| 967 | transduction | | 07 | 04 | |
| GO: 0051 | positive regulation of cellular | 13 | 6.34E− | 4.13E− | 7.85E−03 |
| 130 | component organization | | 07 | 04 | |
| GO: 0022 | regulation of anatomical structure | 13 | 8.87E− | 5.49E− | 1.10E−02 |
| 603 | morphogenesis | | 07 | 04 | |
| GO: 0072 | divalent inorganic cation | 9 | 9.96E− | 5.70E− | 1.23E−02 |
| 507 | homeostasis | | 07 | 04 | |
| GO: 0023 | positive regulation of signaling | 16 | 1.12E− | 5.70E− | 1.38E−02 |
| 056 | | | 06 | 04 | |
| GO: 0032 | positive regulation of cellular | 15 | 1.13E− | 5.70E− | 1.40E−02 |
| 270 | protein metabolic process | | 06 | 04 | |
| GO: 0048 | gland development | 9 | 1.13E− | 5.70E− | 1.40E−02 |
| 732 | | | 06 | 04 | |
| GO: 0010 | positive regulation of cell | 16 | 1.18E− | 5.70E− | 1.46E−02 |
| 647 | communication | | 06 | 04 | |
| GO: 0051 | regulation of protein metabolic | 20 | 1.20E− | 5.70E− | 1.48E−02 |
| 246 | process | | 06 | 04 | |
| GO: 0051 | regulation of cellular component | 19 | 1.51E− | 6.91E− | 1.87E−02 |
| 128 | organization | | 06 | 04 | |
| GO: 0071 | cellular response to organic | 19 | 1.89E− | 8.34E− | 2.34E−02 |
| 310 | substance | | 06 | 04 | |
| GO: 0042 | positive regulation of | 13 | 2.51E− | 1.07E− | 3.10E−02 |
| 327 | phosphorylation | | 06 | 03 | |
| GO: 1901 | response to nitrogen compound | 13 | 2.90E− | 1.18E− | 3.59E−02 |
| 698 | | | 06 | 03 | |
| GO: 0009 | response to hormone | 13 | 2.95E− | 1.18E− | 3.65E−02 |
| 725 | | | 06 | 03 | |
| GO: 0048 | positive regulation of response to | 18 | 3.30E− | 1.24E− | 4.08E−02 |
| 584 | stimulus | | 06 | 03 | |
| GO: 0042 | regulation of cell proliferation | 17 | 3.36E− | 1.24E− | 4.16E−02 |
| 127 | | | 06 | 03 | |
| GO: 0070 | cellular response to chemical | 21 | 3.40E− | 1.24E− | 4.21E−02 |
| 887 | stimulus | | 06 | 03 | |
| GO: 0010 | posttranscriptional regulation of | 10 | 3.65E− | 1.29E− | 4.52E−02 |
| 608 | gene expression | | 06 | 03 | |
| GO: 0043 | positive regulation of catalytic | 15 | 3.78E− | 1.30E− | 4.68E−02 |
| 085 | activity | | 06 | 03 | |

### Example 4

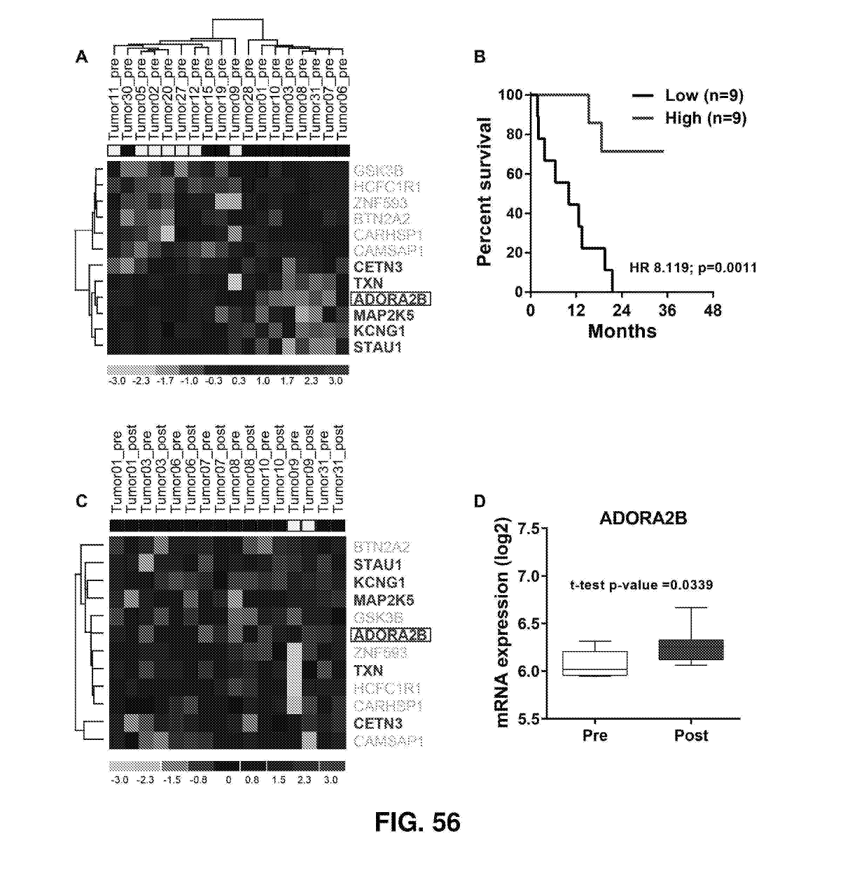

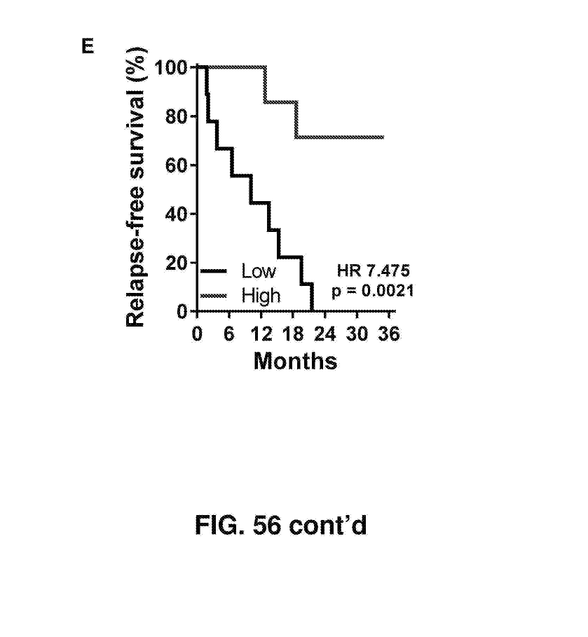

The study by Westin et al. (Lancet Oncol, 2014. vol 15(1)) performed gene expression profiling on 18 follicular lymphoma patients before receiving pidilizumab in combination with rituximab. The expression of the genes in the iBCR signature was investigated for association with progression free survival (PFS) in these patients. Twelve genes showed a strong association with PFS (FIG. 5(A) (all the genes that associated with survival belonged to the TN component of the iBCR test). As shown in FIG. 56B, a score calculated based on the iBCR signature was highly predictive of patient survival after pidilizumab+rituximab immunotherapy. The study also profiled eight of the patients 15 days post treatment. The expression of the genes in the signature was compared in these patients before and after treatment. Apart from a trend towards an inversion of the expression profile in general which was most obvious for the one patient who survived (FIG. 56C—patient number 9). one gene (ADORA2B) was significantly different in tumours after treatment compared to that before treatment (FIG. 56D). This gene could be used to confirm response after selection of patients based on the iBCR test.

The data presented here indicate the iBCR test can be a companion diagnostic for certain immunotherapy which is not surprising since the TN component includes several immune related genes in addition to genes involved in redox reactions and kinases.

### Example 5

A meta-analysis was performed in Oncomine™ using breast cancer datasets irrespective of subtypes or gene expression array platforms used. The global gene expression profiles of breast tumors that led to metastatic or death event within 5 years were compared to those that did not and the top overexpressed (OE) and underexpressed genes (UE) in these comparisons were selected. The commonly deregulated genes in the primary tumors that led to metastatic and death events (depending on the annotation of each dataset) were then interrogated using the online tool KM-Plotter™ (n>4000 patients with some overlap with the datasets in Oncomine™). Genes which associated with relapse-free survival of breast cancer patients were selected.

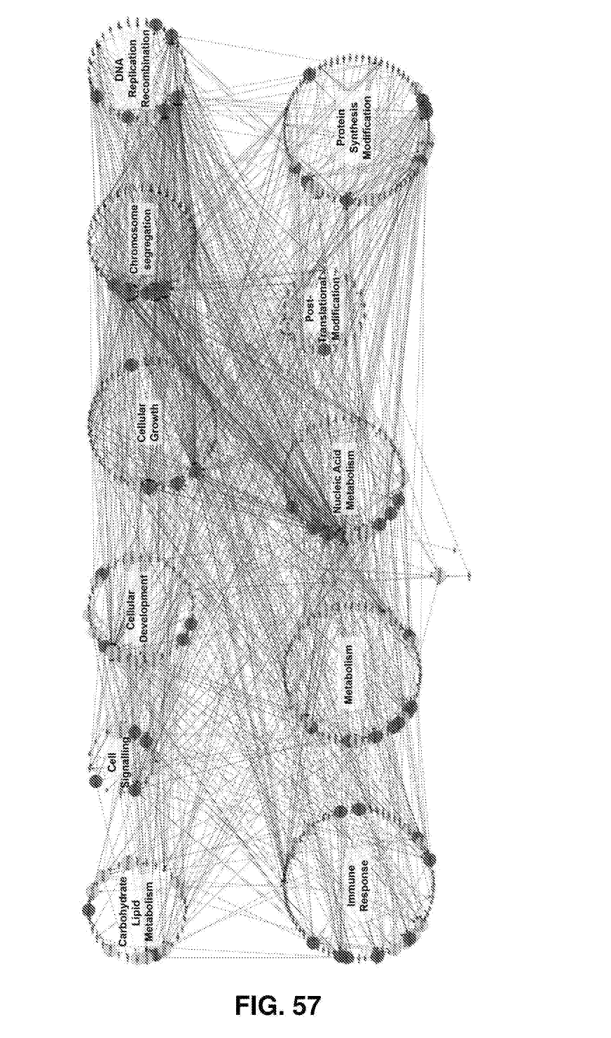

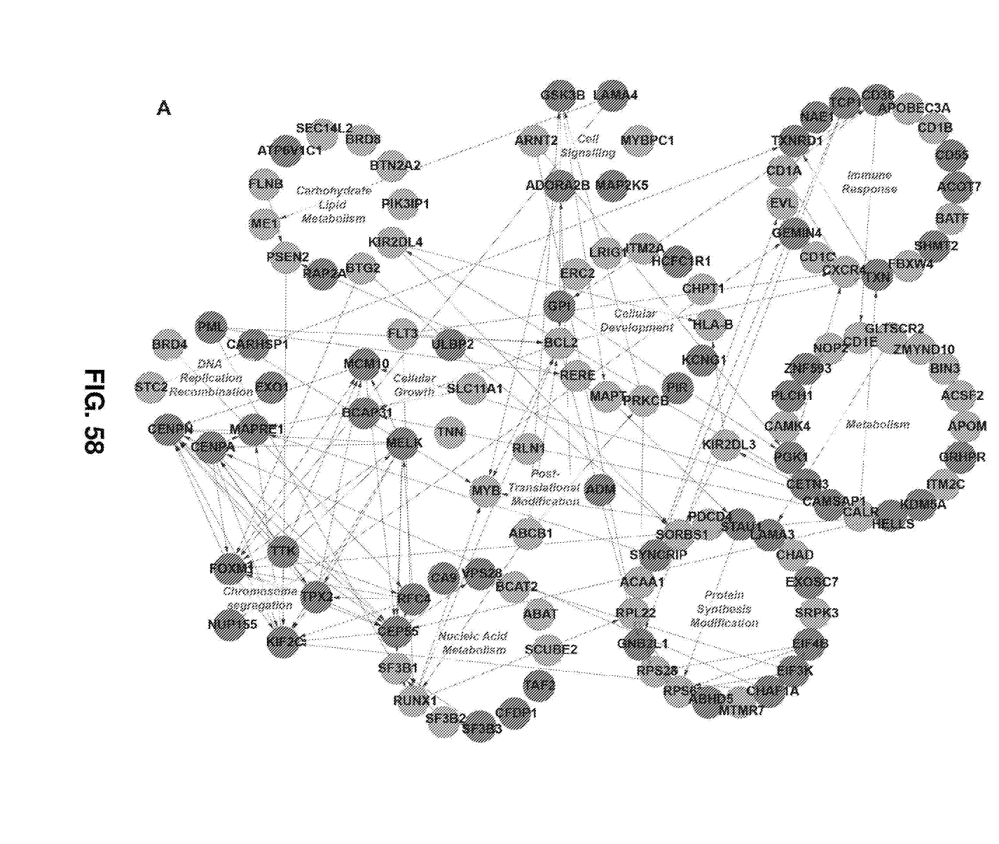

The 860 genes identified from this analysis were then subjected to network analysis using the Ingenuity Pathway Analysis (IPA®) software to identify functional networks within this gene list (see Table 21), FIG. 57 shows the eleven functional networks that contain the 860 genes identified from the meta-analysis where the function of each network is specified and the interactions amongst these networks are depicted with the connecting lines. Genes whose overexpression is associated with poorer survival are marked in red and those whose underexpression is associated with poorer survival are marked in green, Larger circles mark genes with highest association with patient survival in any given network.

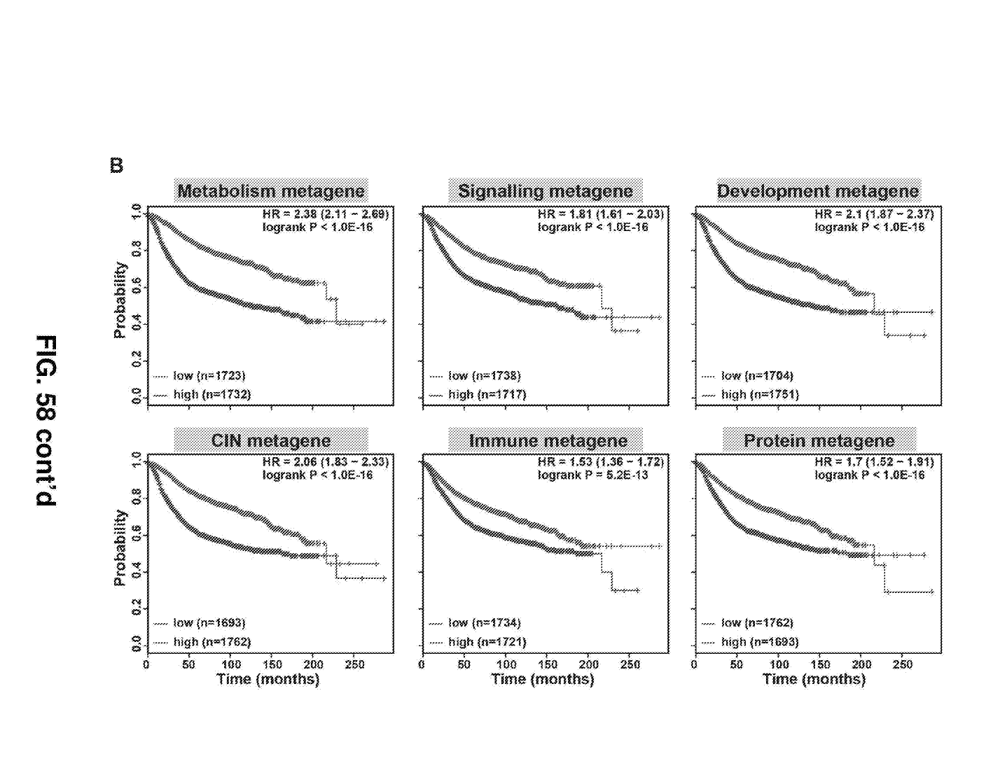

These 860 genes identified from the meta-analysis were then filtered for genes with the highest association with patient survival in each of the eleven functional networks. From this, the selected 133 genes (listed in Table 22) from the eleven functional networks are shown in FIG. 58 (panel A) where the function of each network is displayed. Based on these networks, the 133 genes were classified to six functional metagenes (listed in Table 22) which include: Metabolism, Signalling, Development and Growth, Chromosome segregation/Replication, Immune response and Protein synthesis/Modification metagenes. The association of each of these metagenes with relapse-free survival of breast cancer patients in the KM Plotter dataset is shown in panel B of FIG. 58. Each of these metagenes were scored by calculating the ratio of the expression level (sum or average) of the overexpressed genes in the metagene to the expression level (sum or average) of the underexpressed genes in the metagene. The green lines (with better survival) denote lower score (ratio of the overexpressed to the underexpressed genes) of the metagene whereas the red line (with worse survival) denote high score (ratio of the overexpressed genes to the underexpressed genes).

TABLE 21
860 genes associated with relapse-free survival of breast cancer
patients.

| Carbohydrate/Lipid | | Cell | | | |
|---|---|---|---|---|---|
| Metabolism | | Signalling | Cellualar Development | | |
| ARHGEF3 | ATP6V0A1 | AGBL2 | ABCA8 | KIF5C | ZNF211 |
| ASAH1 | ATP6V1C1 | ARFRP1 | APBB2 | LRIG1 | AP3B1 |
| ASB1 | COX4I1 | ARNT2 | ART4 | MADD | DYNC1LI2 |
| ATP2A2 | DHRS7 | CCR1 | ATHL1 | MAPT | ESRP1 |
| BRD8 | EPCAM | DST | BCL2 | MIER2 | GMPS |
| BTG2 | HN1 | EEF1A1 | BEND5 | MIS18A | GPI |
| BTN2A2 | IDH3A | LUZP1 | CABYR | MR1 | HCCS |
| C1QB | IDH3G | MYBPC1 | CASP10 | N4BP1 | HCFC1R1 |
| CERS6 | LAMTOR2 | PIP | CHPT1 | NEDD4L | KCNG1 |
| CYP2C9 | LAMTOR3 | S1PR1 | CYBRD1 | OGN | NAPG |
| ELOVL2 | MATR3 | SNED1 | ERC2 | PRKCB | NDRG1 |
| ELOVL5 | NPR3 | TAZ | FHL5 | PROL1 | NDUFB6 |
| ERBB4 | NRIP1 | TP63 | GAB1 | RERE | NDUFS6 |
| FLNB | PFKP | ADORA2B | GDNF | SETBP1 | NME1 |
| HIF3A | RAP2A | CMC4 | GLRB | SGCD | OIP5 |
| KIR2DL4 | SLC16A3 | DDX39A | GOLGB1 | SGSM2 | PGAM1 |
| LRP2 | TK1 | GAPDH | GOSR1 | SLC45A2 | PIR |
| LRP8 | VDAC1 | GSK3B | GPR12 | SOD2 | PRRG1 |
| ME1 | RAPGEF6 | HIF1A | HLA-B | SPAG8 | RTCA |
| NCOA1 | RBM38 | HSPA14 | ITM2A | SPG20 | S100A11 |
| NR1H3 | SEC14L2 | LAMA4 | KIAA0247 | SSPN | SMS |
| PBXIP1 | SRSF5 | MAP2K5 | KIAA0430 | SSX2 | TARS |
| PIK3IP1 | STARD13 | | STX18 | XBP1 | TRAK2 |
| PSEN2 | | | TRAK1 | ZC3H14 | |
| | | | TRAPPC10 | ZMYM5 | |

| | |
|---|---|
| | Chromosome |
| Cellular Growth | segregation |

| | | | | |
|---|---|---|---|---|
| ASF1B | SLC11A1 | BCAP31 | AFF1 | AURKB |
| BBS1 | SMARCA2 | BYSL | ATP1A2 | BUB1 |
| CCL13 | SNX1 | CCNA2 | CDC14A | BUB1B |
| CCND2 | SORL1 | CCNE2 | CDC27 | BUB3 |
| CDKN2A | SPDEF | CDC25A | CSPG4 | C20orf24 |
| DIRAS3 | STAT5B | CDC45 | FOXK2 | CCNB1 |
| DIXDC1 | TAOK3 | CDC6 | MAGI1 | CCNB2 |
| DOCK1 | TGOLN2 | CDCA3 | MLLT10 | CDC20 |
| DOK1 | THPO | CDCA8 | MTUS1 | CDK1 |
| EPOR | TIMELESS | CHEK1 | NUP62 | CENPE |
| FLT3 | TNN | DERL1 | NXF1 | CENPF |
| FOSB | TNXB | DHFR | PKMYT1 | CKS1B |
| GGA2 | TYRO3 | E2F8 | RAPGEF2 | CKS2 |
| HAVCR1 | ULK2 | ECT2 | SLC25A12 | FOXM1 |
| IL1RAPL1 | VPS39 | GINS3 | SLC8A1 | KIF2C |
| IL6ST | PIM1 | RAD51 | KIF4A | NUP93 |
| JAK2 | POLD1 | RRM2 | MAD2L1 | NUSAP1 |
| LEPR | PLK4 | SKP2 | MXI1 | NUTF2 |
| LIG1 | PSMD10 | UBE2C | NCAPG | PLK1 |
| LZTFL1 | MCM6 | ULBP2 | NDC80 | PRC1 |
| MTF1 | MELK | WDHD1 | NUP155 | PTTG1 |
| PCM1 | MMP1 | IL1RAP | TPX2 | SPC25 |
| PIK3R4 | MYBL2 | MCM10 | TTK | TACC3 |
| POU6F1 | ORC6 | MCM2 | ZWINT | |
| NF1 | PDAP1 | MCM4 | | |

| DNA Replication/ | | | | | | |
|---|---|---|---|---|---|---|
| Recombination | | Immune system | | | | |
| ALDH3A2 | ADRM1 | ABCA1 | DTX3 | SARM1 | PBK | ACOT7 |
| ATAD5 | BIRC5 | AHSG | DYNC2H1 | SIRT3 | PFDN5 | ANP32E |
| ATF5 | CARHSP1 | ANK3 | EFCAB6 | SMPDL3B | PSMA2 | APOBEC3B |
| BLM | CENPA | APOBEC3A | EFNB3 | SNN | RNASE4 | CAST |
| BRD4 | CENPI | BATF | ERAP1 | TTC28 | RNF141 | CCT5 |
| BRF2 | CENPN | BECN1 | EVL | WFDC2 | S100A9 | CCT6A |
| BTN3A2 | CENPU | BUD31 | FBXO41 | ZMYM6 | SHMT2 | CCT7 |
| CLASP2 | DLGAP5 | C2 | FBXW4 | ZNF516 | SLC7A5 | CD36 |
| FANCA | ERCC6L | C3 | FCGBP | IGHG3 | SOX11 | CD55 |
| FBLN1 | EXO1 | CACNA1D | FCGR1A | IGHM | TBPL1 | CDK8 |
| KIF18B | FANCI | CARD10 | FCGR1B | IGK | TCP1 | CHD1 |
| NPR2 | H2AFX | CD163 | FOS | IGKC | TOPORS | CXCL8 |
| PLXNA3 | H2AFZ | CD1A | FRZB | IGSF9B | TREM1 | DHCR7 |
| PSMD2 | IMPDH2 | CD1B | GAS7 | IL16 | TXN | DSCC1 |
| STC2 | MAPRE1 | CD1C | GCH1 | KCNMA1 | TXNRD1 | ELF3 |
| TCF3 | MSH6 | CD22 | GLI3 | KIF13B | WNT5A | GEMIN4 |
| TCF7L1 | PML | CD68 | GPRASP1 | KL | GM2A | |
| TCF7L2 | POMP | CD80 | GREB1 | LAD1 | GPSM2 | |
| TXNIP | PSMB4 | CDK5R1 | IGH | LAT | GSPT1 | |
| RYBP | PSMB5 | CFB | IGHG1 | LFNG | HMGB3 | |
| TOP2A | PSMB7 | CHL1 | NBPF10 | MED12 | HMMR | |
| UBE2A | PSMD14 | CIITA | NUMA1 | MOG | HNRNPAB | |
| UBE2B | PSMD3 | CR1 | PDE6B | MX2 | HPSE | |
| PSMD7 | | CRP | PGR | MCCC2 | HRASLS | |
| | | CST3 | PHLDA2 | MRPL12 | IDH2 | |
| | | CXCL14 | PPY | NAE1 | KIAA0101 | |
| | | CXCR4 | RLN2 | NXN | LGALS1 | |

Metabolic Disease

| | | | | | |
|---|---|---|---|---|---|
| AASS | ENOSF1 | MMRN2 | SESN1 | CALM1 | NME1- |
| ABCC8 | FAM105A | MPP2 | SFI1 | CAMSAP1 | NME2 |
| ACAP2 | FAM117A | MYO19 | SLC35A2 | CETN3 | PARPBP |
| ACSF2 | FAM120A | N4BP2L1 | SLC6A5 | CFAP20 | PGK1 |
| AHCYL1 | FAM129A | NBEA | SLCO1A2 | CMC2 | PLCH1 |
| ALDH1A2 | FAM49B | NCAPD3 | SPATA6 | CNOT8 | RAB22A |
| ANKHD1- | FAM86B1 | NDUFAF5 | TBRG4 | COG8 | SFXN1 |
| EIF4EBP3 | FCER1A | NFATC1 | TCTN1 | COQ9 | SHMT1 |
| ANKRD11 | GCC2 | NOP2 | TLDC1 | CORO1C | SMC4 |
| APOM | GLTSCR2 | NSUN5 | TLE4 | DKC1 | SNRPA1 |
| ARL3 | GTPBP2 | OSBPL1A | TMC6 | DONSON | STIL |
| BIN3 | HAUS5 | PADI1 | TSKS | EMC8 | SUGCT |
| BSDC1 | HDC | PDK3 | TSR1 | ENY2 | TMEM208 |
| BTD | HOOK2 | PHF8 | TTC12 | FKBP3 | TPD52L2 |
| BTN2A1 | HOXA4 | PIEZO1 | VAMP1 | GGH | TRIP13 |
| BTN3A3 | HPN | PPIL2 | VAMP2 | GLT8D1 | WDR41 |
| C12orf49 | HS3ST1 | PPP3R1 | WDR19 | GRHPR | YIPF3 |
| CALR | HTN1 | PSD4 | ZCCHC24 | GTSE1 | ZNF593 |
| CAMK2B | HYI | PUM1 | ZFP36L2 | HELLS | |
| CAMK4 | INADL | RAB30 | ZMYND10 | HJURP | |
| CASC1 | ITM2C | RAB6B | ZNF22 | KCMF1 | |
| CCDC176 | ITPR1 | RAI2 | ZNF506 | KDM5A | |
| CCDC25 | IVD | RALGAPA1 | ZNF778 | KIF14 | |
| CD1E | KIAA0930 | RAPGEF3 | ZSCAN32 | MRPL18 | |
| CNTRL | KIAA1549L | RCAN1 | ZZEF1 | MRPL9 | |
| CPSF7 | LAP3 | RPS6KA6 | ACOT13 | MRPS17 | |
| CROCC | ME3 | SERHL2 | B9D2 | NFATC3 | |
| CTDSPL | | | | | |

| | |
|---|---|
| | Post-Translational |
| Nucleic Acid Metabolism | Modification |

| | | | | |
|---|---|---|---|---|
| ABAT | RECQL5 | HEATR3 | ABCB1 | RTN1 |
| AHNAK | RUNX1 | KIF18A | ACAN | TENC1 |
| ALPK1 | SCUBE2 | KIF23 | AMN | TGFB3 |
| BCAT2 | SF3B1 | KPNA2 | COL4A6 | TGFBR3 |
| BMP8A | SF3B2 | PAPOLA | CSF1 | ADAM9 |
| BTRC | SLC27A2 | RAD51AP1 | DDX11 | ADM |
| CACNA1G | SLC6A2 | RFC4 | FGFR1 | CALB2 |
| CALCOCO1 | SMARCC2 | RPN1 | FGFR2 | CTSV |
| CBX7 | SNRNP70 | SEC61G | GSTM1 | DBNDD1 |
| COL14A1 | SRSF7 | SF3B3 | GUSB | FAM96B |
| DCLRE1C | SSX3 | SMAD5 | IGF1 | IGF1R |
| ESR1 | SYMPK | SMYD2 | LRRN3 | KIF11 |
| FBXO4 | SYNC | SPAG5 | MAP3K12 | KIF210A |
| FMO5 | TMC5 | SRPK1 | MST1 | LAPTM4B |
| GART | USP19 | SUB1 | MYB | MMP15 |
| H6PD | USP4 | TAF11 | NTRK2 | RAB2A |
| JADE2 | WSB1 | TAF2 | RBM5 | SERPINH1 |
| KIRG1 | ACTR3 | TCEB1 | RLN1 | TCEB2 |
| KMT2A | AQP9 | USP10 | | |
| MAFG | ARPC4 | VPS28 | | |
| MAPRE2 | ATAD2 | WWTR1 | | |
| MYOF | AURKA | XPOT | | |
| NOVA1 | CA9 | | | |
| NSMCE4A | CDK7 | | | |
| POLE2 | CEP55 | | | |
| PTGDS | CFDP1 | | | |
| PTGER3 | DSN1 | | | |

| Protein Synthesis/Modification | | | | | Multiple networks | |
|---|---|---|---|---|---|---|
| ACAA1 | MTMR3 | RPS28 | EIF6 | SLC25A5 | ABHD14A | RPS4XP2 |
| ACKR1 | MTMR7 | RPS4X | EPRS | SLC52A2 | C1orf21 | RPS4XP3 |
| ACSL6 | MXD4 | RPS6 | ETFA | SPIN1 | C3orf18 | SLC35D2 |
| ADRA2A | MYOZ3 | SAMD4A | EXOSC4 | SQLE | C4A | SLC38A7 |
| AGTR2 | MYT1 | SIRPA | EXOSC7 | STAU1 | CCDC30 | SPATA6L |
| AUNIP | NME5 | SLC16A5 | GNB2L1 | SYNCRIP | CFAP69 | SSX7 |
| C2CD2 | NMT1 | SLC4A7 | GPR56 | TKT | CLUL1 | TNXA |
| CCDC170 | NPY1R | SLC7A6 | GTPBP4 | TMEM194A | FCGR3B | TPSAB1 |
| CELSR2 | NPY5R | SORBS1 | ILF2 | TUBA1B | GUSBP11 | TPSB2 |
| CHAD | OSGEPL1 | SQSTM1 | KARS | UBE2V1 | IGHD | UGT1A8 |
| CREBL2 | P2RY4 | SRPK3 | LAMA3 | YWHAZ | IGHJ3 | WDR78 |
| CSDE1 | P2RY6 | THEMIS2 | LRPPRC | | IGHV3-20 | ZNF710 |
| CX3CR1 | PAPPA | TTLL1 | NDUFC1 | | IGHV3-23 | ZNRD1- |
| CYR61 | PDCD2 | ZNF395 | NELFE | | IGLJ3 | AS1 |
| DDX3X | PDCD4 | ABHD5 | NOP56 | | KIAA0040 | BOLA2 |
| DHTKD1 | PER3 | ADRBK2 | QARS | | KIR2DL1 | MRPL23 |
| EGOT | PNPLA4 | AIMP1 | RACGAP1 | | KIR2DL3 | |
| EIF1 | PTCD3 | ALG3 | RAD21 | | LINC01260 | |
| EML2 | PTPN1 | BRIX1 | RAD23B | | LOC389906 | |
| EPHX2 | PTPRO | CDKN3 | RC3H2 | | LRRC48 | |
| FAM134A | PTPRT | CHAF1A | RPL14 | | NBPF8 | |
| FRS3 | PURA | EIF3A | RPL15 | | NSUN7 | |
| ICA1 | RAMP2 | EIF3B | RPL29 | | PGAP2 | |
| LAMA2 | RGS5 | EIF3K | RPS9 | | PGPEP1 | |
| LPAR2 | RHBDD3 | EIF4B | RPSA | | RBMY1J | |
| LZTS1 | RPL10 | EIF4E | SFPQ | | RBMY2MP | |
| MAOA | RPL22 | EIF4G1 | SHCBP1 | | RGPD6 | |

Genes whose overexpression is associated with poorer survival are in bold and those whose underexpression is associated with poorer survival are underlined

TABLE 22
133 genes associated with relapse-free survival of breast cancer
patients.

| ID | SEQ ID NO: | Network | Metagene |
|---|---|---|---|
| BRD8 | 1 | Carbohydrate/Lipid Metabolism | Metabolism |
| BTG2 | 2 | Carbohydrate/Lipid Metabolism | |
| BTN2A2 | 3 | Carbohydrate/Lipid Metabolism | |
| KIR2DL4 | 4 | Carbohydrate/Lipid Metabolism | |
| ME1 | 5 | Carbohydrate/Lipid Metabolism | |
| PIK3IP1 | 6 | Carbohydrate/Lipid Metabolism | |
| SEC14L2 | 7 | Carbohydrate/Lipid Metabolism | |
| PSEN2 | 8 | Carbohydrate/Lipid Metabolism | |
| FLNB | 9 | Carbohydrate/Lipid Metabolism | |
| ACSF2 | 10 | Metabolic Disease | |
| APOM | 11 | Metabolic Disease | |
| BIN3 | 12 | Metabolic Disease | |
| CALR | 13 | Metabolic Disease | |
| CAMK4 | 14 | Metabolic Disease | |
| GLTSCR2 | 15 | Metabolic Disease | |
| ITM2C | 16 | Metabolic Disease | |
| NOP2 | 17 | Metabolic Disease | |
| NSUN5 | 18 | Metabolic Disease | |
| ZMYND10 | 19 | Metabolic Disease | |
| ABAT | 20 | Nucleic Acid Metabolism | |
| BCAT2 | 21 | Nucleic Acid Metabolism | |
| SCUBE2 | 22 | Nucleic Acid Metabolism | |
| SF3B1 | 23 | Nucleic Acid Metabolism | |
| RUNX1 | 24 | Nucleic Acid Metabolism | |
| ZNRD1- | 25 | Nucleic Acid Metabolism | |
| AS1 | | | |
| ATP6V1C1 | 26 | Carbohydrate/Lipid Metabolism | |
| RAP2A | 27 | Carbohydrate/Lipid Metabolism | |
| CALM1 | 28 | Metabolic Disease | |
| CAMSAP1 | 29 | Metabolic Disease | |
| CETN3 | 30 | Metabolic Disease | |
| COG8 | 31 | Metabolic Disease | |
| GRHPR | 32 | Metabolic Disease | |
| HELLS | 33 | Metabolic Disease | |
| KDM5A | 34 | Metabolic Disease | |
| PGK1 | 35 | Metabolic Disease | |
| PLCH1 | 36 | Metabolic Disease | |
| ZNF593 | 37 | Metabolic Disease | |
| CA9 | 38 | Nucleic Acid Metabolism | |
| CEP55 | 39 | Nucleic Acid Metabolism | |
| CFDP1 | 40 | Nucleic Acid Metabolism | |
| RFC4 | 41 | Nucleic Acid Metabolism | |
| TAF2 | 42 | Nucleic Acid Metabolism | |
| VPS28 | 43 | Nucleic Acid Metabolism | |
| SF3B3 | 44 | Nucleic Acid Metabolism | |
| LRRC48 | 45 | Cell Signaling | Signaling |
| ARNT2 | 46 | Cell Signaling | |
| MYBPC1 | 47 | Cell Signaling | |
| ADORA2B | 48 | Cell Signaling | |
| GSK3B | 49 | Cell Signaling | |
| LAMA4 | 50 | Cell Signaling | |
| MAP2K5 | 51 | Cell Signaling | |
| BCL2 | 52 | Cellular Development | Development&Growth |
| CHPT1 | 53 | Cellular Development | |
| ERC2 | 54 | Cellular Development | |
| ITM2A | 55 | Cellular Development | |
| LRIG1 | 56 | Cellular Development | |
| MAPT | 57 | Cellular Development | |
| PRKCB | 58 | Cellular Development | |
| RERE | 59 | Cellular Development | |
| ABHD14A | 60 | Cellular Development | |
| FLT3 | 61 | Cellular Growth | |
| SLC11A1 | 62 | Cellular Growth | |
| TNN | 63 | Cellular Growth | |
| GPI | 64 | Cellular Development | |
| HCFC1R1 | 65 | Cellular Development | |
| KCNG1 | 66 | Cellular Development | |
| PIR | 67 | Cellular Development | |
| BCAP31 | 68 | Cellular Growth | |
| MCM10 | 69 | Cellular Growth | |
| MELK | 70 | Cellular Growth | |
| ULBP2 | 71 | Cellular Growth | |
| BRD4 | 72 | DNA | Chromosome |
| | | Replication/Recombination | segregation/Replication |
| STC2 | 73 | DNA | |
| | | Replication/Recombination | |
| FOXM1 | 74 | Chromosome segregation | |
| KIF2C | 75 | Chromosome segregation | |
| NUP155 | 76 | Chromosome segregation | |
| TPX2 | 77 | Chromosome segregation | |
| TTK | 78 | Chromosome segregation | |
| CARHSP1 | 79 | DNA | |
| | | Replication/Recombination | |
| CENPA | 80 | DNA | |
| | | Replication/Recombination | |
| CENPN | 81 | DNA | |
| | | Replication/Recombination | |
| EXO1 | 82 | DNA | |
| | | Replication/Recombination | |
| MAPRE1 | 83 | DNA | |
| | | Replication/Recombination | |
| PML | 84 | DNA | |
| | | Replication/Recombination | |
| APOBEC3A | 65 | Immune system | Immune response |
| BATF | 86 | Immune system | |
| CD1A | 87 | Immune system | |
| CD1B | 88 | Immune system | |
| CD1C | 89 | Immune system | |
| CD1E | 90 | Immune system | |
| CFB | 91 | Immune system | |
| CXCR4 | 92 | Immune system | |
| EVL | 93 | Immune system | |
| FBXW4 | 94 | Immune system | |
| HLA-B | 95 | Immune system | |
| IGH | 96 | Immune system | |
| KIR2DL3 | 97 | Immune system | |
| SMPDL3B | 98 | Immune system | |
| ACOT7 | 99 | Immune system | |
| CD36 | 100 | Immune system | |
| CD55 | 101 | Immune system | |
| GEMIN4 | 102 | Immune system | |
| NAE1 | 103 | Immune system | |
| SHMT2 | 104 | Immune system | |
| TCP1 | 105 | Immune system | |
| TXN | 106 | Immune system | |
| TXNRD1 | 107 | Immune system | |
| ABCB1 | 108 | Post-Translational Modification | Protein synthesis/Modification |
| MYB | 109 | Post-Translational Modification | |
| RLN1 | 110 | Post-Translational Modification | |
| ACAA1 | 111 | Protein Synthesis/Modification | |
| CHAD | 112 | Protein Synthesis/Modification | |
| MTMR7 | 113 | Protein Synthesis/Modification | |
| PDCD4 | 114 | Protein Synthesis/Modification | |
| RPL10 | 115 | Protein Synthesis/Modification | |
| RPS28 | 116 | Protein Synthesis/Modification | |
| RPS4X | 117 | Protein Synthesis/Modification | |
| RPS6 | 118 | Protein Synthesis/Modification | |
| SORBS1 | 119 | Protein Synthesis/Modification | |
| SRPK3 | 120 | Protein Synthesis/Modification | |
| RPL22 | 121 | Protein Synthesis/Modification | |
| RPS4XP3 | 122 | Protein Synthesis/Modification | |
| ADM | 123 | Post-Translational Modification | |
| ABHD5 | 124 | Protein Synthesis/Modification | |
| CHAF1A | 125 | Protein Synthesis/Modification | |
| EIF3K | 126 | Protein Synthesis/Modification | |
| EIF4B | 127 | Protein Synthesis/Modification | |
| EXOSC7 | 128 | Protein Synthesis/Modification | |
| GNB2L1 | 129 | Protein Synthesis/Modification | |
| LAMA3 | 130 | Protein Synthesis/Modification | |
| NDUFC1 | 131 | Protein Synthesis/Modification | |
| STAU1 | 132 | Protein Synthesis/Modification | |
| SYNCRIP | 133 | Protein Synthesis/Modification | |

Genes whose overexpression is associated with poorer survival are in bold and those whose underexpression is associated with poorer survival are underlined

### Example 6

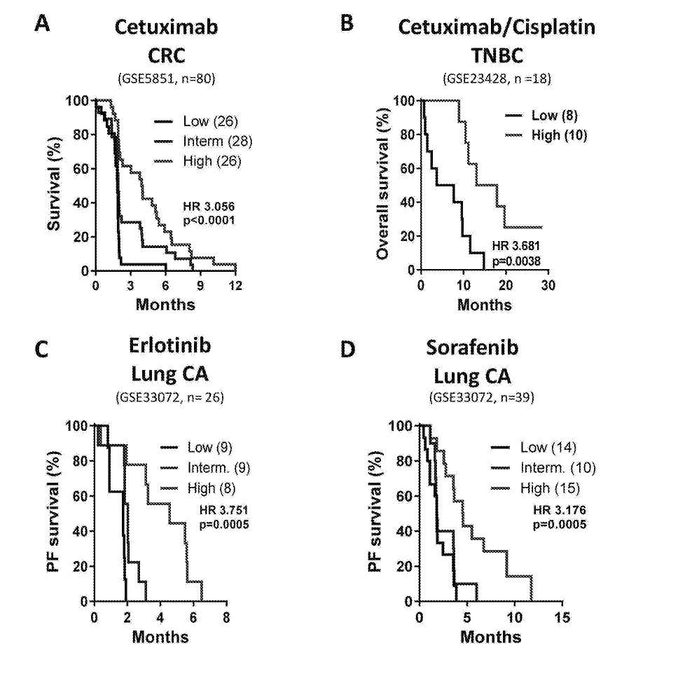

The preceding example identified 133 genes, associated with 12 oncogenic functions, the expression of which is strongly associated with cancer aggressiveness and clinical outcome (Table 22). The expression of genes from this list was investigated for association with survival in (i) follicular lymphoma patients before receiving pidilizurnab in combination with rituximab (Westin et al. Lancet Oncol, 2014, vol 15(1)) (ii) colorectal cancer patients treated with cetuximab (GSE5851); (iii) triple negative breast cancer patients treated with cetuximab and cisplatin (GSE23428); (iv) lung cancer patients treated with. erlotinib (GSE33072): and (v) lung cancer patients treated with sorafenib (GSE33072). This analysis identified new sets of genes, with partial overlap to the iBCR signature, the expression of which was highly associated with survival in the different treatment groups (Table 23). Scores for each patient group, which were calculated based on these gene signatures were shown to be highly predictive of survival in these patient groups (pidilizumab+rituximab: FIG. 56E; all other treatments FIG. 59).

TABLE 23
iBCR gene signatures associated with survival in patients receiving
anticancer therapy.

| Follicular | | | | |
|---|---|---|---|---|
| Lymphoma | | | Colorectal | Triple negative |
| (pidilizumab + | Lung Cancer | Lung Cancer | cancer | breast cancer |
| rituximab) | (erlotinib) | (sorafenib) | (cetuximab) | (cetuximab) |
| APOBEC3A | CD1C | NOP2 | ARNT2 | SF3B3 |
| BCL2 | CD1E | CALR | NDUFC1 | CETN3 |
| BTN2A2 | CD1B | MAPRE1 | BCL2 | SYNCRIP |
| CAMK4 | KDM5A | KCNG1 | ABHD14A | TAF2 |
| FBXW4 | BATF | PGK1 | EVL | CENPN |
| PSEN2 | EVL | SRPK3 | ULBP2 | ATP6V1C1 |
| MYB | PRKCB | RERE | BIN3 | CD55 |
| ADORA2B | HCFC1R1 | ADM | MAPRE1 | ADORA2B |
| CD36 | CARHSP1 | LAMA3 | BRD4 | RPL22 |
| KCNG1 | CHAD | KIR2DL4 | STAU1 | ABAT |
| LAMA3 | KIR2DL4 | ULBP2 | TAF2 | BTN2A2 |
| MAP2K5 | ABHD5 | LAMA4 | GSK3B | CD1B |
| NAE1 | ABHD14A | CA9 | PDCD4 | ITM2A |
| PGK1 | ACAA1 | BCAP31 | KCNG1 | BCL2 |
| STAU1 | SRPK3 | SCUBE2 | ZNRD1-AS1 | CXCR4 |
| CFDP1 | CFB | CHPT1 | EIF4B | ARNT2 |
| SF3B3 | NAE1 | CD1C | HELLS | |
| | GSK3B | BTG2 | | |
| | TAF2 | ADORA2B | | |
| | | BCL2 | | |

Genes whose underexpression is associated with a response to treatment are in bold and those whose overexpression is associated with a response to treatment are underlined

1. A method of determining the aggressiveness of a cancer in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level.

2. A method of determining a cancer prognosis for a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis.

3. The method of claim 1, wherein the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or a plurality of genes listed in Table 21.

4. The method claim 2, wherein the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or a plurality of genes listed in Table 21.

5. A method of determining the aggressiveness of a cancer in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level.

6. A method of determining a cancer prognosis for a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis.

7. The method of claim 6, wherein the Metabolism metagene, the Signalling metagene, the Development and Growth metagene, the Chromosome Segregation/Replication metagene, the Immune Response metagene and/or the Protein Synthesis/Modification metagene comprise one or more genes listed in Table 22.

8. The method of claim 5, wherein the Metabolism metagene, the Signalling metagene, the Development and Growth metagene, the Chromosome Segregation/Replication metagene, the Immune Response metagene and/or the Protein Synthesis/Modification metagene comprise one or more genes listed in Table 22.

9-13. (canceled)

14. A method of determining the aggressiveness of a cancer in a mammal, said method including the step of comparing an expression level of one or a plurality of overexpressed genes associated with chromosomal instability and/or an expression level of one or a plurality of underexpressed genes associated with estrogen receptor signalling in one or a plurality of cancer cells, tissues or organs of the mammal, wherein: a higher relative expression level of the one or plurality of overexpressed genes associated with chromosomal instability compared to the one or plurality of underexpressed genes associated with estrogen receptor signalling indicates or correlates with higher aggressiveness of the cancer; and/or a lower relative expression level of the one or plurality of overexpressed genes associated with chromosomal instability compared to the one or plurality of underexpressed genes associated with estrogen receptor signalling indicates or correlates with lower aggressiveness of the cancer compared to a mammal having a higher expression level.

15. A method of determining a cancer prognosis for a mammal, said method including the step of comparing an expression level of one or a plurality of overexpressed genes associated with chromosomal instability and/or an expression level of one or a plurality of underexpressed genes associated with estrogen receptor signalling in one or a plurality of cancer cells, tissues or organs of the mammal, wherein: a higher relative expression level of the one or plurality of overexpressed genes associated with chromosomal instability compared to the one or plurality of underexpressed genes associated with estrogen receptor signalling indicates or correlates with a less favourable cancer prognosis; and/or a lower relative expression level of the one or plurality of overexpressed genes associated with chromosomal instability compared to the one or plurality of underexpressed genes associated with estrogen receptor signalling indicates or correlates with a more favourable cancer prognosis.

16-23. (canceled)

24. The method of claim 14, wherein the genes associated with chromosomal instability are of a CIN metagene.

25. The method of claim 24, wherein the CIN metagene comprises a plurality of genes listed in Table 4.

26. The method of claim 15, wherein the genes associated with chromosomal instability are of a CIN metagene.

27. The method of claim 26, wherein the CIN metagene coprises a plurality of genes listed in Table 4.

28. The method of claim 14, wherein the genes associated with estrogen receptor signalling are of an ER metagene.

29. The method of claim 28, wherein the genes are selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3.

30. The method of claim 15, wherein the genes associated with estrogen receptor signalling are of an ER metagene.

31. The method claim 30, wherein the genes are selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3.

32-37. (canceled)

38. A method of determining the aggressiveness of a cancer in a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein: a higher relative expression level of the one or plurality of overexpressed genes compared to the one or plurality of underexpressed genes indicates or correlates with higher aggressiveness of the cancer; and/or a lower relative expression level of the one or plurality of overexpressed genes compared to the one or plurality of underexpressed genes indicates or correlates with lower aggressiveness of the cancer compared to a mammal having a higher expression level.

39. A method of determining a cancer prognosis for a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein: a higher relative expression level of the one or plurality of overexpressed genes compared to the one or plurality of underexpressed genes indicates or correlates with a less favourable cancer prognosis; and/or a lower relative expression level of the one or plurality of overexpressed genes compared to the one or plurality of underexpressed genes indicates or correlates with a more favourable cancer prognosis compared to a mammal having a higher expression level.

40-52. (canceled)

53. A method of determining the aggressiveness of a cancer in a mammal, said method including the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein: a higher relative expression level of the one or plurality of overexpressed proteins compared to the one or plurality of underexpressed proteins indicates or correlates with higher aggressiveness of the cancer; and/or a lower relative expression level of the one or plurality of overexpressed proteins compared to the one or plurality of underexpressed proteins indicates or correlates with lower aggressiveness of the cancer compared to a mammal having a higher expression level.

54. A method of determining a cancer prognosis for a mammal, said method including the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein: a higher relative expression level of the one or plurality of overexpressed proteins compared to the one or plurality of underexpressed proteins indicates or correlates with a less favourable cancer prognosis; and/or a lower relative expression level of the one or plurality of overexpressed proteins compared to the one or plurality of underexpressed proteins indicates or correlates with a more favourable cancer prognosis compared to a mammal having a higher expression level.

55-58. (canceled)

59. A method of predicting the responsiveness of a cancer to an anti-cancer treatment in a mammal, said method including the step of determining an expression level of one or plurality of genes associated with chromosomal instability in one or a plurality of non-mitotic cells of the mammal, wherein a higher expression level indicates or correlates with relatively increased responsiveness of the cancer to the anti-cancer treatment.

60. (canceled)

61. The method of claim 59, wherein the one or plurality of genes associated with chromosomal instability are listed in Table 4 and/or include one or more genes associated with aneuploidy.

62-64. (canceled)

65. A method of predicting the responsiveness of a cancer to an anti-cancer treatment in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Carbohydrate/Lipid Metabolism metagene, a Cell Signalling metagene, a Cellular Development metagene, a Cellular Growth metagene, a Chromosome Segregation metagene, a DNA Replication/Recombination metagene, an Immune System metagene, a Metabolic Disease metagene, a Nucleic Acid Metabolism metagene, a Post-Translational Modification metagene, a Protein Synthesis/Modification metagene and a Multiple Networks metagene, wherein an altered or modulated relative expression level of the overexpressed genes compared to the underexpressed genes indicates or correlates with relatively increased or decreased responsiveness of the cancer to the anti-cancer treatment.

66. (canceled)

67. The method of claim 65, wherein the Carbohydrate/Lipid Metabolism metagene, the Cell Signalling metagene, the Cellular Development metagene, the Cellular Growth metagene, the Chromosome Segregation metagene, the DNA Replication/Recombination metagene, the Immune System metagene, the Metabolic Disease metagene, the Nucleic Acid Metabolism metagene, the Post-Translational Modification metagene, the Protein Synthesis/Modification metagene and/or the Multiple Networks metagene comprise one or more genes listed in Table 21.

68. A method of predicting the responsiveness of a cancer to an anti-cancer treatment in a mammal, said method including the step of comparing an expression level of one or a plurality of overexpressed genes and/or an expression level of one or a plurality of underexpressed genes in one or a plurality of cancer cells, tissues or organs of the mammal, wherein the overexpressed genes and the underexpressed genes are from one or a plurality of metagenes selected from the group consisting of a Metabolism metagene, a Signalling metagene, a Development and Growth metagene, a Chromosome Segregation/Replication metagene, an Immune Response metagene and a Protein Synthesis/Modification metagene, wherein an altered or modulated relative expression level of the overexpressed genes compared to the underexpressed genes indicates or correlates with relatively increased or decreased responsiveness of the cancer to the anti-cancer treatment.

69. (canceled)

70. The method of claim 68, wherein the Metabolism metagene, the Signalling metagene, the Development and Growth metagene, the Chromosome Segregation/Replication metagene, the Immune Response metagene and/or the Protein Synthesis/Modification metagene comprise one or more genes listed in Table 22.

71-75. (canceled)

76. A method of predicting the responsiveness of a cancer to an anti-cancer treatment in a mammal, said method including the step of comparing an expression level of a one or plurality of overexpressed genes associated with chromosomal instability and/or an expression level of one or a plurality of underexpressed genes associated with estrogen receptor signalling in one or a plurality of cancer cells, tissues or organs of the mammal, wherein an altered or modulated relative expression level of the overexpressed genes associated with chromosomal instability compared to the underexpressed genes associated with estrogen receptor signalling indicates or correlates with relatively increased or decreased responsiveness of the cancer to the anti-cancer treatment.

77. The method of claim 76, wherein the genes associated with chromosomal instability are of a CIN metagene.

78. The method of claim 77, wherein the CIN metagene comprises a plurality of genes listed in Table 4.

79-80. (canceled)

81. The method claim 76, wherein the genes associated with estrogen receptor signalling are of an ER metagene.

82. The method of claim 81, wherein the genes are selected from the group consisting of: BTG2, PIK3IP1, SEC14L2, FLNB, ACSF2, APOM, BIN3, GLTSCR2, ZMYND10, ABAT, BCAT2, SCUBE2, RUNX1, LRRC48, MYBPC1, BCL2, CHPT1, ITM2A, LRIG1, MAPT, PRKCB, RERE, ABHD14A, FLT3, TNN, STC2, BATF, CD1E, CFB, EVL, FBXW4, ABCB1, ACAA1, CHAD, PDCD4, RPL10, RPS28, RPS4X, RPS6, SORBS1, RPL22 and RPS4XP3.

83-96. (canceled)

97. A method of predicting the responsiveness of a cancer to an anti-cancer treatment in a mammal, said method including the step of comparing an expression level of one or a plurality of overexpressed genes selected from the group consisting of CAMSAP1, CETN3, GRHPR, ZNF593, CA9, CFDP1, VPS28, ADORA2B, GSK3B, LAMA4, MAP2K5, HCFC1R1, KCNG1, BCAP31, ULBP2, CARHSP1, PML, CD36, CD55, GEMIN4, TXN, ABHD5, EIF3K, EIF4B, EXOSC7, GNB2L1, LAMA3, NDUFC1 and STAU1, and/or an expression level of one or a plurality of underexpressed genes selected from the group consisting of BRD8, BTN2A2. KIR2DL4. ME1, PSEN2, CALR, CAMK4, ITM2C, NOP2, NSUN5, SF3B1, ZNRD1-AS1, ARNT2, ERC2, SLC11A1, BRD4, APOBEC3A, CD1A, CD1B, CD1C, CXCR4, HLA-B, IGH, KIR2DL3, SMPDL3B, MYB, RLN1, MTMR7, SORBS1 and SRPK3, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the anti-cancer treatment.

98. The method of claim 97, wherein the one or plurality of overexpressed genes are selected from the group consisting of ABHD5, ADORA2B, BCAP31, CA9, CAMSAP1, CARHSP1, CD55, CETN3, EIF3K, EXOSC7, GNB2L1, GRHPR, GSK3B, HCFC1R1, KCNG1, MAP2K5, NDUFC1, PML, STAU1, TXN and ZNF593 and/or the one or plurality of underexpressed genes are selected from the group consisting of BTN2A2, ERC2, IGH, ME1, MTMR7, SMPDL3B and ZNRD1-AS1.

99-110. (canceled)

111. A method of predicting the responsiveness of a cancer to an anti-cancer treatment in a mammal, said method including the step of comparing an expression level of one or a plurality of overexpressed proteins selected from the group consisting of DVL3, PAI-1, VEGFR2, INPP4B, EIF4EBP1, EGFR, Ku80, HER3, SMAD1, GATA3, ITGA2, AKT1, NFKB1, HER2, ASNS and COL6A1, and/or an expression level of one or a plurality of underexpressed proteins selected from the group consisting of VEGFR2, HER3, ASNS, MAPK9, ESR1, YWHAE, RAD50, PGR, COL6A1, PEA15 and RPS6, in one or a plurality of cancer cells, tissues or organs of the mammal, wherein an altered or modulated relative expression level of the one or plurality of overexpressed proteins compared to the one or plurality of underexpressed proteins indicates or correlates with relatively increased or decreased responsiveness of the cancer to the anti-cancer treatment.

112-119. (canceled)

120. A method of predicting the responsiveness of a cancer to an immunotherapeutic agent in a mammal, said method including the step of comparing an expression level of one or a plurality of overexpressed genes selected from the group consisting of ADORA2B, CD36, CETN3, KCNG1, LAMA3, MAP2K5, NAE1, PGK1, STAU1, CFDP1, SF3B3 and TXN, and/or an expression level of one or a plurality of underexpressed genes selected from the group consisting of APOBEC3A, BCL2, BTN2A2, CAMSAP1, CAMK4, CARHSP1, FBXW4, GSK3B, HCFC1R1, MYB, PSEN2 and ZNF593, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the immunotherapeutic agent.

121. (canceled)

122. The method of claim 120, wherein the immunotherapeutic agent is an immune checkpoint inhibitor.

123. The method of claim 122, wherein the immune checkpoint inhibitor is or comprises an anti-PD1 antibody or an anti-PDL1 antibody.

124. A method of predicting the responsiveness of a cancer to an epidermal growth factor receptor (EGFR) inhibitor in a mammal, said method including the step of comparing an expression level of one or a plurality of overexpressed genes selected from the group consisting of NAE1, GSK3B, TAF2, MAPRE1, BRD4, STAU1, TAF2, PDCD4, KCNG1, ZNRD1-AS1, EIF4B, HELLS, RPL22, ABAT, BTN2A2, CD1B, ITM2A, BCL2, CXCR4, and ARNT2 and/or an expression level of one or a plurality of underexpressed genes selected from the group consisting of CD1C, CD1E, CD1B, KDM5A, BATF, EVL, PRKCB, HCFC1R1, CARHSP1, CHAD, KIR2DL4, ABHD5, ABHD14A, ACAA1, SRPK3, CFB, ARNT2, NDUFC1, BCL2, EVL, ULBP2, BIN3, SF3B3, CETN3, SYNCRIP, TAF2, CENPN, ATP6V1C1, CD55 and ADORA2B, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the immunotherapeutic agent.

125. A method of predicting the responsiveness of a cancer to a multikinase inhibitor in a mammal, said method including the step of comparing an expression level of one or a plurality of overexpressed genes selected from the group consisting of SCUBE, CHPT1, CDC1, BTG2, ADORA2B and BCL2, and/or an expression level of one or a plurality of underexpressed genes selected from the group consisting of NOP2, CALR, MAPRE1, KCNG1, PGK1, SRPK3, RERE, ADM, LAMA3, KIR2DL4, ULBP2, LAMA4, CA9, and BCAP31, in one or a plurality of cancer cells, tissues or organs of the mammal, wherein an altered or modulated relative expression level of the one or plurality of overexpressed genes compared to the one or plurality of underexpressed genes indicates or correlates with relatively increased or decreased responsiveness of the cancer to the multikinase inhibitor.

126. (canceled)

127. A method for identifying an agent for use in the treatment of cancer including the steps of:
(i) contacting a protein product of GRHPR, NDUFC1, CAMSAP1, CETN3, EIF3K, STAU1, EXOSC7, COG8, CFDP1 and/or KCNG1 with a test agent; and
(ii) determining whether the test agent, at least partly, reduces, eliminates, suppresses or inhibits the expression and/or an activity of the protein product.

128-129. (canceled)

130. A method of treating a cancer in a mammal, including the step of administering to the mammal a therapeutically effective amount of the agent identified by the method of claim 127.

131-134. (canceled)
